(19) United States
(12) Patent Application Publication
(10) Pub. No.: US 2018/0344813 A1
(43) Pub. Date: Dec. 6, 2018

(54) ENGINEERED POLYPEPTIDES HAVING ENHANCED DURATION OF ACTION WITH REDUCED IMMUNOGENICITY

(71) Applicants: Mary ERICKSON, San Diego, CA (US); David C. LITZINGER, San Diego, CA (US); Soumitra S. GHOSH, San Diego, CA (US); Zijian GUO, San Diego, CA (US); Manoj P. SAMANT, San Diego, CA (US); Abhinandini SHARMA, San Diego, CA (US); Lala MAMEDOVA, San Diego, CA (US); Odile Esther LEVY, San Diego, CA (US); Caroline EKBLAD, San Diego, CA (US)

(72) Inventors: Mary ERICKSON, San Diego, CA (US); David C. LITZINGER, San Diego, CA (US); Soumitra S. GHOSH, San Diego, CA (US); Zijian GUO, San Diego, CA (US); Manoj P. SAMANT, San Diego, CA (US); Abhinandini SHARMA, San Diego, CA (US); Lala MAMEDOVA, San Diego, CA (US); Odile Esther LEVY, San Diego, CA (US); Caroline EKBLAD, San Diego, CA (US)

(73) Assignees: AMYLIN PHARMACEUTICALS, LLC; ASTRAZENECA PHARMACEUTICALS LP

(21) Appl. No.: 15/177,397

(22) Filed: Jun. 9, 2016

(51) Int. Cl. A61K 38/26; A61K 9/08; A61K 9/19; A61P 3/10

(52) U.S. Cl. CPC A61K 38/26; A61P 3/10; A61K 9/19; A61K 9/08

(57) ABSTRACT

Compounds are provided having inter alia good duration of action, high potency and/or convenient dosing regimens including oral administration, and reduced immunogenicity. The compounds are engineered polypeptides which incorporate an albumin binding domain in combination with one or more biologically active polypeptides. Also provided are pharmaceutical compositions and methods of treatment for diseases and disorders including obesity and overweight, diabetes, dyslipidemia, hyperlipidemia, Alzheimer's disease, fatty liver disease, Short Bowel Syndrome, Parkinson's disease, and cardiovascular disease.

### CROSS-REFERENCES TO RELATED APPLICATIONS

This application claims priority to U.S. Ser. No. 61/505982 filed 8 Jul. 2011 and U.S. Ser. No. and U.S. Ser. No. 61/585577 filed 11 Jan. 2012, both of which are hereby incorporated by reference for all purposes.

### SEQUENCE LISTING

The instant application contains a Sequence Listing which has been submitted in ASCII format via EFS-Web and is hereby incorporated by reference in its entirety. Said ASCII copy, created on Jun. 22, 2012, is named 0263WO1.txt and is 366,392 bytes in size.

### BACKGROUND OF THE INVENTION

The present application relates to compounds having good duration of action, high potency and/or convenient dosing regimens, including oral administration, and method of use thereof. There are provided herein engineered polypeptides which incorporate an albumin binding domain in combination with a biologically active peptide. Without wishing to be bound by any theory, it is believed that because the engineered polypeptides described herein can bind albumin, the compounds can be sequestered (e.g., bound to albumin) while in the circulation leading to increased duration of action, due for example to decreased renal clearance and/or degradation. Diseases amendable to such treatment include obesity and overweight, diabetes, dyslipidemia, hyperlipidemia, short bowel syndrome, Alzheimer's disease, fatty liver disease, Parkinson's disease, cardiovascular disease, and other disorders of the central nervous system, or combinations thereof.

There remains a need to develop polypeptides useful in the above described metabolic diseases, conditions and disorders. Accordingly, it is an object of the present invention to provide engineered polypeptides with extended half-lives useful to treat the above conditions and methods for producing and using them.

Each patent, patent application, and publication cited herein is hereby incorporated herein by reference in its entirety and for all purposes.

### BRIEF SUMMARY OF THE INVENTION

There are provided engineered polypeptide compounds having binding affinity for albumin and an additional therapeutic utility. The compounds are engineered polypeptides which include an albumin binding domain (ABD) polypeptide as defined herein capable of binding albumin and a hormone domain (HD) polypeptide, which HD polypeptides can be biologically active and can elicit a beneficial biological response, in covalent linkage with the ABD. Any of the ABD or HD polypeptides described herein can be optionally covalently bonded in the engineered polypeptide through a linker L, for example L1 as described herein. Without wishing to be bound by any theory, it is believed that because the engineered polypeptides described herein can bind albumin, the compounds can be sequestered in a subject leading to increased duration of action in the subject.

Tn a first aspect, there is provided an engineered polypeptide as described herein. The engineered polypeptide includes an albumin binding domain polypeptide (ABD) as described herein and a hormone domain (HD1). The hormone domain includes a polypeptide which is an exendin, a fragment of an exendin, or analog of an exendin.

In another aspect, there is provided a method for treating a disease or disorder in a subject in need of treatment. The method includes administering an engineered polypeptide as described herein to the subject.

In yet another aspect, there is provided a pharmaceutical composition which includes an engineered polypeptide compound described herein in combination with a pharmaceutically acceptable excipient.

In yet another aspect are polynucleotides encoding the engineered polypeptide and their intermediates, expression vectors bearing such polynucleotides, host cells expressing such polynucleotides, and means for their expression, synthesis, post-translational modification and isolation.

One advantage of the present invention is that the engineered polypeptides can be synthesized completely by recombinant methods, avoiding complex or additional synthetic or chemical steps and associated reactive reagents and catalysts. Consequently, the polypeptides of the present invention can be much less expensive to synthesize than chemically derivatized compounds of prolonged duration of action. In addition to a long duration of action (e.g., at least one week in a human subject, albeit once daily can also be achieved if desired), a further advantage is their relatively small size, which can allow for oral delivery to improve patient compliance.

The compounds disclosed herein demonstrate surprising efficacy in an OGTT DOA (oral glucose tolerance test for duration of action) test of at least 24 hours and even longer to two days in mice, which translates to five to seven days or longer in humans, a robust glycemic control, body weight loss, a dose-dependent reduction of food intake and body weight. In rats compound exposure upon a single administration lasts for as 4 to 7 days, which translates to at least once a week exposure in humans. Compounds are stable in plasma and to plasma proteases, are active while bound to serum albumin and can provide in vivo maximal efficacy similar to or greater than exendin-4. Even more surprisingly the compounds are suitable for oral delivery.

### BRIEF DESCRIPTION OF THE DRAWINGS

FIG. 1 is a listing of the amino acid sequences of examples of albumin binding polypeptides (SEQ ID NO:301-452, SEQ ID NO:455-461) useful in the engineered polypeptides disclosed herein, the GA3 domain from protein G of Streptococcus strain G148 (SEQ ID NO:453) extended by a N-terminal glycine residue and an albumin binding polypeptide derived from G148-GA3 as previously described by Jonsson et al (Protein Eng. Design & Selection, 2008, 21:515-527); SEQ ID NO:454).

FIG. 2 shows the result of binding analysis performed in a Biacore instrument for investigating the binding of the albumin binding polypeptide PEP07912 (SEQ ID NO: 457) to human serum albumin. Three different concentrations of purified protein (40 nM, fat gray line; 10 nM, black line; and 2.5 nM, gray line) were injected over a surface with 955 RU of immobilized human serum albumin.

FIGS. 3A-C show the result of binding analysis performed by ELISA for investigating the binding of the albumin binding polypeptides PEP07913 (SEQ ID NO:453), PEP06923 (SEQ ID NO:454), PEP07271 (SEQ ID NO:455), PEP07912 (SEQ ID NO:457), PEP07554 (SEQ ID NO:456), PEP07914 (SEQ ID NO:458), PEP07968 (i.e. DOTA conjugated to PEP07911 (SEQ ID NO:459)) and PEP07844 (SEQ ID NO:461), to IgG molecules present in 126 individual normal human sera, where A) shows the average OD-value, B) shows the percentage of negative sera (defined as OD<0.15), and C) shows the percentage of positive sera (defined as OD>1.0).

FIGS. 4A-C are diagrams showing an immunogenicity assessment of albumin binding polypeptides PEP07913 (SEQ ID NO:453), PEP07912 (SEQ ID NO:457), PEP07914 (SEQ ID NO:458) and PEP07968 (i.e. DOTA conjugated to PEP07911 (SEQ ID NO:459)) in a CD3+ CD4+ T cell proliferation assay. A) shows the number of individuals responding to the albumin binding polypeptides compared to recombinant human albumin in a cohort of 52 Caucasian donors. B) shows the average stimulation indices (SI) for PEP07913, PEP07912, PEP07914 and PEP07968 compared to the negative control containing recombinant human albumin. C) shows the number of responding individuals against all proteins in the study as compared to the buffer control.

FIGS. 5A-5C. Effect of albumin-binding engineered polypeptides in ob/ob mice after four weeks of administration. FIG. 5A: change in HbA1c (hemoglobin A1c) at the indicated day after twice per week injection of Cmpd 088 at 25 or 250 nmol/kg compared to continuously infused (CSI) exendin-4 at 7.2 or 100 nanomoles/kg/day. FIG. 5B: change in blood glucose after dosage as described for FIG. 5A. FIG. 5C: change in body weight after dosage as described for FIG. 5A. *p<0.5 vs. Vehicle control; Anova, Dunnett's test.

FIGS. 6A-6C. Pharmacokinctic (PK) profile and biological activity of an exemplary engineered polypeptide Cmpd 11 dosed in normal Harlan Sprague-Dawley (HSD) rats. FIG. 6A depicts effect of compounds to reduce food intake. FIG. 6B depicts effect of compounds to reduce body weight. FIG. 6C depicts a PK profile of the compound after a single dose. In the figures, vehicle is solid square and engineered polypeptide is open inverted triangle.

FIGS. 7A-7C. Pharmacokinetic (PK) profile and biological activity of an exemplary engineered polypeptide Cmpd 9 dosed in normal Harlan Sprague-Dawley (HSD) rats. FIG. 7A depicts effect of compounds to reduce food intake. FIG. 7B depicts effect of compounds to reduce body weight. FIG. 7C depicts a PK profile of the compound after a single dose. In the figures, vehicle is solid square and engineered polypeptide is closed triangle.

FIGS. 8A-8C. FIGS. 8A-8C depict pharmacokinetic (PK) profile and biological activity of an exemplary engineered polypeptide Cmpd 11 compared to an unconjugated exendin analog dosed intravenously in normal Harlan Sprague-Dawley (HSD) rats. FIG. 8A depicts effect of compound to reduce food intake. FIG. 8B depicts effect of compound to reduce body weight. FIG. 8C depicts a PK profile of the exemplary compound after a single intravenous dose. Results presented as picomolar plasma levels. Legend: vehicle (diamond); [¹⁴Leu]exendin-4 at 2 nmol/kg IV (box); exendin-4 at 2 nmol/kg IV (circle); Cmpd 11 at 2 nmol/kg IV (open and closed triangle).

FIGS. 9A-9F. FIGS. 9A-9F depict pharmacokinetic (PK) profile and biological activity of an exemplary engineered polypeptide Cmpd 11 administered sub-chronically either daily or twice weekly. Cmpd 11 was subcutaneously administered at 25 nmol/kg over 14 days, either twice weekly (BTW; open inverted trangles) as indicated by the down arrows or daily (QD; open square) and compared to vehicle (closed circle). FIG. 9A depicts cumulative food intake. FIG. 9B depicts percent change in daily food intake. FIG. 9C depicts percent change in cumulative food intake. FIG. 9D depicts total body weight. FIG. 9E depicts percent change in body weight. FIG. 9F depicts a PK profile of Cmpd 11 given BIW or QD.

FIG. 10. This figure depicts a biological activity time course of polypeptide (Cmpd 088) compared to unconjugated exendin analog to lower blood glucose after oral delivery. See Examples. Mean pre-treatment glucose: ˜623 mg/dL. Legend: vehicle (closed box); exendin-4 analog (open box); Cmpd 088 (diamond).

### DETAILED DESCRIPTION OF THE INVENTION

### I. DEFINITIONS

“Obesity” and “overweight” refer to mammals having a weight greater than normally expected, and may be determined by, e.g., physical appearance, body mass index (BMI) as known in the art, waist-to-hip circumference ratios, skinfold thickness, waist circumference, and the like. The Centers for Disease Control and Prevention (CDC) define overweight as an adult human having a BMI of 25 to 29.9; and define obese as an adult human having a BMI of 30 or higher. Additional metrics for the determination of obesity exist. For example, the CDC states that a person with a waist-to-hip ratio greater than 1.0 is overweight.

“Lean body mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA) as known in the art.

“Mammal” refers to warm-blooded animals that generally have fur or hair, that give live birth to their progeny, and that feed their progeny with milk. Mammals include humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a human having type 2 diabetes. In one embodiment, the mammal is an obese diabetic mammal, e.g., an obese mammal having type 2 diabetes. The term “subject” in the context of methods described herein refers to a mammal.

“Fragment” in the context of polypeptides refers herein in the customary chemical sense to a portion of a polypeptide. For example, a fragment can result from N-terminal deletion or C-terminal deletion of one or more residues of a parent polypeptide, and/or a fragment can result from internal deletion of one or more residues of a parent polypeptide. “Fragment” in the context of an antibody refers to a portion of an antibody which can be linked to a biologically active molecule to modulate solubility, distribution within a subject, and the like. For example, exendin-4(1-30) describes a biologically active fragment of exendin-4 where the exendin C-terminal “tail” of amino acids 31-39 is deleted. The term “parent” in the context of polypeptides refers, in the customary sense, to a polypeptide which serves as a reference structure prior to modification, e.g., insertion, deletion and/or substitution. The term “conjugate” in the context of engineered polypeptides described herein refers to covalent linkage between component polypeptides, e.g., ABD, HD1 and the like. The term “fusion” in the context of engineered polypeptides described herein refers to covalent linkage between component polypeptides, e.g., ABD, HD1 and the like, via either or both terminal amino or carboxy functional group of the peptide backbone. Engineered polypeptides can be synthetically or recombinantly made. Typically, fusions are made using recombinant biotechnology, however, can also be made by chemical synthesis and conjugation methods.

“Analog” as used herein in the context of polypeptides refers to a compound that has insertions, deletions and/or substitutions of amino acids relative to a parent compound. “Analog sequence” as used herein in the context of polypeptides refers to an amino acid sequence that has insertions, deletions and/or substitutions of amino acids relative to a parent amino acid sequence (e.g., wild-type sequence, native sequence). An analog may have superior stability, solubility, efficacy, half-life, and the like. In some embodiments, an analog is a compound having at least 50%, for example 50%, 55%, 60%, 65%, 70%, 75%, 80%, 85%, 90%, 95%, 98%, or even higher, sequence identity to the parent compound. In a preferred embodiment the analog has from 1 to 5 amino acid modifications selected independently from any one or combination of an insertion, deletion, addition and substitution. In any of the embodiments herein, the exendin analog can have from 1 to 5 amino acid modifications selected independently from any one or combination of an insertion, deletion, addition and substitution, and preferably retains at least 50%, for example 50%, 55%, 60%, 65%, 70%, 75%, 80%, 85%, 90%, 95%, 98%, or even higher, sequence identity to the parent compound, and even more preferably at least 80%, 85%, 90%, 95%, 98%, or even higher, sequence identity to the parent compound, and preferably the parent compound is exendin-4, exendin-4(1-38), exendin-4(1-37), exendin-4(1-36), exendin-4(1-35), exendin-4(1-34), exendin-4(1-33), ex en din-4(1 -32), exendin-4(1-31), exendin-4(1-30), exendin-4(1-29) or exendin-4(1-28), or their Leu-14 substitution counterparts, e.g Leu14 exendin-4(1-38), Leu14 exendin-4(1-38), Leu14 exendin-4(1-37), Leu14 exendin-4(1-36), Leu14 exendin-4(1-35), Leu14 exendin-4(1-34), Leu14 exendin-4(1-33), Leu14 exendin-4(1-32), Leu14 exendin-4(1-31), Leu14 exendin-4(1-30), Leu14 exendin-4(1-29) or Leu14 exendin-4(1-28), and most preferably the parent compound has the sequence of exendin-4 or Leu14 exendin-4. In a preferred embodiment the exendin analog fragment is not an exendin-4(1-28) or its amino acid substitution analog such as Leu14 exendin-4(1-28). Preferably the exendin analog fragment is at least 29 amino acids in length.

In one embodiment at least amino acids corresponding to positions 1, 4, 6, 7 and 9 of exendin-4 are those as in native exendin-4, and further the one to five modifications are conservative amino acid substitutions at positions other than positions 1, 4, 6, 7 and 9 of exendin-4. For example, in yet a further embodiment of the embodiments herein, an exendin analog retains the amino acid at least as found in position 3, 4, 6, 5, 7, 8, 9, 10, 11, 13, 15, 18, 19, 22, 23, 25, 26, and/or 30 of exendin-4, and further preferably has no more than 1 to 5 of the remaining positions substituted with another amino acid, most preferably a chemically conservative amino acid. In all of the analogs herein, any substitution or modification at positions 1 and/or 2 will retain resistance to DPP-IV cleavage while retaining or improving insulinotropic activity as is known in the art for exendin-4 analogs, such as desamino-histidyl-exendin-4. As customary in the art, the term “conservative” in the context of amino acid substitutions refers to substitution which maintains properties of charge type (e.g., anionic, cationic, neutral, polar and the like), hydrophobicity or hydrophilicity, bulk (e.g., van der Waals contacts and the like), and/or functionality (e.g., hydroxy, amine, sulfhydryl and the like). The term “non-conservative” refers to an amino acid substitution which is not conservative.

“Identity,” “sequence identity” and the like in the context of comparing two or more nucleic acids or polypeptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See e.g., Current Protocols in Molecular Biology (Ausubel et al., eds. 1995 supplement)). Preferred examples of algorithms that are suitable for determining percent sequence identity and sequence similarity include the BLAST and BLAST 2.0 algorithms, which are described in Altschul et al., 1977, Nuci. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins of the invention. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The term “about” in the context of a numeric value refers to +/−10% of the numeric value.

The terms “peptide” and “polypeptide” in the context of components of the engineered polypeptides described herein are synonymous.

### II. COMPOUNDS

In a first aspect, engineered polypeptide compounds are provided with a sequence which includes an albumin binding domain (ABD) polypeptide sequence and at least one polypeptide hormone domain (HD1) sequence. The terms “albumin binding domain,” “ABD” and the like refer to polypeptides capable of binding albumin as described herein.

The terms “hormone domain,” “hormone domain polypeptide” and the like refer to a GLP-1 receptor agonist polypeptide capable of eliciting a biological response in a subject. Exemplary hormone domains include, but are not limited to, an exendin, an exendin fragment, or an exendin analog.

It was surprisingly found that an exendin, exendin analog or active fragment can be fused to an very-high-affinity albumin binding domain (ABD) derived from and having substantial amino acid sequence identity to the albumin-binding domains of bacterial protein

G of Streptococcus strain G148, while retaining sufficient exendin-4 biological activity and having an extended duration of action, for example of at least 3 days and even 5 days in a rodent, which translates to at least a one week duration or longer in a human subject. “Duration of action” refers in the customary sense to allowing for more infrequent dosing in a therapeutical regimen. Thus, a prolonged duration of action will allowed for less frequent and/or more convenient dosing schedules. This was surprising in part because such ABD peptides have not been extensively demonstrated to be a robust platform as a therapeutic protein carrier, they are relatively hydrophobic which could interact adversely with an attached therapeutic peptide, and were not able to act as a carrier for at least one family of peptide hormones. Specifically, rat amylin when conjugated or fused to the ABDs described herein did not display any significant or long-acting in vivo activity in the same rodent models in which various exendin-ABD constructs were found to be active and with long duration of action.

Furthermore, the therapeutic conjugate or fusion compounds herein surprisingly have retained albumin binding affinity and specificity while having lower immunogenicity and exendin-4 therapeutic activity. The compounds are surprisingly active despite the absence of a plasma-protease cleavage site between the exendin and the ABD. Further surprising, the therapeutic compounds are believed active even when bound to albumin. The ABD compounds described herein provide albumin binding affinity and specificity while having lower immunogenicity than previously described ABD compounds, which were based on the albumin binding region of Streptococcal protein G strain 148 (G148) and in Jonsson et al. (Protein Eng. Design & Selection, 2008, 21:515-527). Recently, a few T- and B-cell epitopes were experimentally identified within the albumin binding region of Streptococcal protein G strain 148 (G148) (Goetsch et al, Clin. Diagn. Lab. Immunol. 10:125-32, 2003). The authors were interested in utilizing the T-cell epitopes of G148 in vaccines, i.e. to utilize the inherent immune-stimulatory property of the albumin binding region. Goetsch et al. additionally found a B-cell epitope, i.e. a region bound by antibodies after immunization, in the sequence of G148. In pharmaceutical compositions for human administration no immune-response is desired. Therefore, the albumin binding domain G148 is as such not preferred for use in such compositions due to its abovementioned immune-stimulatory properties.

### Biologically Active Components.

Biologically active compound components contemplated for use in the compounds and methods described herein include the exendins. The terms “biologically active compound” and the like refer in the customary sense to compounds, e.g., polypeptides and the like, which can elicit a biological response.

Exendins. The exendins are peptides that are found in the salivary secretions of the Gila monster and the Mexican Bearded Lizard, reptiles that are endogenous to Arizona and Northern Mexico. Exendin-3 is present in the salivary secretions of Heloderma horridum (Mexican Beaded Lizard), and exendin-4 is present in the salivary secretions of Heloderma suspectum (Gila monster). See Eng et al, 1990, J. Biol. Chem., 265:20259-62; Eng et al, 1992, J. Biol. Chem., 267:7402-7405. The sequences of exendin-3 and exendin-4, respectively, follow:

(SEQ ID NO: 1)

HSDGTFTSDLSKQMEEEAVRLFIEWLKNGGPSSGAPPPS-NH₂;

(SEQ ID NO: 2)

HGEGTFTSDLSKQMEEEAVRLFIEWLKNGGPSSGAPPPS-NH₂.

Hargrove et al. (Regulatory Peptides, 2007, 141:113-119) reported an exendin-4 peptide analog that is a full-length C-terminally amidated exendin-4 peptide analog with a single nucleotide difference at position 14 compared to native exendin-4. The sequence of [¹⁴Leu]Exendin-4 is as follows:
HGEGTFTSDLSKQLEEEAVRLFTEWLKNGGPSSGAPPPS-NH₂ (SEQ TD NO:3). The Leu14 exendin-4 is a preferred analog for use in the engineered polypeptides and their uses described herein. Another exendin-4 peptide analog is a chimera of the first 32 amino acids of exendin-4 having amino acid substitutions at positions 14 and 28 followed by a 5 amino acid sequence from the C-terminus of a non-mammalian (frog) GLP1: [Leu¹⁴,Gln²⁸]Exendin-4(1-32)-fGLP-1(33-37). This compound has the following sequence:
HGEGTFTSDLSKQLEEEAVRLFIEWLKQGGPSKEIIS (SEQ ID NO:4). Also known in the art are C-terminally truncated, biologically active forms of exendin-4, such as exendin-4(1-28), exendin-4(1-29), exendin-4(1-30), exendin-4(1-31), exendin-4(1-32) and their amidated forms. All of these exendin analogs are suitable as components of the engineered polypeptides of the present invention. As is customary in the art, square brackets (i.e., “H”) in a peptidic compound name indicates substitution of the residue or chemical feature within the square brackets. For example, [¹⁴Leu]Exendin-4, [¹⁴Leu]Ex-4, and the like refer to exendin-4 having leucine at position 14. The numeric position of an amino acid can be indicated by prepended or postpended numbers in a variety of ways routinely employed in the art. For example, the terms ¹⁴Leu, Leu14, 14Leu, Leu¹⁴ and the like, are synonymous in referring to leucine at position 14.

It is understood that in some embodiments a C-terminal amide, or other C-terminal capping moiety can be present in compounds described herein.

Although the exendins have some sequence similarity to several members of the glucagon-like peptide family, with the highest homology, 53%, being to GLP-1(7-36)NH₂ (Goke et al, 1993, J. Biol. Chem., 268:19650-55) [sequence of GLP-1(7-37)NH₂: HAEGTFTSDVSSYLEGQAAKEFIAWLVKGRG (SEQ ID NO:5], also sometimes referred to as “GLP-1”) which has an insulinotropic effect stimulating insulin secretion from pancreatic beta-cells, exendins are not GLP-1 homologs.

Pharmacological studies have led to reports that exendin-4 can act at GLP-1 receptors in vitro on certain insulin-secreting cells, however, it has also been reported that exendin-4 may act at receptors not acted upon by GLP-1. Further, exendin-4 shares some but not all biological properties in vivo with GLP-1, and it has a significantly longer duration of action than GLP-1. Based on their insulinotropic activities, the use of exendin-3 and exendin-4 for the treatment of diabetes mellitus and the prevention of hyperglycemia has been proposed (Eng, U.S. Pat. No. 5,424,286, incorporated herein by reference in its entirety and for all purposes), and in fact, exendin-4 has been approved in the United States and in Europe for use as a therapeutic for treating type 2 diabetes.

Indeed, it is believed that exendins are not the species homolog of mammalian GLP-1 as was reported by Chen and Drucker who cloned the exendin gene from the Gila monster (J. Biol. Chem. 272:4108-15 (1997)). The observation that the Gila monster also has separate genes for proglucagons (from which GLP-1 is processed), that are more similar to mammalian proglucagon than exendin, indicated that exendins are not merely species homologs of GLP-1.

Methods for regulating gastrointestinal motility using exendin agonists are described in U.S. Pat. No. 6,858,576 (i.e., based on U.S. application Ser. No. 08/908,867 filed Aug. 8, 1997, which is a continuation-in-part of U.S. application Ser. No. 08/694,954 filed Aug. 8, 1996). Methods for reducing food intake using exendin agonists are described in U.S. Pat. No. 6,956,026 (i.e., based on U.S. application Ser. No. 09/003,869, filed Jan. 7, 1998, which claims the benefit of U.S. Application Nos. 60/034,905 filed Jan. 7, 1997, 60/055,404 filed Aug. 7, 1997, 60/065,442 filed Nov. 14, 1997, and 60/066,029 filed Nov. 14, 1997.

Novel exendin agonist compound sequences useful in the engineered polypeptides described herein are described in WO 99/07404 (i.e., PCT/US98/16387 filed Aug. 6, 1998), in WO 99/25727 (i.e., PCT/U S98/24210, filed Nov. 13, 1998), in WO 99/25728 (i.e., PCT/US98/24273, filed Nov. 13, 1998), in WO 99/40788, in WO 00/41546, and in WO 00/41548, which are incorporated herein by reference and for all purposes along with their granted U.S. patent counterparts. Methods to assay for exendin activities in vitro and in vivo, as known in the art, including insulinotropic, food intake inhibition activity and weight loss activity, are described herein and also in the above references and other references recited herein.

Certain exemplary exendins, exendin agonists, and exendin analog agonists include: exendin fragments exendin-4 (1-30) (His Gly Glu Gly Thr Phe Thr Ser Asp Leu Scr Lys Gln Met Glu Glu Glu Ala Val Arg Leu Phe Ile Glu Trp Leu Lys Asn Gly Gly) (SEQ ID NO: 680); exendin-4(1-28), exendin-4(1-29), exendin-4(1-30), exendin-4(1-31) and exendin-4(1-32). Analogs include substitution at the ¹⁴Met position (i.e., ¹⁴Met) with a non-oxidizing amino acid such as leucine. Examples include [¹⁴Leu]exendin-4, [¹⁴Leu]exendin-4(1-30), [¹⁴Leu]exendin-4(1-28) and [¹⁴Leu,²⁵Phe]exendin-4.

Exendin analog agonists for use in the engineered polypeptides described herein include those described in U.S. Pat. No. 7,223,725 (incorporated herein by reference and for all purposes), such as compounds of the formula: Xaa₁ Xaa₂ Xaa₃ Gly Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Ala Xaa₁₉ Xaa₂₀Xaa₂₁Xaa₂₂Xaa₂₃Xaa₂₄ Xaa₂₅ Xaa₂₆ Xaa₂₇ Xaa₂₈-Z₁; wherein Xaa₁ is His, Arg or Tyr; Xaa₂ is Ser, Gly, Ala or Thr; Xaa₃ is Ala, Asp or Glu; Xaa₅ is Ala or Thr; Xaa₆ is Ala, Phe, Tyr; Xaa₇ is Thr or Ser; Xaa₈ is Ala, Ser or Thr; Xaa₉ is Asp or Glu; Xaa₁₀ is Ala, Leu, Ile, Val, or Met; Xaa₁₁ is Ala or Ser; Xaa₁₂ is Ala or Lys; Xaa₁₃ is Ala or Gln; Xaa₁₄ is Ala, Leu, Ile, Val or Met; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Ala, Phe, Tyr; Xaa₂₃ is Ile, Val, Leu, or Met; Xaa₂₄ is Ala, Glu or Asp; Xaa₂₅ is Ala, Trp, Phe, Tyr; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂ (SEQ ID NO: 681), Gly Gly Xaa₃₁ Ser Ser-Z₂ (SEQ ID NO: 682), Gly Gly Xaa₃₁ Ser Ser Gly-Z₂ (SEQ ID NO: 683), Gly Gly Xaa₃₁ Ser Ser Gly Ala-Z₂ (SEQ ID NO: 684), Gly Gly Xaa₃₁ Ser Ser Gly Ala Xaa₃₆-Z₂ (SEQ ID NO: 685), Gly Gly Xaa₃₁ Ser Ser Gly Ala Xaa₃₆ Xaa₃₇-Z₂ (SEQ ID NO: 686) or Gly Gly Xaa₃₁ Ser Ser Gly Ala Xaa₃₆ Xaa₃₇ Xaa₃₈-Z₂ (SEQ ID NO: 687); Xaa₃₁, Xaa₃₆, Xaa₃₇ and Xaa₃₈ are independently Pro or are absent; and Z₂ is -OH or -NH₂. In any and each of the exendin analogs described above, also specifically contemplated are those wherein a replacement for the histidine corresponding to Xaal is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein arc exendin analogs described herein wherein a replacement for the glycine at Xaa2 is made with any of D-Ala, Val, Leu, Lys, Aib, (1-amino cyclopropyl) carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-amino cyclooctyl)carboxylic acid.

According to one embodiment, exemplary compounds include those of the above formula wherein: Xaa₁ is His or Arg; Xaa₂ is Gly or Ala; Xaa₃ is Asp or Glu; Xaa₅ is Ala or Thr; Xaa₆ is Ala or Phe; Xaa₇ is Thr or Ser; Xaa₈ is Ala, Ser or Thr; Xaa₉ is Asp or Glu; Xaa₁₀ is Ala, or Leu; Xaa₁₁ is Ala or Ser; Xaa₁₂ is Ala or Lys; Xaa₁₃ is Ala or Gln; Xaa₁₄ is Ala or Leu; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe; Xaa₂₃ is Ile, Val; Xaa₂₄ is Ala, Glu or Asp; Xaa₂₅ is Ala, Tip or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂ (SEQ ID NO: 681), Gly Gly Xaa₃₁ Ser Ser-Z₂ (SEQ ID NO: 682), Gly Gly Xaa₃₁ Ser Ser Gly-Z₂ (SEQ ID NO: 683), Gly Gly Xaa₃₁ Ser Ser Gly Ala-Z₂ (SEQ ID NO: 684), Gly Gly Xaa₃₁ Ser Ser Gly Ala Xaa₃₆-Z₂ (SEQ ID NO: 685), Gly Gly Xaa₃₁ Ser Ser Gly Ala Xaa₃₆ Xaa₃₇-Z₂ (SEQ ID NO: 686), Gly Gly Xaa₃₁ Ser Ser Gly Ala Xaa₃₆ Xaa₃₇ Xaa₃₈-Z₂ (SEQ ID NO: 687); Xaa₃₁, Xaa₃₆, Xaa₃₇ and Xaa being independently Pro or is absent and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. In any and each of the exendin analogs described above, also specifically contemplated are those wherein a replacement for the histidine corresponding to position Xaa1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at Xaa 2 is made with any of D-Ala, Val, Leu, Lys, Aib, (1-aminocyclopropyl) carboxylic acid, (1-amino cyclobutyl)carboxylic acid, 1-aminocyclopentyl)carboxylic acid, (1-aminocyclohexyl)carboxylic acid, (1-amino cycloheptyl)carboxylic acid, or (1-aminocyclooctyl)carboxylic acid.

Other exemplary compounds include those set forth in WO 99/25727 identified therein as compounds 2-23. According to another embodiment, provided are compounds where Xaa₁₄ is Leu, Ile, or Val more preferably Leu, and/or Xaa₂₅ is Trp, Phe or Tyr, more preferably Trp or Phe. These compounds will be less susceptive to oxidative degradation, both in vitro and in vivo, as well as during synthesis of the compound.

Additional examples of exendin analogs suitable for use in the present fusion polypeptides include those described in U.S. Pat. No. 6,528,486 published Mar. 4, 2003 (incorporated herein by reference and for all purposes). Specifically, exendin analogs include those consisting of an exendin or exendin analog having at least 90% homology to exendin-4 having optionally between one and five deletions at positions 34-39, and a C-terminal extension of a peptide sequence of 4-20 amino acid units covalently bound to said exendin wherein each amino acid unit in said peptide extension sequence is selected from the group consisting of Ala, Leu, Ser, Thr, Tyr, Asn, Gln, Asp, Glu, Lys, Arg, His, and Met. More preferably the extension is a peptide sequence of 4-20 amino acid residues, e.g., in the range of 4-15, more preferably in the range of 4-10 in particular in the range of 4-7 amino acid residues, e.g., of 4, 5, 6, 7, 8 or 10 amino acid residues, where 6 amino acid residues are preferred. Most preferably, according to U.S. Pat. No. 6,528,486 the extension peptide contains at least one Lys residue, and is even more preferably from 3 to 7 lysines and even most preferably 6 lysines.

For example, one analog is HGEGTFTSDLSKQMEEEAVRLFIEWLKNGG PSSGAPPSKKKKKK (SEQ ID NO:118) (also designated ([des-³⁶Pro]exendin-4(1-39)-Lys₆). Additional exemplary analogs include Lys₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Lys)₆ (H-Lys₆-des Pro ³⁶exendin-4(1-39)-Lys₆) (SEQ ID NO: 688); His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Trp-Leu-Lys-Asn-Gly-Gly-Pro-Ser-Ser-Gly-Ala-Ser (H-[des ³⁶Pro, ^{37,38}Pro]exendin-4(1-39)-NH₂) (SEQ ID NO: 689); Lys-Lys-Lys-Lys-Lys-Lys-His-Gly-Glu-Gly-Thr-Phe-Thr-Ser-Asp-Leu-Ser-Lys-Gln-Met-Glu-Glu-Glu-Ala-Val-Arg-Leu-Phe-Ile- Glu-Trp-Leu-Lys-Asn-Gly-Gly-Pro-Ser-Ser-Gly-Ala-Ser (H-(Lys)₆-[des ³⁶Pro, ^{37,38}Pro]exendin-4(1-39) (SEQ ID NO: 690); Asn-Glu-Glu-Glu-Glu-Glu-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Ser (H-Asn-(Glu)₅-[des ³⁶Pro, ^{37,38}Pro]exendin-4(1-39) (SEQ ID NO: 691); His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ ([des ³⁶Pro, ^{37,38}Pro]exendin-4(1-39)-(Lys)₆) (SEQ ID NO: 692); (Lys)₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ (H-(Lys)₆-[des ³⁶Pro, ^{37,38}Pro]exendin-4(1-39)-(Lys)₆) (SEQ ID NO: 693); and Asn-Glu-Glu-Glu-Glu-Glu-His-Gly-Glu-Gly-Thr-Phe-Thr-Ser-Asp-Leu-Ser-Lys-Gln-Met-Glu-Glu-Glu-Ala- Val-Arg-Leu-Phe-Ile-Glu-Trp-Leu-Lys-Asn-Gly-Gly-Pro-Ser-Ser-Gly-Ala-Ser-(Lys)₆ (Asn-(Glu) ₅-[des ³⁶Pro, ^{37,38}Pro]exendin-4(1-39)-(Lys)₆) (SEQ ID NO: 694). As customary in the art, repetition of an amino acid can be indicated by a subscripted number setting forth the number of repetitions; i.e., Lys₆ (SEQ ID NO: 695), (Lys)₆ (SEQ ID NO: 695) and the like refer to hexalysyl (SEQ ID NO: 695) In any and each of the exendin analogs described above, specifically contemplated are those wherein a replacement for the histidine corresponding to position 1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (or imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl) carboxylic acid, or (1-aminocyclooctyl) carboxylic acid.

Further examples of exendin analogs suitable for use in the engineered polypeptide constructs are those described in published PCT application WO2004035623 (incorporated herein by reference and for all purposes), particularly those comprised of naturally-occurring amino acids, which describes exendin analogs having at least one modified amino acid residue particularly at positions ¹³Gln, ¹⁴Met, ²⁵Trp ²⁸Asn with reference to the corresponding positions of exendin-4(1-39). According to that publication are additional such analogs further comprising a 1-7 amino acid C-terminal extension that comprises at least one Lys amino acid and more preferably at least five Lys amino acid units such as six or seven Lys amino acid units.

Yet further examples of exendin analogs suitable for use in the engineered polypeptide constructs are those described in published PCT application WO/2010/120476, entitled “N-Terminus Conformationally Constrained GLP-1 Receptor Agonist Compounds” (incorporated herein by reference and for all purposes), which describes exendin analogs having modified amino acid residues in the N-terminal portion of an exendin or exendin analog to create a high beta-turn characteristic in that region. For example, analogs are designed to mimic amino acid residues His1 Gly2 Glu3 by creating a conformationally constrained region, include exendin analogs containing a thiazolidine-proline peptide mimetic at His1 Gly2 Glu3 (see for example compounds described in FIGS. 17A-F therein), which can be used as a modification in exendin-4, lixisenatide, or other analogs described herein.

In any and each of the exendins, e.g. exendin-4, analogs and formulas described herein, specifically contemplated are those wherein a replacement for the histidine corresponding to position 1 is made with any of L-histidine, D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. For example, preferred exendin analogs for use in engineered polypeptide conjugates as described herein wherein the His1 position is modified are (4-imidazoacetyl) exendin-4, (des-amino-histidyl) exendin-4 (or (imidazopropionyl) exendin-4), (beta-hydroxy-imidazopropionyl) exendin-4, (N-dimethyl-histidyl) exendin-4 and (beta-carboxy-imidazopropionyl) exendin-4. Further specifically contemplated herein are exendins or exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl)carboxylic acid, or (1-aminocyclooctyl) carboxylic acid. Thus for example, such an engineered polypeptide would include (4-imidazoacetyl)exendin-4-Gly-PEP07986, where the exendin-4 analog is linked via a peptide bound at its C-terminal alpha carboxy group to a glycine as linker via a peptide bond to the N-terminus of the PEP07986 sequence.

Any of the above exendin analogs or their active fragments are suitable for use in the present engineered polypeptides, with or without a linker to the ABD.

Albumin binding domain (ABD) peptides. For previously disclosed albumin binding domains derived from Streptococcal protein G strain 148 (G148) and for some variants having a high affinity to albumin, e.g. WO09/016043, the higher affinity was achieved at the cost of reduced thermal stability. In addition, it has been reported that T- and B-cell epitopes were experimentally identified within the albumin binding region of G148 (Goetsch et al, Clin Diagn Lab Immunol 10:125-32, 2003). The authors behind the study were interested in utilizing the T-cell epitopes of 6148 in vaccines, i.e. to utilize the inherent immune-stimulatory property of the albumin binding region. Goctsch et al. additionally found a B-cell epitope, i.e. a region bound by antibodies after immunization, in the sequence of G148. Therefore, the albumin binding domain G148 and polypeptides derived from G148, and thus fusion/conjugates containing them, risk the abovementioned immune-stimulatory properties. In pharmaceutical compositions for human administration no (or reduced) immune-response is desired.

The above drawbacks and deficiencies of such fusions and/or conjugates are overcome or reduced by the use of the improved albumin binding domain (ABD) peptides disclosed herein for use in the engineered polypeptides of the invention. Such ABDs are those with comparably high affinity for albumin and derive from albumin-binding domain of bacterial protein G of Streptococcus strain G148 and have substantial amino acid sequence identity thereto, yet are modified as described herein to further provide desirable immunological properties, e.g. reduced immunogenicity. Accordingly, the albumin binding domain polypeptide comprising the long-duration engineered polypeptide conjugate or fusion described herein is a three-helix bundle protein domain, which comprises an albumin binding motif and additional sequences comprising the three-helix configuration. The ABD peptides described herein and contemplated for the engineered polypeptides described herein are superior to those having the albumin binding sequence as described by Jonsson et al. (Protein Eng. Design & Selection, 2008, 21:515-527) as well as the ABD peptides described therein, and those ABD peptides further described in PCT Published Appl. No. WO2009/016043. To the ABD polypeptide described herein is fused an exendin or analog or active fragment thereof to create the engineered polypeptide described herein. An albumin binding domain polypeptide suitable for conjugation or fusion to an exendin compound can comprise the improved ABD amino acid sequence which comprises a sequence selected from:

formula (i)

(SEQ ID NO: 300)

LA X3 AK X6 X7 AN X10 ELD X14 YGVSDF YKRL1 X26
KAKTVEGVEALK X39 X40 IL X43 X44 LP

wherein independently of each other

X3 is selected from E, S, Q and C;

X6 is selected from E, S and C;

X7 is selected from A and S;

X14 is selected from A, S, C and K;

X10 is selected from A, S and R;

X26 is selected from D and E;

X39 is selected from D and E;

X40 is selected from A and E;

X43 is selected from A and K;

X44 is selected from A, S and E;

the leucine at position 45 is present or absent; and

the proline at position 46 is present or absent; and

formula (ii) an amino acid sequence which has at least 95% identity to the sequence defined in (i),
with the proviso that X₇ is not L, E or D;
or alternatively,

with the proviso that the amino acid sequence is not defined by the following sequence, as defined in PCT Published Application No. WO 2009/016043: LAEAK Xₐ X_{b} A X_{c} X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO: 679) wherein independently of each other,

Xₐ is selected from V and E;

X_{b} is selected from L, E and D;

X_{c} is selected from N, L and I;

X_{d} is selected from R and K;

Xₑ is selected from D and K; and

X₅ is selected from Y and F;

X₈ is selected from N, R and S;

X₉ is selected from V, I, L, M, F and Y;

X₁₁ is selected from N, S, E and D;

X₁₂ is selected from R, K and N;

X₁₄ is selected from K and R;

X₂₀ is selected from D, N, Q, E, H, S, R and K;

X₂₃ is selected from K, I and T;

X₂₄ is selected from A, S, T, G, H, L and D; and

X₂₅ is selected from H, E and D.

In a further embodiment of the albumin binding polypeptide according to the first aspect above—the formula (i) or (ii), X6 is E. In another embodiment of the albumin binding polypeptide according to this aspect, X6 is S. In another embodiment of the albumin binding polypeptide according to this aspect, X3 is S. In another embodiment of the albumin binding polypeptide according to this aspect, X3 is E. In another embodiment of the albumin binding polypeptide according to this aspect, X7 is A. In another embodiment of the albumin binding polypeptide according to this aspect, X7 is S. In another embodiment of the albumin binding polypeptide according to this aspect, X14 is S. In another embodiment of the albumin binding polypeptide according to this aspect, X14 is C. In another embodiment of the albumin binding polypeptide according to this aspect, X14 is A. In another embodiment of the albumin binding polypeptide according to this aspect, X14 is K. In another embodiment of the albumin binding polypeptide according to this aspect, X10 is A. In another embodiment of the albumin binding polypeptide according to this aspect, X10 is S. In another embodiment of the albumin binding polypeptide according to this aspect, X26 is D. In another embodiment of the albumin binding polypeptide according to this aspect, X26 is E. In another embodiment of the albumin binding polypeptide according to this aspect X39 is D. In another embodiment of the albumin binding polypeptide according to this aspect X39 is E. In another embodiment of the albumin binding polypeptide according to this aspect X40 is A. In another embodiment of the albumin binding polypeptide according to this aspect X40 is E. In another embodiment of the albumin binding polypeptide according to this aspect X43 is A. In another embodiment of the albumin binding polypeptide according to this aspect X43 is K. In another embodiment of the albumin binding polypeptide according to this aspect X44 is A. In another embodiment of the albumin binding polypeptide according to this aspect X44 is S. In another embodiment of the albumin binding polypeptide according to this aspect X44 is E. In another embodiment of the albumin binding polypeptide according to this aspect leucine at position 45 is present. In another embodiment of the albumin binding polypeptide according to this aspect leucine at position 45 is absent. In a further embodiment the proline at position 46 is present. In a further embodiment the proline at position 46 at is absent.

In a further preferred embodiment albumin binding domain polypeptide suitable for conjugation or fusion to an exendin compound can comprise the improved ABD amino acid sequence selected from:

formula (iii)

(SEQ ID NO: 678)

LA X3 AK X6 X7 AN X10 ELD X14 YGVSDF YKRLIDKAKT
VEGVEALKDA ILAALP

wherein independently of each other

X3 is selected from E, S, Q and C;

X6 is selected from E, S and C;

X7 is selected from A and S;

X10 is selected from A, S and R;

X14 is selected from A, S, C and K;

the leucine at position 45 is present or absent; and

the proline at position 46 is present or absent; and

formula (iv) an amino acid sequence which has at least 95% identity to the sequence defined in (iii),

with the proviso that X₇ is not L, E or D;

or alternatively,

with the proviso that the amino acid sequence is not defined by the following sequence, as defined in PCT Published Application No. WO 2009/016043: LAEAK Xₐ X_{b} A X_{c} X_{d} EL Xₑ KY GVSD X₅ YK X₈ X₉ T X₁₁ X₁₂ A X₁₄ TVEGV X₂₀ AL X₂₃ X₂₄ X₂₅ TLAALP (SEQ ID NO: 679) wherein independently of each other,

Xₐ is selected from V and E;

X_{b} is selected from L, E and D;

X_{c} is selected from N, L and I;

X_{d} is selected from R and K;

Xₑ is selected from D and K; and

X₅ is selected from Y and F;

X₈ is selected from N, R and S;

X₉ is selected from V, I, L, M, F and Y;

X₁₁ is selected from N, S, E and D;

X₁₂ is selected from R, K and N;

X₁₄ is selected from K and R;

X₂₀ is selected from D, N, Q, E, H, S, R and K;

X₂₃ is selected from K, I and T;

X₂₄ is selected from A, S, T, G, H, L and D; and

X₂₅ is selected from H, E and D.

In a further embodiment of the albumin binding polypeptide according to this aspect formula (iii) or (iv), X6 is E. In another embodiment of the albumin binding polypeptide according to this aspect, X6 is S. In another embodiment of the albumin binding polypeptide according to this aspect, X3 is S. In another embodiment of the albumin binding polypeptide according to this aspect, X3 is E. In another embodiment of the albumin binding polypeptide according to this aspect, X7 is A. In another embodiment of the albumin binding polypeptide according to this aspect, X7 is S. In another embodiment of the albumin binding polypeptide according to this aspect, X14 is S. In another embodiment of the albumin binding polypeptide according to this aspect, X14 is C. In another embodiment of the albumin binding polypeptide according to this aspect, X14 is A. In another embodiment of the albumin binding polypeptide according to this aspect, X14 is K. In another embodiment of the albumin binding polypeptide according to this aspect, X10 is A. In another embodiment of the albumin binding polypeptide according to this aspect, X10 is S. In another embodiment of the albumin binding polypeptide according to this aspect leucine at position 45 is present. In another embodiment of the albumin binding polypeptide according to this aspect leucine at position 45 is absent. In a further embodiment the proline at position 46 is present. In a further embodiment the proline at position 46 at is absent.

In a further embodiment of any one of the formulas (i) to (iv) the ABD comprises a one or more N-terminal helix-capping amino acids, and in a further embodiment the helix-capping amino acid may be serine, or may be glycine-serine. Accordingly for each albumin binding domain sequence disclosed herein, including those in the figures and sequenced listing, also specifically contemplated for all aspects as disclosed herein in the engineered polypeptide, are albumin binding domains corresponding to the ABD of any one of the formulas (i) to (iv) contained therein, their Ser-ABD, Gly-Ser-ABD, Gly-ABD, Ala-ABD and their des-C-terminal-proline sequences.

Thus, modified variants of (i) or (iii), which are such that the resulting sequence is at least 95% identical to a sequence belonging to the class defined by (i) or (iii), are also encompassed. For example, it is possible that an amino acid residue belonging to a certain functional grouping of amino acid residues (e.g. hydrophobic, hydrophilic, polar etc) could be exchanged for another amino acid residue from the same functional group.

The above defined class of sequence related ABD polypeptides having a binding affinity for albumin is derived from a common parent polypeptide sequence, which folds into a three alpha helix bundle domain. More specifically, the polypeptides as described above are derived from a model building based on a structure of a complex between serum albumin and the albumin binding domain G148-GA3 (Lejon et al, J. Biol. Chem. 279:42924-8, 2004), as well as analyses of binding and structural properties of a number of mutational variants of the common parent polypeptide sequence. The above defined amino acid sequence of any one of formulas (i) to (iv) comprises amino acid substitutions, as compared to the parent polypeptide sequence, that result in a class of polypeptides which are expected to fold into an almost identical three helix bundle domain. While the parent polypeptide sequence already comprises a binding surface for interaction with albumin, that binding surface is modified by some of the substitutions according to the above definition. The substitutions according to the above definition provide an improved albumin binding ability as compared to the parent polypeptide sequence. Importantly and surprisingly, the substitutions according to the above definition provide enhanced immunological properties, in addition to retaining and/or improving strong affinity for albumin.

Accordingly, the improved ABD polypeptides according to the first aspect of the disclosure exhibit a set of characteristics, which, for example, make them suitable for use as fusion or conjugate partners for therapeutic molecules for human administration. Importantly and surprisingly, the improved ABD according to the present disclosure demonstrate, for example in comparison with related albumin binding polypeptides such as the albumin binding domain G148-GA3 and the albumin binding polypeptides disclosed in WO09/016043, at least five of the following six characteristics:
(1) The ABD polypeptides display a different surface compared to, for example, G148-GA3 and other bacterially derived albumin binding domains. The difference may decrease or eliminate any risk for antibody reactions in a subject, such as a human, which has been previously exposed to such bacterial proteins.
(2) The ABD polypeptides comprise fewer potential T-epitopes than, for example, G148-GA3 and other related, but different, mutational variants of the common parent polypeptide sequence, and hence exhibit low and/or lower immunogenicity when administered to a subject, such as a human.
(3) The polypeptides display lower reactivity with circulating antibodies when administered to a subject, such as a human. Thus, by amino acid substitutions in the surface of the polypeptides exposed to circulating antibodies, i.e. in the polypeptide surface not involved in the binding interaction with albumin, antibody cross-reactivity is reduced as compared to, for example, antibody cross-reactivity caused by G148-GA3 as measured in a test set of human sera.
(4) The polypeptides have a high albumin binding ability, both in terms of a higher binding affinity, as defined by a K_{D} value, and in terms of a slower off-rate, as defined by a koff value, than, for example, known naturally occurring albumin binding polypeptides, such as the albumin binding domains derived from bacterial proteins.
(5) The polypeptides comprise fewer amino acid residues that are associated with stability problems of polypeptides than, for example, known naturally occurring albumin binding polypeptides, such as the albumin binding domains derived from bacterial proteins. Thus, the polypeptides comprise, for example, no oxidation-prone methionines or tryptophans and only one asparagine.
(6) The polypeptides have a higher structural stability, as defined by a melting point of above 55° C., than previous albumin binding polypeptides, such as those disclosed in WO09/016043.

In one embodiment, the albumin binding polypeptide of the conjugate/fusions according to the first aspect display all six of the above listed characteristics. In another embodiment, the albumin binding polypeptide according to the first aspect displays, when bound to albumin, a more hydrophilic profile than, for example, previous albumin binding polypeptides, such as those disclosed in WO09/016043. The surface of the albumin binding polypeptide which is exposed to the surroundings when the polypeptide interacts with albumin comprises fewer amino acid residues that confer surface hydrophobicity.

Further for each of the embodiments herein of the ABD sequence, the C-terminal proline (corresponding to position 46 above) can be optionally absent. Even further for each embodiment of the ABD sequence, the leucine at position 45 can be optionally present or absent. “ABD sequence” is a sequence of an ABD compound that is monovalent or divalent, as appropriate, that forms part of an engineered polypeptide disclosed herein. “Peptide hormone domain (HD1) sequence” is a sequence of a peptide hormone domain (HD1) compound that is monovalent or divalent, as appropriate, that forms part of an engineered polypeptide disclosed herein. “Exendin sequence” is a sequence of an exendin compound that is monovalent or divalent, as appropriate, that forms part of an engineered polypeptide disclosed herein. “Exendin analog sequence” is a sequence of an exendin analog compound that is monovalent or divalent, as appropriate, that forms part of an engineered polypeptide disclosed herein. “Exendin active fragment sequence” is a sequence of an exendin active fragment compound that is monovalent or divalent, as appropriate, that forms part of an engineered polypeptide disclosed herein. “Exendin analog active fragment sequence” is a sequence of an exendin analog active fragment compound that is monovalent or divalent, as appropriate, that forms part of an engineered polypeptide disclosed herein. “Albumin binding motif (ABM) sequence” is a sequence of an ABM that is monovalent or divalent, as appropriate, that forms part of an engineered polypeptide disclosed herein. Unless stated otherwise, it is understood that where an engineered polypeptide “comprises” a compound (e.g., an ABD or HD1), the sequence of the engineered polypeptide includes the sequence of the compound (e.g. an ABD sequence or an HD1 sequence).

Because of the presence of an albumin binding motif, the ABD peptide binds to albumin with a K_{D} value of the interaction that is at most 1×10⁻⁶ M and even more preferably at most 1×10⁻⁹ M (even tighter affinity). More preferably the K_{D} value of the interaction that is at most 1×10⁻¹⁰ M, even more preferably is at most 1×10⁻¹¹ M, yet even more preferably is at most 1×10⁻¹² M, and even further is at most 1×10⁻¹³ M. The values are most preferably for affinity to human serum albumin (“HSA”).

The terms “albumin binding” and “binding affinity for albumin” as used herein refer to a property of a polypeptide which may be tested for example by the use of surface plasmon resonance technology, such as in a Biacore instrument as known in the art. For example, as described in the examples below, albumin binding affinity may be tested in an experiment in which albumin, or a fragment thereof, is immobilized on a sensor chip of the instrument, and the sample containing the polypeptide to be tested is passed over the chip. Alternatively, the polypeptide to be tested is immobilized on a sensor chip of the instrument, and a sample containing albumin, or a fragment thereof, is passed over the chip. Albumin may, in this regard, be a serum albumin from a mammal, such as human serum albumin. The skilled person may then interpret the results obtained by such experiments to establish at least a qualitative measure of the binding affinity of the polypeptide for albumin. If a quantitative measure is desired, for example to determine a K_{D} value for the interaction, surface plasmon resonance methods may also be used. Binding values may for example be defined in a Biacore2000 instrument (GE Healthcare). Albumin is suitably immobilized on a sensor chip of the measurement, and samples of the polypeptide whose affinity is to be determined are prepared by serial dilution and injected. K_{D} values may then be calculated from the results using for example the 1:1 Langmuir binding model of the BIAevaluation 4.1 software provided by the instrument manufacturer (GE Healthcare).

In one embodiment, the albumin binding polypeptide according to this aspect binds to albumin such that the k_{off} value of the interaction is at most 5×10⁻⁵ s⁻¹, such as at most 5×10⁻⁶ s⁻¹.

In another embodiment, the amino acid sequence of the albumin binding polypeptide is selected from any one of SEQ ID NO:301-344. More specifically, the amino acid sequence is selected from SEQ ID NO:304-305, SEQ ID NO:307-308, SEQ ID NO:310-311, SEQ ID NO:313-314, SEQ ID NO:316-317, SEQ ID NO:319-320, SEQ ID NO:322-323, SEQ ID NO:325-326, SEQ ID NO:328-329, SEQ ID NO:331-332, SEQ ID NO:334-335, SEQ ID NO:337-338, SEQ ID NO:341-342 and SEQ ID NO:349-350.

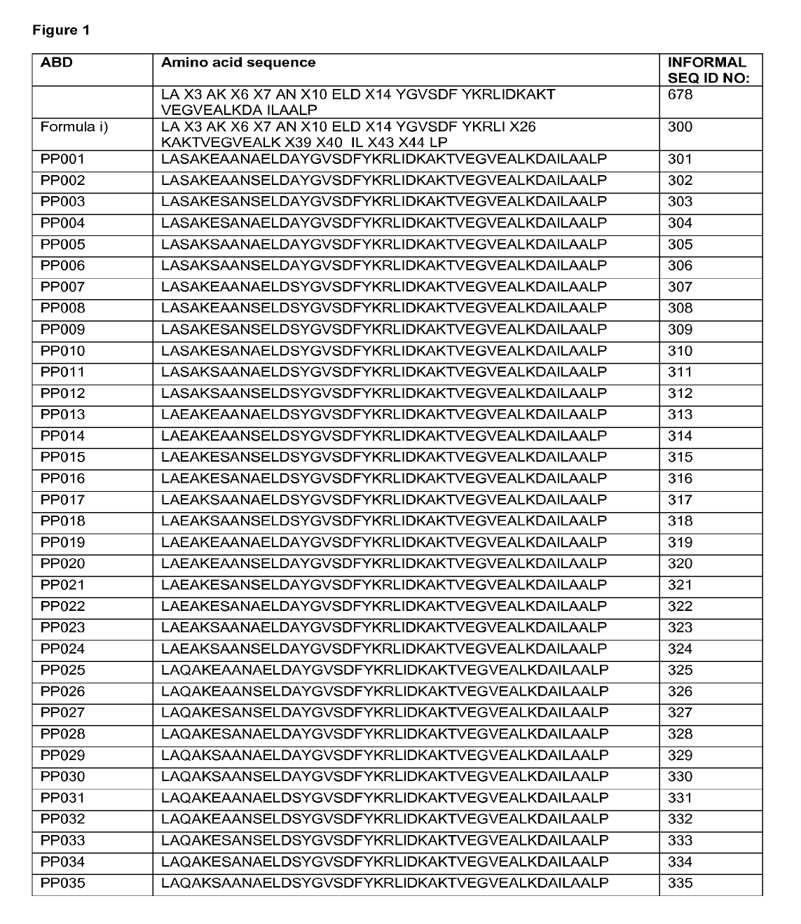

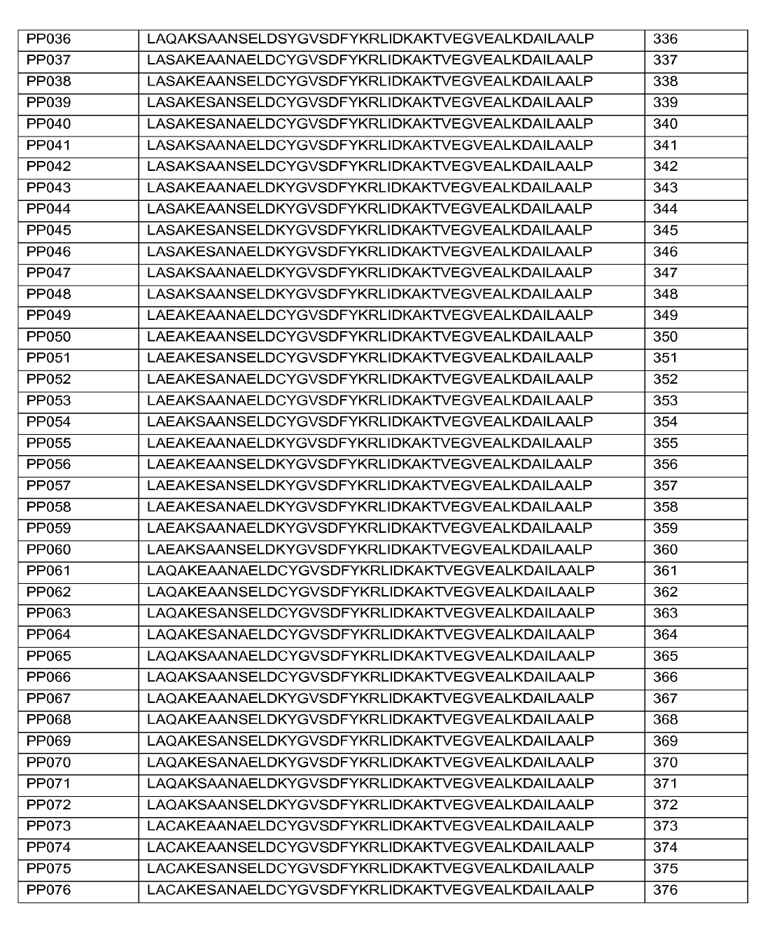

In another preferred embodiment of the ABD used in the engineered polypeptides described herein, the amino acid sequence of the albumin binding polypeptide portion of an engineered polypeptide includes an ABD selected from any one of the sequences described herein, including those from Table 1 or FIG. 1, the sequence listing herein and further including their des-Pro46 and/or des-Leu45 forms.

In one embodiment, the albumin binding polypeptide according to this aspect further includes one or more additional amino acid residues positioned at the N- and/or the C-terminal of the ABD sequence defined in (i) or (iii). These additional amino acid residues may play a role in further enhancing the binding of albumin by the polypeptide, and improving the conformational stability of the folded albumin binding domain, but may equally well serve other purposes, related for example to one or more of production, purification, stabilization in vivo or in vitro, coupling, labeling or detection of the polypeptide, as well as any combination thereof. Such additional amino acid residues may include one or more amino acid residue(s) added for purposes of chemical coupling, e.g. to the HD1.

For example, the amino acids directly preceding or following the alpha helix at the N-or C-terminus of the ABD amino acid sequence (i) or (iii) may thus in one embodiment affect the conformational stability. One example of an amino acid residue which may contribute to improved conformational stability is a serine residue positioned at the N-terminal of the ABD amino acid sequence (i) or (iii) as defined above. The N-terminal serine residue may in some cases form a canonical S—X—X-E capping box, by involving hydrogen bonding between the gamma oxygen of the serine side chain and the polypeptide backbone NH of the glutamic acid residue. This N-terminal capping may contribute to stabilization of the first alpha helix of the three helix domain constituting the albumin binding polypeptide according to the first aspect of the disclosure.

Thus, in one embodiment, the additional amino acids include at least one serine residue at the N-terminal of the polypeptide. The ABD amino acid sequence is in other words preceded by one or more serine residue(s). In another embodiment of the albumin binding polypeptide, the additional amino acids include a glycine residue at the N-terminal of the ABD sequence. It is understood that the ABD amino acid sequence (i) or (iii) may be preceded by one, two, three, four or any suitable number of amino acid residues. Thus, the ABD amino acid sequence may be preceded by a single serine residue, a single glycine residue or a combination of the two, such as a glycine-serine (GS) combination or a glycine-serine-serine (GSS) combination. Examples of albumin binding polypeptides comprising additional amino residues at the N-terminal are set out in SEQ ID NO:445-463, such as in SEQ ID NO:445-448 and SEQ ID NO:462-463, and in Table 1 and FIG. 1. In yet another embodiment, the additional amino acid residues comprise a serine at the N-terminal of the polypeptide as defined by the sequence formula (i) or (iii). An example of one such ABD having a N-terminal serine is SGSLAEAKEAANAELDSYGVSDFYKRLIDKAKTVEGVEALKDAILAALP (SEQ ID NO. 696). The corresponding des-proline form is SGSLAEAKEAANAELDSYGVSDFYKRLIDKAKTVEGVEALKDAILAAL (SEQ ID NO: 697). The corresponding des-Leu form is SGSLAEAKEAANAELDSYGVSDFYKRLIDKAKTVEGVEALKDAILAAP (SEQ ID NO: 698). The corresponding des-Pro des-Leu form is SGSLAEAKEAANAELDSYGVSDFYKRLIDKAKTVEGVEALKDAILAA (SEQ ID NO: 699).

In yet another embodiment, the additional amino acid residue or residues include an alanine acid at the N-terminal of the ABD polypeptide defined herein, or in combination with serine as an alanine-serine sequence at the N-terminal of the ABD sequences above. In yet another embodiment, the additional amino acid residue or residues include a glutamic acid at the N-terminal of the ABD polypeptide defined herein. In yet another embodiment, the additional amino acid residue or residues includes a cysteine at the N-terminal of the ABD polypeptide defined herein. Such additional residues when present are preferably from 1 to 5 amino acids.

Similarly, C-terminal capping may be exploited to improve stability of the third alpha helix of the three helix domain constituting the albumin binding polypeptide. The C-terminal proline residue present at the C-terminal of the ABD amino acid sequence defined in (i) or (iii) may at least partly function as a capping residue. A lysine residue following the proline residue at the C-terminal may contribute to further stabilization of the third helix of the albumin binding polypeptide, by hydrogen bonding between the epsilon amino group of the lysine residue and the carbonyl groups of the amino acids located two and three residues before the lysine in the polypeptide backbone, e.g. the carbonyl groups of the leucine and alanine residues of the ABD amino acid sequence defined in (i) or (iii). Thus, in one embodiment, the additional amino acids include a lysine residue at the C-terminal of the polypeptide. Such additional residues when present are preferably from 1 to 5 amino acids.

As discussed above, the additional amino acids may be related to the production of the albumin binding polypeptide. In particular, one or more optional amino acid residues following the C-terminal proline may provide advantages when the albumin binding polypeptide according to the first aspect is produced by chemical peptide synthesis. Such additional amino acid residues may for example prevent formation of undesired substances, such as diketopiperazine at the dipeptide stage of the synthesis. One example of such an amino acid residue is glycine. Thus, in one embodiment, the additional amino acids include a glycine residue at the C-terminal of the polypeptide, directly following the proline residue or following an additional lysine and/or glycine residue as accounted for above. Alternatively, polypeptide production may benefit from amidation of the C-terminal proline residue of the ABD amino acid sequence (i) or (iii). In this case, the C-terminal proline includes an additional amine group at the carboxyl carbon.

Examples of albumin binding polypeptides comprising additional amino acid residues at the C-terminal are set out in SEQ ID NO:445-452, such as in SEQ ID NO:449-450, and in Table 1 and FIG. 1. The skilled person is aware of methods for accomplishing C-terminal modification, such as by different types of pre-made matrices for peptide synthesis.

In another embodiment, the additional amino acid residues includes a cysteine residue at the N- and/or C-terminal of the polypeptide. Such a cysteine residue may directly precede and/or follow the ABD amino acid sequence as defined in (i) or (iii) or may precede and/or follow any other additional amino acid residues as described above. Examples of albumin binding polypeptides comprising a cysteine residue at the N- and/or C-terminal of the polypeptide chain are set out in SEQ ID NO:449-450 (C-terminal) and SEQ TD NO:451-452 (N-terminal), and in Table 1 and FIG. 1. By the addition of a cysteine residue to the polypeptide chain, a thiol group for site directed conjugation of the albumin binding polypeptide may be obtained. Alternatively, a selenocysteine residue may be introduced at the C-terminal of the polypeptide chain, in a similar fashion as for the introduction of a cysteine residue, to facilitate site-specific conjugation (Cheng et al, Nat Prot 1:2, 2006).

In one embodiment, the albumin binding polypeptide includes no more than two cysteine residues. In another embodiment, the albumin binding polypeptide includes no more than one cysteine residue.

In another embodiment, the additional amino acid residues of the albumin binding polypeptide includes a “tag” for purification or detection of the polypeptide, such as a hexahistidyl (His₆) tag (SEQ ID NO: 49), or a “myc” (“c-Myc”) tag or a “FLAG” tag for interaction with antibodies specific to the tag and/or to be used in purification. The skilled person is aware of other alternatives.

Exemplary ABD species include, but are not limited to, the compounds set forth in Table 1 following and the Examples.

TABLE 1
Selected ABD peptides

| | | SEQ |
|---|---|---|
| | | ID |
| Designation | ABD peptide sequence | NO: |
| PEP07271 | GSSLASAKEAANAELDAYGVSDFYKRLIDKAKTVEGVEALKDAILAALP | 455 |
| PEP07554 | GSSLASAKEAANAELDSYGVSDFYKRLIDKAKTVEGVEALKDAILAALP | 456 |
| PEP07912 | GLASAKEAANAELDSYGVSDFYKRLIDKAKTVEGVEALKDAILAALP | 457 |
| PEP07914 | GLAEAKEAANAELDSYGVSDFYKRLIDKAKTVEGVEALKDAILAALP | 458 |
| PEP07911 | GLASAKEAANAELDCYGVSDFYKRLIDKAKTVEGVEALKDAILAALP | 459 |
| PEP07834 | ALASAKEAANAELDCYGVSDFYKRLIDKAKTVEGVEALKDAILAALP | 460 |
| PEP07844 | GSSLASAKEAANAELDKYGVSDFYKRLIDKAKTVEGVEALKDAILAALP | 461 |
| PEP07983 | GSLASAKEAANAELDSYGVSDFYKRLIDKAKTVEGVEALKDAILAALP | 462 |
| PEP07986 | GSLAEAKEAANAELDSYGVSDFYKRLIDKAKTVEGVEALKDAILAALP | 463 |
| PEP08185 | GSLAEAKEAANAELDCYGVSDFYKRLIDKAKTVEGVEALKDAILAALPG | 448 |
| | LAEAKEAANAELDSYGVSDFYKRLIDKAKTVEGVEALKDAILAALP | 313 |
| | SLAEAKEAANAELDSYGVSDFYKRLIDKAKTVEGVEALKDAILAALP | 500 |
| | LAEAKEAANAELDCYGVSDFYKRLIDKAKTVEGVEALKDAILAALPG | 501 |
| | SLAEAKEAANAELDCYGVSDFYKRLIDKAKTVEGVEALKDAILAALPG | 502 |
| (des C- | GSLAEAKEAANAELDSYGVSDFYKRLIDKAKTVEGVEALKDAILAAL | 700 |
| terminal | | |
| Pro) | | |
| PEP07986 | | |
| (des C- | GSLAEAKEAANAELDCYGVSDFYKRLIDKAKTVEGVEALKDAILAAL | 701 |
| terminal | | |
| Pro-Gly) | | |
| PEP08185 | | |
| | SLAEAKEAANAELDSYGVSDFYKRLIDKAKTVEGVEALKDAILAAL | 702 |
| | SLAEAKEAANAELDCYGVSDFYKRLIDKAKTVEGVEALKDAILAAL | 703 |
| | LAEAKEAANAELDSYGVSDFYKRLIDKAKTVEGVEALKDAILAAL | 704 |
| | LAEAKEAANAELDCYGVSDFYKRLIDKAKTVEGVEALKDAILAAL | 705 |
| | GSSLASAKEAANAELDAYGVSDFYKRLIDKAKTVEGVEALKDAILAALP | 455 |
| | GSSLASAKEAANAELDKYGVSDFYKRLIDKAKTVEGVEALKDAILAA | 712 |
| | GSLASAKEAANAELDSYGVSDFYKRLIDKAKTVEGVEALKDAILAA | 713 |
| | GSLAEAKEAANAELDSYGVSDFYKRLIDKAKTVEGVEALKDAILAA | 714 |
| | GSLAEAKEAANAELDCYGVSDFYKRLIDKAKTVEGVEALKDAILAA | 715 |
| | LAEAKEAANAELDSYGVSDFYKRLIDKAKTVEGVEALKDAILAAL | 704 |
| | SLAEAKEAANAELDSYGVSDFYKRLIDKAKTVEGVEALKDAILAA | 717 |
| | LAEAKEAANAELDCYGVSDFYKRLIDKAKTVEGVEALKDAILAA | 718 |
| | SLAEAKEAANAELDCYGVSDFYKRLIDKAKTVEGVEALKDAILAA | 719 |
| | GSLAEAKEAANAELDSYGVSDFYKRLIDKAKTVEGVEALKDAILAA | 714 |
| | GSLAEAKEAANAELDCYGVSDFYKRLIDKAKTVEGVEALKDAILAA | 715 |
| | SLAEAKEAANAELDSYGVSDFYKRLIDKAKTVEGVEALKDAILAA | 717 |
| | SLAEAKEAANAELDCYGVSDFYKRLIDKAKTVEGVEALKDAILAA | 719 |
| | LAEAKEAANAELDSYGVSDFYKRLIDKAKTVEGVEALKDAILAA | 716 |
| | LAEAKEAANAELDCYGVSDFYKRLIDKAKTVEGVEALKDAILAA | 718 |

For example, in preferred engineered polypeptide embodiments the ABD comprises LAEAKEAANAELDSYGVSDFYKRLIDKAKTVEGVEALKDAILAALP (SEQ ID NO: 313), and its N-terminally extended ABD sequence forms including SLAEAKEAANAELDSYGVSDFYKRLIDKAKTVEGVEALKDAILAALP (SEQ ID NO: 500) and GSLAEAKEAANAELDSYGVSDFYKRLTDKAKTVEGVEALKDATLAALP (SEQ ID NO:463; PEP07986). The serine in position 2 is capping the sequence, raising Tm approximately 2° C. compared to having a glycine or an alanine in this position. An alanine can also immediately precede the serine as in AGSLAEAKEAANAELDSYGVSDFYKRLIDKAKTVEGVEALKDAILAALP (SEQ ID NO. 720). Also preferred are the corresponding polypeptides where the C-terminal proline, glycine or both is absent in each of the above ABD sequences. Accordingly, also preferred are sequences where the ABD includes the des-proline forms, which can improve yields compared to the parent forms:

(SEQ ID NO: 704)

LAEAKEAANAELDSYGVSDFYKRLIDKAKTVEGVEALKDAILAAL,
and its N-terminally extended ABD sequence forms
including

(SEQ ID NO: 702)

SLAEAKEAANAELDSYGVSDFYKRLIDKAKTVEGVEALKDAILAAL
and

(SEQ ID NO: 700)

GSLAEAKEAANAELDSYGVSDFYKRLIDKAKTVEGVEALKDAILAAL.
Also preferred is the des-Leu45 form of each ABD.

In preferred engineered polypeptide embodiments where Cys-conjugation is desired the preferred ABD can comprise LAEAKEAANAELDCYGVSDFYKRLIDKAKTVEGVEALKDAILAALPG (SEQ ID 501) and its N-terminally extended ABD sequence forms including SLAEAKEAANAELDCYGVSDFYKRLIDKAKTVEGVEALKDAILAALPG (SEQ ID 502) and GSLAEAKEAANAELDCYGVSDFYKRLIDKAKTVEGVEALKDAILAALPG (SEQ ID NO: 448; PEP08185). Also preferred are the polypeptides where the C-terminal proline or glycine or both are absent in each of the above ABD sequences.

In one aspect with any of the ABD sequences disclosed herein, the linker to cxcndin-4 or exendin analog sequence is a glycine including linker as disclosed herein, for example G, GGG, GGS, GGGS (SEQ ID NO:192), TGGGGAS (SEQ ID NO:193), TGGGGGAS (SEQ ID NO:194), or TGGGGSAS (SEQ ID NO:195).

In one embodiment of the engineered polypeptides described herein, particularly those ending at its C-terminus with proline or other amino acid known to racemize during peptide synthesis, a glycine can be added to the C-terminus to counter potential problems with racemization of the C-terminal amino acid residue. Alternatively the C-terminal amino acid can in its (alpha-amino group) amidated form, e.g. proline versus proline amide, rather than ending with a glycine. However, if the amidatcd polypeptide is desired to be produced by recombinant rather than chemical synthesis, then amidation of the C-terminal amino acid can be performed by several methods known in the art, e.g. use of amidating PAM enzyme. An engineered polypeptide obtainable by recombinant production is preferred.

The ABD herein fold completely reversibly, that is they can be denatured and will refold spontaneously to the desired active tertiary structure. This was assessed by circular dichroism spectra analysis, for example of ABD SEQ ID NO:463, where one compares spectrum taken at 20° C. (folded state) and a second spectrum taken after heating to 90° C. (heat denaturation) a third spectrum taken following return to 20° C. (refolded state). During this procedure the Tm can be determined.

Another aspect of the engineered polypeptides is that the ABD can provide an increase in the solubility in aqueous solution of a poor or low soluble exendin variant. This property can be imparted by the ABD itself or because of the ensuing complex of the engineered polypeptide bound to highly soluble albumin in vivo or in vitro, which association increases the solubility of the engineered polypeptide in aqueous solution. Thus, in an embodiment of this further aspect, there is provided a composition, including an exendin compound which per se has a solubility in water of no more than lmg/ml, or no more than 2 mg/ml or no more than 5 mg/ml, covalently coupled to an albumin binding domain as a fusion protein or conjugate as described herein, wherein the compound and the albumin binding polypeptide, fusion protein or conjugate are covalently coupled and the solubility of the engineered polypeptide is greater than that of the unfused (or not conjugated) native exendin compound.

Binding to Albumin. Serum albumin is the most abundant protein in mammalian sera (40 g/L; approximately 0.7 mM in humans) where it binds a variety of molecules including but not limited to lipids and bilirubin (Peters T, 1985, Advances in Protein Chemistry 37:161). It has been observed that the half-life of serum albumin is directly proportional to the size of the animal, where for example human serum albumin (HSA) has a half-life of 19 days and rabbit serum albumin has a half-life of about 5 days (McCurdy T R et al., J. Lab. Clin. Med. 143:115,2004). Human serum albumin is widely distributed throughout the body, in particular in the intestinal and blood compartments, where it is mainly involved in the maintenance of osmolarity. Structurally, albumins are single-chain proteins including three homologous domains and totaling 584 or 585 amino acids (Dugaiczyk L et al., 1982, Proc. Natl. Acad. Sci. USA 79:71). Albumins contain 17 disulfide bridges and a single reactive thiol, C34, but lack N-linked and 0-linked carbohydrate moieties (Peters, 1985, Id.; Nicholson J P et al., 2000, Br J Anaesth 85:599). The lack of glycosylation simplifies recombinant expression of albumin. This property of albumin, together with the fact that its three-dimensional structure is known (He, X M and Carter, D C, Nature 358:209 1992), has made it an attractive candidate for use in recombinant fusion proteins. Such fusion proteins generally combine a therapeutic protein (which would be rapidly cleared from the body upon administration of the protein per se) and a plasma protein (which exhibits a natural slow clearance) in a single polypeptide chain (Sheffield W P, Curr. Drug Targets Cardiovacs. Haematol. Disord. 1:1 2001). Such fusion proteins may provide clinical benefits in requiring less frequent injection and higher levels of therapeutic protein in vivo. However, the engineered polypeptides herein are not conjugated to albumin, but instead contain motifs that allow non-covalent binding to albumin.

Albumin half-life. It has been observed that the half-life of albumin in different species generally adheres to allometric scaling based on animal weight. For example, the albumin half-life in mouse, rat, rabbit and human has been estimated as 1, 1.9, 5.6 and 19 days, respectively. Indeed, power fitting analysis (Davies & Morris, 1993, Pharm. Res. (NY) 10:1093-1095) provides the equation:

Albumin half-life (days)=3.75*body weight(kg)^{0.368}.

Further embodiments. It is understood that each of the polypeptides disclosed herein are also contemplated to include a methionine at the N-terminus in frame with the naturally-occurring first amino acid thereof, e.g., Met-exendin-4, which is exendin-4 with an added N-terminal methionine. It is further understood that where a C-terminal Gly appears in a engineered polypeptide sequence set forth herein, the residue may be lost during subsequent amidation. Some embodiments are intermediates in synthesis, for example, such as those having a “His tag” which is used for affinity purification as is known in the art, and that can optionally be subsequently removed to yield a mature engineered polypeptide suitable for therapeutic use.

In some embodiments of any of the engineered polypeptides described herein, an exendin analog can have at least 70%, for example 70%, 75%, 80%, 85%, 90%, 95%, 98% or even higher, sequence identity relative to a parent exendin sequence. In some embodiments, the parent exendin is exendin-4, and the exendin analog may have at least 70%, for example 70%, 75%, 80%, 85%, 90%, 95%, 98% or even higher, sequence identity relative to exendin-4. As known the art, GLP-1 (glucagon-like peptide 1) is not an exendin; and the sequence of GLP-1 is specifically excluded from exendin sequences suitable for the engineered polypeptides described herein.

In some embodiments, compounds are provided having a linker, for example L1, as described herein, covalently linking a polypeptide hormone domain with an ABD peptide. In some embodiments, a first linker (L1) covalently links HD1 within the engineered polypeptide. In some embodiments, L1 is a bond. In some embodiments, the polypeptide hormone domain (e.g., HD1 as described herein) can be covalently linked to the ABD peptide via a peptide linker. Any linker is optional; i.e., any linker may simply be a bond. When present the chemical structure of a linker is not critical because it serves mainly a spacer function. In one embodiment the linker includes from 1 to 30 or less amino acids linked by peptide bonds. The amino acids can be selected from the 20 naturally occurring (i.e., physiological) amino acids. Alternatively, non-natural amino acids can be incorporated either by chemical synthesis, post-translational chemical modification or by in vivo incorporation by recombinant expression in a host cell. Some of these amino acids may be glycosylated. In another embodiment the 1 to 30 or less amino acids are selected from glycine, alanine, proline, asparagine, glutamine, and lysine, and further from aspartate and glutamate. In a further embodiment the linker is made up of a majority of amino acids that are sterically unhindered, such as glycine, alanine and/or serine. “Sterically unhindered” refers, in the customary sense, to a amino acid having a small side chain, e.g., 0-2 non-hydrogen atoms, such that steric hinderance is minimized relative to amino acids having larger side chains, e.g., Leu, Trp, Tyr, Phe, and the like. Polyglycines are particularly useful, e.g. (Gly)₃, (Gly)₄ (SEQ ID NO:125), (Gly)₅ (SEQ ID NO:126), as are polyalanines, poly(Gly-Ala) and poly(Gly-Ser). Charged polyglycines can be useful, and include e.g., poly (Glyₙ-Glu) (SEQ ID NO:127), poly(Glyₙ-Lys) (SEQ ID NO:128), poly(Glyₙ-Asp) (SEQ ID NO:129), and poly(Glyₙ-Arg) (SEQ ID NO:130) motifs (where n can be 1 to 6). Other specific examples of linkers are (Gly)₃Lys(Gly)₄ (SEQ ID NO:131); (Gly)₃AsnGlySer(Gly)₂ (SEQ ID NO:132); (Gly)₃Cys(Gly)₄ (SEQ ID NO:133); and GlyProAsnGlyGly (SEQ ID NO:134). Combinations of Gly and Ala are particularly useful as are combination of Gly and Ser. Thus, in a further embodiment the peptide linker is selected from the group consisting of a glycine rich peptide, e.g., Gly-Gly-Gly; the sequences [Gly-Ser]ₙ(SEQ ID NO:135), [Gly-Gly-Ser]ₙ(SEQ ID NO:136), [Gly-Gly-Gly-Ser]ₙ (SEQ ID NO:137) and [Gly-Gly-Gly-Gly-Ser]ₙ (SEQ ID NO:138), where n is 1, 2, 3, 4, 5 or 6, for example [Gly-Gly-Gly-Gly Ser]₃ (SEQ ID NO: 721). “Glycine rich peptide” refers to a polypeptide which includes a plurality of glycine residues, preferably a majority of glycine residues, more preferably a preponderance of glycine residues.

In certain embodiments, charged linkers may be used. Such charges linkers may be contain a significant number of acidic residues (e.g., Asp, Glu, and the like), or may contain a significant number of basic residues (e.g., Lys, Arg, and the like), such that the linker has a pI lower than 7 or greater than 7, respectively. As understood by the artisan, and all other things being equal, the greater the relative amount of acidic or basic residues in a given linker, the lower or higher, respectively, the pI of that linker will be. Such linkers may impart advantageous properties to the engineered polypeptides disclosed herein, such as modifying the peptides pI (isoelectric point) which can in turn improve solubility and/or stability characteristics of such polypeptides at a particular pH, such as at physiological pH (e.g., between pH 7.2 and pH 7.6, inclusive), or in a pharmaceutical composition including such polypeptides. As is known in the art, solubility for a peptide can be improved by formulation in a composition having a pH that is at least or more than plus or minus one pH unit from the pI of the peptide.

For example, an “acidic linker” is a linker that has a pI of less than 7; between 6 and 7, inclusive; between 5 and 6, inclusive; between 4 and 5, inclusive; between 3 and 4, inclusive; between 2 and 3, inclusive; or between 1 and 2, inclusive. Similarly, a “basic linker” is a linker that has a pI of greater than 7; between 7 and 8, inclusive; between 8 and 9, inclusive; between 9 and 10, inclusive; between 10 and 11, inclusive; between 11 and 12 inclusive, or between 12 and 13, inclusive. In certain embodiments, an acidic linker will contain a sequence that is selected from the group of [Gly-Glu]ₙ (SEQ ID NO:139); [Gly-Gly-Glu]ₙ(SEQ ID NO:140); [Gly-Gly-Gly-Glu]ₙ (SEQ ID NO:141); [Gly-Gly-Gly-Gly-Glu]ₙ (SEQ ID NO:142), [Gly-Asp]ₙ (SEQ ID NO:143); [Gly-Gly-Asp]ₙ (SEQ ID NO:144); [Gly-Gly-Gly-Asp]ₙ (SEQ ID NO:145); [Gly-Gly-Gly-Gly-Asp]ₙ (SEQ ID NO:146), where n is 1, 2, 3, 4, 5, 6, 7, 8, 9, 10, or more; for example, [Gly-Gly-Glu]₆ (SEQ ID NO: 722). In certain embodiments, a basic linker will contain a sequence that is selected from the group of [Gly-Lys]ₙ (SEQ ID NO:147); [Gly-Gly-Lys]ₙ(SEQ ID NO:148); [Gly-Gly-Gly-Lys]ₙ (SEQ ID NO:149); [Gly-Gly-Gly-Gly-Lys]ₙ (SEQ ID NO:150), [Gly-Arg]ₙ (SEQ ID NO:151); [Gly-Gly-Arg]ₙ (SEQ ID NO:152); [Gly-Gly-Gly-Arg]ₙ (SEQ ID NO:153); [Gly-Gly-Gly-Gly-Arg]ₙ (SEQ ID NO:154) where n is 1, 2, 3, 4, 5, 6, 7, 8, 9, 10, or more; for example, [Gly-Gly-Lys]₆ (SEQ ID NO: 723).

Additionally, linkers may be prepared which possess certain structural motifs or characteristics, such as an alpha helix. For example, such a linker may contain a sequence that is selected from the group of [Glu-Ala-Ala-Ala-Lys]ₙ(SEQ ID NO:155), where n is 1, 2, 3, 4, 5, 6, 7, 8, 9, 10, or more; for example, [Glu-Ala-Ala-Ala-Lys]₃ (SEQ ID NO: 724), [Glu-Ala-Ala-Ala-Lys]₄ (SEQ ID NO: 725), or [Glu-Ala-Ala-Ala-Lys]₅ (SEQ ID NO: 726). One in the art can readily determine helix content of any particular linker sequence.

A biocompatible linker other than a peptide linker may be used to covalently attach the C-terminus of an exendin to the N-terminus of the ABD sequence. The linker can be a biocompatible polymer, preferably water soluble, and more preferably about 50 kD to about 5000 kD, or about 50 KD to 500 kD, or about 100 kD to 500 kD. An exemplary biocompatible, water soluble polymer linker is a PEG linker, such as —(CH₂—CH₂—O)ₙ— where n is such that the PEG linker can have a molecular weight of 100 to 5000 kD, preferably 100 to 500 kD. Such a linker may be —NH—CH₂—CH₂—(O—CH₂—CH₂)ₙ—O—CH₂—CO—, where n is such that the PEG linker molecular weight is 100 kD to 5000 kD, preferably 10 kD to 500 kD. Other biocompatible polymers can be used, such as including but not limited to polysaccharides, polypropylene glycol, and co-polymers of propylene and ethylene glycols. Typically such a linker will include a reactive group at each end that can be the same or different reactive group. Such linkers with reactive groups are known and available. Preferably the reactive group is reactive with either an N-terminal amino or C-terminal carboxy group of a peptide. For example, a reactive group can be an a butylaldehyde, a propionaldehyde, an aldehyde, a succinimide or a maleimide moiety, as is known in the art. Less preferred are alkyl linkers such as —NH—(CH₂)ₙ—C(O)—, wherein n=2-20, and which can be further substituted by any group that does not sterically-hinder peptide function, such as a lower alkyl (e.g., C₁-C₆), lower acyl, halogen, CN, and NH₂.

It is also to be understood that linkers suitable for use in accordance with the invention may possess one or more of the characteristics and motifs described above and herein. For example, a linker may include an acidic linker as well as a structural motif, such as an alpha helix. Similarly, a linker may include a basic linker and a structural motif, such as an alpha helix. A linker may include an acidic linker, a basic linker, and a structural motif, such as an alpha helix. Additionally, it is also to be understood that engineered polypeptides in accordance with the invention may possess more than one linker, and each such linker may possess one or more of the characteristics described herein.

The linkers described herein are exemplary, and linkers within the scope of this invention may be much longer and may include other residues. In one embodiment, expressly excluded are engineered polypeptides in which the exendin sequence is linked directly to the ABD sequence without a linker.

In some embodiments, the engineered polypeptide includes an ABD sequence at the C-terminal, and a HD1 sequence at the N-terminal. In certain preferred embodiments, the N-terminal is an exendin sequence, an exendin fragment sequence or an exendin analog sequence. Further to embodiments which include an ABD and a HD1, the engineered polypeptide can have the structure HD1-ABD.

It is understood that absent an express indication of the N-terminus and/or C-terminus of a engineered polypeptide set forth herein, the engineered polypeptide is to be read in the N-terminus to C-terminus orientation. For example, where HD1 has the sequence of an exendin compound or analog thereof, the terms HD1-ABD, HD1-L1-ABD, HD1-ABD, and the like mean, in the absence of an express indication of the N-terminus and/or the C-terminus, that the exendin sequence or analog thereof resides at the N-terminus of the engineered polypeptide, and the ABD resides at the C-terminus. Conversely, if the N-terminus and/or C-terminus is expressly indicated, then the engineered polypeptide is to be read according to the express indication of the termini. For example, the terms HD1_{C-term}-ABD, HD1-L1-ABD_{N-term} and the like mean that the ABD resides at the N-terminus of the engineered polypeptide, and HD1 resides at the C-terminus.

In some embodiments, the engineered polypeptide described herein has an affinity for serum albumin which is different than the affinity of the ABD polypeptide alone, i.e., in the absence of a fused hormone domain. In order to obtain effective association, the engineered polypeptide can have a binding affinity for serum albumin such that the dissociation constant K_{D} is, for example, less than about 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M or even 10⁻¹⁵ M. In some embodiments, the affinity is not excessively tight such that the engineered polypeptide can dissociate from the albumin and elicit a biological response, for example binding to a receptor, for example, a GLP-1 receptor. The affinity can be measured as described in PCT Published Appl. No. WO2009/016043, preferably to human serum albumin, which is incorporated herein by reference in its entirety and for all purposes, including without limitation assays and synthesis methods.

In some embodiments, a engineered polypeptide described herein is superior to a corresponding compound having a different moiety that can extend plasma half-life (e.g., PEG or of Fc or albumin) conjugated with a hormone domain(s). In this context, the term “superior” refers to a variety of functional properties which could be weighed in the evaluation of a treatment for a disease or disorder. For example, the engineered polypeptide described herein could require less biologically active (hormone domain) component, for example 1×, 2×, 3×, 4×, 5×, or even less, than the corresponding compound having a different moiety conjugated with the hormone domain(s). For further example, the engineered polypeptide described herein could have higher potency, for example, 1.5×, 2×, 3×, 4×, 5×, 10×, 20×, 50×, or even higher potency.

Engineered polypeptide compounds contemplated herein include the compounds as set forth in Table 2 following. Preferred compounds are Cmpd 5, Cmpd 9 and Cmpd 11.

TABLE 2
Selected exemplary engineered polypeptides

| | | SEQ |
|---|---|---|
| Cmpd | Sequence | ID NO: |
| 5 | HGEGTFTSDLSKQLEEEAVRLFIEWLKQGGPSKEIISTGGGGSASGSLAEAKE | 727 |
| | AANAELDSYGVSDFYKRLIDKAKTVEGVEALKDAILAALP | |
| 6 | HGEGTFTSDLSKQLEEEAVRLFIEWLKNGGPSSGAPPKSTGGGGSASGSLAE | 728 |
| | AKEAANAELDSYGVSDFYKRLIDKAKTVEGVEALKDAILAALP | |
| 7 | HGEGTFTSDLSKQLEEEAVRLFIEWLKNGGPSSGAPPPSTGGGGSASGSLAE | 729 |
| | AKEAANAELDSYGVSDFYKRLIDKAKTVEGVEALKDAILAAL | |
| 8 | HGEGTFTSDLSKQMEEEAVRLFIEWLKNGGPSSGAPPPSTGGGGSASGSLAE | 730 |
| | AKEAANAELDSYGVSDFYKRLIDKAKTVEGVEALKDAILAALP | |
| 9 | HGEGTFTSDLSKQMEEEAVRLFIEWLKNGGPSSGAPPPSGGSLAEAKEAAN | 731 |
| | AELDSYGVSDFYKRLIDKAKTVEGVEALKDAILAALP | |
| 10 | HGEGTFTSDLSKQLEEEAVRLFIEWLKNGGPSSGAPPPSTGGGGSASGSLAE | 732 |
| | AKEAANAELDSYGVSDFYKRLIDKAKTVEGVEALKDAILAALP | |
| 11 | HGEGTFTSDLSKQLEEEAVRLFIEWLKNGGPSSGAPPPSGGSLAEAKEAANA | 733 |
| | ELDSYGVSDFYKRLIDKAKTVEGVEALKDAILAALP | |
| | HGEGTFTSDLSKQLEEEAVRLFIEWLKQGGPSKEIISTGGGGSASGSLAEAKE | 734 |
| | AANAELDSYGVSDFYKRLIDKAKTVEGVEALKDAILAAL | |
| | HGEGTFTSDLSKQLEEEAVRLFIEWLKNGGPSSGAPPKSTGGGGSASGSLAE | 735 |
| | AKEAANAELDSYGVSDFYKRLIDKAKTVEGVEALKDAILAAL | |
| | HGEGTFTSDLSKQLEEEAVRLFIEWLKNGGPSSGAPPPSTGGGGSASGSLAE | 736 |
| | AKEAANAELDSYGVSDFYKRLIDKAKTVEGVEALKDAILAA | |
| | HGEGTFTSDLSKQMEEEAVRLFIEWLKNGGPSSGAPPPSTGGGGSASGSLAE | 737 |
| | AKEAANAELDSYGVSDFYKRLIDKAKTVEGVEALKDAILAAL | |
| | HGEGTFTSDLSKQMEEEAVRLFIEWLKNGGPSSGAPPPSGGSLAEAKEAAN | 738 |
| | AELDSYGVSDFYKRLIDKAKTVEGVEALKDAILAAL | |
| | HGEGTFTSDLSKQLEEEAVRLFIEWLKNGGPSSGAPPPSGGSLAEAKEAANA | 739 |
| | ELDSYGVSDFYKRLIDKAKTVEGVEALKDAILAAL | |
| | HGEGTFTSDLSKQLEEEAVRLFIEWLKQGGPSKEIISTGGGGSASGSLAEAKE | 740 |
| | AANAELDSYGVSDFYKRLIDKAKTVEGVEALKDAILAA | |
| | HGEGTFTSDLSKQLEEEAVRLFIEWLKNGGPSSGAPPKSTGGGGSASGSLAE | 741 |
| | AKEAANAELDSYGVSDFYKRLIDKAKTVEGVEALKDAILAA | |
| | HGEGTFTSDLSKQMEEEAVRLFIEWLKNGGPSSGAPPPSTGGGGSASGSLAE | 742 |
| | AKEAANAELDSYGVSDFYKRLIDKAKTVEGVEALKDAILAA | |
| | HGEGTFTSDLSKQMEEEAVRLFIEWLKNGGPSSGAPPPSGGSLAEAKEAAN | 743 |
| | AELDSYGVSDFYKRLIDKAKTVEGVEALKDAILAA | |
| | HGEGTFTSDLSKQLEEEAVRLFIEWLKNGGPSSGAPPPSTGGGGSASGSLAE | 744 |
| | AKEAANAELDSYGVSDFYKRLIDKAKTVEGVEALKDAILAA | |
| | HGEGTFTSDLSKQLEEEAVRLFIEWLKNGGPSSGAPPPSGGSLAEAKEAANA | 745 |
| | ELDSYGVSDFYKRLIDKAKTVEGVEALKDAILAA | |

Additional engineered polypeptide compounds specifically contemplated herein as if set forth specifically, have any on an HD1 and an ABD component, optionally with any of the L1 sequences disclosed herein, and include the compounds having the structure of any of the engineered polypeptides of the tables and listing herein, including those disclosed in Table 3 following:

TABLE 3
Selected exemplary engineered polypeptides

| | | SEQ ID |
|---|---|---|
| Cmpd | Sequence | NO: |
| 5 | HGEGTFTSDLSKQLEEEAVRLFIEWLKQGGPSKEIISTGGGGSASGSLAEAKE | 727 |
| | AANAELDSYGVSDFYKRLIDKAKTVEGVEALKDAILAALP | |
| | HGEGTFTSDLSKQLEEEAVRLFIEWLKQGGPSKEIISTGGGGSASGSLAEAKE | 734 |
| | AANAELDSYGVSDFYKRLIDKAKTVEGVEALKDAILAAL | |
| | HGEGTFTSDLSKQLEEEAVRLFIEWLKQGGPSKEIISTGGGGSASSLAEAKEA | 746 |
| | ANAELDSYGVSDFYKRLIDKAKTVEGVEALKDAILAALP | |
| | HGEGTFTSDLSKQLEEEAVRLFIEWLKQGGPSKEIISTGGGGSASGSLAEAKE | 740 |
| | AANAELDSYGVSDFYKRLIDKAKTVEGVEALKDAILAA | |
| | HGEGTFTSDLSKQLEEEAVRLFIEWLKQGGPSKEIISTGGGGSASSLAEAKEA | 747 |
| | ANAELDSYGVSDFYKRLIDKAKTVEGVEALKDAILAAL | |
| | HGEGTFTSDLSKQLEEEAVRLFIEWLKQGGPSKEIISTGGGGSASLAEAKEAA | 748 |
| | NAELDSYGVSDFYKRLIDKAKTVEGVEALKDAILAALP | |
| | HGEGTFTSDLSKQLEEEAVRLFIEWLKQGGPSKEIISTGGGGSASLAEAKEAA | 749 |
| | NAELDSYGVSDFYKRLIDKAKTVEGVEALKDAILAAL | |
| | HGEGTFTSDLSKQLEEEAVRLFIEWLKQGGPSKEIISGGGGSLAEAKEAANAE | 750 |
| | LDSYGVSDFYKRLIDKAKTVEGVEALKDAILAALP | |
| | HGEGTFTSDLSKQLEEEAVRLFIEWLKQGGPSKEIISGGGGSLAEAKEAANAE | 751 |
| | LDSYGVSDFYKRLIDKAKTVEGVEALKDAILAAL | |
| | HGEGTFTSDLSKQLEEEAVRLFIEWLKQGGPSKEIISGGGSLAEAKEAANAEL | 752 |
| | DSYGVSDFYKRLIDKAKTVEGVEALKDAILAALP | |
| | HGEGTFTSDLSKQLEEEAVRLFIEWLKQGGPSKEIISGGGSLAEAKEAANAEL | 753 |
| | DSYGVSDFYKRLIDKAKTVEGVEALKDAILAAL | |
| | HGEGTFTSDLSKQLEEEAVRLFIEWLKQGGPSKEIISGGGLAEAKEAANAELD | 754 |
| | SYGVSDFYKRLIDKAKTVEGVEALKDAILAALP | |
| | HGEGTFTSDLSKQLEEEAVRLFIEWLKQGGPSKEIISGGGLAEAKEAANAELD | 755 |
| | SYGVSDFYKRLIDKAKTVEGVEALKDAILAAL | |
| | HGEGTFTSDLSKQLEEEAVRLFIEWLKQGGPSKEIISGGSLAEAKEAANAELD | 756 |
| | SYGVSDFYKRLIDKAKTVEGVEALKDAILAALP | |
| | HGEGTFTSDLSKQLEEEAVRLFIEWLKQGGPSKEIISGGSLAEAKEAANAELD | 757 |
| | SYGVSDFYKRLIDKAKTVEGVEALKDAILAAL | |
| | HGEGTFTSDLSKQLEEEAVRLFIEWLKQGGPSKEIISGSLAEAKEAANAELDS | 985 |
| | YGVSDFYKRLIDKAKTVEGVEALKDAILAALP | |
| | HGEGTFTSDLSKQLEEEAVRLFIEWLKQGGPSKEIISGSLAEAKEAANAELDS | 986 |
| | YGVSDFYKRLIDKAKTVEGVEALKDAILAAL | |
| | HGEGTFTSDLSKQLEEEAVRLFIEWLKQGGPSKEIISGLAEAKEAANAELDSY | 987 |
| | GVSDFYKRLIDKAKTVEGVEALKDAILAALP | |
| | HGEGTFTSDLSKQLEEEAVRLFIEWLKQGGPSKEIISGLAEAKEAANAELDSY | 988 |
| | GVSDFYKRLIDKAKTVEGVEALKDAILAAL | |
| 6 | HGEGTFTSDLSKQLEEEAVRLFIEWLKNGGPSSGAPPKSTGGGGSASGSLAE | 728 |
| | AKEAANAELDSYGVSDFYKRLIDKAKTVEGVEALKDAILAALP | |
| | HGEGTFTSDLSKQLEEEAVRLFIEWLKNGGPSSGAPPKSTGGGGSASGSLAE | 735 |
| | AKEAANAELDSYGVSDFYKRLIDKAKTVEGVEALKDAILAAL | |
| | HGEGTFTSDLSKQLEEEAVRLFIEWLKNGGPSSGAPPKSTGGGGSASSLAEA | 758 |
| | KEAANAELDSYGVSDFYKRLIDKAKTVEGVEALKDAILAALP | |
| | HGEGTFTSDLSKQLEEEAVRLFIEWLKNGGPSSGAPPKSTGGGGSASSLAEA | 759 |
| | KEAANAELDSYGVSDFYKRLIDKAKTVEGVEALKDAILAAL | |
| | HGEGTFTSDLSKQLEEEAVRLFIEWLKNGGPSSGAPPKSTGGGGSASLAEAK | 760 |
| | EAANAELDSYGVSDFYKRLIDKAKTVEGVEALKDAILAALP | |
| | HGEGTFTSDLSKQLEEEAVRLFIEWLKNGGPSSGAPPKSTGGGGSASLAEAK | 761 |
| | EAANAELDSYGVSDFYKRLIDKAKTVEGVEALKDAILAAL | |
| | HGEGTFTSDLSKQLEEEAVRLFIEWLKNGGPSSGAPPKSGGGGSLAEAKEAA | 762 |
| | NAELDSYGVSDFYKRLIDKAKTVEGVEALKDAILAALP | |
| | HGEGTFTSDLSKQLEEEAVRLFIEWLKNGGPSSGAPPKSGGGGSLAEAKEAA | 763 |
| | NAELDSYGVSDFYKRLIDKAKTVEGVEALKDAILAAL | |
| | HGEGTFTSDLSKQLEEEAVRLFIEWLKNGGPSSGAPPKSGGGSLAEAKEAAN | 764 |
| | AELDSYGVSDFYKRLIDKAKTVEGVEALKDAILAALP | |
| | HGEGTFTSDLSKQLEEEAVRLFIEWLKNGGPSSGAPPKSGGGSLAEAKEAAN | 765 |
| | AELDSYGVSDFYKRLIDKAKTVEGVEALKDAILAAL | |
| | HGEGTFTSDLSKQLEEEAVRLFIEWLKNGGPSSGAPPKSGGGLAEAKEAANA | 766 |
| | ELDSYGVSDFYKRLIDKAKTVEGVEALKDATLAALP | |
| | HGEGTFTSDLSKQLEEEAVRLFIEWLKNGGPSSGAPPKSGGGLAEAKEAANA | 767 |
| | ELDSYGVSDFYKRLIDKAKTVEGVEALKDAILAAL | |
| | HGEGTFTSDLSKQLEEEAVRLFIEWLKNGGPSSGAPPKSGGSLAEAKEAANA | 768 |
| | ELDSYGVSDFYKRLIDKAKTVEGVEALKDAILAALP | |
| | HGEGTFTSDLSKQLEEEAVRLFIEWLKNGGPSSGAPPKSGGSLAEAKEAANA | 769 |
| | ELDSYGVSDFYKRLIDKAKTVEGVEALKDAILAAL | |
| | HGEGTFTSDLSKQLEEEAVRLFIEWLKNGGPSSGAPPKSGSLAEAKEAANAE | 770 |
| | LDSYGVSDFYKRLIDKAKTVEGVEALKDAILAALP | |
| | HGEGTFTSDLSKQLEEEAVRLFIEWLKNGGPSSGAPPKSGSLAEAKEAANAE | 771 |
| | LDSYGVSDFYKRLIDKAKTVEGVEALKDAILAAL | |
| | HGEGTFTSDLSKQLEEEAVRLFIEWLKNGGPSSGAPPKSGLAEAKEAANAEL | 772 |
| | DSYGVSDFYKRLIDKAKTVEGVEALKDAILAALP | |
| | HGEGTFTSDLSKQLEEEAVRLFIEWLKNGGPSSGAPPKSGLAEAKEAANAEL | 773 |
| | DSYGVSDFYKRLIDKAKTVEGVEALKDAILAAL | |
| | HGEGTFTSDLSKQMEEEAVRLFIEWLKNGGPSSGAPPKSTGGGGSASGSLAE | 774 |
| | AKEAANAELDSYGVSDFYKRLIDKAKTVEGVEALKDAILAALP | |
| | HGEGTFTSDLSKQMEEEAVRLFIEWLKNGGPSSGAPPKSTGGGGSASGSLAE | 775 |
| | AKEAANAELDSYGVSDFYKRLIDKAKTVEGVEALKDAILAAL | |
| | HGEGTFTSDLSKQMEEEAVRLFIEWLKNGGPSSGAPPKSTGGGGSASSLAEA | 776 |
| | KEAANAELDSYGVSDFYKRLIDKAKTVEGVEALKDAILAALP | |
| | HGEGTFTSDLSKQMEEEAVRLFTEWLKNGGPSSGAPPKSTGGGGSASSLAEA | 777 |
| | KEAANAELDSYGVSDFYKRLIDKAKTVEGVEALKDAILAAL | |
| | HGEGTFTSDLSKQMEEEAVRLFIEWLKNGGPSSGAPPKSTGGGGSASLAEAK | 778 |
| | EAANAELDSYGVSDFYKRLIDKAKTVEGVEALKDAILAALP | |
| | HGEGTFTSDLSKQMEEEAVRLFIEWLKNGGPSSGAPPKSTGGGGSASLAEAK | 779 |
| | EAANAELDSYGVSDFYKRLIDKAKTVEGVEALKDAILAAL | |
| | HGEGTFTSDLSKQMEEEAVRLFIEWLKNGGPSSGAPPKSGGGGSLAEAKEAA | 780 |
| | NAELDSYGVSDFYKRLIDKAKTVEGVEALKDAILAALP | |
| | HGEGTFTSDLSKQMEEEAVRLFIEWLKNGGPSSGAPPKSGGGGSLAEAKEAA | 781 |
| | NAELDSYGVSDFYKRLIDKAKTVEGVEALKDAILAAL | |
| | HGEGTFTSDLSKQMEEEAVRLFIEWLKNGGPSSGAPPKSGGGSLAEAKEAAN | 782 |
| | AELDSYGVSDFYKRLIDKAKTVEGVEALKDAILAALP | |
| | HGEGTFTSDLSKQMEEEAVRLFIEWLKNGGPSSGAPPKSGGGSLAEAKEAAN | 783 |
| | AELDSYGVSDFYKRLIDKAKTVEGVEALKDATLAAL | |
| | HGEGTFTSDLSKQMEEEAVRLFIEWLKNGGPSSGAPPKSGGGLAEAKEAAN | 784 |
| | AELDSYGVSDFYKRLIDKAKTVEGVEALKDAILAALP | |
| | HGEGTFTSDLSKQMEEEAVRLFIEWLKNGGPSSGAPPKSGGGLAEAKEAAN | 785 |
| | AELDSYGVSDFYKRLIDKAKTVEGVEALKDAILAAL | |
| | HGEGTFTSDLSKQMEEEAVRLFIEWLKNGGPSSGAPPKSGGSLAEAKEAANA | 786 |
| | ELDSYGVSDFYKRLIDKAKTVEGVEALKDAILAALP | |
| | HGEGTFTSDLSKQMEEEAVRLFIEWLKNGGPSSGAPPKSGGSLAEAKEAANA | 787 |
| | ELDSYGVSDFYKRLIDKAKTVEGVEALKDAILAAL | |
| | HGEGTFTSDLSKQMEEEAVRLFIEWLKNGGPSSGAPPKSGSLAEAKEAANAE | 788 |
| | LDSYGVSDFYKRLIDKAKTVEGVEALKDAILAALP | |
| | HGEGTFTSDLSKQMEEEAVRLFIEWLKNGGPSSGAPPKSGSLAEAKEAANAE | 789 |
| | LDSYGVSDFYKRLIDKAKTVEGVEALKDAILAAL | |
| | HGEGTFTSDLSKQMEEEAVRLFIEWLKNGGPSSGAPPKSGLAEAKEAANAEL | 790 |
| | DSYGVSDFYKRLIDKAKTVEGVEALKDAILAALP | |
| | HGEGTFTSDLSKQMEEEAVRLFTEWLKNGGPSSGAPPKSGLAEAKEAANAEL | 791 |
| | DSYGVSDFYKRLIDKAKTVEGVEALKDAILAAL | |
| 10 | HGEGTFTSDLSKQLEEEAVRLFIEWLKNGGPSSGAPPPSTGGGGSASGSLAEA | 732 |
| | KEAANAELDSYGVSDFYKRLIDKAKTVEGVEALKDAILAALP | |
| | HGEGTFTSDLSKQLEEEAVRLFIEWLKNGGPSSGAPPPSTGGGGSASSLAEAK | 792 |
| | EAANAELDSYGVSDFYKRLIDKAKTVEGVEALKDAILAALP | |
| | HGEGTFTSDLSKQLEEEAVRLFIEWLKNGGPSSGAPPPSTGGGGSASGSLAEA | 736 |
| | KEAANAELDSYGVSDFYKRLIDKAKTVEGVEALKDAILAA | |
| | HGEGTFTSDLSKQLEEEAVRLFIEWLKNGGPSSGAPPPSTGGGGSASSLAEAK | 793 |
| | EAANAELDSYGVSDFYKRLIDKAKTVEGVEALKDAILAAL | |
| | HGEGTFTSDLSKQLEEEAVRLFIEWLKNGGPSSGAPPPSTGGGGSASLAEAKE | 794 |
| | AANAELDSYGVSDFYKRLIDKAKTVEGVEALKDAILAALP | |
| | HGEGTFTSDLSKQLEEEAVRLFIEWLKNGGPSSGAPPPSTGGGGSASLAEAKE | 795 |
| | AANAELDSYGVSDFYKRLIDKAKTVEGVEALKDAILAAL | |
| | HGEGTFTSDLSKQLEEEAVRLFIEWLKNGGPSSGAPPPSGGGGSLAEAKEAA | 796 |
| | NAELDSYGVSDFYKRLIDKAKTVEGVEALKDAILAALP | |
| | HGEGTFTSDLSKQLEEEAVRLFIEWLKNGGPSSGAPPPSGGGGSLAEAKEAA | 797 |
| | NAELDSYGVSDFYKRLIDKAKTVEGVEALKDAILAAL | |
| | HGEGTFTSDLSKQLEEEAVRLFIEWLKNGGPSSGAPPPSGGGSLAEAKEAAN | 798 |
| | AELDSYGVSDFYKRLIDKAKTVEGVEALKDAILAALP | |
| | HGEGTFTSDLSKQLEEEAVRLFIEWLKNGGPSSGAPPPSGGGSLAEAKEAAN | 799 |
| | AELDSYGVSDFYKRLIDKAKTVEGVEALKDAILAAL | |
| | HGEGTFTSDLSKQLEEEAVRLFIEWLKNGGPSSGAPPPSGGGLAEAKEAANA | 800 |
| | ELDSYGVSDFYKRLIDKAKTVEGVEALKDAILAALP | |
| | HGEGTFTSDLSKQLEEEAVRLFIEWLKNGGPSSGAPPPSGGGLAEAKEAANA | 801 |
| | ELDSYGVSDFYKRLIDKAKTVEGVEALKDAILAAL | |
| 11 | HGEGTFTSDLSKQLEEEAVRLFIEWLKNGGPSSGAPPPSGGSLAEAKEAANA | 733 |
| | ELDSYGVSDFYKRLIDKAKTVEGVEALKDAILAALP | |
| | HGEGTFTSDLSKQLEEEAVRLFIEWLKNGGPSSGAPPPSGGSLAEAKEAANA | 739 |
| | ELDSYGVSDFYKRLIDKAKTVEGVEALKDAILAAL | |
| | HGEGTFTSDLSKQLEEEAVRLFIEWLKNGGPSSGAPPPSGSLAEAKEAANAE | 802 |
| | LDSYGVSDFYKRLIDKAKTVEGVEALKDAILAALP | |
| | HGEGTFTSDLSKQLEEEAVRLFIEWLKNGGPSSGAPPPSGSLAEAKEAANAE | 803 |
| | LDSYGVSDFYKRLIDKAKTVEGVEALKDAILAAL | |
| | HGEGTFTSDLSKQLEEEAVRLFIEWLKNGGPSSGAPPPSGLAEAKEAANAEL | 804 |
| | DSYGVSDFYKRLIDKAKTVEGVEALKDAILAALP | |
| | HGEGTFTSDLSKQLEEEAVRLFIEWLKNGGPSSGAPPPSGGSLAEAKEAANA | 745 |
| | ELDSYGVSDFYKRLIDKAKTVEGVEALKDAILAA | |
| | HGEGTFTSDLSKQLEEEAVRLFIEWLKNGGPSSGAPPPSGLAEAKEAANAEL | 805 |
| | DSYGVSDFYKRLIDKAKTVEGVEALKDAILAAL | |
| 8 | HGEGTFTSDLSKQMEEEAVRLFIEWLKNGGPSSGAPPPSTGGGGSASGSLAE | 730 |
| | AKEAANAELDSYGVSDFYKRLIDKAKTVEGVEALKDAILAALP | |
| | HGEGTFTSDLSKQMEEEAVRLFIEWLKNGGPSSGAPPPSTGGGGSASGSLAE | 737 |
| | AKEAANAELDSYGVSDFYKRLIDKAKTVEGVEALKDAILAAL | |
| | HGEGTFTSDLSKQMEEEAVRLFIEWLKNGGPSSGAPPPSTGGGGSASSLAEA | 806 |
| | KEAANAELDSYGVSDFYKRLIDKAKTVEGVEALKDAILAALP | |
| | HGEGTFTSDLSKQMEEEAVRLFIEWLKNGGPSSGAPPPSTGGGGSASSLAEA | 807 |
| | KEAANAELDSYGVSDFYKRLIDKAKTVEGVEALKDAILAAL | |
| | HGEGTFTSDLSKQMEEEAVRLFIEWLKNGGPSSGAPPPSTGGGGSASLAEAK | 808 |
| | EAANAELDSYGVSDFYKRLTDKAKTVEGVEALKDATLAALP | |
| | HGEGTFTSDLSKQMEEEAVRLFIEWLKNGGPSSGAPPPSTGGGGSASLAEAK | 809 |
| | EAANAELDSYGVSDFYKRLIDKAKTVEGVEALKDAILAAL | |
| | HGEGTFTSDLSKQMEEEAVRLFIEWLKNGGPSSGAPPPSGGGGSLAEAKEAA | 810 |
| | NAELDSYGVSDFYKRLIDKAKTVEGVEALKDAILAALP | |
| | HGEGTFTSDLSKQMEEEAVRLFIEWLKNGGPSSGAPPPSGGGGSLAEAKEAA | 811 |
| | NAELDSYGVSDFYKRLIDKAKTVEGVEALKDAILAAL | |
| | HGEGTFTSDLSKQMEEEAVRLFIEWLKNGGPSSGAPPPSGGGSLAEAKEAAN | 812 |
| | AELDSYGVSDFYKRLIDKAKTVEGVEALKDAILAALP | |
| | HGEGTFTSDLSKQMEEEAVRLFIEWLKNGGPSSGAPPPSGGGSLAEAKEAAN | 813 |
| | AELDSYGVSDFYKRLIDKAKTVEGVEALKDAILAAL | |
| | HGEGTFTSDLSKQMEEEAVRLFIEWLKNGGPSSGAPPPSGGGLAEAKEAANA | 814 |
| | ELDSYGVSDFYKRLIDKAKTVEGVEALKDAILAALP | |
| | HGEGTFTSDLSKQMEEEAVRLFIEWLKNGGPSSGAPPPSGGGLAEAKEAANA | 815 |
| | ELDSYGVSDFYKRLIDKAKTVEGVEALKDAILAAL | |
| 9 | HGEGTFTSDLSKQMEEEAVRLFIEWLKNGGPSSGAPPPSGGSLAEAKEAANA | 731 |
| | ELDSYGVSDFYKRLIDKAKTVEGVEALKDAILAALP | |
| | HGEGTFTSDLSKQMEEEAVRLFIEWLKNGGPSSGAPPPSGGSLAEAKEAANA | 738 |
| | ELDSYGVSDFYKRLIDKAKTVEGVEALKDAILAAL | |
| | HGEGTFTSDLSKQMEEEAVRLFIEWLKNGGPSSGAPPPSGGSLAEAKEAANA | 743 |
| | ELDSYGVSDFYKRLIDKAKTVEGVEALKDAILAA | |
| | HGEGTFTSDLSKQMEEEAVRLFTEWLKNGGPSSGAPPPSGSLAEAKEAANAE | 816 |
| | LDSYGVSDFYKRLIDKAKTVEGVEALKDAILAALP | |
| | HGEGTFTSDLSKQMEEEAVRLFIEWLKNGGPSSGAPPPSGSLAEAKEAANAE | 817 |
| | LDSYGVSDFYKRLIDKAKTVEGVEALKDAILAAL | |
| | HGEGTFTSDLSKQMEEEAVRLFIEWLKNGGPSSGAPPPSGLAEAKEAANAEL | 818 |
| | DSYGVSDFYKRLIDKAKTVEGVEALKDAILAALP | |
| | HGEGTFTSDLSKQMEEEAVRLFIEWLKNGGPSSGAPPPSGLAEAKEAANAEL | 819 |
| | DSYGVSDFYKRLIDKAKTVEGVEALKDAILAAL | |
| | HGEGTFTSDLSKQLEEEAVRLFIEWLKNGGPSSGAPPSKKKKKKTGGGGSAS | 820 |
| | GSLAEAKEAANAELDSYGVSDFYKRLIDKAKTVEGVEALKDAILAALP | |
| | HGEGTFTSDLSKQLEEEAVRLFIEWLKNGGPSSGAPPSKKKKKKTGGGGSAS | 821 |
| | GSLAEAKEAANAELDSYGVSDFYKRLIDKAKTVEGVEALKDAILAAL | |
| | HGEGTFTSDLSKQLEEEAVRLFIEWLKNGGPSSGAPPSKKKKKKTGGGGSAS | 822 |
| | SLAEAKEAANAELDSYGVSDFYKRLTDKAKTVEGVEALKDATLAALP | |
| | HGEGTFTSDLSKQLEEEAVRLFIEWLKNGGPSSGAPPSKKKKKKTGGGGSAS | 823 |
| | SLAEAKEAANAELDSYGVSDFYKRLIDKAKTVEGVEALKDAILAAL | |
| | HGEGTFTSDLSKQLEEEAVRLFIEWLKNGGPSSGAPPSKKKKKKTGGGGSAS | 824 |
| | LAEAKEAANAELDSYGVSDFYKRLIDKAKTVEGVEALKDAILAALP | |
| | HGEGTFTSDLSKQLEEEAVRLFIEWLKNGGPSSGAPPSKKKKKKTGGGGSAS | 825 |
| | LAEAKEAANAELDSYGVSDFYKRLIDKAKTVEGVEALKDAILAAL | |
| | HGEGTFTSDLSKQLEEEAVRLFIEWLKNGGPSSGAPPSKKKKKKGGGGSLAE | 826 |
| | AKEAANAELDSYGVSDFYKRLIDKAKTVEGVEALKDAILAALP | |
| | HGEGTFTSDLSKQLEEEAVRLFIEWLKNGGPSSGAPPSKKKKKKGGGGSLAE | 827 |
| | AKEAANAELDSYGVSDFYKRLIDKAKTVEGVEALKDAILAAL | |
| | HGEGTFTSDLSKQLEEEAVRLFIEWLKNGGPSSGAPPSKKKKKKGGGSLAEA | 828 |
| | KEAANAELDSYGVSDFYKRLIDKAKTVEGVEALKDAILAALP | |
| | HGEGTFTSDLSKQLEEEAVRLFIEWLKNGGPSSGAPPSKKKKKKGGGSLAEA | 829 |
| | KEAANAELDSYGVSDFYKRLIDKAKTVEGVEALKDAILAAL | |
| | HGEGTFTSDLSKQLEEEAVRLFTEWLKNGGPSSGAPPSKKKKKKGGGLAEAK | 830 |
| | EAANAELDSYGVSDFYKRLIDKAKTVEGVEALKDAILAALP | |
| | HGEGTFTSDLSKQLEEEAVRLFIEWLKNGGPSSGAPPSKKKKKKGGGLAEAK | 831 |
| | EAANAELDSYGVSDFYKRLIDKAKTVEGVEALKDAILAAL | |
| | HGEGTFTSDLSKQLEEEAVRLFIEWLKNGGPSSGAPPSKKKKKKGGSLAEAK | 832 |
| | EAANAELDSYGVSDFYKRLIDKAKTVEGVEALKDAILAALP | |
| | HGEGTFTSDLSKQLEEEAVRLFIEWLKNGGPSSGAPPSKKKKKKGGSLAEAK | 833 |
| | EAANAELDSYGVSDFYKRLIDKAKTVEGVEALKDAILAAL | |
| | HGEGTFTSDLSKQLEEEAVRLFIEWLKNGGPSSGAPPSKKKKKKGSLAEAKE | 834 |
| | AANAELDSYGVSDFYKRLIDKAKTVEGVEALKDAILAALP | |
| | HGEGTFTSDLSKQLEEEAVRLFIEWLKNGGPSSGAPPSKKKKKKGSLAEAKE | 835 |
| | AANAELDSYGVSDFYKRLIDKAKTVEGVEALKDAILAAL | |
| | HGEGTFTSDLSKQLEEEAVRLFIEWLKNGGPSSGAPPSKKKKKKGLAEAKEA | 836 |
| | ANAELDSYGVSDFYKRLIDKAKTVEGVEALKDATLAALP | |
| | HGEGTFTSDLSKQLEEEAVRLFIEWLKNGGPSSGAPPSKKKKKKGLAEAKEA | 837 |
| | ANAELDSYGVSDFYKRLIDKAKTVEGVEALKDAILAAL | |

Specifically contemplated are compounds of the above sequences in which any N-terminal methionine is absent. The N-terminal methionine can be present primarily as a convenience for bacterial expression. However, engineered peptides of the present invention can be expressed in a eukaryotic host cell (e.g. yeast (e.g. Pichia), mammalian, baculovirus) or other host cell having post-translational N-terminal proteolytic processing to yield an N-terminal amino acid as found in a naturally occurring mature peptide counterpart of the desired hormone or ABD sequence, i.e. without the added methionine or other leader sequence. Alternatively, an N-terminal sequence used for expression and/or secretion (and even purification) can be one that can be removed post-translationally, e.g. as by use of a protease such as TEV.

### III. METHODS OF DESIGN AND PRODUCTION

Design of constructs. The engineered polypeptides described herein can be designed at the amino acid level. These sequences can then be back translated using a variety of software products known in the art such that the nucleotide sequence is optimized for the desired expression host, e.g. based protein expression, codon optimization, restriction site content. For example, the nucleotide sequence can be optimized for E. coli based protein expression and for restriction site content. Based on the nucleotide sequence of interest, overlapping oligonucleotides can be provided for multistep PCR, as known in the art. These oligonucleotides can be used in multiple PCR reactions under conditions well known in the art to build the cDNA encoding the protein of interest. For one example is 1×Amplitaq Buffer, 1.3 mM MgCl₂, 200 uM dNTPs, 4 U Amplitaq Gold, 0.2 uM of each primer (AmpliTaq Gold, ABI), with cycling parameters: (94 C:30 s, 58 C:1 min, 72 C: 1 min), 35 cycles.

Restriction sites can be added to the ends of the PCR products for use in vector ligation as known in the art. Specific sites can include Nde1 and Xho1, such that the cDNA can then be in the proper reading frame in a pET45b expression vector (Novagcn). By using these sites, any N-terminal His Tag that are in this vector can be removed as the translation start site would then be downstream of the tag. Once expression constructs are completed, verification can be conduct by sequencing using e.g., T7 promoter primer, T7 terminator primer and standard ABI BigDye Term v3.1 protocols as known in the art. Sequence information can be obtained from e.g., an ABI 3730 DNA Analyzer and can be analyzed using Vector NTI v.10 software (Invitrogen). Expression constructs can be designed in a modular manner such that linker sequences can be easily cut out and changed, as known in the art.

Protease recognition sites, known in the art or described herein, can be incorporated into constructs useful for the design, construction, manipulation and production of recombinant engineering polypeptides described herein.

Exemplary constructs. Constructs useful in the production of engineered polypeptides contemplated herein include constructs encoding the polypeptides set forth in Table 4 following.

TABLE 4
Selected exemplary constructs for recombinant
production of engineered polypeptides

MAHHHHHHVGTGSNENLYFQHGEGTFTSDLSKQLEEEAVRLFIEWLKQGG
PSKEIISTGGGGSASGSLAEAKEAANAELDSYGVSDFYKRLIDKAKTVEG
VEALKDAILAALP (SEQ ID NO: 838)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQLEEEAVRLFIEWLKQGGPSKEIISTGGG
GSASGSLAEAKEAANAELDSYGVSDFYKRLIDKAKTVEGVEALKDAILAA
LP (SEQ ID NO: 839)
MAHHHHHHVGTGSNENLYFQHGEGTFTSDLSKQLEEEAVRLFIEWLKNGG
PSSGAPPPSTGGGGSASGSLAEAKEAANAELDSYGVSDFYKRLIDKAKTV
EGVEALKDAILAALP (SEQ ID NO: 840)
MSDKTTHLTDDSFDTDVLKADGATLVDFWAEWCGPCKMTAPTLDETADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQLEEEAVRLFIEWLKNGGPSSGAPPPSTG
GGGSASGSLAEAKEAANAELDSYGVSDFYKRLIDKAKTVEGVEALKDAIL
AALP (SEQ ID NO: 841)
MAHHHHHHVGTGSNENLYFQHGEGTFTSDLSKQMEEEAVRLFIEWLKNGG
PSSGAPPPSTGGGGSASGSLAEAKEAANAELDSYGVSDFYKRLIDKAKTV
EGVEALKDAILAALP (SEQ ID NO: 842)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQMEEEAVRLFIEWLKNGGPSSGAPPPSTG
GGGSASGSLAEAKEAANAELDSYGVSDFYKRLIDKAKTVEGVEALKDAIL
AALP (SEQ ID NO: 843)

General methods of production. The engineered polypeptide compounds described herein may be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties and for all purposes. Other methods for preparing the compounds are set forth herein.

The engineered polypeptides compounds described herein may be prepared using standard solid-phase peptide synthesis techniques, such as an automated or semiautomated peptide synthesizer. Briefly and generally, the ABD and therapeutic hormonal peptide can be made separately and then conjugated together or can be made as a single polypeptide. Thus, the albumin binding polypeptide, therapeutic hormone or engineered polypeptide may alternatively be produced by non-biological peptide synthesis using amino acids and/or amino acid derivatives having reactive side-chains protected, the non-biological peptide synthesis including step-wise coupling of the amino acids and/or the amino acid derivatives to form a polypeptide according to the first aspect having reactive side-chains protected, removing the protecting groups from the reactive side-chains of the polypeptide, and folding of the polypeptide in aqueous solution. Thus, normal amino acids (e.g. glycine, alanine, phenylalanine, isoleucine, leucine and valine) and pre-protected amino acid derivatives are used to sequentially build a polypeptide sequence, in solution or on a solid support in an organic solvent. When a complete polypeptide sequence is built, the protecting groups are removed and the polypeptide is allowed to fold in an aqueous solution.

Each polypeptide according to the present disclosure reversibly folds, with the ABD domain reversibly folding into a three helix bundle domain without added factors, and hence folds spontaneously. The engineered conjugate may be produced by a method including producing an albumin binding polypeptide according to any method, e.g. as described herein, such as by non-biological peptide synthesis, and conjugating the produced ABD polypeptide with the therapeutic hormone defined herein. The ABDs herein fold completely reversibly. This was assessed by circular dichroism spectra analysis; one spectrum taken at 20° C. and a second spectrum after heating to 90° C. followed by return to 20° C. During this procedure the Tm, as known in the art, was determined and found to be unchanged after the folding of the denatured polypeptide.

Typically, using such techniques, an alpha-N-carbamoyl protected amino acid and an amino acid attached to the growing peptide chain on a resin are coupled at RT in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from Applied Biosystems Inc. (Foster City, Calif.).

For chemical synthesis solid phase peptide synthesis can be used for the engineered polypeptides, since in general solid phase synthesis is a straightforward approach with excellent scalability to commercial scale, and is generally compatible with relatively long engineered polypeptides. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See APPLIED BIOSYSTEMS USER'S MANUAL FOR THE ABI 430A PEPTIDE SYNTHESIZER, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., Introduction to Cleavage Techniques, Applied Biosystems, Inc., 1990, pp. 6-12). Peptides may also be assembled using an Advanced Chem Tech Synthesizer (Model MPS 350, Louisville, Ky.).

A chemical synthesis method that provided better yields is exemplified as follows for Cmpd 11. Solid phase synthesis was performed using a Prelude 6 channel peptide synthesizer (Protein Technologies, Inc., Tucson, Az., USA) using Fomc-Pro-Novasyn TGT resin (0.2 mmole/g) using default “double coupling” settings. However for VS (amino acid positions 59-60) and KT (amino acid positions 71-72) sequences pseudoproline double coupling was used, and for amino acids V19, R20, 123, and P37 triple coupling was used. For the exendin portion from His1 to Ser39, HATU/DTEA double or triple coupling (˜60 min each, 6× excess of reagents) was performed unless otherwise indicated with deblocking with 20% piperidine 2×15min. For the linker and ABD portion, HATU/DIEA double coupling was performed unless otherwise indicated (˜60 min each, 3× excess of reagents) with deblocking with 20% piperidine 2×15 min. Polypeptide purification was performed using RP-HPLC purification on a C5 column using acetonitrile as solvent, with eluted samples identified by analysis on an analytical RP-HPLC on a C18 column using acetonitrile as solvent, followed by preparative RP-HPLC on a C18 column using a more narrow gradient than in the first RP-HPLC and acetonitrile as solvent. Fractions containing desired engineered polypeptide were pooled and lyophilized.

The compounds (exendins, ABDs, linkers, engineered polypeptides) described herein may also be prepared using recombinant DNA techniques using methods known in the art, such as Sambrook et al., 1989, MOLECULAR CLONING: A LABORATORY MANUAL, 2d Ed., Cold Spring Harbor. Non-peptide compounds may be prepared by art-known methods. For example, phosphate-containing amino acids and peptides containing such amino acids, may be prepared using methods known in the art, such as described in Bartlett et al, 1986, Biorg. Chem., 14:356-377. Compounds can be conjugated using art methods or as described herein.

The engineered polypeptides may alternatively be produced by recombinant techniques well known in the art. See, e.g., Sambrook et al., 1989 (Id.). These engineered polypeptides produced by recombinant technologies may be expressed from a polynucleotide. One skilled in the art will appreciate that the polynucleotides, including DNA and RNA, that encode such engineered polypeptides may be obtained from the wild-type cDNA, e.g. exendin-4, taking into consideration the degeneracy of codon usage, and may further engineered as desired to incorporate the indicated substitutions. These polynucleotide sequences may incorporate codons facilitating transcription and translation of mRNA in microbial hosts. Such manufacturing sequences may readily be constructed according to the methods well known in the art. See, e.g., WO 83/04053, incorporated herein by reference in its entirety and for all purposes. The polynucleotides above may also optionally encode an N-terminal methionyl residue. Non-peptide compounds useful in the present invention may be prepared by art-known methods. For example, phosphate-containing amino acids and peptides containing such amino acids may be prepared using methods known in the art. See, e.g., Bartlett and Landen, 1986, Bioorg. Chem. 14: 356-77.

A variety of expression vector/host systems may be utilized to contain and express a engineered polypeptide coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Exemplary protocols for the recombinant expression of the protein are described herein and/or are known in the art.

As such, polynucleotide sequences are useful in generating new and useful viral and plasmid DNA vectors, new and useful transformed and transfected procaryotic and eucaryotic host cells (including bacterial, yeast, and mammalian cells grown in culture), and new and useful methods for cultured growth of such host cells capable of expression of the present engineered polypeptides. The polynucleotide sequences encoding engineered polypeptides herein may be useful for gene therapy in instances where underproduction of engineered polypeptides would be alleviated, or the need for increased levels of such would be met.

The present invention also provides for processes for recombinant DNA production of the present engineered polypeptides. Provided is a process for producing the engineered polypeptides from a host cell containing nucleic acids encoding the engineered polypeptide including: (a) culturing the host cell containing polynucleotides encoding the engineered polypeptide under conditions facilitating the expression of the DNA molecule; and (b) obtaining the engineered polypeptides.

Host cells may be prokaryotic or eukaryotic and include bacteria, mammalian cells (such as Chinese Hamster Ovary (CHO) cells, monkey cells, baby hamster kidney cells, cancer cells or other cells), yeast cells, and insect cells.

Mammalian host systems for the expression of the recombinant protein also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing, which cleaves a “prepro” form of the protein, may also be important for correct insertion, folding and/or function. Different host cells, such as CHO, HeLa, MDCK, 293, W138, and the like, have specific cellular machinery and characteristic mechanisms for such post-translational activities, and may be chosen to ensure the correct modification and processing of the introduced foreign protein.

Alternatively, a yeast system may be employed to generate the engineered polypeptides of the present invention. The coding region of the engineered polypeptides DNA is amplified by PCR. A DNA encoding the yeast pre-pro-alpha leader sequence is amplified from yeast genomic DNA in a PCR reaction using one primer containing nucleotides 1-20 of the alpha mating factor gene and another primer complementary to nucleotides 255-235 of this gene (Kurjan and Herskowitz, 1982, Cell, 30: 933-43). The pre-pro-alpha leader coding sequence and engineered polypeptide coding sequence fragments are ligated into a plasmid containing the yeast alcohol dehydrogenase (ADH2) promoter, such that the promoter directs expression of a fusion protein consisting of the pre-pro-alpha factor fused to the mature engineered polypeptide. As taught by Rose and Broach, (Rose & Broach, 1990, Meth. Enz., 185: 234-79, Goeddel ed., Academic Press, Inc., San Diego, Calif.), the vector further includes an ADH2 transcription terminator downstream of the cloning site, the yeast “2-micron” replication origin, the yeast leu-2d gene, the yeast REP1 and REP2 genes, the E. coli beta-lactamase gene, and an E. coli origin of replication. The beta-lactamase and leu-2d genes provide for selection in bacteria and yeast, respectively. The leu-2d gene also facilitates increased copy number of the plasmid in yeast to induce higher levels of expression. The REP1 and REP2 genes encode proteins involved in regulation of the plasmid copy number.

The DNA construct described in the preceding paragraph is transformed into yeast cells using a known method, e.g., lithium acetate treatment (Steams et al., 1990, Meth. Enz. 185: 280-297). The ADH2 promoter is induced upon exhaustion of glucose in the growth media (Price et al., 1987, Gene 55:287). The pre-pro-alpha sequence effects secretion of the fusion protein from the cells. Concomitantly, the yeast KEX2 protein cleaves the pre-pro sequence from the mature engineered polypeptides (Bitter et al., 1984, Proc. Natl. Acad. Sci. USA 81:5330-5334).

Engineered polypeptides of the invention may also be recombinantly expressed in yeast, e.g. Pichia, using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted engineered polypeptide is purified from the yeast growth medium by, e.g., the methods used to purify said engineered polypeptide from bacterial and mammalian cell supernatants.

Alternatively, the DNA encoding a engineered polypeptide may be cloned into a baculovirus expression vector, e.g. pVL1393 (PharMingen, San Diego, Calif.). This engineered-polypeptide-encoding vector is then used according to the manufacturer's directions (PharMingen) or known techniques to infect Spodoptera frugiperda cells, grown for example in sF9 protein-free media, and to produce recombinant protein. The protein is purified and concentrated from the media using methods known in the art, e.g. a heparin-Sepharose column (Pharmacia, Piscataway, N.J.) and sequential molecular sizing columns (Amicon, Beverly, Mass.), and resuspended in appropriate solution, e.g. PBS. SDS-PAGE analysis can be used to characterize the protein, for example by showing a single band that confirms the size of the desired engineered polypeptide, as can full amino acid amino acid sequence analysis, e.g. Edman sequencing on a Proton 2090 Peptide Sequencer, or confirmation of its N-terminal sequence.

For example, the DNA sequence encoding the predicted mature engineered polypeptide may be cloned into a plasmid containing a desired promoter and, optionally, a leader sequence (see, e.g., Better et al., 1988, Science 240:1041-1043). The sequence of this construct may be confirmed by automated sequencing. The plasmid is then transformed into E. coli, strain MC1061, using standard procedures employing CaCl2 incubation and heat shock treatment of the bacteria (Sambrook et al., Id.). The transformed bacteria are grown in LB medium supplemented with carbenicillin, and production of the expressed protein is induced by growth in a suitable medium. If present, the leader sequence will affect secretion of the mature engineered polypeptide and be cleaved during secretion. The secreted recombinant engineered polypeptide is purified from the bacterial culture media by the method described herein.

Alternatively, the engineered polypeptides may be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The engineered polypeptide coding sequence is cloned into a nonessential region of the virus, such as the polyhedrin gene, and placed under control of the polyhedrin promoter. Successful insertion of a engineered polypeptide will render the polyhedrin gene inactive and produce recombinant virus lacking coat protein coat. The recombinant viruses are then used to infect S. frugiperda cells or Trichoplusia larvae in which engineered polypeptide of the present invention is expressed (Smith et al.,1983, J. Virol. 46:584; Engelhard et al., 1994, Proc. Natl. Acad. Sci. USA 91:3224-3227).

In another example, the DNA sequence encoding the engineered polypeptides may be amplified by PCR and cloned into an appropriate vector, for example, pGEX-3X (Pharmacia, Piscataway, N.J.). The pGEX vector is designed to produce a fusion protein including glutathione-S-transferase (GST), encoded by the vector, and a protein encoded by a DNA fragment inserted into the vector's cloning site. The primers for the PCR may be generated to include, for example, an appropriate cleavage site. The recombinant fusion protein may then be cleaved from the GST portion of the fusion protein. The pGEX-3X/engineered polypeptide construct is transformed into E. coli XL-1 Blue cells (Stratagene, La Jolla, Calif.), and individual transformants are isolated and grown at 37 degrees C. in LB medium (supplemented with carbenicillin) to an optical density at wavelength 600 nm of 0.4, followed by further incubation for 4 hours in the presence of 0.5 mM Isopropyl beta-D-Thiogalactopyranoside (Sigma Chemical Co., St. Louis, Mo.). Plasmid DNA from individual transformants is purified and partially sequenced using an automated sequencer to confirm the presence of the desired engineered polypeptide-encoding gene insert in the proper orientation.

The fusion protein, when expected to be produced as an insoluble inclusion body in the bacteria, may be purified as described above or as follows. Cells are harvested by centrifugation; washed in 0.15 M NaCl, 10 mM Tris, pH 8, 1 mM EDTA; and treated with 0.1 mg/mL lysozyme (Sigma Chemical Co.) for 15 min. at RT. The lysate is cleared by sonication, and cell debris is pelleted by centrifugation for 10 min. at 12,000×g. The fusion protein-containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg and Ca The fusion protein is further purified by fractionating the resuspended pellet in a denaturing SDS polyacrylamide gel (Sambrook et al., supra). The gel is soaked in 0.4 M KCl to visualize the protein, which is excised and electrocluted in gel-running buffer lacking SDS. If the GST/engineered polypeptide fusion protein is produced in bacteria as a soluble protein, it may be purified using the GST Purification Module (Pharmacia Biotech).

The fusion protein may be subjected to digestion to cleave the GST from the mature engineered polypeptide. The digestion reaction (20-40 μg fusion protein, 20-30 units human thrombin (4000 U/mg (Sigma) in 0.5 mL PBS) is incubated 16-48 hrs. at RT and loaded on a denaturing SDS-PAGE gel to fractionate the reaction products. The gel is soaked in 0.4 M KCl to visualize the protein bands. The identity of the protein band corresponding to the expected molecular weight of the engineered polypeptide may be confirmed by partial amino acid sequence analysis using an automated sequencer (Applied Biosystems Model 473A, Foster City, Calif.).

In a particularly exemplary method of recombinant expression of the engineered polypeptides of the present invention, mammalian 293 cells may be co-transfected with plasmids containing the engineered polypeptides cDNA in the pCMV vector (5′ CMV promoter, 3′ HGH poly A sequence) and pSV2neo (containing the neo resistance gene) by the calcium phosphate method. In one embodiment, the vectors should be linearized with Seal prior to transfection. Similarly, an alternative construct using a similar pCMV vector with the neo gene incorporated can be used. Stable cell lines are selected from single cell clones by limiting dilution in growth media containing 0.5 mg/mL G418 (neomycin-like antibiotic) for 10-14 days. Cell lines are screened for engineered polypeptides expression by ELISA or Western blot, and high-expressing cell lines are expanded for large scale growth.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection, and its presence allows growth and recovery of cells that successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside, G418; also, that confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, beta-glucuronidase and its substrate, GUS, and luciferase and its substrate, luciferin.

The engineered polypeptides of the present invention may be produced using a combination of both automated peptide synthesis and recombinant techniques. For example, either or both the exendin compound and the ABD, and optionally a linker, can be made synthetically or recombinantly and then ligated together using methods known in the art, such as “native chemical ligation” and known variations thereof in which an amide bond is formed joining the parent compounds. See, e.g., U.S. Pat. No. 6,326,468, which is incorporated herein by reference and for all purposes. Alternatively, for example, an engineered polypeptide of the present invention may contain a combination of modifications including deletion, substitution, insertion and derivatization by PEGylation (or other moiety, e.g. polymer, fatty acyl chain, C-terminal amidation). Such an engineered polypeptide may be produced in stages. In the first stage, an intermediate engineered polypeptide containing the modifications of deletion, substitution, insertion, and any combination thereof, may be produced by recombinant techniques as described. Then after an optional purification step as described herein, the intermediate engineered polypeptide is PEGylated (or subjected to other chemical derivatization, e.g., acylation, C-terminal amidation) through chemical modification with an appropriate PEGylating reagent (e.g., from NeKtar Transforming Therapeutics, San Carlos, Calif.) to yield the desired engineered polypeptide derivative. One skilled in the art will appreciate that the above-described procedure may be generalized to apply to a engineered polypeptide containing a combination of modifications selected from deletion, substitution, insertion, derivation, and other means of modification well known in the art and contemplated by the present invention.

C-terminal amidation can be achieved by use of a glycine amino acid-C-terminally extended precursor, synthesized for example in yeast (e.g. Pichia) as alpha-factor fusion protein that will be secreted into culture medium. After purification, the C-terminal glycine of the engineered polypeptide precursor can be converted to amide by enzymatic amidation, e.g. peptidylglycine alpha-amidating monooxygenase (PAM). See e.g., Cooper et al., 1989, Biochem. Biophys. Acta, 1014:247-258. See also U.S. Pat. No. 6,319,685, which is incorporated herein by reference in its entirety and for all purposes, which teaches methods for enzymatic amidation, including an alpha-amidating enzyme from rat being sufficiently pure in alpha-amidating enzyme to exhibit a specific activity of at least about 25 mU per mg of protein, and being sufficiently free of proteolytic impurities to be suitable for use with substrates purified from natural sources or produced by recombinant DNA techniques.

Peptides may be purified by any number of methods known in the art, including as described herein In one method peptides are purified by RP-HPLC (preparative and analytical) using a Waters Delta Prep 3000 system. A C4, C8 or C18 preparative column (10 micron, 2.2×25 cm; Vydac, Hesperia, Calif.) may be used to isolate peptides, and purity may be determined using a C4, C8 or C18 analytical column (5 micron, 0.46×25 cm; Vydac). Solvents (A=0.1% TFA/water and B=0.1% TFA/CH₃CN) may be delivered to the analytical column at a flow rate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen et al, THE PICO TAG METHOD: A MANUAL OF ADVANCED TECHNIQUES FOR AMINO ACID ANALYSIS, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ton 20 mass spectrometer.

Engineered polypeptide expression assay. Methods are available for assaying the level of protein expression by a host cell. Procedures useful for assaying the level of protein expression by a host cell are exemplified in the following typical protocol. About 25 ul BL21 E. coli cells are transformed with 2 ul plasmid DNA (expression vector for the engineered polynucleotide). Cells can be plated and incubated overnight at 37 degrees C. or at room temperature (RT) over a 48-hr period. A single colony can be selected and used to grow starter culture in 4 ml LB media with appropriate antibiotic for ˜6 hrs. Glycerol stocks call be prepared by adding 100 ul 80% sterile glycerol to 900 ul stock, which can then be mixed gently and stored at −80 C. A 250 ul sample can be removed for TCP uninduced sample. An aliquot, for example, 2 ml of Magic media containing appropriate antibiotic can be inoculated with 5 ul starter culture, which can then be incubated overnight (up to 24 hrs) at 37 C, 300 rpm. As known in the art, Magic Media is autoinducing. Alternatively, 60 ml Magic Media containing appropriate antibiotic can be inoculated with 60 ul starter culture in a 250 ml or 125 ml Thompson flask, which can then be incubated overnight (up to 24 hrs) at 30 C, 300 rpm. After incubation, 250 ul culture can be removed from each tube and the cells pelleted. The cell can be resuspended in 1 ml 50 mM Tris pH 8, 150mM NaCl, to which can be added 0.1 volumes (100 ul) POP culture reagent and 1 ul r-lysozyme (1:750 dilution in r-lysozyme buffer). The mixture can be mixed well and incubated at least 10 min at RT. The preparation can then be centrifuge 10 min at 14000×G. The supernatant (soluble fraction) can be removed and retained, and samples can be prepared for gel analysis (15 ul 5 ul LDS). The remaining inclusion body pellet can be resuspended in lml 1% SDS with sonication. The sample can be prepared for gel analysis (15 ul+5 ul LDS). For uninduced samples, 1.0 volumes POP culture reagent and 1 ul r-lysozyme (1:750 dilution in r-lysozyme buffer) can be added. The mixture can be mixed well and incubated at least 10 min at RT. These samples may not need to be centrifuged. The sample can then be prepared for gel analysis (15 ul +5 ul LDS). NU-PAGE gels (4-12%) non-reduced in 1×MES buffer can be run and stained with SimplyBlue microwave protocol. Destaining can be conducted overnight, as known in the art. A gel image can be retained, and analyzed to determine protein expression levels.

Engineered polypeptides can be and were expressed and isolated as follows. A protein sequence of the desired engineered polypeptide was designed and back translated using commercial software to a DNA sequence for cloning into an E. coli expression vector. Nucleic acid sequences were either obtained as oligonucleotides and ligated using standard PCR amplification techniques, or were digested from existing expression constructs using standard restriction enzymes and then ligated together. Sequences expressing the protein of interest were placed in plasmid pET45 with a T7 promoter for inducible expression. After constructs were verified by sequencing, the vector DNA was purified and transformed into an expression host, typically BL21(DE3). A single colony was selected to grow a starter culture in 4 ml LB media for ˜6 hrs. Glycerol stocks were prepared by adding 100 ul 80% glycerol to 900 ul stock and stored at −80 C. Optionally, 500 ul of un-induced sample was retained for gel analysis. A 60 ml culture (e.g. MagicMedia™ E. coli Expression Medium; Invitrogen, USA; see Glenn et al., J. Biol. Chem. 2008, 283(19):12717-29) was inoculated using 60 ul starter culture in a 125 ml Thompson flask and incubated at 30 degrees C. overnight. Removed 250 ul sample for analysis. The cells were collected as a pellet by centrifuging, and frozen for later processing. Preparation of cell extract and first pass purification with Nickel resin was performed as follows. E. coli cell pellets were completely resuspended in a volume of lysis buffer (50 mM TrisHCl, 150 mM NaCl, pH 8.0) equal to the starting culture volume. Cells were then subjected to a microfluidizer (Microfluidics, MA) at 100 psi for three times. Cell extracts were centrifuged for 30 minutes at 16,000×g to remove debris. EGTA (150 mM stock) was added to the cell extract to a final concentration of 3 mM EGTA. The lysate was then applied to a Ni-NTA Superflow column that had been washed and pre-equilibrated. Protein bound to the column was then washed with lysis buffer plus EGTA (50 mM TrisHCl, 150 mM NaCl, pH8.0, 3 mM EGTA) before the bound protein was eluted with 50 mL of elution buffer (25 mM TrisHCl, 50 mM NaCl, 250 mM Imidazol, pH8.0). Cleavage of His-Tag and subsequent purification was as follows. The eluted protein was concentrated with Amicon-Ultra15 centrifugal filter unit (Millipore, USA) and then diluted with 25 mM TrisHCl, pH8.0, 50 mM NaCl to prepare for protease digestion which removes the HisTag from the N-terminus of the desired protein. Added was 0.1% of β-mercaptoethanol and 1% of Turbo TEV protease (2 mg/mL, 10,000 units/mg; Excellgen, USA) to the protein solution, which was mixed and incubated at room temperature for 4 hours and then at 4° C. over night. An Ni-NTA Superflow column (Qiagen, USA) was pre-equilibrated with 50 mM TrisHCl, 100 mM NaCl, 45 mM imidazole, pH8.0. The TEV digest reaction was diluted 2-fold with 50 mM TrisHCl, 150 mM NaCl, pH8.0. The diluted digest reaction was carefully applied to the top of Ni-NTA column and flow-through was collected. To the column was added 10 mL of 50 mM trisHCl, 100 mM NaCl, 45 mM imidazole, pH8.0 to elute any unbound protein. The eluted proteins from the column were collected and combined, and then polished using size exclusion chromatography (2× with Superdex 75 HiLoad 26/60 column; GE Healthcare Biosciences, USA). Any remaining bacterial endotoxin was removed using EndoTrap Red (Lonza, Switzerland) according to manufacturer's instructions.

Inclusion Body preparation. For engineered polypeptides that are found in the inclusion body fraction, the following procedure can be beneficial. The cell pellet can be resuspended in a minimum of 100 ml Lysis buffer for each 50 ml culture. Upon the addition of 30 ml, a 10 ml pipette can be used to resuspend, then the tube can be washed out with an additional 70 ml. The resuspended cell solution can be multiply run, e.g., 4 passes, through a microfluidizer(a), 100 PSI (min) taking care to keep chamber in ice water through the entire process. The fluidized slurry can be centrifuged at 14000×g, 20 min (e.g., JLA 10.5, 10,000 rpm, using 250 ml nalgene bottles). The inclusion body pellet can be resuspended on ice in chilled lysis buffer with stir bar and stir plate for 1 hour at 4 C after disruption with pipette tip. The pellet can be resuspended a second time in distilled H₂O with stir bar and stir plate for 1 hour at 4 C after disruption with pipette tip, followed by centrifugation at 14000×g, 15 min. The supernatant can be removed and discarded. The resultant can be stored at −80 C.

Protein purification. As described herein, numerous methods are known for isolation of expressed polypeptides. Preferred are secreted engineered polypeptides. However, the following is one example if inclusion bodies are formed. Inclusion body pellets can be solubilized in appropriate volume of solubilization buffer (8M urea or 8M guanidine, 50 mM Tris, 10 mM DTT, pH 7.75) for 1 hour at RT. The solubilized pellets can be centrifuged for 20 min at 27 000 g. Filtered (e.g., 0.4 um) supernatant can be transferred drop by drop into appropriate volume of refolding buffer (50 mM Tris-HCl, 1 M urea, 0.8 M arginine, 4 mM cysteine, 1 mM cystamine; pH 8) at RT. The result can then be placed at 4° C. overnight or longer with gentle mixing. Samples can be concentrated and run on a gel filtration column (Superdex75 26/60) at 1-2 ml/min in 4 C environment using a GE Healthsciences AKTA FPLC. Appropriate protein containing fractions can be identified via SDS-PAGE, pooled and run through a second gel filtration column. Pooled protein can then be concentrated in Amicon filter to appropriate concentration and assayed for endotoxin levels using, e.g., Endosafe PTS Reader (Charles River), as known in the art. Once a protein sample has passed the endotoxin criteria, it can be sterile filtered, dispensed into aliquots and run through quality control assays. Quality control assays can include analytical HPLC-SEC, non reducing SDS PAGE and RP HPLC—MS to obtain approximate mass. Proteins can be obtained in 1×PBS (137 mM sodium chloride, 2.7 mM potassium chloride, 4.3 mM disodium phosphate, 1.4 mM monopotassium phosphate, pH7.2), distributed into aliquots and flash frozen for storage at −70 to −80° C.

### IV. METHODS OF USE AND TREATING DISEASE

Indications. A variety of diseases and disorders are contemplated to be beneficially treated by the polypeptide compounds and methods described herein, primarily based upon those amenable to treatment by interaction with the GLP-1 receptor, such as by exendin-4.

Obesity and overweight. Obesity and its associated disorders including overweight arc common and serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. See, e.g., Kopelman, 2000, Nature 404:635-43.

Obesity reduces life-span and carries a serious risk of the co-morbidities listed above, as well disorders such as infections, varicose veins, acanthosis nigricans, eczema, exercise intolerance, insulin resistance, hypertension hypercholesterolemia, cholelithiasis, orthopedic injury, and thromboembolic disease. See e.g., Rissanen et al, 1990, Br. Med. J., 301:835-7. Obesity is also a risk factor for the group of conditions called insulin resistance syndrome, or “Syndrome X” and metabolic syndrome. The worldwide medical cost of obesity and associated disorders is enormous.

The pathogenesis of obesity is believed to be multi-factoral. A problem is that, in obese subjects, nutrient availability and energy expenditure do not come into balance until there is excess adipose tissue. The central nervous system (CNS) controls energy balance and coordinates a variety of behavioral, autonomic and endocrine activities appropriate to the metabolic status of the animal. The mechanisms or systems that control these activities arc broadly distributed across the forebrain (e.g., hypothalamus), hindbrain (e.g., brainstem), and spinal cord. Ultimately, metabolic (i.e., fuel availability) and cognitive (i.e., learned preferences) information from these systems is integrated and the decision to engage in appetitive (food seeking) and consummatory (ingestion) behaviors is either turned on (meal procurement and initiation) or turned off (meal termination). The hypothalamus is thought to be principally responsible for integrating these signals and then issuing commands to the brainstem. Brainstem nuclei that control the elements of the consummatory motor control system (e.g., muscles responsible for chewing and swallowing). As such, these CNS nuclei have literally been referred to as constituting the “final common pathway” for ingestive behavior.

Neuroanatomical and pharmacological evidence support that signals of energy and nutritional homeostasis integrate in forebrain nuclei and that the consummatory motor control system resides in brainstem nuclei, probably in regions surrounding the trigeminal motor nucleus. There are extensive reciprocal connection between the hypothalamus and brainstem. A variety of CNS-directed anti-obesity therapeutics (e.g., small molecules and peptides) focus predominantly upon forebrain substrates residing in the hypothalamus and/or upon hindbrain substrates residing in the brainstem.

Obesity remains a poorly treatable, chronic, essentially intractable metabolic disorder. Accordingly, a need exists for new therapies useful in weight reduction and/or weight maintenance in a subject. Such therapies would lead to a profound beneficial effect on the subject's health.

Diabetes and cardiovascular disease. Diabetes mellitus is recognized as a complex, chronic disease in which 60% to 70% of all case fatalities among diabetic patients are a result of cardiovascular complications. Diabetes is not only considered a coronary heart disease risk equivalent but is also identified as an independent predictor of adverse events, including recurrent myocardial infarction, congestive heart failure, and death following a cardiovascular incident. The adoption of tighter glucose control and aggressive treatment for cardiovascular risk factors would be expected to reduce the risk of coronary heart disease complications and improve overall survival among diabetic patients. Yet, diabetic patients are two to three times more likely to experience an acute myocardial infarction than non-diabetic patients, and diabetic patients live eight to thirteen years less than non-diabetic patients.

Understanding the high risk nature of diabetic/acute myocardial infarction patients, the American College of Cardiology/American Heart Association (“ACC/AHA”) clinical practice guidelines for the management of hospitalized patients with unstable angina or non-ST-elevation myocardial infarction (collectively referred to as “ACS”) recently recognized that hospitalized diabetic patients are a special population requiring aggressive management of hyperglycemia. Specifically, the guidelines state that glucose-lowering therapy for hospitalized diabetic/ACS patients should be targeted to achieve preprandial glucose less than 10 mg/dL, a maximum daily target than 180 mg/dL, and a post-discharge hemoglobin A1c less than 7%.

In a nationwide sample of elderly ACS patients, it was demonstrated that an increase in 30-day mortality in diabetic patients corresponded with the patients having higher glucose values upon admission to the hospital. See “Diabetic Coronary Artery Disease & Intervention,” Coronary Therapeutics 2002, Oak Brook, Ill., Sep. 20, 2002. There is increasing evidence that sustained hyperglycemia rather than transient elevated glucose upon hospital admission is related to serious adverse events. Although the ideal metric for hyperglycemia and vascular risk in patients is not readily known, it appears that the mean glucose value during hospitalization is most predictive of mortality. In a separate study of ACS patients form over forty hospitals in the United States, it was found that persistent hyperglycemia, as opposed to random glucose values upon admission to the hospital, was more predictive of in-hospital mortality. See Acute Coronary Syndrome Summit: A State of the Art Approach, Kansas City, Mo., Sep. 21, 2002. Compared with glucose values upon admission, a logistic regression model of glucose control over the entire hospitalization was most predictive of mortality. There was nearly a two-fold increased risk of mortality during hospitalization for each 10 mg/dL increase in glucose over 120 mg/dL. In a smaller cohort of consecutive diabetic/ACS patients, there was a graded increase in mortality at one year with increasing glucose levels upon hospital admission. In the hospital setting, the ACC/AHA guidelines suggest initiation of aggressive insulin therapy to achieve lower blood glucose during hospitalization.

Lipid regulation diseases. Dyslipidemia is a disruption in the normal lipid component in the blood. It is believed that prolonged elevation of insulin levels can lead to dyslipidemia. Hyperlipidemia is the presence of raised or abnormal levels of lipids and/or lipoproteins in the blood. Fatty liver disease, e.g., nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes).

Additionally, without wishing to be bound by any theory, it is believed that relative insulin deficiency in type 2 diabetes, glucose toxicity, and increased hepatic free fatty acid burden through elevated delivery from intra-abdominal adipose tissue via the portal vein, are implicated as possible causes in fatty liver disorders. Indeed, it has been hypothesized that eating behavior is the key factor driving the metabolic syndrome of obesity with its many corollaries, including NASH. Accordingly, treatments aimed at decreasing food intake and increasing the number of small meals, as has already been demonstrated in type 2 diabetes, may effectively treat and prevent NASH. Drugs that promote insulin secretion and weight loss, and delay gastric emptying are also effective at improving glucose tolerance and thus may improve fatty liver with its attendant hyperinsulinemia. Thus, use of exendins, exendin analog agonists, exendin derivative agonists, particularly exendin-4, can be well suited as a treatment modality for this condition. Accordingly, engineered polypeptides described herein which include an exendin or biologically active (hormone domain) peptide component, or fragment or analog thereof, can be useful in the treatment of fatty liver disorders.

Alzheimer's disease. Alzheimer's disease (AD), as known in the art, is associated with plaques and tangles in the brain which include dysregulation of the A-beta protein. Stimulation of neuronal GLP-1 receptors has been reported to play an important role in regulating neuronal plasticity and cell survival. Stimulation of GLP-1 receptor has been reported to induce neurite outgrowth and to protect against excitotoxic cell death and oxidative injury in cultured neuronal cells. GLP-1 and exendin-4 were reported to reduce endogenous levels of amyloid-beta peptide (A-beta protein) in mouse brain and to reduce levels of beta-amyloid precursor protein (beta-APP) in neurons. See, e.g., Perry et al., 2004, Curr. Drug Targets 5(6):565-571. Treatment with the engineered compounds disclosed herein can provide benefit to the therapeutic targets associated with Alzheimer's disease.

Parkinson's disease. Parkinson's disease (PD) is the synonym of “primary parkinsonism”, i.e. isolated parkinsonism due to a neurodegenerative process without any secondary systemic cause. Parkinsonism is characterized by symptoms of tremor, stiffness, and slowing of movement caused by loss of dopamine. Without wishing to be bound by any theory, it is believed that exendin-4 may act as a survival factor for dopaminergic neurons by functioning as a microglia-deactivating factor and suggest that exendin-4 may be a valuable therapeutic agent for neurodegenerative diseases such as PD.

Metabolic syndrome X. Metabolic Syndrome X is characterized by insulin resistance, dyslipidemia, hypertension, and visceral distribution of adipose tissue, and plays a pivotal role in the pathophysiology of type 2 diabetes. It has also been found to be strongly correlated with NASH, fibrosis, and cirrhosis of the liver. Accordingly, engineered polypeptides described herein can be useful in the treatment of metabolic syndrome X.

Steroid induced diabetes. Glucocorticoids are well known to affect carbohydrate metabolism. In response to exogenous glucocorticoid administration, increased hepatic glucose production and reduced insulin secretion and insulin-stimulated glucose uptake in peripheral tissues is observed. Furthermore, glucocorticoid treatment alters the proinsulin(P1)/immunoreactive insulin(IRI) ratio, as known in the art. Typical characteristics of the hyperglycemia induced by glucocorticoids in subjects without diabetes include a minimal elevation of fasting blood glucose, exaggerated postprandial hyperglycemia, insensitivity to exogenous insulin, and non-responsiveness to metformin or sulfonylurea therapy. Accordingly, engineered polypeptides described herein which include an exendin biologically active (hormone domain) peptide component, or fragment or analog thereof, can be useful in the treatment of steroid induced diabetes.

Human Immunodeficiency Virus (HIV) Treatment-Induced Diabetes. Shortly after the introduction of human immunodeficiency virus (HIV)-1 protease inhibitors (PIs) into routine clinical use, reports linking PT use with the development of hyperglycemia began to appear. While approximately 1% to 6% of HIV-infected subjects who are treated with PIs will develop diabetes mellitus, a considerably larger proportion will develop insulin resistance and impaired glucose tolerance. Accordingly, engineered polypeptides described herein which include an exendin biologically active (hormone domain) peptide component, or fragment or analog thereof, can be useful in the treatment of HIV treatment-induced diabetes.

Latent Autoimmune Diabetes in Adults (LADA). Progressive autoimmune diabetes, also known as latent autoimmune diabetes in adults (LADA), is thought to be present in approximately 10% of patients diagnosed with type 2 diabetes. LADA patients have circulating antibodies to either islet cell cytoplasmic antigen or, more frequently, glutamic acid decarboxylase. These subjects exhibit clinical features characteristic of both type 1 and type 2 diabetes. Although insulin secretion is better preserved in the slowly progressing than in the rapidly progressing form of autoimmune diabetes, insulin secretion tends to deteriorate with time in LADA subjects. Accordingly, engineered polypeptides described herein which include an exendin biologically active (hormone domain) peptide component, or fragment or analog thereof, can be useful in the treatment of LADA.

Hypoglycemia Unawareness (HU). Defective glucose counterregulation can occur even after only a single recent episode of hypoglycemia. Subjects who experience repeated episodes of hypoglycemia often lose their capacity to recognize the symptoms typically associated with hypoglycemia or impending insulin shock, a condition called “hypoglycemia unawareness”. Because the-patient doesn't appreciate his or her own status, blood glucose levels can then fall so low that serious neurological problems ensue, including coma and seizure. Accordingly, engineered polypeptides described herein which include an exendin biologically active (hormone domain) peptide component, or fragment or analog thereof, can be useful in the treatment of HU.

Restrictive Lung Disease. GLP 1 receptor has been localized in the lung. Exendins can elicit a biological response via GLP-1 receptor. In particular, sarcoidosis is a systemic granulomatous disease that frequently involves the lung. Although classically thought of as a restrictive lung disease, airway obstruction has become a recognized feature of the disease in the past years. Sarcoidosis can affect the airway at any level and when the involvement includes small airways, it can resemble more common obstructive airway diseases, such as asthma and chronic bronchitis. Accordingly, engineered polypeptides described herein which include an exendin biologically active (hormone domain) peptide component, or fragment or analog thereof, can be useful in the treatment of restrictive lung disease because such hormone domain peptide can improve elasticity of lung or delay rigidity.

Short Bowel Syndrome (SBS). Exendin-4 has been reported as effective for the treatment of short bowel syndrome. See Kunkel et al. Neurogastroenterol. Motil. (2011). SBS is a serious clinical disorder characterized by diarrhea and nutritional deprivation. Glucagon-like peptide-1 (GLP-1), produced by L-cells in the ileum, regulates proximal gut transit. When extensive ileal resection occurs, as in SBS, GLP-1 levels may be deficient. Exenatide improved the nutritional state and intestinal symptoms of patients with SBS. Accordingly, SBS patients are amenable to treatment with the engineered polypeptides described herein. Improvement in bowel frequency and form and obtaining bowel movements that are no longer meal-related can be achieved. An additional benefit is that total parenteral nutrition can be stopped. These compounds herein will provide substantial improvement in the bowel habits, nutritional status and quality of life of SBS patients, and further may reduce the need for parenteral nutrition and small bowel transplant.

Accordingly, in one aspect, there is provided a method for treating a disease or disorder in a subject. The subject is in need of treatment for the disease or disorder. In some embodiments, the subject is need of treatment is obese. The disease or disorder is diabetes, overweight, obesity, Alzheimer's disease, fatty liver disease, dyslipidemia, coronary artery disease, stroke, SBS or hyperlipidemia, or other diseases discussed herein. Diabetes can include type I, type II, gestational or pre-diabetes as well as HIV or steroid induced diabetes. The method of treatment includes administration to the subject of a engineered polypeptide as described herein in an amount effective to treatment the disease or disorder. Particularly useful for these diseases are compounds described herein having glucose lowering activity (e.g. exendin-4 or its fragments or analogs linked to an ABD), having reduction of body weight or reduction of food intake activity, lowering of HbA1c, delaying of gastric emptying, lowering of plasma glucagon, and/or intestinal motility benefit.

In some embodiments, the disease or disorder is diabetes, overweight or obesity, or dyslipidemia or hyperlipidemia. The engineered polypeptide can include ABD and HD1 polypeptides, and optionally a linker K1, where HD1 is an exendin or fragment or analog thereof. Accordingly, the engineered polypeptide can have one of the following structures: HD1-ABD or HD1-L1-ABD. In some embodiments, the exendin is preferably exendin-4 or Leu14 exendin-4.

In some embodiments, the disease or disorder is diabetes, overweight, obesity, dyslipidemia, Alzheimer's disease, fatty liver disease, SBS or hyperlipidemia. The engineered polypeptide may include an exendin or fragment or analog thereof. Accordingly, the engineered polypeptide can have one of the following structures: HD1-ABD or HD1-L1-ABD. In some embodiments, the exendin in the engineered polypeptide is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, lowering of HbA1c, delaying of gastric emptying, lowering of plasma glucagon, or intestinal motility benefit.

In some embodiments, the disease or disorder is diabetes, overweight, obesity, dyslipidemia, Alzheimer's disease, fatty liver disease, SBS or hyperlipidemia. The engineered polypeptide may include an exendin or fragment or analog thereof. Accordingly, the engineered polypeptide can have one of the following structures: HD1 ABD or HD1 L1 ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, delaying of gastric emptying, lowering of plasma glucagon, or intestinal motility benefit.

The disease or disorder can be diabetes, overweight, obesity, dyslipidemia, Alzheimer's disease, fatty liver disease, SBS, hyperlipidemia, Parkinson's disease or cardiovascular disease or other diseases described herein. The engineered polypeptide may include an exendin or fragment or analog thereof. Accordingly, the engineered polypeptide can have one of the following structures: HD1 ABD or HD1 L1 ABD. In some embodiments, the exendin preferably is exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, a lowering of HbA1c, delaying of gastric emptying, lowering of plasma glucagon, or intestinal motility benefit.

Additional diseases and disorders which can be treated by the compounds and methods described herein include steroid-induced diabetes, HIV treatment-induced diabetes, latent autoimmune diabetes in adults (LADA), Nonalcoholic steatohepatitis (NASH) and nonalcoholic fatty liver disease (NAFLD), hypoglycemia unawareness (HU), restrictive lung disease including sarcoidosis, and metabolic syndrome X. The engineered polypeptide may include an exendin or fragment or analog thereof. Accordingly, the engineered polypeptide can have one of the following structures: HD1-ABD or HD1-L1-ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, delaying of gastric emptying, lowering of HbA lc, lowering of plasma glucagon, or intestinal motility benefit. The engineered polypeptide can include only exendin, or analog or fragment thereof, as a hormone domain. The disease or disorder can be diabetes, overweight, obesity, dyslipidemia, Alzheimer's disease, fatty liver disease, SBS, hyperlipidemia, Parkinson's disease or cardiovascular disease or other diseases described herein. The engineered polypeptide may include an exendin or fragment or analog thereof. Accordingly, the engineered polypeptide can have one of the following structures: HD1-ABD or HD1-L1-ABD. exendin-4 or its fragments or analogs linked to an ABD), having reduction of body weight or reduction of food intake activity, delaying of gastric emptying, lowering of plasma glucagon, or intestinal motility benefit.

Additional diseases and disorders which can be treated by the compounds and methods described herein include steroid-induced diabetes, HIV treatment-induced diabetes, latent autoimmune diabetes in adults (LADA), Nonalcoholic steatohepatitis (NASH) and nonalcoholic fatty liver disease (NAFLD), hypoglycemia unawareness (HU), restrictive lung disease including sarcoidosis, and metabolic syndrome X. The engineered polypeptide preferably has one of the following structures: HD1-analogs linked to an ABD or HD1-L1-ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4.

### V. ASSAYS

Methods for production and assay of engineered polypeptides described herein are generally available to the skilled artisan. Further, specific methods are described herein as well as in the patent publications and other references cited herein, which are incorporated by reference for this additional purpose.

GLP-1 receptor binding and functional assays: GLP-1 receptor binding activity and affinity may be measured in any number of known methods. For example, in one method binding activity is measured using a binding displacement assay in which the receptor source is RINm5F cell membranes, and the ligand is [¹²⁵I]GLP-1 or iodinated exendin(1-39) or iodinated exendin(9-39). Homogenized RINm5F cell membranes are incubated in 20 mM HEPES buffer with 40,000 cpm [¹²⁵I]GLP-1 (or exendin) tracer, and varying concentrations of test compound for 2 hours at 23° C. with constant mixing. Reaction mixtures arc filtered through glass filter pads presoaked with 0.3% PEI solution and rinsed with ice-cold phosphate buffered saline. Bound counts are determined using a scintillation counter. Binding affinities are calculated using GraphPad Prism® software (GraphPad Software, Inc., San Diego, Calif.).

In vitro assays for functional GLP-1 receptor activation can be performed using known methods and cells and tissues. For example, exendin-4 stimulation of GLP-1 receptor bearing cells can induce an increase in adenylate cyclase activation, cAMP synthesis, membrane depolarization, rise in intracellular calcium and increase in glucose-induced insulin secretion. See e.g., Holz et al., 1995, J. Biol. Chem. 270(30):17749-57. Cell-based assays using the rMTC 6-23 (clone 6) cell line can be used to determine GLP-1 receptor agonist activity of a compound based on the cAMP generated. In one embodiment of the bioassay the GLP-1 receptor agonist activity of a compound is quantitatively determined by correlations to cAMP production in cell-based assays with 6-23 (clone 6) cells. The cell-based assay uses living 6-23 (clone 6) cells. The 6-23 (clone 6) cells are available from the American Type Culture Collection as ATCC® No. CRIL-1607™ and the European Collection of Cell Cultures as ECACC No. 87042206. In another embodiment the cell-based assay is a homogeneous time-resolved fluorescence assay (HTRF®). HTRF® kits are commercially available from Cisbio International (Bedford, Mass.). Methods for using HTRF® kits are known in the art and the kits generally include instruction manuals, e.g., on how to prepare samples, standards, calibration curves, and conduct experiments. Homogeneous time-resolved fluorescence cell-based assays are described in U.S. Pat. No. 5,527,684, the disclosure of which is incorporated by reference herein, and Document Reference No. 62AM4PEB rev02 (August 2007) available from Cisbio HTRF® Product Center. See www.htrEcom/products/gper/camp/, the disclosure of which is incorporated by reference herein. In a preferred method the bioassay uses the rat thyroid carcinoma 6-23 (clone 6) cells in a cell-based assay using the HTRF® cAMP dynamic 2 1,000 assay kit, available from Cisbio as Catalog No. 62AM4PEB. The HTRF® standards and calibrations are prepared following the instructions in the kit. Assays may be performed with or without the presence of albumin.

In vivo assays for activity and duration of action and pharmacokinetics can be done using known methods. For example, duration can be performed using an oral glucose tolerance test (OGTT) in which the drug is administered to the subject at a desired time point before the glucose is administered orally(to measure drug duration of action; OGTT DOA) and glucose blood levels are measured (e.g. readily done in mice). Activity and duration can also be measured using an intravenous glucose tolerance test (IVGTT) in which the drug is administered to the subject at a desired time point before the glucose is administered IV (IVGTT DOA) and blood glucose levels are measured (e.g. can readily be done in rats). Preferred engineered compounds have a desired effect on blood glucose of at least 24 hours duration after a single dose of drug, preferably at least 3 days, at least 4 days, at least 5 days, at least 6 days, and at least 1 week after the single dose of drug is given.

For example, test polypeptide is injected subcutaneously at t=0 immediately following a baseline sample into NIH/Swiss female mice. Blood samples are taken at desired time periods such as t=2, 4, and 8 hours during day 1 and then daily through day 5 or through to day 7 or longer. Blood glucose is measured with a OneTouch® Ultra® (LifeScan, Inc., a Johnson & Johnson Company, Milpitas, Calif.). For a duration of activity (DOA) determination, such as for glucose control activity of a drug, an OGTT or IVGTT can be performed at the desired point after drug administration. Body weight can also be measured, as well as food intake, or other pharmacological or pharmacokinetic parameter. For example, female NIH/Swiss mice (8-20 weeks old) are group housed with a 12:12 hour light:dark cycle with lights on at 0600. Water and a standard pelleted mouse chow diet were available ad libitum, except as noted. The morning of the experiment, animals are divided into experimental groups and fasted starting at approximately 0630 hrs. In a typical study, n=2 cages with 3 mice/cage. At time=0 min, a blood glucose sample is taken and immediately followed by an intraperitoneal injection of vehicle or compound in an amount ranging from about 1 nmol/kg to 25 nmol/kg. Blood glucose can be measured at 30, 60, 120, 180, and 240 min and daily for a week or longer after the single dose. In a variation of the experiment, doses are provided daily or even weekly over a longer period such as 14 or 28 days. Percent pre-treatment is calculated by dividing the blood glucose at the measured time point, e.g. 60 minutes or 1 day, by the blood glucose at time=0 min. Significant treatment effects were identified by ANOVA (p<0.05). Where a significant difference exists, test means are compared to the control mean using Dunnett's test (Prism® v. 4.01, GraphPad Software Inc., San Diego, Calif.). Blood glucose can measured with a OneTouch® Ultra® (LifeScan, Inc., a Johnson & Johnson Company, Milpitas, Calif.). *p<0.05 vs. vehicle control; ANOVA, Dunnett's test. Other parameters can also be measured.

In vivo assay for food intake inhibition: The engineered polypeptides may be tested for their duration and extent of appetite suppression and for their duration and extent of effect on body weight loss in various known methods. For example, the polypeptides may be tested for appetite suppression in the mouse food intake assay and for their effect on body weight gain in diet-induced obesity (DIO) mice. An experimental protocol for such assays are described below.

For example, female NIH/Swiss mice (8-24 weeks old) are group housed with a 12:12 hour light: dark cycle with lights on at 0600. Water and a standard pelleted mouse chow diet are available ad libitum, except as noted. Animals are fasted starting at approximately 1500 hrs, 1 day prior to experiment. The morning of the experiment, animals are divided into experimental groups. In a typical study, n=4 cages with 3 mice/cage. At time=0 min, all animals are given an intraperitoneal injection of vehicle or test compound, typically in an amount ranging from about 2 nmol/kg to 75 nmol/kg, and immediately given a pre-weighed amount (10-15 g) of standard chow. Food is removed and weighed at various times, typically 30, 60, and 120 minutes or longer, such as daily, to determine the amount of food consumed (Morley, Flood et al., 1994, Am. J. Physiol. 267: R178-R184). Food intake is calculated by subtracting the weight of the food remaining at the e.g., 30 or 60 minute time point, from the weight of the food provided initially at time=0. Significant treatment effects are identified by ANOVA (p<0.05). Where a significant difference exists, test means are compared to the control mean using Dunnett's test (Prism® v. 2.01, GraphPad Software Inc., San Diego, Calif.). Body weight can also be measured.

Body Weight, fat redistribution, and lean body mass Assays: Assays for body weight and related effects can also be performed as follows. Diet-induced obesity (DIO) in the in the Sprague-Dawley rat is a valuable model for the study of obesity and regulation of energy homeostasis. These rats were developed from a line of (Crl:CD®(SD)BR) rats that are prone to become obese on a diet relatively high in fat and energy. See, for example, Levin, 1994, Am. J. Physiol. 267:R527-R535, Levin et al., 1997, Am. J. Physiol. 273:R725-R730. DIO male rats are obtained from Charles River Laboratories, Inc. (Wilmington, Mass.). The rats are housed individually in shoebox cages at 22° C. in a 12/12-hour light dark cycle. Rats are maintained ad-libitum on a moderately high fat diet (32% kcal from fat; Research Diets D1226B). The animals typically achieve a mean body weight of about 500 g. Levin DIO rats are habituated to caging environment for 7 days. During the 3 nights of habituation, animals receive a single intraperitoneal (IP) injection of vehicle. On test day, rats are administered a single IP injection of compound or vehicle (e.g. 10% DMSO) at the onset of the dark cycle. Food intake is measured by an automated food intake measuring system (BioDAQ, Research Diets) at 5 sec intervals throughout the course of the study. Body weight is recorded nightly.

Body composition can be measured prior to and after drug treatment using NMR (Echo Medical Systems, Houston, Tex.). For body composition measurements, rats are briefly placed (˜1 min) in a well-ventilated plexiglass tube that was then inserted into a specialized rodent NMR machine. This enabled the calculation of changes in actual grams of fat and dry lean tissue (e.g., grams of body fat after treatment—grams of body fat at baseline=change in grams of body fat) and changes in % body composition for fat and dry lean tissue (e.g., % body fat after treatment −% body fat at baseline=change in % body fat). All data are represented as mean±SEM. Analysis of variance (ANOVA) and post-hoc tests are used to test for group difference. A P-value <0.05 is considered significant. Statistical analysis and graphing are performed using PRISM® 4 for Windows (GraphPad Software, Inc., San Diego, Calif.). Graphs and results are typically presented as vehicle-corrected changes in percent body weight, body fat and changes in body protein

### VI. PHARMACEUTICAL COMPOSITIONS

In one aspect, there are provided pharmaceutical compositions including compounds described herein in combination with a pharmaceutically acceptable excipient (e.g., carrier). The term “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (e.g., Ringer's solution and the like), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

In a further aspect, there is provided a pharmaceutical composition which includes a engineered polypeptide as described herein in combination with a pharmaceutically acceptable excipient. In one embodiment, the pharmaceutical composition is an oral pharmaceutical composition, as described herein. In some embodiments, the pharmaceutical composition is a long lasting pharmaceutical composition. The term “long lasting” in the context of administration of a pharmaceutical composition refers to duration of action. Accordingly, a long lasting pharmaceutical composition may be administered at intervals of, for example, 1 hr, 2 hr, 4 hr, 8 hr, 12 hr, 1 day, 2 days, 3 days, 4 days, 5 days, 6 days, 1 week, 2 weeks, 3 weeks, 1 month or even longer. In one embodiment, administration is twice a day (i.e., “twice daily”). In a preferred embodiment, administration is once a day (i.e., “once daily”). In a more preferred embodiments, administration is once a week (i.e., “once weekly”). In some embodiments, the engineered polypeptide is selected from the engineered polypeptides set forth in Tables 2 and 3 herein. In some embodiments, the engineered polypeptide is selected from the engineered polypeptides set forth in Table 2 herein. Preferably the compound is Cmpd 5, 9 or 11, or has at least 95% amino acid sequence identity thereto.

A. Formulations

The engineered polypeptides described herein can be administered alone or can be co-administered to a subject. Co-administration is meant to include simultaneous or sequential administration of the compounds individually or in combination (more than one compound). For example, it has been found that obesity can be beneficially treated with a combination therapy including leptin (e.g., metreleptin) and an amylin (e.g., pramlintide). See e.g., U.S. Published Appl. No. 2008/0207512. Accordingly, an engineered polypeptide described herein including an ABD and an exendin compound useful for treatment of e.g., obesity and overweight, can be administered alone to achieve such treatment or co-administered with either a leptin or leptin agonist, e.g. metreleptin, and/or an amylin or amylin agonist, e.g. pramlintide.

In some embodiments, the formulations and methods described herein further provide that the exendin, exendin analog or exendin analog agonist engineered polypeptide is co-administered with one or more anti-diabetic agents, such as anti-hyperglycemia agents, e.g. insulin (including regular, short acting, long-acting, and basal insulins), amylins, pramlintide, metformin and thiazolidinediones (including rosiglitazone and pioglitazone).

In some embodiments, the formulations and methods described herein further provide that the exendin, exendin analog or exendin analog agonist engineered polypeptide is co-administered with one or more cholesterol and/or triglyceride lowering agents. Exemplary agents include HMG CoA reductase inhibitors (e.g., atorvastatin, fluvastatin, lovastatin, pravastatin, rosuvastatin, simvastatin); bile ace sequestrants (e.g., colesevelam, cholestyramine, colestipol); fibrates (e.g., fenofibrate, clofibrate, gemfibrozil); ezetimibe, nicotinic acid, probucol, a lovastatin/niacin combination; an atorvastatin/amlodipine combination; and a simvastatin/ezetimibe combination.

The present disclosure provides the composition for use as a medicament, i.e. for use in therapy, since the exendin compound is a therapeutically active compound, and surprisingly retains activity when fused to ABD. Compositions including an engineered polypeptide, either liquid or dry form, and optionally at least one pharmaceutically acceptable carrier and/or excipient are also specifically contemplated and are exemplified herein.

The composition has an ability to associate with albumin in vivo or in vitro. In certain cases, it may be of benefit to form a complex of the composition with albumin outside of a living organism, i.e. to add exogenous albumin to the composition. Such a composition may be lyophilized, providing a formulation that is suitable for storage at ambient temperature. Thus, the present disclosure also provides a composition as defined above which further includes albumin, such as human serum albumin, and which may optionally be in dry form.

Co-administration can be achieved by separately administering the exendin, exendin agonist, or exendin analog agonist engineered polypeptide with the second agent, or by administering a single pharmaceutical formulation including the exendin, exendin agonist, or exendin analog agonist engineered polypeptide and the second agent. Appropriate dosage regimens for the second agents are generally known in the art.

The preparations can also be co-administered, when desired, with other active substances (e.g. to reduce metabolic degradation) as known in the art or other therapeutically active agents. An exendin engineered polypeptide described herein can be administered with other active anti-diabetes or anti-obesity agents, such as leptin or leptin agonists and amylin or amylin agonist compounds, e.g. the amylins, including davalintide and their analogs.

Amylins. Amylin is a peptide hormone synthesized by pancreatic β-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin, as known in the art. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic and equipotent analogue of human amylin, reduces postprandial glucose excursions by suppressing inappropriately elevated postprandial glucagon secretion and slowing gastric emptying. The sequences of rat amylin, human amylin and pramlintide follow:

(SEQ ID NO: 6)

KCNTATCATQRLANFLVRSSNNLGPVLPPTNVGSNTY;

(SEQ ID NO: 7)

KCNTATCATQRLANFLVHSSNNFGAILSSTNVGSNTY;

(SEQ ID NO: 8)

KCNTATCATQRLANFLVHSSNNFGPILPPTNVGSNTY.

Davalintide. Davalintide, also known as “AC-2307” is a potent amylin agonist useful in the treatment of a variety of disease indications. See WO 2006/083254 and WO 2007/114838, each of which is incorporated by reference herein in its entirety and for all purposes. Davalintide is a chimeric peptide, having an N-terminal loop region of amylin or calcitonin and analogs thereof, an alpha-helical region of at least a portion of an alpha-helical region of calcitonin or analogs thereof or an alpha-helical region having a portion of an amylin alpha-helical region and a calcitonin alpha-helical region or analog thereof, and a C-terminal tail region of amylin or calcitonin. The sequences of human calcitonin, salmon calcitonin and davalintide follow:

(SEQ ID NO: 9)

CGNLSTCMLGTYTQDFNKFHTFPQTAIGVGAP;

(SEQ ID NO: 10)

CSNLSTCVLGKLSQELHKLQTYPRTNTGSGTP;

(SEQ ID NO: 11)

KCNTATCVLGRLSQELHRLQTYPRTNTGSNTY.

Without wishing to be bound by any theory, it is believed that amylins and davalintide, and fragment and analogs thereof, can require C-terminal amidation to elicit a full biological response. It is understood that amylin compounds such as those described herein which include amylins and/or davalintide, and fragment and analogs thereof, can be amidated at the C-terminal.

“Amylin agonist compounds” include native amylin peptides, amylin analog peptides, and other compounds (e.g., small molecules) that have amylin agonist activity. The “amylin agonist compounds” can be derived from natural sources, can be synthetic, or can be derived from recombinant DNA techniques. Amylin agonist compounds have amylin agonist receptor binding activity and may include amino acids (e.g., natural, unnatural, or a combination thereof), peptide mimetics, chemical moieties, and the like. The skilled artisan will recognize amylin agonist compounds using amylin receptor binding assays or by measuring amylin agonist activity in soleus muscle assays. In one embodiment, amylin agonist compounds will have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein in their entireties and for all purposes. In one embodiment, amylin agonist compounds will have an EC₅₀ of about 20 nM or less, about nM 15 or less, about nM 10 or less, or about nM 5 or less in a soleus muscle assay, such as that described herein and in U.S. Pat. No. 5,686,411. In one embodiment, the amylin agonist compound has at least 90% or 100% sequence identity to ^{25,28,29}Pro-human-amylin. In one embodiment, the amylin agonist compound is a peptide chimera of amylin (e.g., human amylin, rat amylin, and the like) and calcitonin (e.g., human calcitonin, salmon calcitonin, and the like). Suitable and exemplary amylin agonist compounds are also described in US Publication No. 2008/0274952, the disclosure of which is incorporated by reference herein in its entirety and for all purposes.

When co-administered with another active agent, the compounds can be administered simultaneously or sequentially, together or separately formulated. Since the engineered compounds herein are inherently long-acting, they are suitable for once daily, once weekly or longer administration. Accordingly, the other agent may be administered either in one or multiple doses, e.g. once daily, twice daily, three times daily, once weekly, as needed, during the period of dosing for the exendin engineered polypeptide, e.g. once weekly.

Single and multiple-use formulations of other agents such as amylin compounds have been reported. For example, pramlintide has been formulated for and successfully administered for once, twice and three times daily administration for treating diabetes and for treating obesity.

These pharmaceutical compounds may be formulated with pharmaceutically acceptable carriers or diluents as well as any other known adjuvants and excipients in accordance with conventional techniques such as those disclosed in Remington Pharmaceutical Sciences by E. W. Martin. See also Wang et al. (1988) J. of Parenteral Sci. and Tech., Technical Report No. 10, Supp. 42:2 S.

In general, the engineered polypeptides may be formulated into a stable, safe pharmaceutical composition for administration to a patient. Pharmaceutical formulations contemplated for use in the methods of the invention may include approximately 0.01 to 1.0% (w/v), in certain cases 0.05 to 1.0%, of the engineered polypeptide, approximately 0.02 to 0.5% (w/v) of an acetate, phosphate, citrate or glutamate buffer allowing a pH of the final composition of from about 3.0 to about 7.0; approximately 1.0 to 10% (w/v) of a carbohydrate or polyhydric alcohol tonicifier and, optionally, approximately 0.005 to 1.0% (w/v) of a preservative selected from the group of m-cresol, benzyl alcohol, methyl, ethyl, propyl and butyl parabens and phenol. Such a preservative is generally included if the formulated peptide is to be included in a multiple use product.

In particular embodiments, a pharmaceutical formulation of the present engineered polypeptides may contain a range of concentrations of the compound(s), e.g., between about 0.01% to about 98% w/w, or between about 1 to about 98% w/w, or preferably between 80% and 90% w/w, or preferably between about 0.01% to about 50% w/w, or more preferably between about 10% to about 25% w/w in these embodiments. A sufficient amount of water for injection may be used to obtain the desired concentration of solution.

Additional tonicifying agents such as sodium chloride, as well as other known excipients, may also be present, if desired. In some cases, such excipients are useful in maintenance of the overall tonicity of the compound. An excipient may be included in the presently described formulations at various concentrations. For example, an excipient may be included in the concentration range from about 0.02% to about 20% w/w, preferably between about 0.02% and 0.5% w/w, about 0.02% to about 10% w/v, or about 1% to about 20% w/w. In addition, similar to the present formulations themselves, an excipient may be included in solid (including powdered), liquid, semi-solid or gel form.

The pharmaceutical formulations may be composed in various forms, e.g., solid, liquid, semisolid or liquid. The term “solid”, as used herein, is meant to encompass all normal uses of this term including, for example, powders and lyophilized formulations. The presently described formulations may be lyophilized.

The terms buffer, buffer solution and buffered solution, when used with reference to hydrogen-ion concentration or pH, refer to the ability of a system, particularly an aqueous solution, to resist a change of pH on adding acid or alkali, or on dilution with a solvent. Characteristic of buffered solutions, which undergo small changes of pH on addition of acid or base, is the presence either of a weak acid and a salt of the weak acid, or a weak base and a salt of the weak base. An example of the former system is acetic acid and sodium acetate. The change of pH is slight as long as the amount of hydronium or hydroxyl ion added does not exceed the capacity of the buffer system to neutralize it.

As described herein, a variety of liquid vehicles are suitable for use in the formulations of engineered polypeptides, for example, water or an aqueous/organic solvent mixture or suspension.

The stability of a engineered polypeptide formulation for use as described herein is enhanced by maintaining the pH of the formulation in a range determined by methods known in the art. In certain embodiments, the pH of the formulation is maintained in the range of about 3.5 to 5.0, or about 3.5 to 6.5, in some embodiments from about 3.7 to 4.3, or about 3.8 to 4.2. In some embodiments, pH may be about 4.0, about 5.0, about 6.0, about 7.0, about 8.0, about 9.0, or even higher. In some embodiments, pH may be in the physiological range, pH 6-8, preferably pH 7-7.6.

In certain embodiments, the buffer with the engineered polypeptide is an acetate buffer (preferably at a final formulation concentration of from about 1-5 to about 60 mM), phosphate buffer (preferably at a final formulation concentration of from about 1-5 to about to about 30 mM) or glutamate buffer (preferably at a final formulation concentration of from about 1-5 to about to about 60 mM). In some embodiments, the buffer is acetate (preferably at a final formulation concentration of from about 5 to about 30 mM).

A stabilizer may be included in the formulations but is not necessarily needed. If included, however, a stabilizer useful in the practice of the present invention is a carbohydrate or a polyhydric alcohol. A suitable stabilizer useful in the practice of the present invention is approximately 1.0 to 10% (w/v) of a carbohydrate or polyhydric alcohol. The polyhydric alcohols and carbohydrates share the same feature in their backbones, i.e., —CHOH—CHOH—, which is responsible for stabilizing the proteins. The polyhydric alcohols include such compounds as sorbitol, mannitol, glycerol, and polyethylene glycols (PEGs). These compounds are straight-chain molecules. The carbohydrates, such as mannose, ribose, sucrose, fructose, trehalose, maltose, inositol, and lactose, on the other hand, are cyclic molecules that may contain a keto or aldehyde group. These two classes of compounds have been demonstrated to be effective in stabilizing protein against denaturation caused by elevated temperature and by freeze-thaw or freeze-drying processes. Suitable carbohydrates include: galactose, arabinose, lactose or any other carbohydrate which does not have an adverse affect on a diabetic patient, i.e., the carbohydrate is not metabolized to form unacceptably large concentrations of glucose in the blood. Such carbohydrates are well known in the art as suitable for diabetics. Sucrose and fructose are suitable for use with the compound in non-diabetic applications (e.g. treating obesity).

In certain embodiments, if a stabilizer is included, the compound is stabilized with a polyhydric alcohol such as sorbitol, mannitol, inositol, glycerol, xylitol, and polypropylene/ethylene glycol copolymer, as well as various polyethylene glycols (PEG) of molecular weight 200, 400, 1450, 3350, 4000, 6000, 8000 and even higher). Mannitol is the preferred polyhydric alcohol in some embodiments. Another useful feature of the lyophilized formulations of the present invention is the maintenance of the tonicity of the lyophilized formulations described herein with the same formulation component that serves to maintain their stability. In some embodiments, mannitol is the preferred polyhydric alcohol used for this purpose.

The United States Pharmacopeia (USP) states that anti-microbial agents in bacteriostatic or fungistatic concentrations must be added to preparations contained in multiple dose containers. They must be present in adequate concentration at the time of use to prevent the multiplication of microorganisms inadvertently introduced into the preparation while withdrawing a portion of the contents with a hypodermic needle and syringe, or using other invasive means for delivery, such as pen injectors. Antimicrobial agents should be evaluated to ensure compatibility with all other components of the formula, and their activity should be evaluated in the total formula to ensure that a particular agent that is effective in one formulation is not ineffective in another. It is not uncommon to find that a particular antimicrobial agent will be effective in one formulation but not effective in another formulation.

A preservative is, in the common pharmaceutical sense, a substance that prevents or inhibits microbial growth and may be added to pharmaceutical formulations for this purpose to avoid consequent spoilage of the formulation by microorganisms. While the amount of the preservative is not great, it may nevertheless affect the overall stability of the peptide.

While the preservative for use in the pharmaceutical compositions can range from 0.005 to 1.0% (w/v), in some embodiments range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in Remington's Pharmaceutical Sciences (Id.)

Engineered polypeptides may not have a tendency to adsorb onto the glass in a glass container when in a liquid form, therefore, a surfactant may not be required to further stabilize the pharmaceutical formulation. However, with regard to compounds which do have such a tendency when in liquid form, a surfactant should be used in their formulation. These formulations may then be lyophilized. Surfactants frequently cause denaturation of protein, both of hydrophobic disruption and by salt bridge separation. Relatively low concentrations of surfactant may exert a potent denaturing activity, because of the strong interactions between surfactant moieties and the reactive sites on proteins. However, judicious use of this interaction can stabilize proteins against interfacial or surface denaturation. Surfactants which could further stabilize the engineered polypeptide may optionally be present in the range of about 0.001 to 0.3% (w/v) of the total formulation and include polysorbate 80 (i.e., polyoxyethylene(20) sorbitan monooleate), CHAPS® (i.e., 3-[(3-cholamidopropyl) dimethylammonio]1-propanesulfonate), Brij® (e.g., Brij® 35, which is (polyoxyethylene (23) lauryl ether), poloxamer, or another non-ionic surfactant.

It may also be desirable to add sodium chloride or other salt to adjust the tonicity of the pharmaceutical formulation, depending on the tonicifier selected. However, this is optional and depends on the particular formulation selected. Parenteral formulations preferably may be isotonic or substantially isotonic.

A preferred vehicle for parenteral products is water. Water of suitable quality for parenteral administration can be prepared either by distillation or by reverse osmosis. Water for injection is the preferred aqueous vehicle for use in the pharmaceutical formulations.

It is possible that other ingredients may be present in the pharmaceutical formulations. Such additional ingredients may include, e.g., wetting agents, emulsifiers, oils, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Additionally, polymer solutions, or mixtures with polymers provide the opportunity for controlled release of the peptide. Such additional ingredients, of course, should not adversely affect the overall stability of the pharmaceutical formulation of the present invention.

Containers are also an integral part of the formulation of an injection and may be considered a component, for there is no container that is totally inert, or does not in some way affect the liquid it contains, particularly if the liquid is aqueous. Therefore, the selection of a container for a particular injection must be based on a consideration of the composition of the container, as well as of the solution, and the treatment to which it will be subjected. Adsorption of the peptide to the glass surface of the vial can also be minimized, if necessary, by use of borosilicate glass, for example, Wheaton Type I borosilicate glass #33 (Wheaton Type 1-33) or its equivalent (Wheaton Glass Co.). Other vendors of similar borosilicate glass vials and cartridges acceptable for manufacture include Kimbel Glass Co., West Co., Bunder Glas GMBH and Form a Vitrum. The biological and chemical properties of the compound may be stabilized by formulation and lyophilization in a Wheaton Type 1-33 borosilicate serum vial to a final concentration of 0.1 mg/ml and 10 mg/ml of the compound in the presence of 5% mannitol, and 0.02% Tween 80.

For formulations to be delivered by injection, in order to permit introduction of a needle from a hypodermic syringe into a multiple-dose vial and provide for resealing as soon as the needle is withdrawn, the open end of each vial is preferably sealed with a rubber stopper closure held in place by an aluminum band.

Stoppers for glass vials, such as, West 4416/50, 4416/50 (Teflon faced) and 4406/40, Abbott 5139 or any equivalent stopper can be used as the closure for pharmaceutical for injection. For formulations including peptidic anti-obesity agents, these stoppers are compatible with the peptide as well as the other components of the formulation. The inventors have also discovered that these stoppers pass the stopper integrity test when tested using patient use patterns, e.g., the stopper can withstand at least about 100 injections. Alternatively, the peptide can be lyophilized in to vials, syringes or cartridges for subsequent reconstitution. Liquid formulations of the present invention can be filled into one or two chambered cartridges, or one or two chamber syringes.

Each of the components of the pharmaceutical formulation described above is known in the art and is described in Pharmaceutical Dosage Forms: Parenteral Medications, Vol. 1, 2nd ed., Avis et al. Ed., Mercel Dekker, New York, N.Y. 1992, which is incorporated by reference in its entirety herein.

The manufacturing process for the above liquid formulations generally involves compounding, sterile filtration and filling steps. The compounding procedure involves dissolution of ingredients in a specific order (preservative followed by stabilizer/tonicity agents, buffers and peptide) or dissolving at the same time.

Alternative formulations, e.g., non-parenteral, may not require sterilization. However, if sterilization is desired or necessary, any suitable sterilization process can be used in developing the peptide pharmaceutical formulation of the present invention. Typical sterilization processes include filtration, steam (moist heat), dry heat, gases (e.g., ethylene oxide, formaldehyde, chlorine dioxide, propylene oxide, beta-propiolactone, ozone, chloropicrin, peracetic acid methyl bromide and the like), exposure to a radiation source, and aseptic handling. Filtration is the preferred method of sterilization for liquid formulations of the present invention. The sterile filtration involves filtration through 0.45 um and 0.22 um (1 or 2) which may be connected in series. After filtration, the solution is filled into appropriate vials or containers.

In certain embodiments, the engineered polypeptides described herein are administered peripherally to the subjects. In some embodiments, the liquid pharmaceutical formulations of the present invention are intended for parenteral administration. Suitable routes of administration include intramuscular, intravenous, subcutaneous, intradermal, intraarticular, intrathecal and the like. In some embodiments, the subcutaneous route of administration is preferred. In certain embodiments, mucosal delivery is also preferred. These routes include, but are not limited to, oral, nasal, sublingual, pulmonary and buccal routes which may include administration of the peptide in liquid, semi-solid or solid form. For formulations including engineered polypeptides, administration via these routes can require substantially more compound to obtain the desired biological effects due to decreased bioavailability compared to parenteral delivery.

In addition, parenteral controlled release delivery can be achieved by forming polymeric microcapsules, matrices, solutions, implants and devices and administering them parenterally or by surgical means. Examples of controlled release formulations are described in U.S. Pat. Nos. 6,368,630, 6,379,704, and 5,766,627, which are incorporated herein by reference. These dosage forms may have a lower bioavailability due to entrapment of some of the peptide in the polymer matrix or device. See e.g., U.S. Pat. Nos. 6,379,704, 6,379,703, and 6,296,842, each of which is incorporated herein by reference in its entirety and for all purposes.

The compounds may be provided in dosage unit form containing an amount of the engineered polypeptide that will be effective in one or multiple doses.

As will be recognized by those in the field, an effective amount of the engineered polypeptide will vary with many factors including the age and weight of the subject, the subject's physical condition, the condition to be treated, and other factors known in the art. An effective amount of the engineered polypeptides will also vary with the particular combination administered. As described herein, administration of the engineered polypeptides in combination may allow for a reduced amount of any of the administered engineered polypeptides to be an effective amount.

Administration can be by oral route, including transcellular, paracellular or receptor-mediated routes. Without wishing to be bound by any theory, the engineered polypeptides containing an exendin as described herein are orally available, in part because of their relatively small size and relative stability to gut enzymes. It has been reported that tight junctions between intestinal cells opened by absorption/permeation enhancers are less than 20 nm wide. See e.g., Chao et al., 1998, J. Drug Targeting, 6:37-43. Accordingly, a sufficiently small (for example, less than 10 kD or 15 kD) engineered polypeptide as described herein can transit the gut wall and bind albumin in the portal system, thereby gaining access to the circulation. Oral delivery of the engineered polypeptides of the present invention may be twice daily, once daily, once other day, once every three days, once weekly, once in two weeks, one in three weeks, or even once a month. Oral delivery systems suitable for other peptides can be used. In one embodiment the oral delivery system may have a relatively rapid uptake profile, e.g. from 1 to 4 hours, in which case the inherently long-duration of action of the engineered polypeptide provides the extended duration of action desired, such as for once daily or once weekly administration. The duration of action can be selected, for example, by choice of ABD and its affinity for albumin. While not wishing to be bound by theory, it is believed that higher affinity to albumin will yield longer circulation times providing longer duration of action. Oral delivery can be tested using known in vitro and in vivo methods. For example, a mouse can be orally gavaged with a solution containing an engineered polypeptide formulated with or without a permeation/absorption enhancer and/or protease inhibitor in order to test orally availability and effect of any added excipient. Either or both pharmacodynamic (therapeutic effects) and pharmacokinetic (drug properties) can be measured over time, such as drug plasma levels, acute or chronic glucose and/or HbA1c lowering, insulin plasma levels, food intake inhibition, weight loss, and/or lipid levels.

B. Effective Dosages

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat diabetes, such compositions will contain an amount of active ingredient effective to achieve the desired result (e.g. decreasing fasting blood glucose in a subject). When administered in methods to treat obesity, such compositions will contain an amount of active ingredient effective to achieve the desired result (e.g. decrease the body mass).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to compounds described herein; fasting blood glucose); presence of other diseases or other health-related problems; kind of concurrent treatment; and complications from any disease or treatment regimen. Other therapeutic regimens or agents can be used in conjunction with the methods and compounds of the invention.

Therapeutically effective amounts for use in humans may be determined from animal models. For example, a dose for humans can be formulated to achieve a concentration that has been found to be effective in animals. The dosage in humans can be adjusted by monitoring one or more physiological parameters, including but not limited to blood sugar and body mass, and adjusting the dosage upwards or downwards, as described above and known in the art.

Dosages may be varied depending upon the requirements of the patient and the compound being employed. The dose administered to a patient, in the context of the present invention, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

However, typical doses may contain from a lower limit of about 1 ug, 5 ug, 10 ug, 50 ug, 100 ug to 150 ug per day to an upper limit of about to 50 ug, to 100 ug, to 150 ug, to 200 ug or even to 300 ug of the pharmaceutical compound per week in view of the extended half-life of the engineered polypeptides herein. The doses may be delivered in discrete unit doses at the desired interval, e.g. daily or weekly.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

The surprising dose-sparing property of the engineered polypeptides of the present invention, along with their surprisingly long plasma half-life and duration of pharmacological action, provides for a superior pharmaceutical agent. Also surprising in the case of the exendin-containing engineered polypeptides are their oral availability. The superior properties including dose-sparing, allow for lower dosing, thus less or less severe side-effects and improved cost of goods, and/or more cost-effective and simpler formulations for once daily or once weekly administration not currently achieved by the parent compounds alone.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch.1, p. 1, 1975. The exact formulation, route of administration, and dosage can be chosen by the individual physician in view of the patient's condition and the particular method in which the compound is used.

Without wishing to be bound by any theory, it is believed that fusion of an ABD albumin binding domain with a hormone domain as described herein, can provide decreased immunogenicity as judged by a reduction in immune response relative to the hormone domain without ABD fusion. See e.g., WO 2009/016043, incorporated herein by reference in its entirety and for all purposes.

Exemplary HD1, Linker and ABD sequences include:

(SEQ ID NO: 1)

HSDGTFTSDLSKQMEEEAVRLFIEWLKNGGPSSGAPPPS-NH₂;

(SEQ ID NO: 2)

HGEGTFTSDLSKQMEEEAVRLFIEWLKNGGPSSGAPPPS-NH₂;

(SEQ ID NO: 3)

HGEGTFTSDLSKQLEEEAVRLFIEWLKNGGPSSGAPPPS-NH₂;

(SEQ ID NO: 4)

HGEGTFTSDLSKQLEEEAVRLFIEWLKQGGPSKEIIS;

(SEQ ID NO: 5)

HAEGTFTSDVSSYLEGQAAKEFIAWLVKGRG;

(SEQ ID NO: 6)

KCNTATCATQRLANFLVRSSNNLGPVLPPTNVGSNTY;

(SEQ ID NO: 7)

KCNTATCATQRLANFLVHSSNNFGAILSSTNVGSNTY;

(SEQ ID NO: 8)

KCNTATCATQRLANFLVHSSNNFGPILPPTNVGSNTY;

(SEQ ID NO: 9)

CGNLSTCMLGTYTQDFNKFHTFPQTAIGVGAP;

(SEQ ID NO: 10)

CSNLSTCVLGKLSQELHKLQTYPRTNTGSGTP;

(SEQ ID NO: 11)

KCNTATCVLGRLSQELHRLQTYPRTNTGSNTY;

(SEQ ID NO: 12)

HGEGTFTSDLSKQMEEEAVRLFIEWLKN;

(SEQ ID NO: 49)

HHHHHH;

(SEQ ID NO: 111)

HGEGTFTSDLSKQMEEEAVRLFIEWLKQGGPSKEIIS;

(SEQ ID NO: 112)

HGEGTFTSDLSKQLEEEAVRLFIEWLKNGGPSSGAPPS;

(SEQ ID NO: 113)

HGEGTFTSDLSKQMEEEAVRLFIEWLKNGGPSSGAPPS;

(SEQ ID NO: 114)

HGEGTFTSDLSKQLEEEAVRLFIEWLKQGGPSKEIISKKKKKK;

(SEQ ID NO: 115)

HGEGTFTSDLSKQMEEEAVRLFIEWLKNGGPSSGAPPPSKKKKKK;

(SEQ ID NO: 116)

HGEGTFTSDLSKQMEEEAVRLFIEWLKQGGPSKEIISKKKKKK;

(SEQ ID NO: 117)

HGEGTFTSDLSKQLEEEAVRLFIEWLKNGGPSSGAPPSKKKKKK;

(SEQ ID NO: 118)

HGEGTFTSDLSKQMEEEAVRLFIEWLKNGGPSSGAPPSKKKKKK;

(SEQ ID NO: 125)

GGGG;

(SEQ ID NO: 126)

GGGGG;

(SEQ ID NO: 127)

[G]ₙE,
wherein n is 1-6;

(SEQ ID NO: 128)

[G]ₙK,
wherein n is 1-6;

(SEQ ID NO: 129)

[G]ₙD,
wherein n is 1-6;

(SEQ ID NO: 130)

[G]ₙR,
wherein n is 1-6;

(SEQ ID NO: 131)

GGGKGGGG;

(SEQ ID NO: 132)

GGGNGSGG;

(SEQ ID NO: 133)

GGGCGGGG;

(SEQ ID NO: 134)

GPNGG;

(SEQ ID NO: 135)

[GS]ₙ,
wherein n is 1-6;

(SEQ ID NO: 136)

[GGS]ₙ,
where n is 1-6;

(SEQ ID NO: 137)

[GGGS]ₙ,
where n is 1-6;

(SEQ ID NO: 138)

[GGGGS]ₙ,
where n is 1-6;

(SEQ ID NO: 139)

[GE]ₙ,
where n is 1-10;

(SEQ ID NO: 140)

[GGE]ₙ,
where n is 1-10;

(SEQ ID NO: 141)

[GGGE]ₙ,
where n is 1-10;

(SEQ ID NO: 142)

[GGGGE]ₙ,
where n is 1-10;

(SEQ ID NO: 143)

[GD]ₙ,
where n is 1-10;

(SEQ ID NO: 144)

[GGD]ₙ,
where n is 1-10;

(SEQ ID NO: 145)

[GGGD]ₙ,
where n is 1-10;

(SEQ ID NO: 146)

[GGGGD]ₙ,
where n is 1-10;

(SEQ ID NO: 147)

[GK]ₙ,
where n is 1-10;

(SEQ ID NO: 148)

[GGK]ₙ,
where n is 1-10;

(SEQ ID NO: 149)

[GGGK]ₙ,
where n is 1-10;

(SEQ ID NO: 150)

[GGGGK]ₙ,
where n is 1-10;

(SEQ ID NO: 151)

[GR]ₙ,
where n is 1-10;

(SEQ ID NO: 152)

[GGR]ₙ,
where n is 1-10;

(SEQ ID NO: 153)

[GGGR]ₙ,
where n is 1-10;

(SEQ ID NO: 154)

[GGGGR]ₙ,
where n is 1-10;

(SEQ ID NO: 155)

[EAAAK]ₙ,
where n is 1-10;

(SEQ ID NO: 300)

LA X3 AK X6 X7 AN X10 ELD X14 YGVSDF YKRLI X26
KAKTVEGVEALK X39 X40 IL X43 X44 LP,

wherein independently of each other X3 is selected from E, S, Q and C; X6 is selected from E, S and C; X7 is selected from A and S; X14 is selected from A, S, C and K; X10 is selected from A, S and R; X26 is selected from D and E; X39 is selected from D and E; X40 is selected from A and E; X43 is selected from A and K; X44 is selected from A, S and E; the leucine at position 45 is present or absent; and the proline at position 46 is present or absent;

(SEQ ID NO: 678)

LA X3 AK X6 X7 AN X10 ELD X14 YGVSDFYKRLIDKAKTVE
GVEALKDAILAALP

where independently of each other X3 is selected from E, S, Q and C; X6 is selected from E, S and C; X7 is selected from A and S; X10 is selected from A, S and R; and X14 is selected from A, S, C and K; leucine at position 45 is present or absent; and proline at position 46 is present or absent; and

### see Table 1, Table 2, Table 3 and FIG. 1.

Exemplary embodiments of the engineered polypeptides comprising an exendin, an exendin analog or an active fragment thereof, method of use thereof, and pharmaceuticals compositions described herein and include:

Embodiment 1. An engineered polypeptide comprising: an albumin binding domain polypeptide (ABD) sequence, and a first peptide hormone domain (HD1) sequence selected from an exendin sequence, an exendin analog sequence or an active fragment sequence thereof. Embodiment 2. The engineered polypeptide according to embodiment 1, further comprising a first linker (LI) covalently linking said HD1 sequence and said ABD sequence. 3. The engineered polypeptide according to embodiment 1 or 2, wherein said engineered polypeptide comprises said ABD sequence as a C-terminal moiety and said HD1 sequence as an N-terminal moiety. 4. The engineered polypeptide according to embodiment 3, comprising the structure: HD1-ABD. 5. The engineered polypeptide according to embodiment 3, comprising the structure: HD1-L1-ABD. 6. The engineered polypeptide according to any one of embodiments 1 to 5, wherein said HD1 sequence is said exendin or exendin analog sequence. 7. The engineered polypeptide according to embodiment 6, wherein said exendin sequence is exendin-4 sequence and the exendin analog sequence is Leu14 exendin-4 sequence. 8. The engineered polypeptide according to embodiment 6, wherein said exendin fragment sequence is the sequence of exendin-4(1-28), exendin-4(1-29), exendin-4(1-30), exendin-4(1-31) or exendin-4(1-32). 9. The engineered polypeptide according to embodiment 6, wherein said exendin or exendin analog sequence is selected from the group of sequences consisting of:

(SEQ ID NO: 3)

HGEGTFTSDLSKQLEEEAVRLFIEWLKNGGPSSGAPPPS,

(SEQ ID NO: 4)

HGEGTFTSDLSKQLEEEAVRLFIEWLKQGGPSKEIIS,

(SEQ ID NO: 2)

HGEGTFTSDLSKQMEEEAVRLFIEWLKNGGPSSGAPPPS,

(SEQ ID NO: 111)

HGEGTFTSDLSKQMEEEAVRLFIEWLKQGGPSKEIIS,

(SEQ ID NO: 112)

HGEGTFTSDLSKQLEEEAVRLFIEWLKNGGPSSGAPPS,

(SEQ ID NO: 113)

HGEGTFTSDLSKQMEEEAVRLFIEWLKNGGPSSGAPPS,

(SEQ ID NO: 114)

HGEGTFTSDLSKQLEEEAVRLFIEWLKQGGPSKEIISKKKKKK,

(SEQ ID NO: 115)

HGEGTFTSDLSKQMEEEAVRLFIEWLKNGGPSSGAPPPSKKKKKK,

(SEQ ID NO: 116)

HGEGTFTSDLSKQMEEEAVRLFIEWLKQGGPSKEIISKKKKKK,

(SEQ ID NO: 117)

HGEGTFTSDLSKQLEEEAVRLFIEWLKNGGPSSGAPPSKKKKKK
and

(SEQ ID NO: 118)

HGEGTFTSDLSKQMEEEAVRLFIEWLKNGGPSSGAPPSKKKKKK.

10. The engineered polypeptide according to any one of the previous embodiments, wherein said exendin or exendin analog comprises a modification at position corresponding to Hisl selected from the group consisting of L-histidine, D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl and beta-carboxy-imidazopropionyl. 11. The engineered polypeptide according to any one of the previous embodiments, wherein said analog comprises exendin-4 comprising a modification at position corresponding to His1 and is selected from the group consisting (4-imidazoacetyl) exendin-4, (des-amino-histidyl) exendin-4 (or (imidazopropionyl) exendin-4), (beta-hydroxy-imidazopropionyl) exendin-4, (N-dimethyl-histidyl) exendin-4 and (beta-carboxy-imidazopropionyl) exendin-4. 12. The engineered polypeptide according to any one of the previous embodiments, wherein said exendin analog sequence has at least 70% amino acid sequence identity with exendin-4, to an exendin analog of embodiment 9 or to the exendin fragment of embodiment 8. 13. The engineered polypeptide according to any one of embodiments 1 to 12, wherein said exendin analog has at least 80% identity with exendin-4, to an exendin analog of embodiment 9 or to the exendin fragment of embodiment 8. 14. The engineered polypeptide according to the previous embodiment, wherein said exendin analog has at least 90% identity with exendin-4, to an exendin analog of embodiment 9 or to the exendin fragment of embodiment 8. 15. The engineered polypeptide according to the previous embodiment, wherein said exendin analog has at least 95% identity with exendin-4, to an exendin analog of embodiment 9 or to the exendin fragment of embodiment 8. 16. The engineered polypeptide according any one of the previous embodiments, wherein said exendin analog sequence has from 1 to 5 amino acid modifications selected independently from any one or combination of an insertion, deletion, addition and substitution. Embodiment 17. The engineered polypeptide according to any one of the preceding embodiments, wherein said ABD sequence comprises an amino acid sequence selected from the amino acid sequence comprising: formula (i) LA X3 AK X6 X7 AN X10 ELD X14 YGVSDF YKRLI X26 KAKTVEGVEALK X39 X40 IL X43 X44 LP (SEQ ID NO: 300) wherein independently of each other X3 is selected from E, S, Q and C; X6 is selected from E, S and C; X7 is selected from A and S; X14 is selected from A, S, C and K; X10 is selected from A, S and R; X26 is selected from D and E; X39 is selected from D and E; X40 is selected from A and E; X43 is selected from A and K; X44 is selected from A, S and E; the leucine at position 45 is present or absent; and the proline at position 46 is present or absent; and formula (ii) an amino acid sequence which has at least 95% identity to the sequence defined in (i), with the proviso that X₇ is not L, E or D; or alternatively, with the proviso that the amino acid sequence is not defined by the following sequence, as defined in PCT Published Application No. WO 2009/016043: LAEAK Xₐ X_{b} A X, X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO: 679) wherein independently of each other, Xₐ is selected from V and E; X_{b} is selected from L, E and D; X, is selected from N, L and I; X_{d} is selected from R and K; X, is selected from D and K; and X₅ is selected from Y and F; X₈ is selected from N, R and S; X₉ is selected from V, I, L, M, F and Y; X₁₁ is selected from N, S, E and D; X₁₂ is selected from R, K and N; X₁₄ is selected from K and R; X₂₀ is selected from D, N, Q, E, H, S, R and K; X₂₃ is selected from K, I and T; X₂₄ is selected from A, S, T, G, H, L and D; and X₂₅ is selected from H, E and D. 18. The engineered polypeptide according to embodiment 17, wherein said ABD sequence comprises an amino acid sequence comprising: formula (i) LA X3 AK X6 X7 AN X10 ELD X14 YGVSDF YKRLI X26 KAKTVEGVEALK X39 X40 IL X43 X44 LP (SEQ ID NO: 300) wherein independently of each other X3 is selected from E, S, Q and C; X6 is selected from E, S and C; X7 is selected from A and S; X14 is selected from A, S, C and K; X10 is selected from A, S and R; X26 is selected from D and E; X39 is selected from D and E; X40 is selected from A and E; X43 is selected from A and K; X44 is selected from A, S and E; the leucine at position 45 is present or absent; and the proline at position 46 is present or absent. 19. The engineered polypeptide according to embodiment 17, wherein said ABD sequence comprises an amino acid sequence comprising formula (ii) an amino acid sequence which has at least 95% identity to the sequence defined in (i), with the proviso that X₇ is not L, E or D; or alternatively, with the proviso that the amino acid sequence is not defined by the following sequence, as defined in PCT Published Application No. WO 2009/016043: LAEAK Xₐ X_{b} A X, X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO: 679) wherein independently of each other, Xₐ is selected from V and E; X_{b} is selected from L, E and D; X, is selected from N, L and I; X_{d} is selected from R and K; Xₑ is selected from D and K; and X₅ is selected from Y and F; X₈ is selected from N, R and S; X₉ is selected from V, I, L, M, F and Y; X₁₁ is selected from N, S, E and D; X₁₂ is selected from R, K and N; X₁₄ is selected from K and R; X₂₀ is selected from D, N, Q, E, H, S, R and K; X₂₃ is selected from K, I and T; X₂₄ is selected from A, S, T, G, H, L and D; and X₂₅ is selected from H, E and D. 20. The engineered polypeptide according to any one of the preceding embodiments, wherein said ABD sequence comprises an amino acid sequence selected from the amino acid sequence comprising: formula (iii) LA X3 AK X6 X7 AN X10 ELD X14 YGVSDF YKRLIDKAKT VEGVEALKDA ILAALP (SEQ ID NO: 678) wherein independently of each other X3 is selected from E, S, Q and C; X6 is selected from E, S and C; X7 is selected from A and S; X10 is selected from A, S and R; X14 is selected from A, S, C and K; the leucine at position 45 is present or absent; and the proline at position 46 is present or absent; and formula (iv) an amino acid sequence which has at least 95% identity to the sequence defined in (iii) with the proviso that X₇ is not L, E or D; or alternatively, with the proviso that the amino acid sequence is not defined by the following sequence, as defined in PCT Published Application No. WO 2009/016043: LAEAK Xₐ X_{b} A X_{c} X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO: 679) wherein independently of each other, Xₐ is selected from V and E; X_{b} is selected from L, E and D; X_{c} is selected from N, L and I; X_{d} is selected from R and K; Xₑ is selected from D and K; and X₅ is selected from Y and F; X₈ is selected from N, R and S; X₉ is selected from V, I, L, M, F and Y; X₁₁ is selected from N, S, E and D; X₁₂ is selected from R, K and N; X₁₄ is selected from K and R; X₂₀ is selected from D, N, Q, E, H, S, R and K; X₂₃ is selected from K, I and T; X₂₄ is selected from A, S, T, G, H, L and D; and X₂₅ is selected from H, E and D. 21. The engineered polypeptide according to embodiment 20, wherein said ABD sequence comprises an amino acid sequence comprising: formula (iii) LA X3 AK X6 X7 AN X10 ELD X14 YGVSDF YKRLIDKAKT VEGVEALKDA ILAALP (SEQ ID NO: 678) wherein independently of each other X3 is selected from E, S, Q and C; X6 is selected from E, S and C; X7 is selected from A and S; X10 is selected from A, S and R; X14 is selected from A, S, C and K; the leucine at position 45 is present or absent; and the proline at position 46 is present or absent. 22. The engineered polypeptide according to embodiment 20, wherein said ABD sequence comprises an amino acid sequence comprising formula (iv) an amino acid sequence which has at least 95% identity to the sequence defined in (iii), with the proviso that X₇ is not L, E or D; or alternatively, with the proviso that the amino acid sequence is not defined by the following sequence, as defined in PCT Published Application No. WO 2009/016043: LAEAK Xₐ X_{b} A X, X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO: 679) wherein independently of each other, X. is selected from V and E; X_{b} is selected from L, E and D; X_{c} is selected from N, L and I; X_{d} is selected from R and K; Xₑ is selected from D and K; and X₅ is selected from Y and F; X₈ is selected from N, R and S; X₉ is selected from V, I, L, M, F and Y; X₁₁ is selected from N, S, E and D; X₁₂ is selected from R, K and N; X₁₄ is selected from K and R; X₂₀ is selected from D, N, Q, E, H, S, R and K; X₂₃ is selected from K, I and T; X₂₄ is selected from A, S, T, G, H, L and D; and X₂₅ is selected from H, E and D. 23. The engineered polypeptide according to any one of the preceding embodiments, wherein X6 is S in the ABD. 24. The engineered polypeptide according to any one of the preceding embodiments, wherein X6 is E in the ABD. 25. The engineered polypeptide according to any one of the preceding embodiments, wherein X3 is S in the ABD. 26. The engineered polypeptide according to any one of the preceding embodiments, wherein X3 is E in the ABD. 27. The engineered polypeptide according to any one of the preceding embodiments, wherein X3 is Q in the ABD. 28. The engineered polypeptide according to any one of the preceding embodiments, wherein X7 is A in the ABD. 29. The engineered polypeptide according to any one of the preceding embodiments, wherein X7 is S in the ABD. 30. The engineered polypeptide according to any one of the preceding embodiments, wherein X10 is A in the ABD. 31.The engineered polypeptide according to any one of the preceding embodiments, wherein X10 is S in the ABD. 32.The engineered polypeptide according to any one of the preceding embodiments, wherein X10 is R in the ABD. 33. The engineered polypeptide according to any one of the preceding embodiments, wherein X14 is S in the ABD. 34. The engineered polypeptide according to any one of the preceding embodiments, wherein X14 is C in the ABD. 35. The engineered polypeptide according to any one of the preceding embodiments, wherein X14 is K in the ABD. 36. The engineered polypeptide according to any one of the preceding embodiments, wherein X14 is A in the ABD. 37. The engineered polypeptide according to any one of the preceding embodiments, wherein X26 is D in the ABD. 38. The engineered polypeptide according to any one of the preceding embodiments, wherein X26 is E in the ABD. 39. The engineered polypeptide according to any one of the preceding embodiments, wherein X39 is D in the ABD. 40. The engineered polypeptide according to any one of the preceding embodiments, wherein X39 is E in the ABD. 41. The engineered polypeptide according to any one of the preceding embodiments, wherein X40 is A in the ABD. 42. The engineered polypeptide according to any one of the preceding embodiments, wherein X40 is E in the ABD. 43. The engineered polypeptide according to any one of the preceding embodiments, wherein X43 is A in the ABD. 44. The engineered polypeptide according to any one of the preceding embodiments, wherein X43 is K in the ABD. 45. The engineered polypeptide according to any one of the preceding embodiments, wherein X44 is A in the ABD. 46. The engineered polypeptide according to any one of the preceding embodiments, wherein X44 is S in the ABD. 47. The engineered polypeptide according to any one of the preceding embodiments, wherein X44 is E in the ABD. 48. The engineered polypeptide according to any one of the preceding embodiments, wherein the leucine at position 45 in the ABD is present. 49. The engineered polypeptide according to any one of the preceding embodiments, wherein the leucine at position 45 in the ABD is absent. 50. The engineered polypeptide according to any one of the preceding embodiments, wherein the proline at position 46 in the ABD is present. 51. The engineered polypeptide according to any one of the preceding embodiments, wherein the proline at position 46 in the ABD is absent. 52. The engineered polypeptide according to any one of the preceding embodiments, wherein the ABD binds to albumin such that the k_{off} value of the interaction is at most 5×10-5 s-1. 53. The engineered polypeptide according to any one of the preceding embodiments, wherein the ABD binds to albumin such the k_{off} value of the interaction is at most 5×10-6 s-1. 54. The engineered polypeptide according to any one of the preceding embodiments, wherein the ABD sequence is selected from any one of the ABD sequences disclosed herein, or from Table 1, or from FIG. 1 or SEQ ID NO: 301-444. 55. The engineered polypeptide according to any one of the preceding embodiments, wherein the ABD sequence is selected from an amino acid sequence selected from any one of SEQ ID NO:304-305, SEQ ID NO:307-308, SEQ ID NO:310-311, SEQ ID NO:313-314, SEQ ID NO:316-317, SEQ ID NO:319-320, SEQ ID NO:322-323, SEQ ID NO:325-326, SEQ ID NO:328-329, SEQ ID NO:331-332, SEQ ID NO:334-335, SEQ ID NO:337-338, SEQ ID NO:341-342 and SEQ ID NO:349-350. 56. The engineered polypeptide according to any one of the preceding embodiments, wherein the ABD sequence comprises a sequence selected from an amino acid sequence from any one of the sequences of Table 1 or FIG. 1. 57. The engineered polypeptide according to any one of the preceding embodiments, wherein the ABD sequence further comprises one or more additional amino acid residues positioned at the N- and/or the C-terminal of the sequence defined in formula (i) or (iii). 58. The engineered polypeptide according to the preceding embodiment, wherein the one or more additional amino acid residues comprise a serine residue at the N-terminal of the ABD sequence. 59. The engineered polypeptide according to any one of the preceding embodiments, wherein the one or more additional amino acid residues comprise a glycine residue at the N-terminal of the ABD sequence. 60. The engineered polypeptide according to any one of the preceding embodiments, wherein the one or more additional amino acid residues comprise a glutamic acid residue or cysteine residue at the N-terminal of the ABD sequence. 61. The engineered polypeptide according to any one of the preceding embodiments, wherein the one or more additional amino acid residues comprise an alanine residue at the N-terminal of the ABD sequence. 62. The engineered polypeptide according to any one of the preceding embodiments, wherein the one or more additional amino acid residues comprise a glycine residue at the C-terminal of the ABD sequence. 63. The engineered polypeptide according to any one of the preceding embodiments, wherein the one or more additional amino acid residues comprise a cysteine residue at the C-terminal of the ABD sequence. 64. The engineered polypeptide according to any one of the preceding embodiments, wherein the ABD comprises an amino acid sequence selected from any one of SEQ ID NO:445-450 and SEQ ID NO:462-463. 65. The engineered polypeptide according to any one of the preceding embodiments, wherein the ABD comprises no more than two cysteine residues. 66. The engineered polypeptide according to the preceding embodiment, wherein the ABD sequence comprises no more than one cysteine residue. 67. The engineered polypeptide according any one of the preceding embodiments, wherein the exendin sequence or exendin analog sequence or fragment thereof is conjugated to the ABD via a thiol group of a cysteine residue at position X₁₄ of the polypeptide. 68. The engineered polypeptide according to embodiment 18, wherein said ABD is GSLAEAKEAANAELDSYGVSDFYKRLIDKAKTVEGVEALKDAILAALP (SEQ ID NO: 463) or GSLAEAKEAANAELDSYGVSDFYKRLIDKAKTVEGVEALKDAILAAL (SEQ ID NO: 700). 69. The engineered polypeptide according to embodiment 18, wherein said

ABD sequence comprises any one of the peptide sequences selected from the group consisting of: the sequences from Table 1, FIG. 1 or any of the tables herein. 70. The engineered polypeptide according to any one of embodiments 1 to 69, wherein said ABD sequence comprises an ABD analog having at least 95% identity with the ABD of formula i) or iii) or with any one of the ABDs of the Table 1, FIG. 1 or any of the tables herein. 71. The engineered polypeptide according to any one of embodiments 1 to 70, wherein said linker L1 is a peptide of from 1 to 30 amino acids or less than 30 amino acids. 72. The engineered polypeptide according to any one of embodiments 1 to 71, wherein said linker L1 is selected from the 20 naturally occurring amino acids. 73. The engineered polypeptide according to any one of embodiments 1 to 72, wherein said linker Ll comprises a non-natural amino acid incorporated by chemical synthesis, post-translational chemical modification or by in vivo incorporation by recombinant expression in a host cell. 74. The engineered polypeptide according to any one of embodiments 1 to 73, wherein said linker L1 amino acids are selected from glycine, alanine, proline, asparagine, glutamine, and lysine. 75. The engineered polypeptide according to any one of embodiments 1 to 74, wherein said linker L1 amino acid is glycine. 76. The engineered polypeptide according to any one of embodiments 1 to 74, wherein said linker L1 comprises a majority of amino acids that are sterically unhindered. 77. The engineered polypeptide according to any one of embodiments 1 to 76, wherein said linker Ll comprises polyglycine, polyalanine, poly(Gly-Ala) or poly(Gly-Ser). 78. The engineered polypeptide according to any one of embodiments 1 to 77, wherein said linker Ll comprises the sequence (Gly)3, (Gly)4 (SEQ ID NO: 125), or (Gly)5 (SEQ ID NO: 126), (Gly)3Lys(Gly)4 (SEQ ID NO: 131); (Gly)3AsnGlySer(Gly)2 (SEQ ID NO: 132); (Gly)3Cys(Gly)4 (SEQ ID NO: 133); GlyProAsnGlyGly (SEQ ID NO: 134), GG, GGG, GGS, or GGGS (SEQ ID NO: 192). 79. The engineered polypeptide according to any one of embodiments 1 to 74, wherein said linker L1 comprises a combination of Gly and Ala. 80. The engineered polypeptide according to any one of embodiments 1 to 74, wherein said linker L1 comprises a combination of Gly and Ser. 81. The engineered polypeptide according to any one of embodiments 1 to 80, wherein said linker L1 is selected from the group consisting of a glycine rich peptide. 82. The engineered polypeptide according to any one of the embodiments 1 to 81, wherein said linker L1 comprises an N-terminal TG dipeptide. 83. The engineered polypeptide according to any one of embodiments 1 to 82, wherein said linker Ll comprises a C-terminal AS dipeptide. 84. The engineered polypeptide according to any one of embodiments 1 to 83, wherein said linker L1 comprises an N-terminal TG dipeptide and a C-terminal AS dipeptide. 85. The engineered polypeptide according to any one of embodiments 1 to 84, wherein said linker L1 is selected from the group consisting of TG-(GGGS)1 (SEQ ID NO: 848), TG-(GGGS)2 (SEQ ID NO: 849), TG-(GGGS)3 (SEQ ID NO: 850), TG-(GGGS)4 (SEQ ID NO: 851), TG-(GGGS)5 (SEQ ID NO: 852), (GGGS)1-AS (SEQ ID NO: 853), (GGGS)2-AS (SEQ ID NO: 854), (GGGS)3-AS (SEQ ID NO: 855), (GGGS)4-AS (SEQ ID NO: 856), (GGGS)5-AS (SEQ ID NO: 857), TG-(GGGS)1-AS (SEQ ID NO: 195), TG-(GGGS)2-AS (SEQ ID NO: 858), TG-(GGGS)3-AS (SEQ ID NO: 859), TG-(GGGS)4-AS (SEQ ID NO: 860), and TG-(GGGS)5-AS (SEQ ID NO: 861). 86. The engineered polypeptide according to any one of embodiments 1 to 85, wherein said linker L1 TG and/or AS are absent or are replaced by a pair of amino acids selected from T, A, S, and G. 87. The engineered polypeptide according to any one of embodiments 1 to 86, having affinity for serum albumin with a dissociation constant less than about 10⁻⁶ mol/L. 88. The engineered polypeptide according to embodiment 87, having affinity for serum albumin with a dissociation constant less than about le mol/L. 89. The engineered polypeptide according to embodiment 88, having affinity for serum albumin with a dissociation constant less than about 10⁻¹² mol/L. 90. The engineered polypeptide according to any one of embodiments 1 to 89, wherein the polypeptide has a duration of action of at least 1 day. 91. The engineered polypeptide according to embodiment 90, wherein the polypeptide has a duration of action of at least 3 days. 92. The engineered polypeptide according to embodiment 91, wherein the polypeptide has a duration of action of at least 6 days. 93. The engineered polypeptide according to embodiment 92, wherein the polypeptide has a duration of action of at least 6 days in a human subject. 94. The engineered polypeptide according to embodiment 91, wherein the polypeptide has a duration of action of at least 7 days. 95. The engineered polypeptide according to embodiment 92, wherein the polypeptide has a duration of action of at least 7 days in a human subject. 96. The engineered polypeptide according to any one of the preceding embodiments, wherein the engineered polypeptide is selected from the group consisting of any one of the sequences of Table 2 or Table 3 herein. 97. The engineered polypeptide according to any one of the preceding embodiments, wherein the engineered polypeptide is selected from the group consisting of:

(SEQ ID NO: 727)

HGEGTFTSDLSKQLEEEAVRLFIEWLKQGGPSKEIISTGGGGSASGSLAE
AKEAANAELDSYGVSDFYKRLIDKAKTVEGVEALKDAILAALP;

(SEQ ID NO: 728)

HGEGTFTSDLSKQLEEEAVRLFIEWLKNGGPSSGAPPKSTGGGGSASGSL
AEAKEAANAELDSYGVSDFYKRLIDKAKTVEGVEALKDAILAALP;

(SEQ ID NO: 729)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAAL;

(SEQ ID NO: 730)

HGEGTFTSDLSKQMEEEAVRLFIEWLKNGGPSSGAPPPSTGGGGSASGSL
AEAKEAANAELDSYGVSDFYKRLIDKAKTVEGVEALKDAILAALP;

(SEQ ID NO: 731)

HGEGTFTSDLSKQMEEEAVRLFIEWLKNGGPSSGAPPPSGGSLAEAKEAA
NAELDSYGVSDFYKRLIDKAKTVEGVEALKDAILAALP;

(SEQ ID NO: 732)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAALP;

(SEQ ID NO: 733)

HGEGTFTSDLSKQLEEEAVRLFIEWLKNGGPSSGAPPPSGGSLAEAKEAA
NAELDSYGVSDFYKRLIDKAKTVEGVEALKDAILAALP;

(SEQ ID NO: 734)

HGEGTFTSDLSKQLEEEAVRLFIEWLKQGGPSKEIISTGGGGSASGSLAE
AKEAANAELDSYGVSDFYKRLIDKAKTVEGVEALKDAILAAL;

(SEQ ID NO: 735)

HGEGTFTSDLSKQLEEEAVRLFIEWLKNGGPSSGAPPKSTGGGGSASGSL
AEAKEAANAELDSYGVSDFYKRLIDKAKTVEGVEALKDAILAAL;

(SEQ ID NO: 736)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;

(SEQ ID NO: 737)

HGEGTFTSDLSKQMEEEAVRLFIEWLKNGGPSSGAPPPSTGGGGSASGSL
AEAKEAANAELDSYGVSDFYKRLIDKAKTVEGVEALKDAILAAL;

(SEQ ID NO: 738)

HGEGTFTSDLSKQMEEEAVRLFIEWLKNGGPSSGAPPPSGGSLAEAKEAA
NAELDSYGVSDFYKRLIDKAKTVEGVEALKDAILAAL;

(SEQ ID NO: 739)

HGEGTFTSDLSKQLEEEAVRLFIEWLKNGGPSSGAPPPSGGSLAEAKEAA
NAELDSYGVSDFYKRLIDKAKTVEGVEALKDAILAAL;

(SEQ ID NO: 740)

HGEGTFTSDLSKQLEEEAVRLFIEWLKQGGPSKEIISTGGGGSASGSLAE
AKEAANAELDSYGVSDFYKRLTDKAKTVEGVEALKDATLAA;

(SEQ ID NO: 741)

HGEGTFTSDLSKQLEEEAVRLFIEWLKNGGPSSGAPPKSTGGGGSASGSL
AEAKEAANAELDSYGVSDFYKRLIDKAKTVEGVEALKDAILAA;

(SEQ ID NO: 742)

HGEGTFTSDLSKQMEEEAVRLFIEWLKNGGPSSGAPPPSTGGGGSASGSL
AEAKEAANAELDSYGVSDFYKRLIDKAKTVEGVEALKDAILAA;

(SEQ ID NO: 743)

HGEGTFTSDLSKQMEEEAVRLFIEWLKNGGPSSGAPPPSGGSLAEAKEAA
NAELDSYGVSDFYKRLIDKAKTVEGVEALKDAILAA;

(SEQ ID NO: 744)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
and

(SEQ ID NO: 745)

HGEGTFTSDLSKQLEEEAVRLFIEWLKNGGPSSGAPPPSGGSLAEAKEAA
NAELDSYGVSDFYKRLIDKAKTVEGVEALKDAILAA.

98. The engineered polypeptide according to any one of the preceding embodiments, wherein the engineered polypeptide is selected from the group consisting of:

(SEQ ID NO: 727)

HGEGTFTSDLSKQLEEEAVRLFIEWLKQGGPSKEIISTGGGGSASGSLAE
AKEAANAELDSYGVSDFYKRLIDKAKTVEGVEALKDAILAALP;

(SEQ ID NO: 728)

HGEGTFTSDLSKQLEEEAVRLFIEWLKNGGPSSGAPPKSTGGGGSASGSL
AEAKEAANAELDSYGVSDFYKRLIDKAKTVEGVEALKDAILAALP;

(SEQ ID NO: 729)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAAL;

(SEQ ID NO: 730)

HGEGTFTSDLSKQMEEEAVRLFIEWLKNGGPSSGAPPPSTGGGGSASGSL
AEAKEAANAELDSYGVSDFYKRLIDKAKTVEGVEALKDAILAALP;

(SEQ ID NO: 731)

HGEGTFTSDLSKQMEEEAVRLFIEWLKNGGPSSGAPPPSGGSLAEAKEAA
NAELDSYGVSDFYKRLIDKAKTVEGVEALKDAILAALP;

(SEQ ID NO: 732)

HGEGTFTSDLSKQLEEEAVRLFIEWLKNGGPSSGAPPPSTGGGGSASGSL
AEAKEAANAELDSYGVSDFYKRLIDKAKTVEGVEALKDAILAALP;

(SEQ ID NO: 733)

HGEGTFTSDLSKQLEEEAVRLFIEWLKNGGPSSGAPPPSGGSLAEAKEAA
NAELDSYGVSDFYKRLIDKAKTVEGVEALKDAILAALP;

(SEQ ID NO: 734)

HGEGTFTSDLSKQLEEEAVRLFIEWLKQGGPSKEIISTGGGGSASGSLAE
AKEAANAELDSYGVSDFYKRLIDKAKTVEGVEALKDAILAAL;

(SEQ ID NO: 735)

HGEGTFTSDLSKQLEEEAVRLFIEWLKNGGPSSGAPPKSTGGGGSASGSL
AEAKEAANAELDSYGVSDFYKRLIDKAKTVEGVEALKDAILAAL;

(SEQ ID NO: 737)

HGEGTFTSDLSKQMEEEAVRLFIEWLKNGGPSSGAPPPSTGGGGSASGSL
AEAKEAANAELDSYGVSDFYKRLIDKAKTVEGVEALKDAILAAL;

(SEQ ID NO: 738)

HGEGTFTSDLSKQMEEEAVRLFIEWLKNGGPSSGAPPPSGGSLAEAKEAA
NAELDSYGVSDFYKRLIDKAKTVEGVEALKDAILAAL;
and

(SEQ ID NO: 739)

HGEGTFTSDLSKQLEEEAVRLFIEWLKNGGPSSGAPPPSGGSLAEAKEAA
NAELDSYGVSDFYKRLIDKAKTVEGVEALKDAILAAL.

99. The engineered polypeptide according to any one of the previous embodiments wherein the engineered polypeptide comprises Cmpd 5, Cmpd 9 or Cmpd 11. 100. The engineered polypeptide according to any one of the previous embodiments wherein the engineered polypeptide has at least 95% sequence identity with said engineered polypeptide. 101. The engineered polypeptide according to any one of the previous embodiments wherein the engineered polypeptide has at least 98% sequence identity with said engineered polypeptide. 102. The engineered polypeptide according to any one of the previous embodiments, which elicits no or a reduced immune response upon administration to the mammal, as compared to the immune response elicited upon administration to the mammal of the exendin that is not attached to the ABD or that is attached to an ABD described in PCT Published Appl. No. WO 2009/016043 that has not been modified for reduced immunogenicity as described herein. 103. The engineered polypeptide according to any one of the previous embodiments, for use as a medicament. 104. The engineered polypeptide according to any one of the previous embodiments, wherein said engineered polypeptide has a plasma half-life suitable for a twice daily administration. 105. The engineered polypeptide according to any one of the previous embodiments, wherein said engineered polypeptide has a plasma half-life suitable for once daily administration. 106. The engineered polypeptide according to any one of the previous embodiments, wherein said engineered polypeptide has a plasma half-life suitable for a twice weekly administration. 107. The engineered polypeptide according to any one of the previous embodiments, wherein said engineered polypeptide has a plasma half-life suitable suitable for a once weekly administration. 108. The engineered polypeptide according to any one of the previous embodiments, wherein said engineered polypeptide has a plasma half-life of at least 40 hours. 109. The engineered polypeptide according to any one of the previous embodiments, wherein said engineered polypeptide has a plasma half-life of at least 60 hours. 110. The engineered polypeptide according to any one of the previous embodiments, wherein said engineered polypeptide has a plasma half-life of at least 72 hours. 111. A composition comprising an engineered polypeptide according to any one of the previous embodiments and a pharmaceutically acceptable excipient. 112. The composition according to embodiment 111, that is aqueous. 113. The composition according to embodiment 111, that is solid. 114. The composition according to any one of the preceding embodiments, for use as a medicament. 115. The composition according to any one of the preceding embodiments wherein the engineered polypeptide is a lyophilized solid. 116. The composition according to any one of the preceding embodiments, further comprising human serum albumin. 117. A pharmaceutical composition comprising an engineered polypeptide according to any one of the previous embodiments. 118. The pharmaceutical composition of embodiment 117, comprising the engineered polypeptide and a pharmaceutically acceptable excipient. 119. The pharmaceutical composition according to any one of the preceding embodiments, that is aqueous. 120. The pharmaceutical composition according to any one of the preceding embodiments, that is solid. 121. The pharmaceutical composition according to any one of the preceding embodiments, for use as a medicament. 122. The pharmaceutical composition according to any one of the preceding embodiments wherein the engineered polypeptide is a lyophilized solid. 123. The pharmaceutical composition according to any one of the preceding embodiments, further comprising human serum albumin. 124. The pharmaceutical composition according to any one of the previous embodiments, wherein said pharmaceutical composition is a pharmaceutical composition for intramuscular, intravenous, subcutaneous, intradermal, intraarticular, intrathecal, mucosal, oral, nasal, sublingual, pulmonary or buccal delivery. 125. The pharmaceutical composition according to any one of the previous embodiments, wherein said pharmaceutical composition is a pharmaceutical composition for subcutaneous delivery. 126. The pharmaceutical composition according to any one of the previous embodiments, wherein said pharmaceutical composition is a pharmaceutical composition for delivery by injection. 127. The pharmaceutical composition according to any one of the previous embodiments, wherein said pharmaceutical composition is a pharmaceutical composition for oral delivery. 128. The pharmaceutical composition according to embodiment 127, wherein said pharmaceutical composition is a pharmaceutical composition for oral delivery and comprises a solid form. 129. The pharmaceutical composition according to embodiment 128, wherein said pharmaceutical composition is a pharmaceutical composition for oral delivery and comprises a tablet, granules, microparticles or a capsule. 130. The pharmaceutical composition according to any one of the previous embodiments, wherein said pharmaceutical composition is a sustained release or long lasting pharmaceutical composition. 131. The pharmaceutical composition according to any one of the previous embodiments, wherein said pharmaceutical composition is a twice daily administered pharmaceutical composition. 132. The pharmaceutical composition according to any one of the previous embodiments, wherein said pharmaceutical composition is a once daily administered pharmaceutical composition. 133. The pharmaceutical composition according to any one of the previous embodiments, wherein said pharmaceutical composition is a twice weekly administered pharmaceutical composition. 134. The pharmaceutical composition according to any one of the previous embodiments, wherein said pharmaceutical composition is a once weekly pharmaceutical composition. 135. A pharmaceutical composition according to any one of the previous embodiments, for treating a disease or disorder in a subject in need of such treatment. 136. The pharmaceutical composition of embodiment 135 wherein the disease or disorder is diabetes, overweight, obesity, Alzheimer's disease, Parkinson's disease, fatty liver disease, dyslipidemia, coronary artery disease, stroke, short bowel syndrome (SBS), hyperlipidemia, nonalcoholic steatohepatitis (NASH), nonalcoholic fatty liver disease (NAFLD), hypoglycemia unawareness (HU), restrictive lung disease including sarcoidosis, or metabolic syndrome X. 137. The pharmaceutical composition of embodiment 136 wherein said disease or disorder is diabetes, overweight, obesity, SBS or Parkinson's disease. 138. The pharmaceutical composition of embodiment 137 wherein said diabetes is type I diabetes, type II diabetes, prediabetes, gestational diabetes, HIV-treatment-induced diabetes, steroid-induced diabetes, or latent autoimmune diabetes in adults (LADA). 139. The engineered polypeptide or pharmaceutical composition of any one of embodiments 1 to 138 wherein the engineered polypeptide provides for once weekly administration. 140. A method for treating a disease or disorder in a subject, comprising administering an engineered polypeptide according to any one of the previous embodiments to a subject in need thereof in an amount effective to treat said disease or disorder. 141. The method according to embodiment 140, wherein said disease or disorder is diabetes, overweight, obesity, Alzheimer's disease, Parkinson's disease, fatty liver disease, dyslipidemia, coronary artery disease, stroke, short bowel syndrome (SBS), hyperlipidemia, nonalcoholic steatohepatitis (NASH), nonalcoholic fatty liver disease (NAFLD), hypoglycemia unawareness (HU), restrictive lung disease including sarcoidosis, or metabolic syndrome X. 142. The method according to embodiment 140, wherein said disease or disorder is diabetes, overweight, obesity, Alzheimer's disease, fatty liver disease, dyslipidemia, coronary artery disease, stroke, SBS, hyperlipidemia or Parkinson's disease. 143. The method according to embodiment 142, wherein said disease or disorder is diabetes, overweight, obesity, SBS or Parkinson's disease. 144. The method according to embodiment 143, wherein said disease or disorder is type T diabetes, type TT diabetes or prediabetes. 145. The method according to embodiment 142, wherein said disease or disorder is type 11 diabetes. 146. The method according to embodiment 142, wherein said disease or disorder is dyslipidemia or hyperlipidemia. 147. A polynucleotide encoding an engineered polypeptide according to any one of the preceding embodiments. 148. An expression vector comprising a polynucleotide according to embodiment 147. 149. A host cell comprising an expression vector according to clam 148. 150. A method of producing the engineered polypeptide of any one of the preceding embodiments, comprising expressing a polynucleotide encoding the engineered polypeptide. 151. A method of producing the engineered polypeptide according to any one the preceding embodiments by non-biological peptide synthesis using amino acids and/or amino acid derivatives having reactive side-chains protected, the non-biological peptide synthesis comprising: step-wise coupling of the amino acids and/or the amino acid derivatives to form a polypeptide according to any one of the preceding embodiments having reactive side-chains protected, removing the protecting groups from the reactive side-chains of the polypeptide, and folding of the resulting polypeptide in aqueous solution. 152. A method of producing the engineered polypeptide according to any one of the preceding embodiments, comprising: producing an exendin, producing an ABD, and conjugating the produced ABD with the produced exendin.

### VII. EXAMPLES

Examples 1-5 are provided to illustrate, amongst other things, the superior properties of the ABDs described herein, e.g. reduced immunogenicity properties, compared to previous ABDs.

### Example 1

### Cloning, Expression, Purification and Characterization of Albumin Binding Polypeptides

In this example, eight different albumin binding polypeptides, PEP07913 (SEQ ID NO:453), PEP07912 (SEQ ID NO:457), PEP07914 (SEQ ID NO:458), PEP07968 (i.e. DOTA conjugated to PEP07911, SEQ ID NO:459), PEP06923 (SEQ ID NO:454), PEP07271 (SEQ ID NO:455), PEP07554 (SEQ ID NO:456) and PEP07844 (SEQ ID NO:461), the amino acid sequences of which are set out in FIG. 1, were cloned, purified and characterized.

### Material and Methods.

### Cloning of Albumin Binding Polypeptide Variants.

Mutations in G148-GA3 were generated using site directed mutagenesis with the appropriate oligonucleotides to obtain the desired albumin binding polypeptide variants. The gene fragments were amplified by PCR with primers adding specific endonuclease sites as well as an N-terminal MGSS sequence (SEQ ID NO: 844) preceding the albumin binding polypeptide variants. The fragments were cleaved with Ndel and Notl, purified and ligated to a cloning vector, the plasmid pAY02556 (containing an origin of replication from pBR322, a kanamycin resistance gene and a T7 promoter for expression of the gene of interest), restricted with the same enzymes. Ligations were transformed to electrocompetent E. coli TOP10 cells. The transformed cells were spread on TBAB plates (30 g/l tryptose blood agar base) supplemented with 50 μg/ml of kanamycin, followed by incubation at 37° C. overnight. The colonies were screened using PCR and sequencing of amplified fragments was performed using the biotinylated oligonucleotide and a BigDye® Terminator v3.1 Cycle Sequencing Kit (Applied Biosystems), used in accordance with the manufacturer's protocol. The sequencing reactions were purified by binding to magnetic streptavidin coated beads using a Magnatrix 8000 (NorDiag AB), and analyzed on ABI PRISM® 3100 Genetic Analyzer (PE Applied Biosystems). All albumin binding polypeptide variants were subcloned as monomers and the constructs encoded by the expression vectors were MGSS-[PP###] (“MGSS” disclosed as SEQ ID NO: 844), where PP### corresponds to the 46 amino acid residues constituting the sequence of the albumin binding polypeptide.

In addition, the gene fragments of G148-GA3, PP007 (SEQ ID NO:307), PP013 (SEQ ID NO:313) and PP037 (SEQ ID NO:337) were amplified by PCR with primers adding specific endonuclease sites as well as a hexahistidin sequence (SEQ ID NO: 49), a TEV protease site and a glycine residue before the 46 amino acid residues constituting the sequence of the albumin binding polypeptide. The polypeptides PEP07913 (SEQ ID NO:453), PEP07912 (SEQ ID NO:457), PEP07914 (SEQ ID NO:458) and PEP07968 (SEQ ID NO:459) correspond to the albumin binding polypeptides G148-GA3, PP007 (SEQ ID NO:307), PP013 (SEQ ID NO:313) and PP037 (SEQ ID NO:337) with glycine residues added. The fragments were cleaved with Xbal and Notl, purified and ligated to a cloning vector, the plasmid pAY02512 (containing an origin of replication from pBR322, a kanamycin resistance gene and a T7 promoter for expression of the gene of interest. The cloning site is preceded by a sequence encoding a peptide containing a hexahistidine tag (SEQ ID NO: 49) followed by a cleavage site for the TEV protease), restricted with the same enzymes. Ligation, transformation and sequence verification were performed as described above. The four albumin binding polypeptide variants G148-GA3, PP007, PP013 and PP037 were subcloned as monomers and the constructs encoded by the expression vectors were MGSSHHHHHHLQSSGVDLGTENLYFQG-[PP###], where MGSSHHHHHHLQSSGVDLGTENLYFQG is SEQ ID NO:600, and “PP###” represents a ABD sequence described herein.

### Protein Expression.

The albumin binding polypeptide variants were expressed in E. coli BL21 (DE3) either with an N-terminal MGSS-extension (“MGSS” disclosed as SEQ ID NO: 844) or with an N-terminal His6-tag (SEQ ID NO: 49) followed by a TEV-protease recognition site and a glycine residue. A colony of each albumin binding polypeptide variant was used to inoculate 4 ml TSB+YE medium supplemented with kanamycin to a concentration of 50 μg/ml. The cultures were grown at 37° C. for approximately 5 hours. 3 ml from each of the cultures was used to inoculate 800 ml TSB+YE supplemented with kanamycin to a concentration of 50 μg/ml in parallel bio reactors (Greta system, Belach Bioteknik AB). The cultivations were performed at 37° C., with aeration at 800 ml/minute and an agitation profile to keep dissolved oxygen levels above 30%, to an OD600 of 2, which was followed by addition of IPTG to a final concentration of 0.5 mM. Cultivation was continued for five hours after which the cultivation was cooled to 10° C., aeration was stopped and agitation lowered to 300 rpm. Cell pellets were harvested by centrifugation (15600×g, 4° C., 20 minutes) and stored at −20° C. until purification.

Purification of Albumin Binding Polypeptide Variants with a His6-tag (SEQ ID NO: 49) and a TEV-Protease Site.

Frozen cell pellets harboring soluble hexahistidine-tagged polypeptides (“hexahistidine” disclosed as SEQ ID NO: 49) PEP07913 (SEQ ID NO:453), PEP07912 (SEQ ID NO:457), PEP07914 (SEQ ID NO:458) and PEP07968 (SEQ ID NO:459) were suspended in 35 ml binding buffer (20 mM sodium phosphate, 0.5 M NaCl, 20 mM imidazole, pH 7.4) with an addition of 1000 U Benzonase® (1.01654.001, Merck) and disrupted by ultrasonication. For each of the polypeptides, the ultrasonicated suspension was clarified by centrifugation (1 h, 37000×g, 4° C.) and the supernatant was loaded onto a His GraviTrap™ column (11-0033-99, GE Healthcare). The column was washed with 10 ml washing buffer (20 mM sodium phosphate, 0.5 M NaCl, 60 mM imidazole, pH 7.4), before eluting the polypeptide with 3 ml elution buffer (20 mM sodium phosphate, 0.5 M NaCl, 0.5 M imidazole, pH 7.4). The buffer was exchanged to a cleavage buffer (50 mM Tris-HCl, 150 mM NaCl, pH 8) using PD-10 desalting column (17-0851-01, GE Healthcare). The amount of polypeptide product was determined by measuring the absorbance at 280 nm before adding DTT to a final concentration of 5 mM. His6-tagged TEV protease (“His6” disclosed as SEQ ID NO: 49) was added to the cleavage buffer at a 1:10 mass ratio relative to the polypeptide product. The cleavage was performed over night under slow mixing at 4° C. Imidazole was added to the cleavage mix, to a concentration of 20 mM, before loading the mix onto a His GraviTrap™ column (11-0033-99, GE Healthcare) for removing cleaved His6-tags (SEQ TD NO: 49), His6-tagged TEV protease (“His6” disclosed as SEQ ID NO: 49) and His6-tagged uncleaved product (“His6” disclosed as SEQ ID NO: 49).

For each variant, the flow-through, containing the albumin binding polypeptide variant, was further purified by reversed phase chromatography (RPC), as follows. The flow-through fraction was loaded on 1 ml Resource 15 RPC column (GE Healthcare), previously equilibrated with RPC A Buffer (0.1% TFA in water). After column wash with 10 column volumes (CV) RPC A Buffer, bound polypeptides were eluted with a linear gradient of 0-50% RPC B Buffer (0.1% TFA in acetonitrile) during 10 CV. The flow rate was 2 ml/min and the absorbance at 280 nm was monitored. Fractions containing albumin binding polypeptide variant were identified by SDS-PAGE analysis and pooled.

The RPC-purified albumin binding polypeptide variants were further purified by gel filtration on 120 ml Superdex 75 (GE Healthcare) packed in an XK16 column (GE Healthcare). The running buffer was 1×PBS, and the flow rate 2 ml/min. Fractions containing pure albumin binding polypeptide variant were pooled and concentrated to approximately 1.3 mg/ml. Finally, the concentrate was purified from trace amounts of remaining endotoxins by using 1 ml columns of AffinityPak Detoxi-Gel Endotoxin removing gel (Pierce, prod#20344), according to the manufacture's recommendations.

The albumin binding polypeptide variant PEP07911 was conjugated with Mal-DOTA before the RPC-purification step, as follows. The buffer of the flow-through fraction from the 1MAC-FT purification step was exchanged to 0.2 M NaAc, pH 5.5, using a disposable PD-10 desalting column (GE Healthcare). Maleimido-mono-amide-DOTA (Macrocyclics, cat. no. B-272) was added at 5-fold molar excess and incubated for 60 minutes at 30° C. under continuous shaking. The resulting polypeptide was denoted PEP07968.

### Purification of Albumin Binding Polypeptide-Variants Without His6-Tag (SEQ ID NO: 49).

Frozen cell pellets harboring soluble albumin binding polypeptide variants PEP06923 (SEQ ID NO:454), PEP07271 (SEQ ID NO:455), PEP07554 (SEQ ID NO:456) and PEP07844 (SEQ ID NO:461) were suspended in 20 mM Tris-HCl, pH 8 and disrupted by ultrasonication. For each of the polypeptide variants, the ultrasonicated suspension was clarified by centrifugation (30 min, 32000×g, 4° C.) and the supernatant was loaded onto a HSA-Sepharose column (GE Healthcare). After washing with TST-buffer (25 mM Tris-HCl, 1 mM EDTA, 200 mM NaCl, 0.05% Tween 20, pH 8.0), followed by 5 mM NH4Ac, pH 5.5, bound albumin binding polypeptide variant was eluted with 0.5 M HAc, pH 3.2.

The albumin binding polypeptide variants were further purified by reversed phase chromatography (RPC), as follows. For each of the variants, the eluate from the HSA-affinity purification step was loaded on 1 ml Resource 15 RPC column (GE Healthcare), previously equilibrated with RPC A Buffer (0.1% TFA in water). After column wash with 10 CV RPC A Buffer, bound polypeptides were eluted with a linear gradient of 0-50% RPC B Buffer (0.1% TFA in acetonitrile) during 10 CV. The flow rate was 2 ml/min and the absorbance at 280 nm was monitored. Fractions containing pure albumin binding polypeptide variants were identified by SDS-PAGE analysis and pooled. Finally, the buffer was exchanged to 1×PBS (2.68 mM KCl, 137 mM NaCl, 1.47 mM KH2PO4, 8.1 mM Na2HPO4, pH 7.4) using a disposable PD-10 desalting column (GE Healthcare).

### Characterization of Purified Albumin Binding Polypeptide-Variants.

The concentration was assessed by measuring the absorbance at 280 nm using a NanoDrop® ND-1000 Spectrophotometer. The proteins were further analyzed with SDS-PAGE and LC-MS.

For the SDS-PAGE analysis, approximately 10 μg of each albumin binding polypeptide variant was mixed with NuPAGE LDS Sample Buffer (Invitrogen), incubated at 70° C. for 15 min and loaded onto NuPAGE 4-12% Bis-Tris Gels (Invitrogen). The gels were run with NuPAGE MES SDS Running Buffer (Invitrogen) in an XCell II SureLock Electrophoresis Cell (Novex) employing the Sharp Prestained Standard (Invitrogen) as molecular weight marker and using PhastGel BlueR (GE Healthcare) for staining.

To verify the identity of the albumin binding polypeptide variants, LC/MS analyses were performed using an Agilent 1100 LC/MSD system, equipped with API-ESI and a single quadruple mass analyzer. Approximately 10 μg of each of the purified albumin binding polypeptide variants was loaded on a Zorbax 3005B-C8 Narrow-Bore column (2.1×150 mm, 3.5 μm, Agilent Technologies) at a flow-rate of 0.5 ml/min. Polypeptides were eluted using a linear gradient of 10-70% solution B for 15 min at 0.5 ml/min. The separation was performed at 30° C. The ion signal and the absorbance at 280 and 220 nm were monitored. The molecular weights of the purified albumin binding polypeptide variants were confirmed by MS.

### Results.

The expression levels of the albumin binding polypeptide variants were 10-30 mg product/g cell pellet, as estimated from SDS-PAGE analysis.

For all variants, the purity, as determined by SDS-PAGE analysis, exceeded 95% and the LC/MS analysis verified the correct molecular weights. After purification, between 1 and 8 mg of pure polypeptide was obtained for each of the eight albumin binding polypeptide variants.

### Example 2

### Affinity Determination for Albumin Binding Polypeptides

In this example, PEP06923 (SEQ TD NO:454), PEP07271 (SEQ TD NO:455), PEP07844 (SEQ ID NO:461), PEP07912 (SEQ ID NO:457), PEP07913 (SEQ ID NO:453), PEP07914 (SEQ ID NO:458) and PEP07968, (i.e. DOTA-conjugated to PEP07911, SEQ ID NO:459), synthesized or expressed and purified in Example 1 were characterized for affinity to human serum albumin (HSA) using a Biacore instrument. PEP07913 corresponds to the amino acid sequence of G148-GA3 with addition of a N-terminal glycine residue, whereas PEP07271, PEP07844, PEP07912, PEP07914 and PEP07968 correspond to the albumin binding polypeptides of PP001 (SEQ ID NO:301), PP043 (SEQ ID NO:343), PP007 (SEQ ID NO:307), PP013 (SEQ ID NO:313) and PP037 (SEQ ID NO:337) with different N-terminal amino acid additions.

### Material and Methods.

Biosensor analysis on a Biacore2000 instrument (GE Healthcare) was performed with HSA (Albucult®, Novozyme), immobilized by amine coupling onto the carboxylated dextran layer of the surfaces of CM-5 chips (research grade; Biacore) according to the manufacturer's recommendations. Surface 1 of the chip was activated and deactivated and used as a reference cell (blank surface) during injections, whereas surface 2 comprised HSA immobilized to 731 resonance units (RU) and surface 4 comprised HSA immobilized to 955 RU. The purified albumin binding polypeptide variants were diluted in running buffer HBS-EP (GE Healthcare) to 2.5 nM, 10 nM and 40 nM, and injected at a constant flow-rate of 50 μl/min for 5 minutes, followed by injection of HBS-EP for 60 minutes. The surfaces were regenerated with one injection of 25 μl HCl, 10 mM. The affinity measurements were performed in two sets; in the first set HBS-EP, PEP06923, PEP07271, PEP07912, PEP07913, PEP07914 and PEP07968 were injected (chip surface 2), and in the second set HBS-EP, PEP06923, PEP07844, PEP07912 and PEP07914 were injected (chip surface 4). PEP06923 was injected twice in each run as a control. The results were analyzed with a BiaEvaluation software (GE Healthcare). Curves of the blank surface were subtracted from the curves of the ligand surfaces.

### Results.

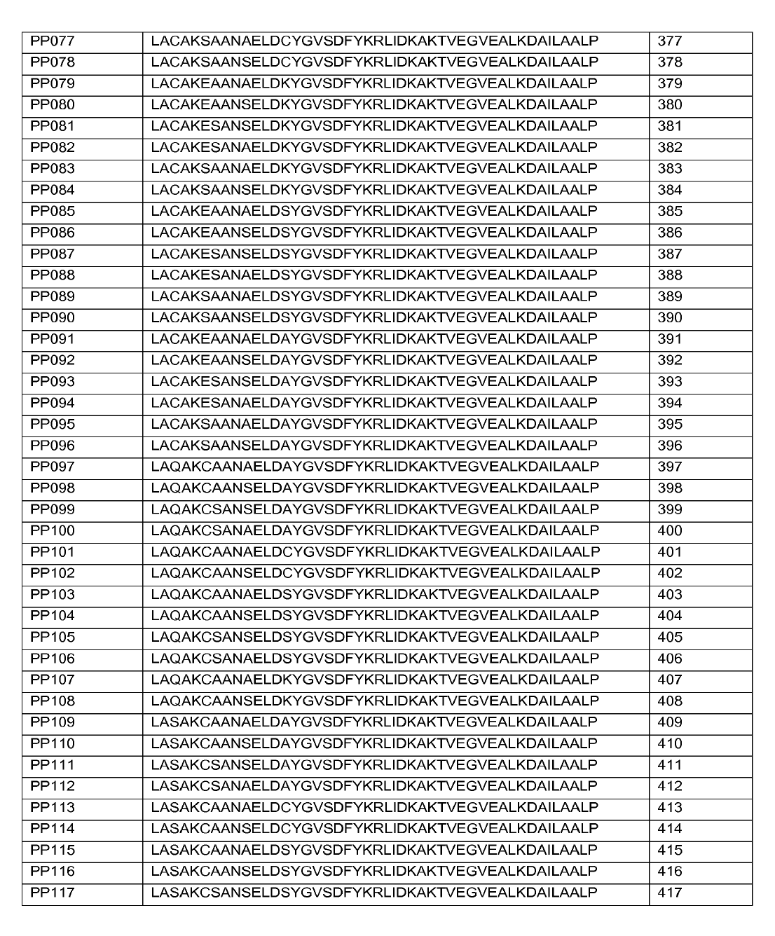

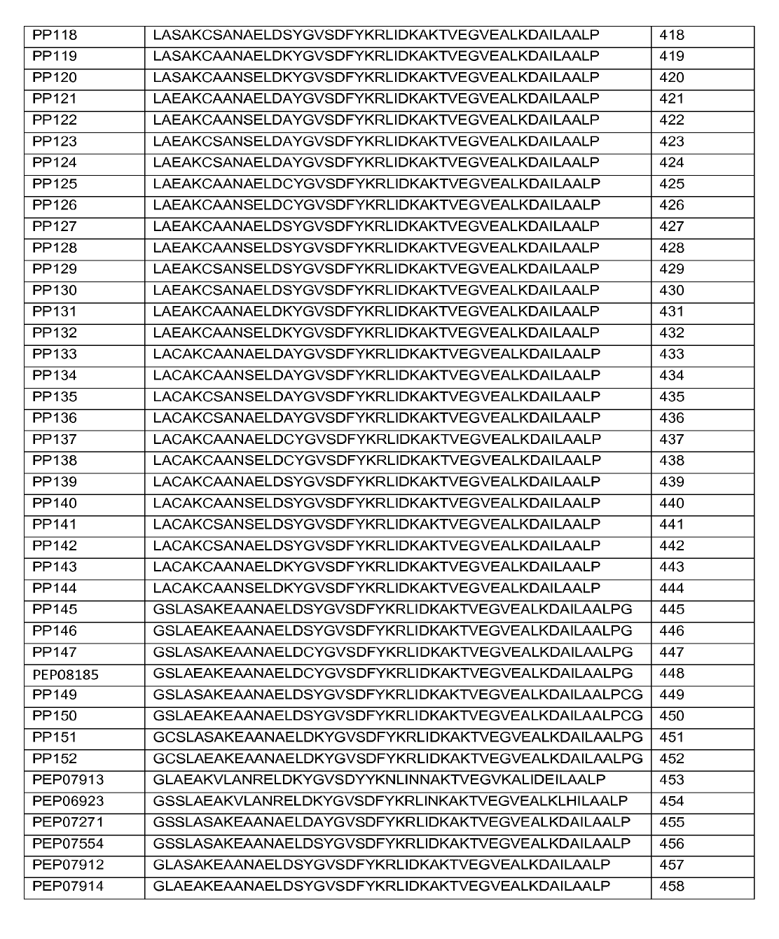

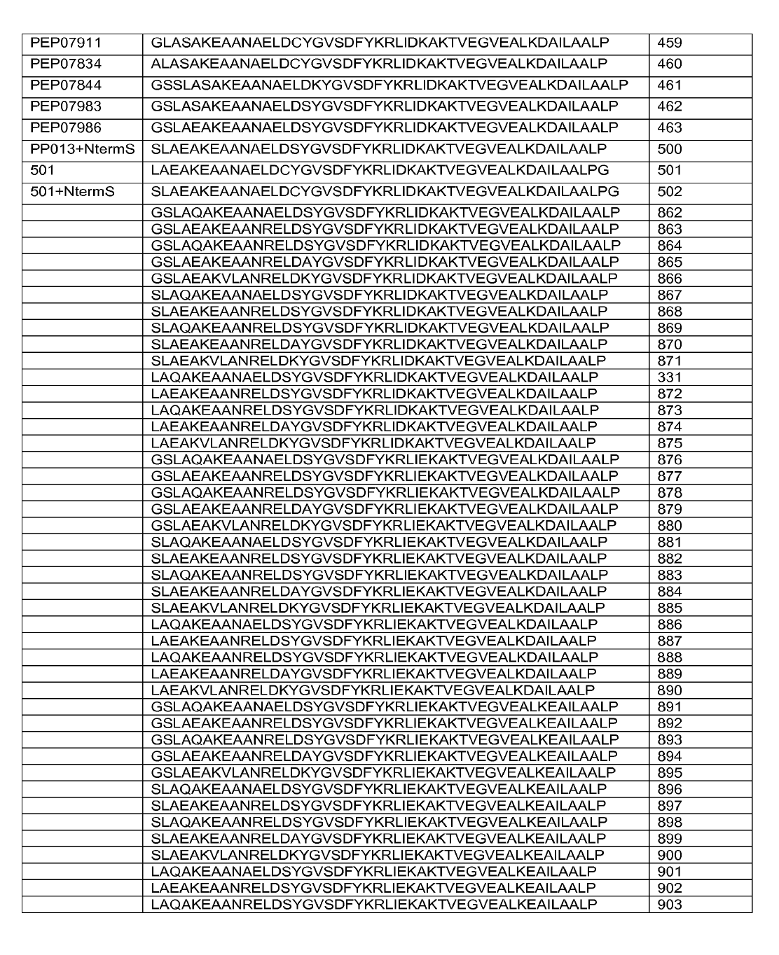

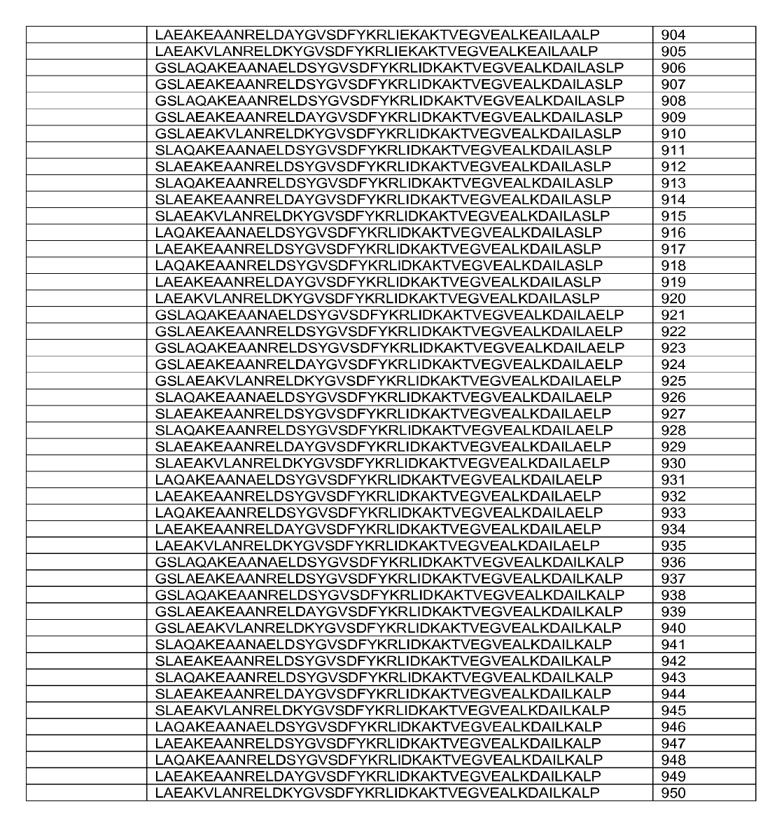

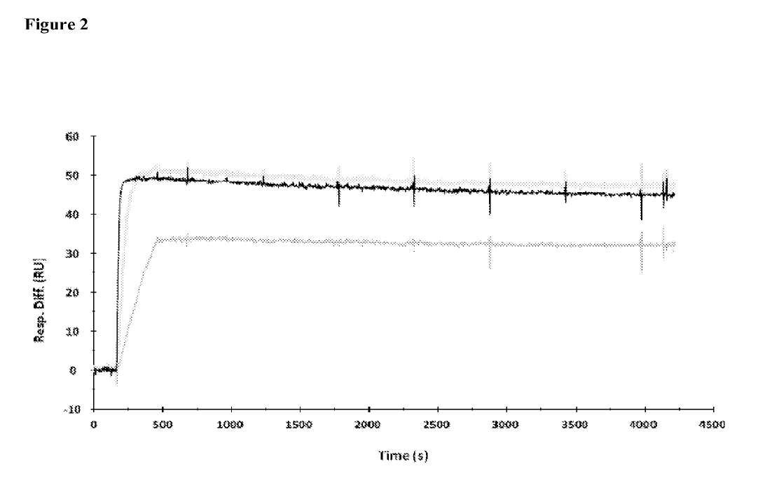

The Biacore 2000 instrument has a technical limitation, hindering measurements of very high affinity. Hence, the purpose of the Biacore study was not to determine the exact kinetic parameters of the albumin binding polypeptide variants' affinity for HSA. However, the results provide a quantitative estimation of the relative affinities of these polypeptides for albumin. After subtraction of reference surface and buffer injection, curves were fitted to a 1:1 (Langmuir) binding model using BlAevaluation software with correction for mass transfer and with RUmax set as a local parameter. Curves are shown in FIG. 2. The relative K_{D}, kₐ (kₒₙ) and k_{d} (k_{off}) values were estimated and are presented in the Tables below.

TABLE 5
Kinetic parameters (kₐ, k_{d} and K_{D}) of albumin
binding polypeptides to HSA, 1st set

| | kₐ (Ms⁻¹) | k_{d} (s⁻¹) | K_{D} (M) |
|---|---|---|---|
| PEP07913 | 5.7 × 10⁵ | 9.3 × 10⁻⁴ | 1.6 × 10⁻⁹ |
| PEP06923 (1) | 2.9 × 10⁷ | 2.9 × 10⁻⁵ | 9.9 × 10⁻¹³ |
| PEP06923 (2) | 2.6 × 10⁷ | 2.8 × 10⁻⁵ | 1.1 × 10⁻¹² |
| PEP07271 | 3.9 × 10⁶ | 2.9 × 10⁻⁵ | 7.5 × 10⁻¹² |
| PEP07912 | 4.6 × 10⁶ | 2.8 × 10⁻⁵ | 6.2 × 10⁻¹² |
| PEP07914 | 3.5 × 10⁶ | 2.5 × 10⁻⁵ | 7.2 × 10⁻¹² |
| PEP07968 | 3.0 × 10⁶ | 2.7 × 10⁻⁵ | 9.0 × 10⁻¹² |

TABLE 6
Kinetic parameters (kₐ, k_{d} and K_{D}) of albumin
binding polypeptides to HSA, 2nd set

| | kₐ (Ms⁻¹) | k_{d} (s⁻¹) | K_{D} (M) |
|---|---|---|---|
| PEP06923 (1) | 2.0 × 10⁷ | 2.6 × 10⁻⁵ | 1.3 × 10⁻¹² |
| PEP06923 (2) | 2.1 × 10⁷ | 2.5 × 10⁻⁵ | 1.2 × 10⁻¹² |
| PEP07912 | 5.4 × 10⁶ | 2.8 × 10⁻⁵ | 5.2 × 10⁻¹² |
| PEP07914 | 3.8 × 10⁶ | 2.6 × 10⁻⁵ | 6.9 × 10⁻¹² |
| PEP07844 | 5.4 × 10⁶ | 2.3 × 10⁻⁵ | 4.4 × 10⁻¹² |

As shown in Table 5 and 6, PEP07271 (SEQ ID NO:455), PEP07844 (SEQ ID NO:461), PEP07912 (SEQ ID NO:457), PEP07914 (SEQ ID NO:458) and PEP07968 (PEP07911 conjugated with DOTA, SEQ ID NO:459) all seem to have approximately the same affinity for HSA, widely exceeding the affinity of the parent G148-GA3 (PEP07913; SEQ ID NO:453). The HSA affinity of these polypeptides is slightly lower compared to PEP06923 (SEQ ID NO:454), despite similar off-rate.

### Example 3

### Determination of Melting Temperature (Tm) for Albumin Binding Polypeptides

In this example, the albumin binding polypeptide variants PEP07913 (SEQ ID NO:453), PEP06923 (SEQ ID NO:454), PEP07271 (SEQ ID NO:455), PEP07554 (SEQ ID NO:456), PEP07912 (SEQ ID NO:457), PEP07914 (SEQ ID NO:458), PEP07968 (PEP07911 conjugated with DOTA, SEQ ID NO:459) and PEP07844 (SEQ ID NO:461), expressed and purified as described in Example 1, and the albumin polypeptide variant PEP07975 (i.e. DOTA-conjugated PEP07834, SEQ ID NO:460, via Cys14 with maleimido-mono-amide-DOTA (Macrocyclics, Cat. No. B-272), were analyzed by CD analysis. PEP07913 corresponds to the sequence of G148-GA3 having an N-terminal glycine residue, PEP06923 is an engineered high affinity derivative previously described by Jonsson et al, supra, whereas PEP07271, PEP07554, PEP07912, PEP07914, PEP07968, PEP07844 and PEP07975 are examples of the 46 amino acid residues albumin binding polypeptides of PP001 (SEQ ID NO:301), PP007 (SEQ ID NO:307), PP013 (SEQ ID NO:313), PP037 (SEQ ID NO:337) and PP043 (SEQ ID NO:343) having different N-terminal amino acid additions according to the present disclosure.

### Material and Methods.

Purified albumin binding polypeptide variants were diluted in 1×PBS, to final concentrations between 0.4 and 0.5 mg/ml. Circular dichroism (CD) analysis was performed on a Jasco J-810 spectropolarimeter in a cell with an optical path-length of 1 mm. In the variable temperature measurements, the absorbance was measured at 221 nm from 20° to 90° C., with a temperature slope of 5° C./min.

### Results.

The melting temperatures (Tm) of the different albumin binding polypeptide variants were calculated by determining the midpoint of the transition in the CD vs. temperature plot. The results are summarized in Table 7 below.

TABLE 7
Determined Tm values of tested albumin
binding polypeptide variants

| | | N-terminal | |
|---|---|---|---|
| Variant | SEQ ID NO: # | sequence³ | Tm (° C.) |
| PEP07913 | SEQ ID NO: 453 | GL | 61 |
| PEP06923 | SEQ ID NO: 454 | GSSL | 57 |
| PEP07271 | SEQ ID NO: 455 | GSSL | 65 |
| PEP07554 | SEQ ID NO: 456 | GSSL | 58 |
| PEP07912 | SEQ ID NO: 457 | GL | 53 |
| PEP07914 | SEQ ID NO: 458 | GL | 59 |
| PEP07968 | SEQ ID NO: 159¹ | GL | 53 |
| PEP07975 | SEQ ID NO: 160^{1, 2} | AL | 50 |
| PEP07844 | SEQ ID NO: 461 | GSSL | 65 |

¹The peptide is conjugated with maleimide-DOTA at the cysteine
² The peptide is amidated at the C-terminus
³Leucine (underlined) is the residue in position 1 of the 46 amino acid sequence of the albumin binding polypeptide as defined in the first aspect of the present disclosure “GSSL” in Table 7 disclosed as SEQ ID NO: 845.

The polypeptide PEP07968 is identical to PEP07912, except for the former having a cysteine residue in position 14, which is conjugated with malcimide DOTA, and the latter a serine residue. Thus, the DOTA modification should not affect the melting temperature. Also PEP07975 is maleiamide-conjugated with DOTA using Cys14, and is identical to PEP07968 except for the C-terminal amide (resulting from the peptide synthesis) and for having an N-terminal alanine instead of a glycine. Furthermore, comparing PEP07912 and PEP07554 reveals that an N-terminal serine gives a higher melting temperature than a glycine in the same position (5° C. difference in Tm). Thus, all albumin binding polypeptide variants according to the present disclosure show Tm above 55° C., except PEP07912 and DOTA-conjugated variants. Taking into consideration the importance of the N-terminal portion, all the tested albumin binding polypeptides are superior to the derivative of Jonsson et al, e.g. PEP06923.

### Example 4

### Serum Response Analysis

The percentage of human serum containing IgG, capable of binding to a set of albumin binding polypeptides as disclosed herein was analyzed by ELISA. In total, 149 serum samples corresponding to 127 individuals were screened.

### Material and Methods.

ELISA plates (96-well, half area plates (Costar, cat. No. 3690)) were coated with 50 μl/well of Albucult® (Novozyme) diluted to 8 μg/ml in coating buffer (Sigma, cat. No. 3041). The plates were coated over night for three days at 4° C. On the day of analysis, the plates were washed twice with tap water and blocked for 2 hours with 100 μl of phosphate buffered saline (PBS) containing 0.05% casein (PBSC). The plates were emptied and 50 μl/well of the albumin binding polypeptides PEP07913 (SEQ ID NO:453), PEP06923 (SEQ ID NO:454), PEP07271 (SEQ ID NO:455), PEP07912 (SEQ ID NO:457), PEP07554 (SEQ ID NO:456), PEP07914 (SEQ ID NO:458), PEP07968 (DOTA conjugated PEP07911, SEQ ID NO:459) and PEP07844 (SEQ ID NO:461), diluted to 2 μg/ml in PBSC were added according to a pre-made plate layout. After incubation for two hours at room temperature (RT), the plates were washed in PBSC four times using an automated ELISA washer. The 149 serum samples from 129 individuals were diluted 50 times in PBSC by adding 24 μl serum to 1174 μl PBSC. 50 μl of the diluted sera was added per well according to the pre-made plate layout. Each serum sample was tested as a singlet. Positive and negative controls were included on each plate and for each albumin binding polypeptide. Albumin binding antibodies (50 μl, 0.5 μl/ml immunoglobulin solution prepared in house from sera from primates immunized with PEP06923) was added as a positive control and 50 μl PBSC was used as a negative control. The plates were incubated for one hour at RT and subsequently washed four times in PBSC using an automated ELISA washer. The bound IgG was detected with 50 μl/well of anti-human IgG (Southern Biotech, cat no 2040-05) diluted 10 000 times in PBSC. After washing four times in PBSC using an automated ELISA washer, 50 μl/well of substrate was added (Pierce cat. No. 34021). The reaction was stopped after 10-15 minutes by the addition of 50 μl H2504 to each well, prior to measuring the absorbance using a multi-well plate reader (Victor3, Perkin Elmer).

### Results.

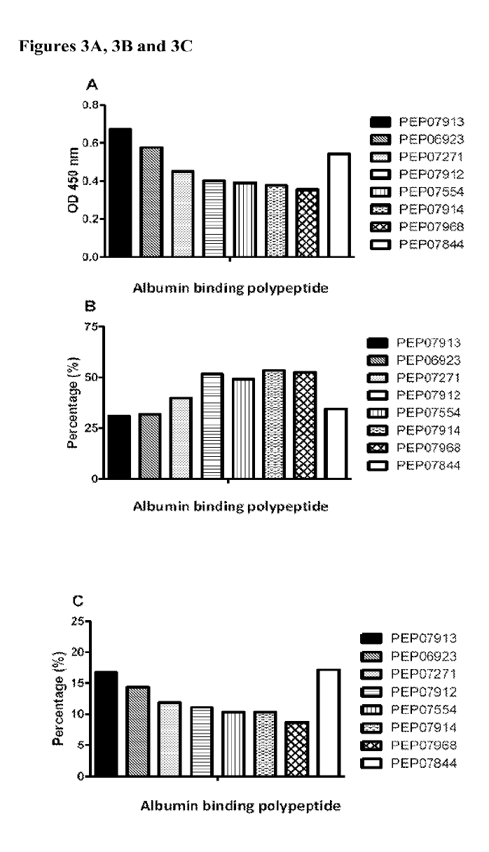

Of the 149 sera screened for IgG binding to the albumin binding polypeptides, 23 were negative for all eight polypeptides (OD-value <0.1), i.e. showed no IgG bound to the polypeptides. The analysis was performed with the 126 sera that were positive for one or more albumin binding polypeptides. The average absorbance was calculated (FIG. 3A) and the percentage of sera with OD-values values either <0.15 (FIG. 3B) or >1.0 (FIG. 3C). The highest average OD-value and the highest percentage of serum with IgG binding were obtained with PEP07913 (SEQ ID NO:453), PEP06923 (SEQ ID NO:454) and PEP07844 (SEQ ID NO:461), whereas least reactivity was found against PEP07968 (DOTA conjugated PEP07911, SEQ ID NO:459), PEP07914 (SEQ ID NO:458) and PEP07954 (SEQ ID NO:456).

Thus, the most reactive albumin binding polypeptides were the parental G148-GA3 (PEP07913, SEQ ID NO:453) and the previously affinity improved derivative (PEP06923, SEQ ID NO:454), having helix 1 retained from G148-GA3. The third of the more reactive polypeptides (PEP07844, SEQ ID NO:461) contains the original lysine in position 14 in helix 1. This residue is intended for conjugation, and will therefore not be exposed when used as such. The identical albumin binding polypeptide variant, except for having an alanine in position 14 (PEP07554, SEQ ID NO:456), is one of the least reactive.

### Example 5

### Immunogenicity Testing of Albumin Binding Polypeptides

PEP07913 (SEQ ID NO:453), PEP07912 (SEQ ID NO:457), PEP07914 (SEQ ID NO:458), and PEP07968 (i.e. DOTA conjugated PEP07911, SEQ ID NO:159) were screened for their ability to induce T cell proliferation in peripheral blood mononuclear cells (PBMC) from 52 human Caucasian individuals (obtained from CRI-Labo Medische Analyse, Gent, Belgium). PEP07913 corresponds to the sequence of G148-GA3 having an N-terminal glycine residue, whereas PEP07912, PEP07914 and PEP07968, are examples of the 46 amino acid residues albumin binding polypeptides of PP007 (SEQ ID NO:7), PP013 (SEQ ID NO:13) and PP037 (SEQ ID NO:37) having different N-terminal amino acid additions according to the present disclosure.

### Materials and Methods.

PBMCs, prepared according to standard cell biological methods, were added to a tissue culture (TC) treated 96-well round bottom plate (Falcon) in an amount of 300 000 PBMCs/ well. The cells were stimulated by addition of 100 μl/well of albumin binding polypeptides PEP07913, PEP07912, PEP07914 and PEP07968 in AIMV medium (Invitrogen) additionally containing 900 μg/ml (3-fold molar excess) of recombinant human albumin (Albucult®, Novozyme). This corresponded to a final concentration of albumin binding polypeptide of 30 μg/ml. The stimulation was done in eight-replicates, i.e. the same albumin binding polypeptide was added to eight wells in identical amounts and under the same conditions. In positive control wells, the cells were stimulated with either 30 μg/ml Keyhole Limpet Hemocyanin (KLH, Calbiochem) or 30 μg/ml tetanus toxoid (TT, Statens Serum Institut). In negative control wells, only AIMV medium with or without 900 μg/ml of albumin were added.

Cell proliferation was assessed after seven days of culturing using Alexa Fluor 488 Click-iT EdU flow cytometry assay kit (Invitrogen). 1 μM/well of EdU incorporation marker was added on day six. On day seven, cells were washed, dissociated from the plate, washed again and stained for 30 minutes with anti-CD3-PerCP reagent (Becton Dickinson) and anti-CD4-Alexa647 reagent (Becton Dickinson). Following staining, the cells were washed, fixed (BD cellfix, BD biosciences), permeabilized (using saponin) and stained for EdU by addition of Click-iT reagent according to the manufacturer's protocol (Invitrogen). After completed staining, cells were washed again and analyzed using flow cytometry (FACSCantoII, BD Biosciences). To assess the number of proliferating cells, a fixed number of fluospheres (Invitrogen) was added to each well before analysis. All staining procedures and washes were performed directly in the 96-well plate.

The raw FACSCantoII data were gated hierarchically on CD3+ CD4+ T cells and the number of gated cells as well as their fluorescence intensity of EdU-Alexa Flour 488 incorporation marker were recorded. The mean values of the number of proliferating cells/eight-plicate of protein treated wells were compared to the positive and negative controls and the resulting ratios, described as stimulation indices (SI), were calculated. Based on the SI and the variation between replicates, threshold SI-values were set to 2.0 and 0.5 for stimulation and inhibition, respectively.

### Results.

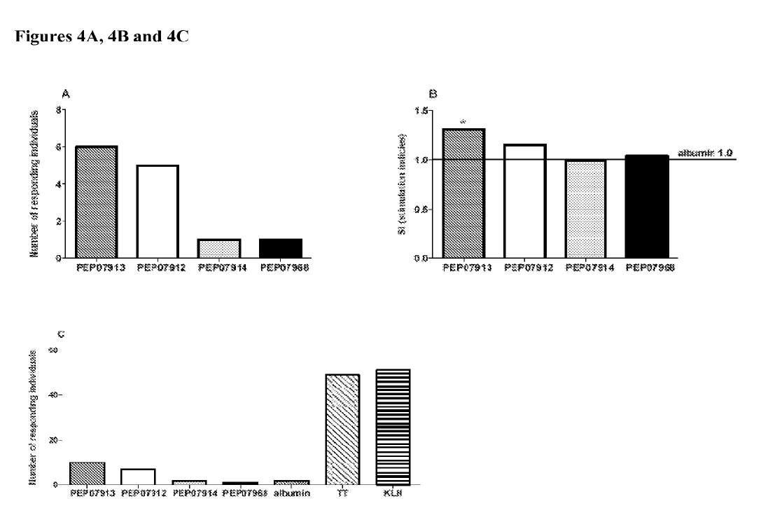

The albumin binding polypeptides PEP07913, PEP07912, PEP07914 and PEP07968 were assessed for their immunogenic potential in the presence of 3-fold excess of recombinant human albumin in a target human population using an in vitro PBMC proliferation assay. Compared to the albumin control, PEP07913 induced CD3+ CD4+ T cells proliferation in 6 of 52 donors, PEP07912 in 5 of 52 donors and PEP07914 and PEP07968 in 1 of 52 donors (FIG. 4A).

The mean stimulation index (SI) for all 52 donors was not significantly different for PEP07914 and PEP07968 compared to the negative control containing recombinant human albumin (p=0.79 and 0.48 respectively, FIG. 4B). The SI for PEP07913 was significantly higher (p=0.002) whereas the SI for PEP07912 was higher but not significant (p=0.03, FIG. 4B).

As compared to buffer only, the number of responding individuals was 10 for PEP07912, 7 for PEP07912, 2 for PEP07914, 1 for PEP07968, 2 for recombinant human albumin, and 49 and 51 for the two positive controls TT and KLH, respectively (FIG. 4C). The albumin binding polypeptides were ranked according to their immunogenicity in the following order: PEP07913>PEP07912>PEP07914>PEP07968. Both PEP07914 and PEP07968 were defined as non-immunogenic. The above results thus demonstrate that the immunogenic potential of the albumin binding polypeptides of the present disclosure is low, as compared to the positive controls.

### Example 6

### A Purification of an Exendin Analog-ABD Type Engineered Polypeptide

Method. Engineered polypeptides have been produced having an N-terminal extension which incorporates a His₆ (SEQ ID NO:49) “tag” as known in the art, for example as in sequence: MAHHHHHHVGTGSNENLYFQHGEGTFTSDLSKQLEEEAVRLFTEWLKQG GPSKEIISTGGGGSASLAEAKVLANRELDKYGVSDFYKRLINKAKTVEGVEALKLHIL AALP (SEQ ID NO: 846) which contains ABD00239 rather than an ABD of the present invention and yields Cmpd 684, provides an example of recombinant synthesis applicable to the engineered polypeptides of the present invention starting from, for example, a similar expression peptide, e.g. MAHHHHHHVGTGSNENLYFQHGEGTFTSDLSKQLEEEAVRLFIEWLKQGGPSKEIIS TGGGGSASGSLAEAKEAANAELDSYGVSDFYKRLIDKAKTVEGVEALKDAILAALP (SEQ ID NO: 838), which yields mature engineered polypeptide HGEGTFTSDLSKQLEEEAVRLFIEWLKQGGPSKEIISTGGGGSASGSLAEAKEAANAE LDSYGVSDFYKRLIDKAKTVEGVEALKDAILAALP (SEQ ID NO: 727) (Cmpd 5).

Preparation of cell extract. In order to prepare the cell extract, cell pellets from 50 mL of cell cultures were completely resuspended in 60 mL of lysis buffer (50 mM TrisHCl, 150 mM NaCl, pH 8.0). Resuspended cells were run through a microfluidizer (Microfluidics, MA) at 100 PSI three times. Cell extracts were centrifuged for 30 min at 16,000×g to remove debris. EGTA (150 mM stock) was added to cell extract to a final concentration of 3 mM.

Ni-NTA chromatography. Ten mL of 50% suspension of Ni-NTA superflow was packed to a 15 mL empty column. The column was washed with 10 mL of water, 50 mL of lysis buffer, and 20 mL of lysis buffer with 3 mM EGTA (50 mM TrisHCl, 150 mM NaCl, pH8.0, 3 mM EGTA). Cell extract was carefully added on the top of Ni-NTA column, and the flow-through was collected. The column was washed with 30 mL of lysis buffer with EGTA (50 mM TrisHCl, 150 mM NaCl, pH8.0, 3 mM EGTA). Ten mL of elution buffer (25 mM TrisHCl, 50 mM NaCl, 250 mM imidazole, pH8.0) was added to the top of column, and the elution fractions (2 mL/fraction) were collected. SDS-PAGE was run to check the flow through and each fraction. Fractions containing the His-tagged compound were pooled.

TEV protease digestion. His₆-tagged compound (“His₆” disclosed as SEQ ID NO: 49) was diluted three fold with 25 mM TrisHCl, 50 mM NaCl, pH8.0. β-mercaptoethanol (0.1%) and 2% of Turbo TEV protease (2 mg/mL, 10,000 units/mg, Accelagen), were added, and the result was mixed and incubated at RT for 2 hours and at 4° C. over night.

Removal of cleaved His-Tag and Turbo TEV with Ni-NTA. Six mL of 50% suspension of Ni-NTA superflow was packed to a 15 mL empty column. The column was washed with 20 mL of water and 20 mL of 50 mM TrisHCl, 100 mM NaCl, 45 mM imidazole, pH8.0. The TEV digest reaction was diluted 2-fold with 50 mM TrisHCl, 150 mM NaCl, pH8.0. Diluted digest reaction was carefully added to the top of Ni-NTA column, and the flow-through was collected. Ten mL of 50 mM TrisHCl, 100 mM NaCl, 45 mM imidazole, pH8.0, was added to the column to elute any unbound protein. The flow-throughs were collected and combined.

First size exclusion chromatography (SEC). The Ni-NTA flow-through was filtered with 0.2 um filter. Superdex 75 HiLoad 26/60 column was pre-equilibrated with 390 mL of PBS. Filtered flow-through was injected to the HiLoad 26/60 column with a sample pump. Protein was eluted with 1.5 CV of PBS, and the monomer peak was pooled.

Second size exclusion chromatograph. The first SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Third size exclusion chromatography. The second SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Removal of residual endotoxin with EndoTrap Red. The third SEC pool still contained ˜20 EU/mg of endotoxin, which was removed by the use of EndoTrap Red. Briefly, 0.5 mL of gel slurry was activated by adding 1 mL of Regeneration Buffer to the slurry and mix by gently shaking the tube for approximately 5 seconds. The supernatant was centrifuged and aspirated. This step was repeated two additional times. One mL of Equilibration Buffer was added, and mixing was conducted by gently shaking the tube for approximately 5 seconds. The supernatant was centrifuged and aspirated. This step was repeated two additional times. Protein sample (5.5 mL) was added to the resin and incubated for 90 minutes at RT, with gentle rocking or rotating of the tube while incubating. The result was centrifuged at 1200×g for 5 minutes, and the supernatant was transferred to a clean tube.

Results. The final purified protein migrated on SDS-PAGE gel as approximately a 6 kD protein under the conditions employed. The LC-MS showed a correct molecular weight of 9827 dalton. The protein yield was 3.3 mg from 50 mL of cell culture.

### Example 7

### Activities of Exendin-ABD Engineered Polypeptides

Exendin-ABD engineered polypeptides of the invention retained sufficient exendin activity in an in vitro cell activation assay. Additionally, the engineered polypeptides provided dramatically improved duration of action for blood glucose lowering and body weight loss, as when compared to exendin-4, when administered as a single dose to a mammal. Surprisingly, duration of action can be extended to at least 1 day, even at least 4 days, and even at least 7 days, or longer, in a rodent model, which can translate to at least one week duration of action in a human subject, thus suitable for twice daily, once daily, three times weekly, twice weekly or even once weekly administration.

### Example 8

### Albumin Binding

Characterization of the binding of engineered polypeptide compounds to albumin can be performed by any number of methods, including Biacore described herein. In this example binding measurements were conducted with a BioRad ProteOn XPR36 system (Bio-Rad Laboratories, Hercules Calif., USA; ProteOn XPR36 Protein Interaction Array System catalog number #176-0100), using a GLC sensor chip at 25 degrees C. For amine coupling the GLC chip was activated for 5 minutes using a 1:1 mixture of sulfo-NHS/EDC diluted 30-fold from the initial stock in water as shown below. Each albumin sample was diluted to 25 ug/ml in 10 mM Na acetate pH 5.0 and injected for 5 minutes over separate sensor surfaces. Each surface was then blocked with 1 M ethanolamine pH 8.5. Each albumin was coupled at a density of 2000-5000 in resonance units. The binding of an engineered polypeptide was tested using 5 nM as the highest concentration in a three-fold dilution series. The running buffer contained 10 mM HEPES pH 7.4, 150 mM NaCl, 3 mM EDTA and 0.005% tween-20. All samples were tested using a 3-fold dilution series. Each concentration series was tested in duplicate. The dissociation phase for the highest concentration was monitored for 3 hours.

The relative K_{D} measured for the engineered polypeptides are presented in Table 8 below. The results show that the albumin binding polypeptides associate with albumins with high affinity. The number in parentheses represents the standard deviation in the last significant digit. As seen from the following table the exendin polypeptides fused to albumin binding domains of ABD00239 (to create polypeptides Cmpd 684 or Cmpd 088) or an ABD of the present invention (to create engineered polypeptides of the invention, e.g. Cmpd 5) retain extremely high affinity for serum albumin from various species, especially human serum albumin, even compared to the unconjugated ABD PEP07986 itself. The engineered polypeptides retain high affinity even compared to the identical exendin analog-linker construct fused to prior ABD00239 in place of the new PEP7986 (compare Cmpd 684 to Cmpd 5).

TABLE 8

| Cmpd | Human SA | | Dog SA | | Monkey SA | | Mouse SA | | Rat SA | |
|---|---|---|---|---|---|---|---|---|---|---|
| ABD00239 | 16.1(4) | pM | 201(2) | pM | 123(1) | pM | 1.24(1) | nM | 18.3(5) | pM |
| PEP07986 | 9.5(2) | pM | 126(2) | pM | 84.0(8) | pM | 160(2) | pM | 5.7(2) | pM |
| Cmpd 088 | 84.7(9) | pM | 397(2) | pM | 77.5(6) | pM | 1.332(6) | nM | 16(1) | pM |
| Cmpd 684 | 68(1) | pM | 513(3) | pM | 90.9(9) | pM | 1.253(8) | nM | 200(200) | pM |
| Cmpd 5 | 160(1) | pM | 606(4) | pM | 140(1) | pM | 300.2(5) | pM | 12.5(2) | pM |
| Cmpd 11 | 251 | pM | 2450 | pM | 431 | pM | 1029 | pM | 51 | pM |

### Example 9

### Activity of Compounds in a GLP-1 Receptor Functional Assay

Functional activity of the compounds can be determined using a cell line expressing GLP-1 receptor. See for example United States Patent Application Publication US20110097751A1, incorporated by reference for the assay method. In this example, functional activity was determined using cells that endogenously express GLP-1R, and cAMP induction is detected as a measure of exendin activity. An HTRF assay kit was used (Cisbio International (Bedford, Mass.). The bioassay used the rat thyroid carcinoma 6-23 (clone 6) cells in the cell-based assay using the HTRF® cAMP dynamic 2 1,000 assay kit, available from Cisbio as Catalog No. 62AM4PEB. The HTRF® standards and calibrations are prepared following the instructions in the kit. Accumulation of cAMP is measured following 30 minutes of compound treatment using the HTRF (CisBio) cell-based cAMP assay kit in 384-well format. Efficacy of peptides is determined relative to cell treatment with 10 uM forskolin (a constitutive activator of adenylate cyclase), and potency (EC50) of peptides is determined by the analysis of a concentration-response curve using non-linear regression analysis fitted to a 4-parameter model. The results of the GLP-1 receptor functional activity (cAMP induction) for potency (EC50) are provided in the following Table 9.

TABLE 9
GLP-1R Functional Activity

| | GLP-1R |
|---|---|
| | Functional activity |
| Description | (EC₅₀) in nM |
| Exendin-4 | 0.004 |
| [Leu¹⁴, Gln²⁸]Exendin-4(1-32)- | 0.016 |
| fGLP-1)33-37) (SEQ ID NO: 4) | |
| Exendin-4 (1-28) amide | 0.011 |
| Cmpd 088 | 0.131 |
| Cmpd 5 | 0.486 |
| Cmpd 6 | 0.560 |
| Cmpd 7 | 0.904 |
| Cmpd 8 | 0.612 |
| Cmpd 9 | 3.21 |
| Cmpd 10 | 0.575 |
| Cmpd 11 | 1.28 |

Characterization of the in vitro activity of the engineered polypeptide compounds in the presence of serum albumin was demonstrated. Assays can be run in the presence and absence of an albumin, particularly human serum albumin. The data above was determined in the presence of about 0.1% bovine serum albumin (BSA). The following Table 10 presents functional activity of receptor activation (cAMP induction) assay described above, but in the presence of serum albumin from various species and at increased amounts. As can be seen, surprisingly, even when Cmpd 11 is bound to scrum albumin, such as to human serum albumin, despite the presence of the ABD and the large serum albumin with its potential for steric hindrance and change in the apparent Stoke's radius of the compounds resulting from albumin binding, the engineered polypeptide retains GLP-1 receptor agonist activity. Given the picomolar affinity of ABD and the engineered polypeptides to some species of serum albumin, e.g. human serum albumin, the engineered polypeptide is believed to be effectively fully bound to albumin present in the assay (and thus also fully bound in vivo in circulating blood). Because of the extremely high affinity of compound binding to albumin (as above) and the presence of a high concentration of serum albumin in the blood, it is expected that the compounds will exist essentially in the bound state in vivo yet surprisingly provide sufficient exendin functions (as demonstrated herein).

TABLE 10
GLP-1R Functional Activity in Varying
Amounts of Serum Albumin

| | GLP-1R Activation: cAMP Induction EC50 (nM) | | | |
|---|---|---|---|---|
| | 0.1% Bovine | 1% Bovine | 1% Human | 1% Rat |
| Cmpd | Albumin | Albumin | Albumin | Albumin |
| GLP-1(7-36) | 0.0306 | 0.0058 | 0.0112 | 0.0179 |
| amide | | | | |
| Cmpd 684 | 0.7854 | 0.2204 | 0.185 | 0.2473 |
| Cmpd 088 | 1.1013 | 0.2234 | 0.2022 | 0.2164 |
| Cmpd 11 | 0.8719 | 0.1774 | 0.2079 | 0.1854 |

### Example 10

### OGTT DOA (Oral Glucose Tolerance Test Duration of Action) In Vivo Activity

The effect of lowering blood glucose prior to oral gavage (1.5 k/kg dextrose) and at 30 min after gavage were investigated 1 day post dose with indicated compounds, dosed at 25 nmol/Kg, with results shown in the following table. Drug was administered to 4-hr fasted NIH/Swiss mice. At 24 hours post drug administration, an OGTT was performed to assess duration of compound activity. Blood glucose was measured with a OneTouch® Ultra® (LifeScan, Inc., a Johnson & Johnson Company, Milpitas, Calif.). *p<0.05 vs. vehicle control; ANOVA, Dunnett's test. Glucose lowering as percent lowering compared to vehicle is given (negative value indicates lowering of glucose) at 30 minutes post gavage. This OGTT DOA indicates drug activity is present at least 24 hours (for the period of time prior to the OGTT) after drug was administered. Results are presented in Table 11. Exendin-4 (unconjugated to ABD), Leu14 exendin-4 (unconjugated to ABD) and unconjugated ABD EP07986 are inactive in this assay when dosed at t-24 hours, even when provided at even higher doses. When the unconjugated exendin compounds are given at 30 nmol/kg immediately before an OGTT, both exendin-4 and Leu14 exendin-4 provide a −41% change in glucose from basal. Cmpd 12, having a C-termnally truncated exendin-4(1-28) fused to an ABD via a relatively short linker (HGEGTFTSDLSKQLEEEAVRLFIEWLKNTGGGGSASGSLAEAKEAANAELDSYGVS DFYKRLIDKAKTVEGVEALKDAILAALP; SEQ ID NO:847) was active in vitro against the GLP-1 receptor, but did not display 24 duration in this duration assay. In this case, increasing the linker length is expected to result in a desirable 24 or longer duration in this assay. This duration of action assay as well as the other in vivo assays presented herein demonstrate that the improved engineered polypeptides are extremely stable to human plasma and human cell membrane proteases.

TABLE 11
OGTT DOA

| | Percent glucose lowering |
|---|---|
| Description | compared to vehicle |
| Cmpd 5 | −19, −22 |
| Cmpd 6 | −22 |
| Cmpd 7 | −16 |
| Cmpd 8 | −18, −17 |
| Cmpd 9 | −12 |
| Cmpd 10 | −12 |
| Cmpd 11 | −23 |

### Example 11

### Effect of an Exendin-Albumin Binding Domain Polypeptide in Diabetic ob/ob Mice

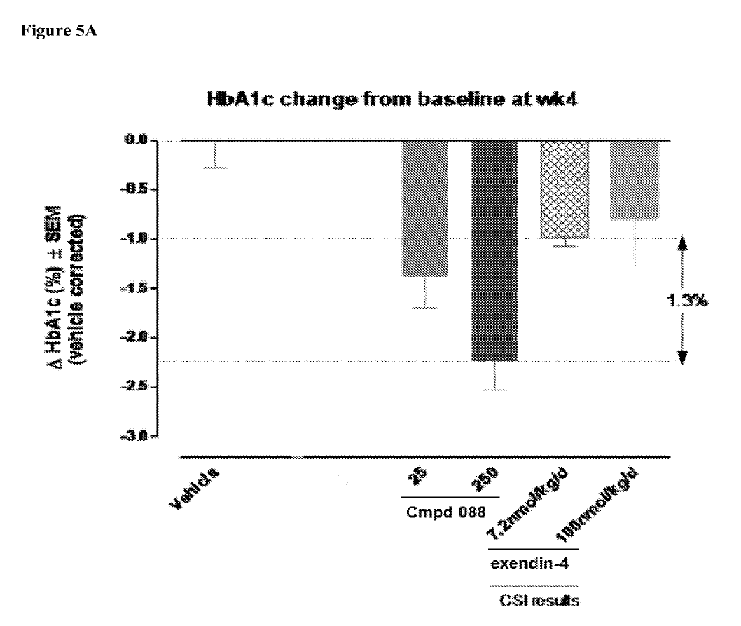

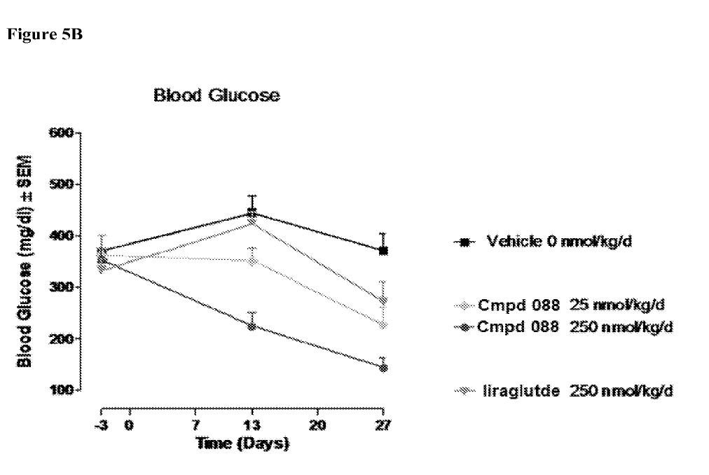

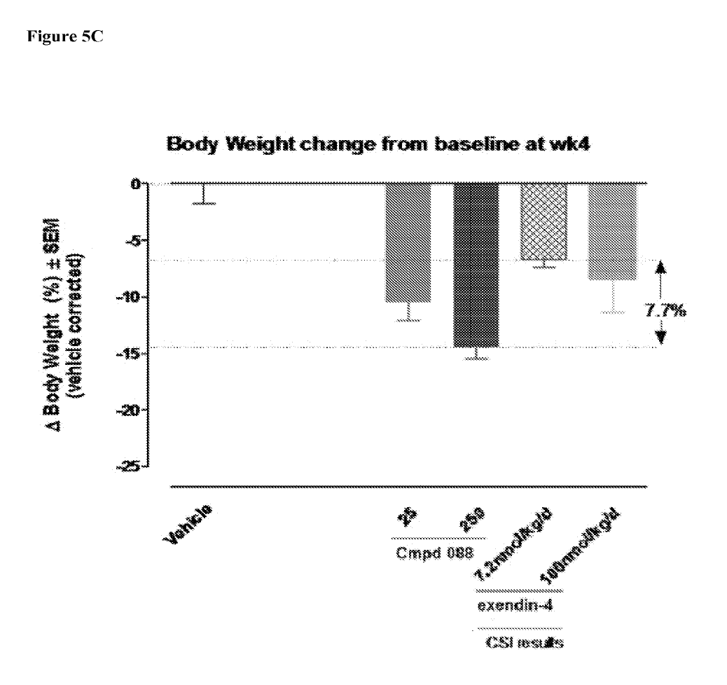

To demonstrate the effect of chronic exposure of an exendin fused to a first generation albumin binding domain polypeptide (as disclosed in PCT Published Application No. WO 2009/016043) on glucose lowering (e.g. HbA1c) and body weight loss, ob/ob/mice (diabetic) were treated with an exendin fused at its C-terminus to an albumin binding domain type polypeptide designated ABD00239 (an ABD previously disclosed in PCT Published Application No. WO 2009/016043 and not an ABD of the present invention), but which provides an example of activities of the present improved engineered polypeptides using the new improved ABDs disclosed herein, e.g. PEP7986. The time course of the effect of the test compound on body weight, glucose lowering and HbA_{1c} lowering in ob/ob mice was investigated post dose, with values at 4 weeks presented in FIGS. 5A to 5C. As depicted in FIG. 5C significant body weight loss attends treatment with 25 and 250 nmol/kg of the compound IP twice each week for 28 days. FIGS. 5A and 5B depict changes in glucose (% pre-treatment) (FIG. 5B) and in HbA1(% pre-treatment) (FIG. 5A). Points represent mean±s.d. (standard deviation). The test compound was injected sc on day=0 immediately following baseline sample collection in non-fasted male ob/ob mice. Unless indicated otherwise, blood glucose measures described herein employed a OneTouch® Ultra® device (LifeScan, Inc. Miliptas. The effect observed for the 25 nmol/kg biw (twice weekly) dose was approximately that observed for exendin-4 given at ˜7.2 nmol/kg/d by continuous infusion (CSI). Thus at a comparable dose, the exendin-4-GGS-ABD00239 compound (Cmpd 088) matched the glycemic and body weight loss effects of the maximally efficacious dose of exendin-4. At 250 nmol/kg, Cmpd 088 was twice as effective as the maximally efficacious dose of exendin-4. Further, surprisingly, at equimolar dosing the Cmpd 088 was more effective than liraglutide [N-epsilon-(gamma-Glu(N-alpha-hexadecanoyl))-Lys26,Arg34]-GLP-1-(7-37)-acid, an albumin binding GLP-1 derivative, for blood glucose lowering, HbA1c lowering and body weight loss (data not shown).

Surprisingly, despite the reduced in vitro potency compared to unconjugated exendin-4 as observed above, the acute (within 6 hours) in vivo activity of an exendin fused to an albumin binding polypeptide disclosed herein is similar to that of unconjugated exendin with regard to maximum efficacy and only slightly less (several fold) with regard to potency (ED50; e.g.), such as when measured by reduction of food intake in mice (data not shown). Even more surprisingly the effect of chronic exposure of an exendin fused to the albumin binding polypeptides disclosed herein was nearly as potent as exendin-4 (continuously infused) but was able to provide a greater maximal effect. Furthermore, in light of the very high affinity for mouse or rat albumin and low off rates, all of the engineered compounds are effectively bound to albumin in the in vivo assays (as well as in the in vitro assays). Thus the improved engineered polypeptides retain GLP-1R functional activity even when bound to albumin. This is surprising in part because albumin compounds, e.g. liraglutide, have been reported as significantly active only when dissociated from albumin. And others have reported a need to remove proteolytically a GLP-1 or exendin from an albumin binding peptide to which it was conjugated in order to obtain GLP-1 receptor function. Accordingly, the in vivo activities of the improved engineered polypeptides described throughout this application are even more impressive.

### Example 12

### Long Duration and Action of the Engineered Polypeptides In Vivo

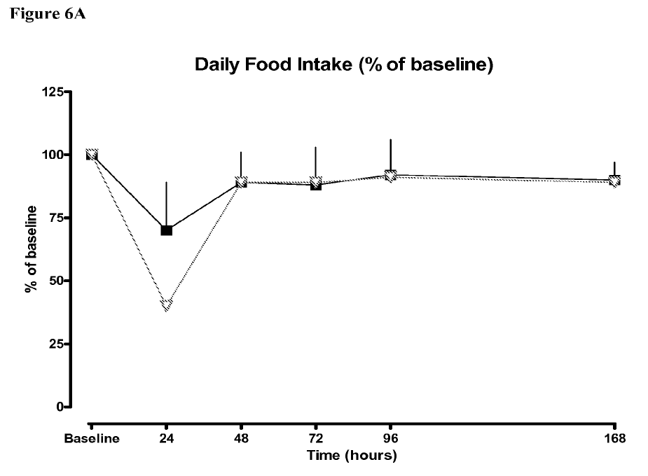

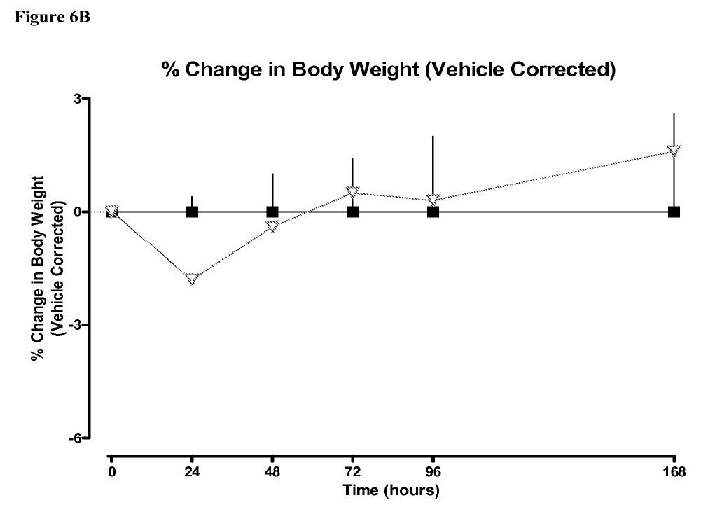

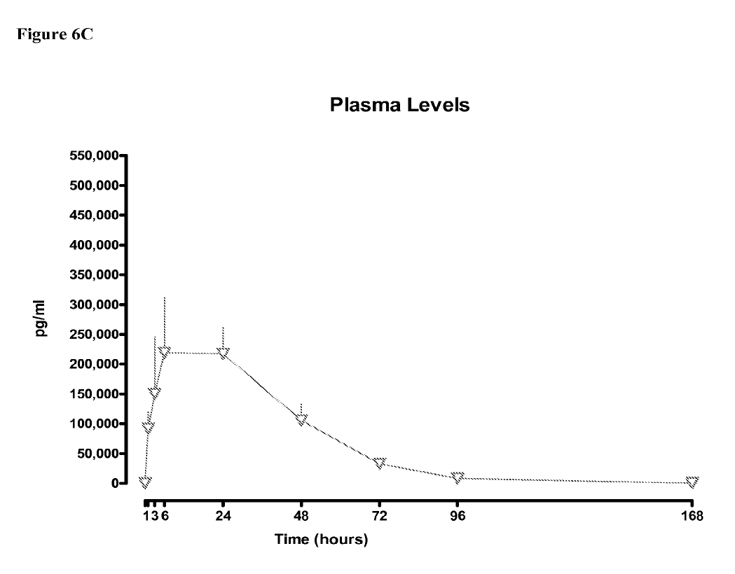

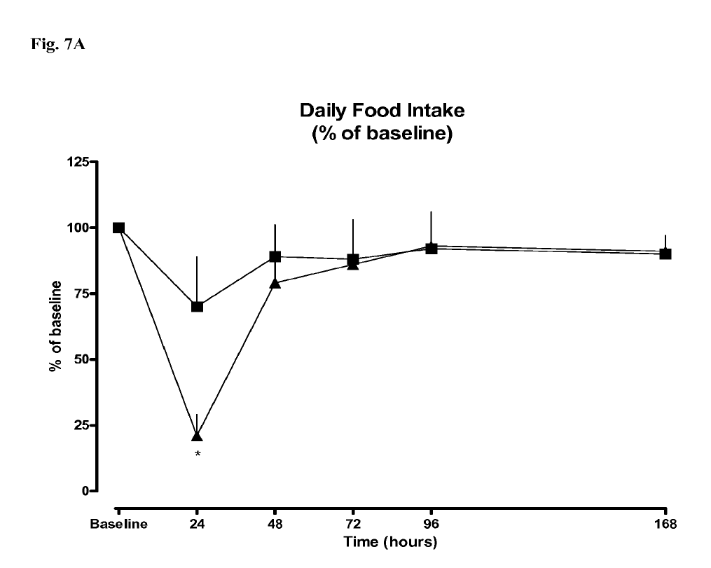

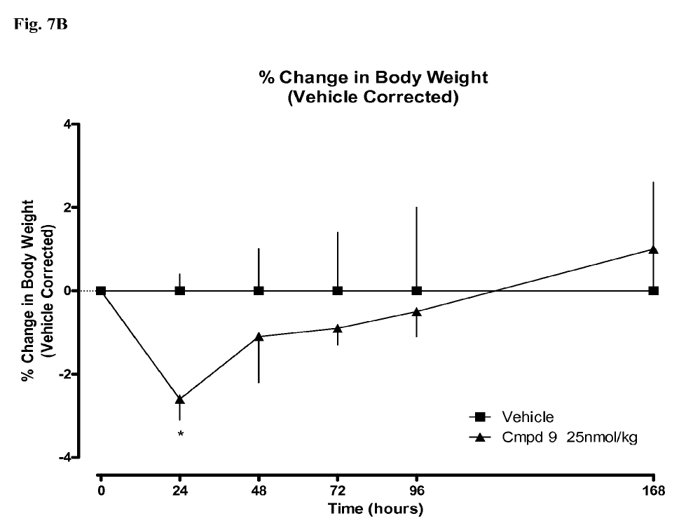

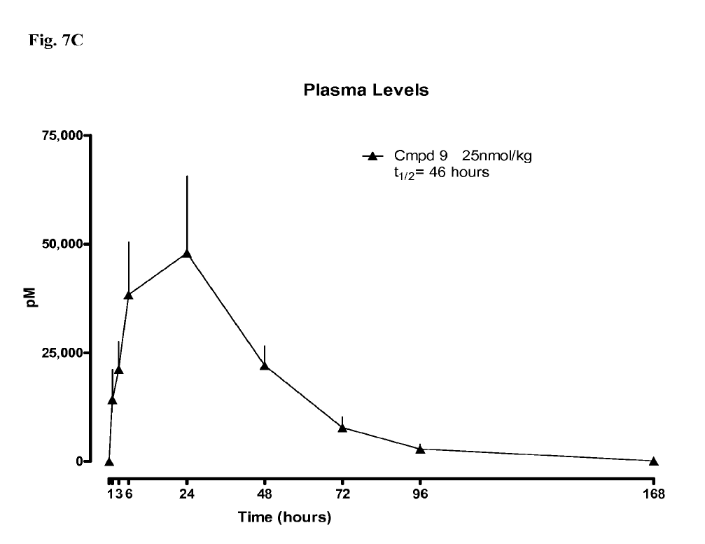

To further demonstrate the long half-life and long duration of activity of the engineered polypeptides described herein, the pharmacokinetic (PK) and pharmacodynamic (PD) properties were determined using rats. Pharmacokinetic profile and biological activity of an exemplary engineered polypeptide subcutaneously dosed in normal Harlan Sprague-Dawley (HSD) rats is presented. The recombinant engineered compounds Cmpd 11 (having Leu14 exendin-4 fused at its C-terminus via a glycine linker to PEP07986) and Cmpd 9 were injected subcutaneously at t=0 at 25 nmol/kg into normal HSD rats. Blood was collected via tail bleed at t=1 hour, 3 hours, 6 hours, 24 hours, 48 hours, 72 hours, 96 hours and 168 hours from fed HSD male rats. Food and body weights were measured daily. FIGS. 6A (Cmpd 11) and 7A (Cmpd 9) depict effect of compound to reduce food intake. FIGS. 6B (Cmpd 11) and 7B (Cmpd 9) depict effect of compound to reduce body weight. FIG. 6C (Cmpd 11) and 7C (Cmpd 9) depict a PK profile of the compound after a single dose. Points represent mean±sd. In the figures, vehicle is solid square, Cmpd 11 is open inverted triangle and Cmpd 9 is closed triangle. The plasma maximal concentration in FIG. 6C is equivalent to about 25,000 picomolar.

Exposure of up to seven (7) days was observed for these exemplary engineered polypeptides Cmpd 11, with a half-life of forty-two (42) hours, and Cmpd 9 with a half-life of forty-six (46) hours, in rats by this route of administration. By allometric scaling and in view of the strong affinity of the engineered polypeptides for human albumin, physical and biological activity duration at least as long and even longer is expected in human subjects. Accordingly, the compounds have use for at least daily, twice weekly, and even weekly administration, especially in human subjects.

### Example 13

### Long Duration and Action and Absolute Plasma Half-Life of the Engineered Polypeptides In Vivo

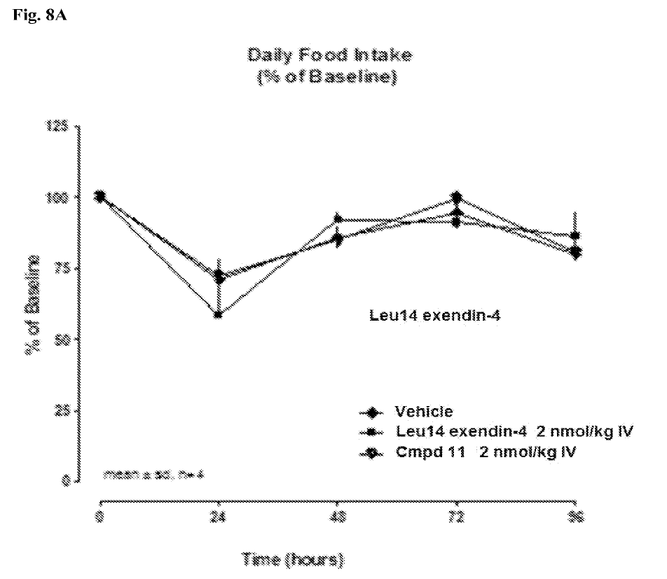

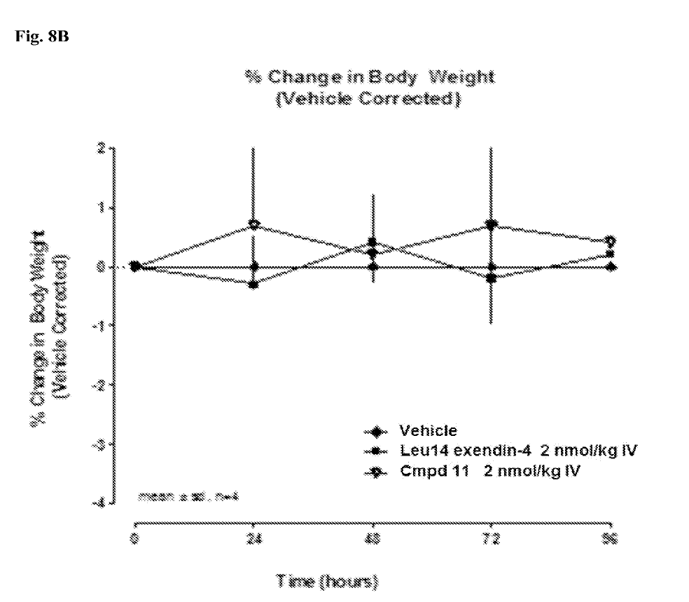

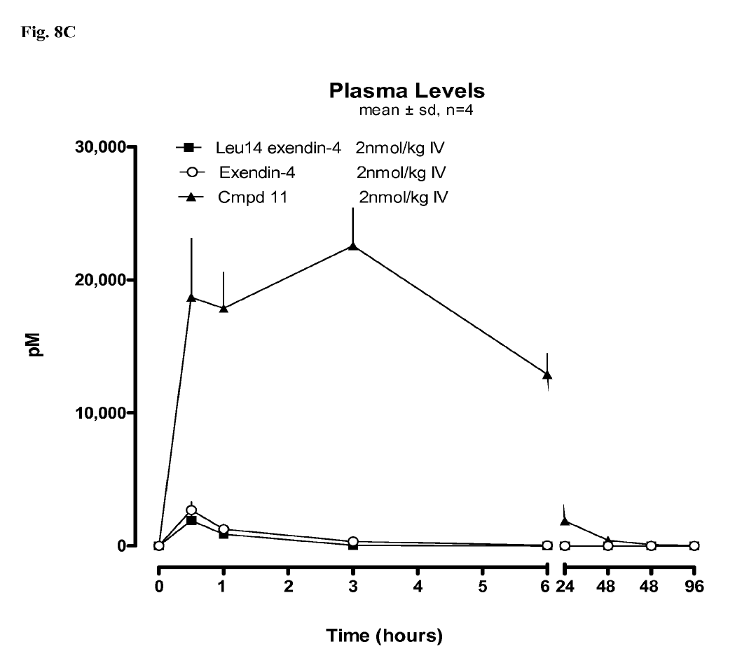

Pharmacokinetic profile and biological activity of an exemplary engineered polypeptide intravenously dosed in normal Harlan Sprague-Dawley (HSD) rats is presented, from which an absolute plasma half-life can be calculated. The recombinant engineered compound Cmpd 11 was injected intravenously at t=0 at 2 nmol/kg into normal HSD rats. Unconjugated exendin-4 and exendin-4 analog Leu14 exendin-4 were injected at 2 nmol/kg intravenously. Blood was collected via tail bleed at t=1 hour, 3 hours, 6 hours, 24 hours, 48 hours, 72 hours, 96 hours and 168 hours from fed HSD male rats. Food and body weights were measured daily. FIG. 8A depicts effect of Cmpd 11 to reduce food intake. FIG. 8B depicts effect of Cmpd 11 to reduce body weight. FIG. 8C depicts a PK profile of Cmpd 11 after a single IV dose. As expected the half-life of the unconjugated exendins is less than 15 minutes. An absolute half-life for the exemplary engineered polypeptide Cmpd 11 is estimated at about at least 12.3 hours. Points represent mean±sd.

Exposure of up to four days was observed for this exemplary engineered polypeptide, even at these relatively low doses, by this route of administration. By allometric scaling and in view of the strong affinity of the engineered polypeptides for human albumin, physical and biological activity duration at least as long and even longer is expected in human subjects. Accordingly, the compounds have use for at least twice daily (e.g. morning and night), at least daily, twice weekly, and even once weekly administration, especially in human subjects.

### Example 14

### Sub-Chronic Dosing Provides Superimpositioning and Exendin-Like Pharmacology

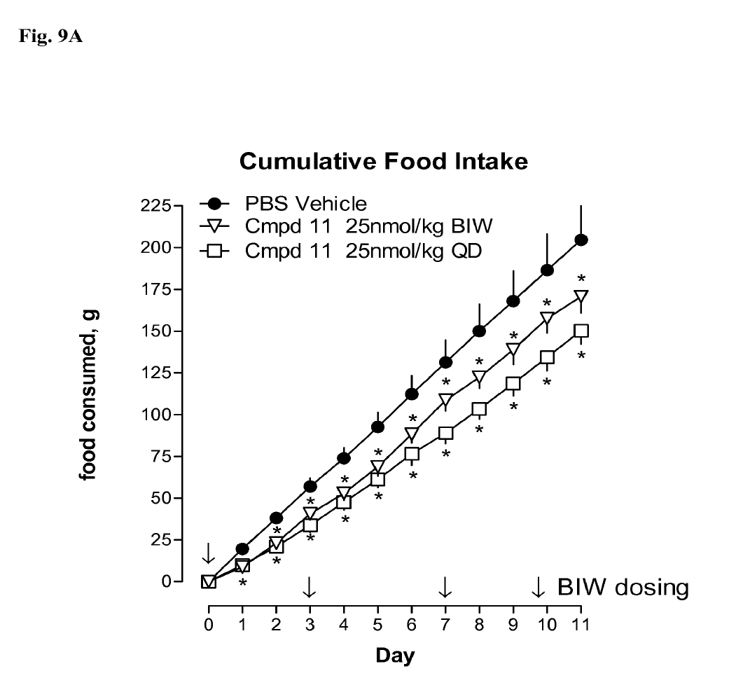

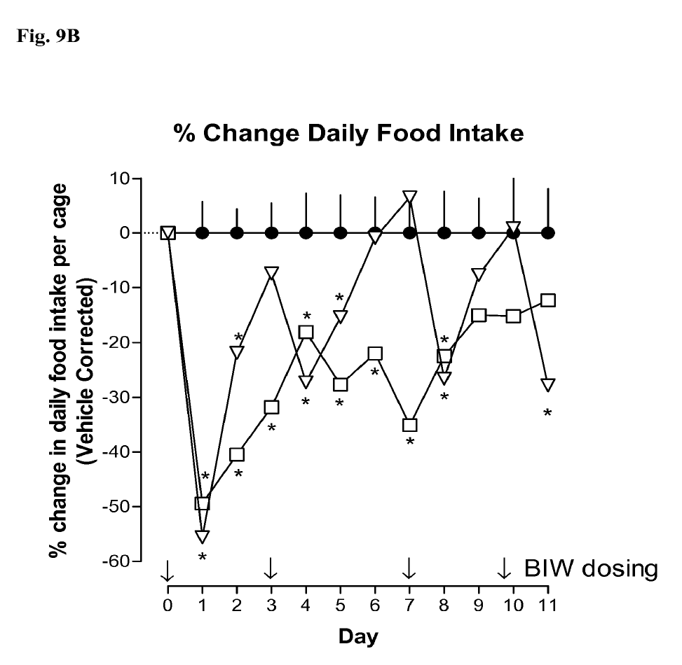

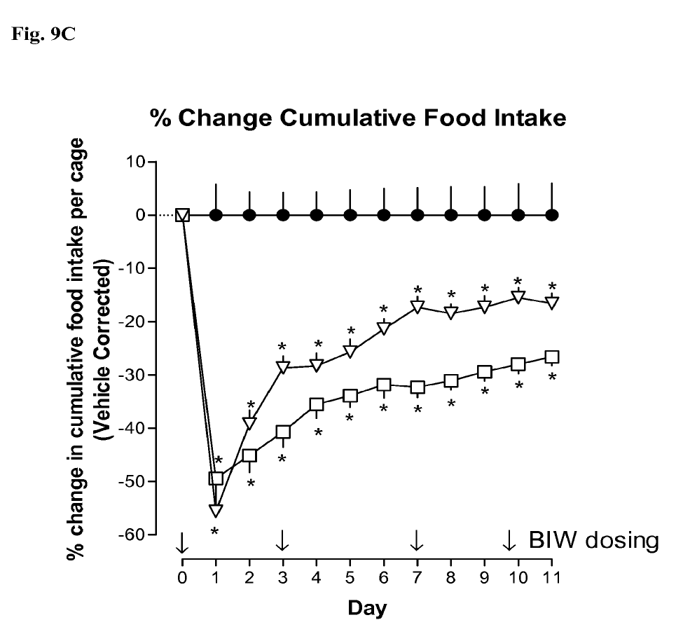

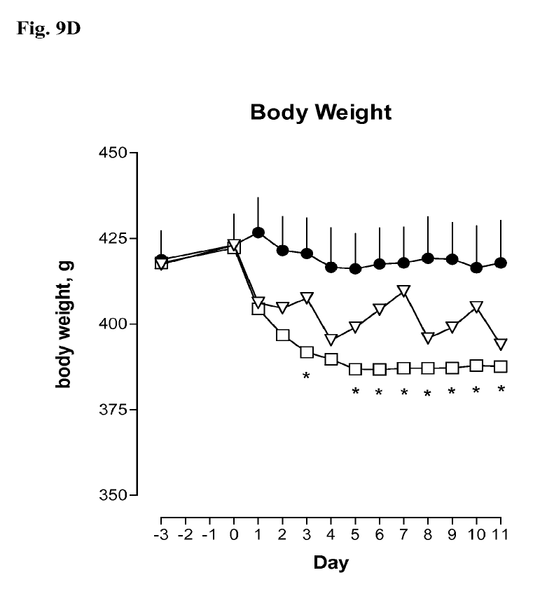

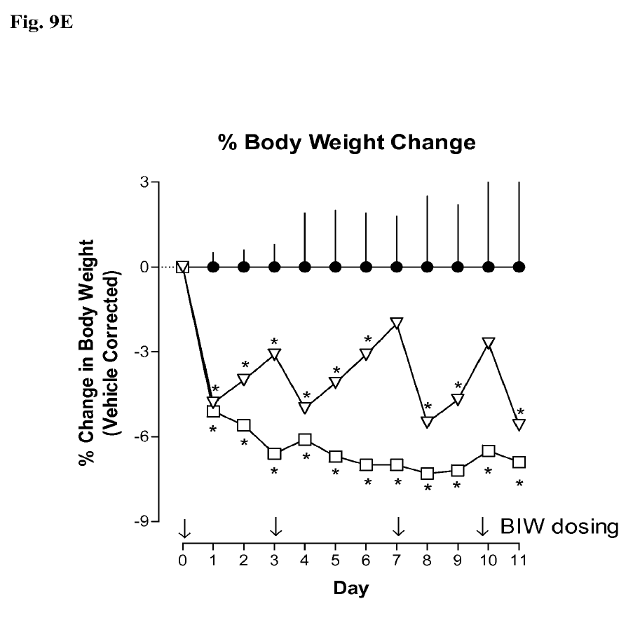

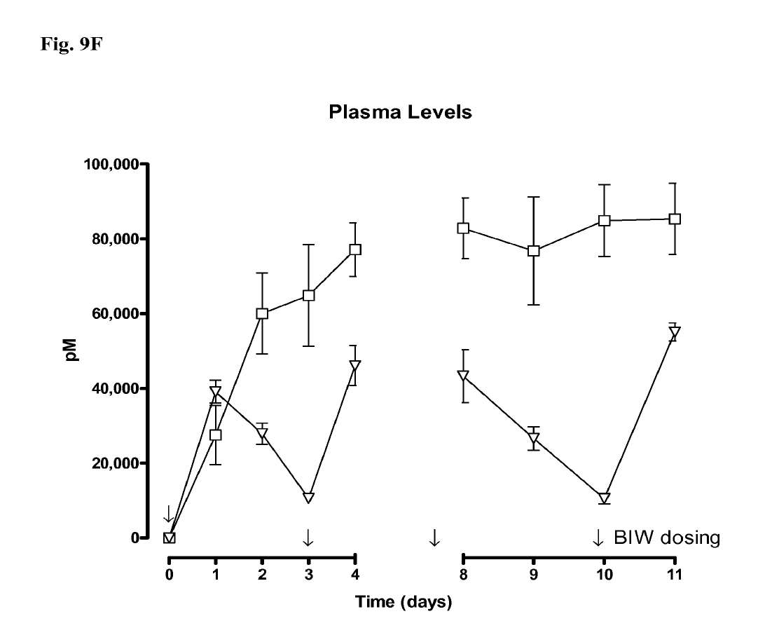

To demonstrate the effect of sub-chronic exposure of an exemplary engineered polypeptide in vivo, Cmpd 11 was administered subcutaneously twice weekly or daily for 14 days. Food intake inhibition, body weight decrease and plasma levels of Cmpd 11 were determined daily. Normal lean HSD rats were treated subcutaneously with 25 nmol/kg Cmpd 11 over 14 days as indicated in FIGS. 9A-9F, either twice weekly (BIW; open inverted trangles) as indicated by the down arrows or daily (QD; open square). Both BIW and QD administration inhibited daily food intake compared to vehicle (closed circle), as shown by cumulative food intake FIG. 9A, percent change in daily food intake FIG. 9B and percent change in cumulative food intake FIG. 9C. Both BIW and QD administration resulted in body weight loss as indicated by the reduction in total body weight FIG. 9D and greater percent negative change in body weight FIG. 9E compared to vehicle. FIG. 9F depicts a PK profile of Cmpd 11 given BIW or QD. Points represent mean±s.d. (standard deviation) with 4 to 6 animals per point. The test compound was injected subcutaneously on day=0 immediately following baseline sample collection.

As can be seen both modes of administration provide a superimpositioning effect leading to higher plasma levels of compound upon each subsequent dose, until a steady state is obtained. With as little as 11 days of treatment, the efficacy observed for the BIW (twice weekly) dose began to approximate that observed for Leu14 exendin-4 given at ˜7.2 nmol/kg/d by continuous infusion (CSI)--about 7% lower vehicle-corrected body weight at steady state level. The QD dosing provides a smoother profile, however, when translated to larger animals and those having longer inherent albumin plasma-half-lifes, smoother plasma levels approximating the QD pattern observed in the rats, are expected for BIW, thrice weekly and even weekly administration of this and any engineered polypeptide described herein. The QD dose with this engineered polypepide achieved or surpassed efficacy of the unconjugated infused exendin or exendin analog during the short treatment period, which is therefore expected to be the case for each of the engineered polypeptides described herein.

### Example 15

### Lack of Vacuolization

With some drugs, such as some pegylated proteins, undesirable vacuoles can form in cytoplasm of epithelial cells lining the proximal convoluted tubules, which is an undesirable toxicity measure. The engineered albumin binding compounds of the present application do not form kidney vacuoles. C57BL6 female mice (n=2 cages, 3 mice/cage) were weighed daily 3 hours prior to lights out. Immediately after weighing, on days 0-6 mice were injected subcutaneously with test compound. Mice were sacrificed on day 7 and kidneys submitted for histopathology. Severity score for cytoplasmic vacuolation of renal cortical tubular epithelial cells was as follows: score 1=minimal (8-15%); 2=mild (16-35%); 3=moderate (36-60%); 4=marked (>60%). A positive control compound known to cause vacuole formation was scored as 3. The ABD polypeptide PEP07986 itself scored 0. The engineered polpeptide Cmpd 5 scored 0.

### Example 16

### Oral Delivery Achieves Systemic Distribution

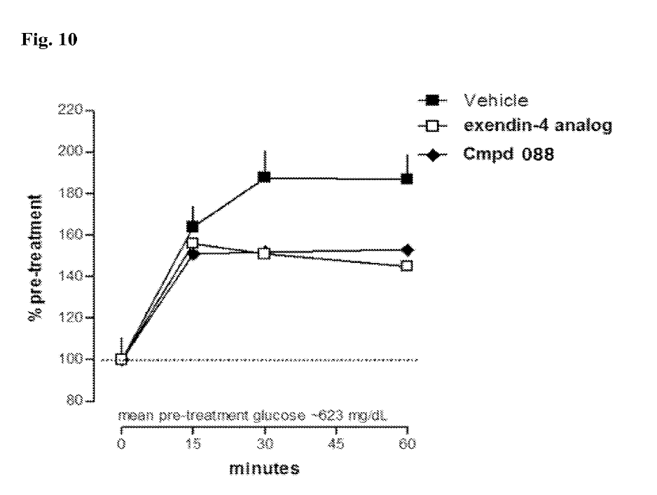

Oral delivery with intestinal uptake was investigated using Cmpd 088. Diabetic db/db mice were dosed orally (peroral via gavage) with 240 nmol/kg of the following compounds, an exendin analog [Leul 4, Gln28]Exendin-4-(1-32)-fGLP-1-(33-37) acid and Cmpd 088. The data demonstrate that the engineered peptides are orally bioavailable, even in a formulation PBS/propylene glycol (50:50) absent other specific excipients that might enhance delivery and uptake. Compared to the exendin analog, Cmpd 088 (both at 1 mg/kg dose) at more than twice the molecular weight of the exendin analog is also orally bioavailable in the same formulation. The results indicate that both compounds were active when dosed orally, and equally efficacious under the conditions tested to 120 minutes. The results are presented in FIG. 10. Points represent mean +/−sd. Peptides were dosed peroral by gavage at t=0 immediately following the taking of a baseline sample. Mice were 2-hour fasted db/db mice. Accordingly, the improved engineered polypeptide compounds presented herein have use for at least twice daily (e.g. morning and night), at least daily, thrice weekly, twice weekly, and even once weekly oral administration, especially in human subjects.

1.-116. (canceled)

117. A pharmaceutical composition comprising an engineered polypeptide; wherein the engineered polypeptide comprises:
an albumin binding domain polypeptide (ABD); and
a first peptide hormone (HD1) comprising an exendin sequence, an exendin analog sequence or an active fragment sequence thereof;
wherein the ABD comprises at least 95% identity to
LAX₃AKX₆X₇ANX₁₀ELDX₁₄YGVSDFYKRLIX₂₆KAKTVEGVEALKX₃₉X₄₀ILX₄₃X₄₄LP (SEQ ID NO: 300) with the proviso that X₇ is not L, E or D;
wherein independently of each other:
X₃ is selected from E, S, Q and C;
X₆ is selected from E, S and C;
X₇ is selected from A and S;
X₁₄ is selected from A, S, C and K;
X₁₀ is selected from A, S and R;
X₂₆ is selected from D and E;
X₃₉ is selected from D and E;
X₄₀ is selected from A and E;
X₄₃ is selected from A and K;
X₄₄ is selected from A, S and E;
the leucine at position 45 is present or absent; and
the proline at position 46 is present or absent.

118. The pharmaceutical composition of claim 117, further comprising a pharmaceutically acceptable excipient.

119. The pharmaceutical composition according to claim 117, wherein the pharmaceutical composition is aqueous.

120. The pharmaceutical composition according to claim 117, wherein the pharmaceutical composition is solid.

121. (canceled)

122. The pharmaceutical composition according to claim 117, wherein the engineered polypeptide is a lyophilized solid.

123. The pharmaceutical composition according to claim 117, further comprising human serum albumin.

124. The pharmaceutical composition according to claim 117, wherein said pharmaceutical composition is a pharmaceutical composition for intramuscular, intravenous, subcutaneous, intradermal, intraarticular, intrathecal, mucosal, oral, nasal, sublingual, pulmonary or buccal delivery.

125.-139. (canceled)

140. A method for treating a disease or disorder in a subject, comprising administering an engineered polypeptide to a subject in need thereof in an amount effective to treat said disease or disorder;
wherein the engineered polypeptide comprises:
an albumin binding domain polypeptide (ABD); and
a first peptide hormone (HD1) comprising an exendin sequence, an exendin analog sequence or an active fragment sequence thereof;
wherein the ABD comprises at least 95% identity to
LAX₃AKX₆X₇ANX₁₀ELDX₁₄YGVSDFYKRLIX₂₆KAKTVEGVEALKX₃₉X₄₀ILX₄₃X₄₄LP (SEQ ID NO: 300) with the proviso that X₇ is not L, E or D;
wherein independently of each other:
X₃ is selected from E, S, Q and C;
X₆ is selected from E, S and C;
X₇ is selected from A and S;
X₁₄ is selected from A, S, C and K;
X₁₀ is selected from A, S and R;
X₂₆ is selected from D and E;
X₃₉ is selected from D and E;
X₄₀ is selected from A and E;
X₄₃ is selected from A and K;
X₄₄ is selected from A, S and E;
the leucine at position 45 is present or absent; and
the proline at position 46 is present or absent.

141. The method according to claim 140, wherein said disease or disorder is diabetes, overweight, obesity, Alzheimer's disease, Parkinson's disease, fatty liver disease, dyslipidemia, coronary artery disease, stroke, short bowel syndrome (SBS), hyperlipidemia, nonalcoholic steatohepatitis (NASH), nonalcoholic fatty liver disease (NAFLD), hypoglycemia unawareness (HU), restrictive lung disease including sarcoidosis, or metabolic syndrome X.

142.-143. (canceled)

144. The method according to claim 143, wherein said disease or disorder is diabetes and said diabetes is type I diabetes, type II diabetes or prediabetes.

145.-150. (canceled)

151. The method according to claim 140, wherein the engineered polypeptide further comprises a first linker (L1) covalently linking said HD1 and said ABD.

152. The method according to claim 140, wherein the HD1 is exendin-4(1-28) (SEQ ID NO: 980), exendin-4(1-29) (SEQ ID NO: 981), exendin-4(1-30) (SEQ ID NO: 680), exendin-4(1-31) (SEQ ID NO: 982) or exendin-4(1-32) (SEQ ID NO: 983).

153. The method according to claim 140, wherein the HD1 is selected from the group of sequences consisting of:
(SEQ ID NO: 2)
HGEGTFTSDLSKQMEEEAVRLFIEWLKNGGPSSGAPPPS;
(SEQ ID NO: 3)
HGEGTFTSDLSKQLEEEAVRLFIEWLKNGGPSSGAPPPS;
(SEQ ID NO: 4)
HGEGTFTSDLSKQLEEEAVRLFIEWLKQGGPSKEIIS;
(SEQ ID NO: 111)
HGEGTFTSDLSKQMEEEAVRLFIEWLKQGGPSKEIIS;
(SEQ ID NO: 112)
HGEGTFTSDLSKQLEEEAVRLFIEWLKNGGPSSGAPPS;
(SEQ ID NO: 113)
HGEGTFTSDLSKQMEEEAVRLFIEWLKNGGPSSGAPPS;
(SEQ ID NO: 114)
HGEGTFTSDLSKQLEEEAVRLFIEWLKQGGPSKEIISKKKKKK;
(SEQ ID NO: 115)
HGEGTFTSDLSKQMEEEAVRLFIEWLKNGGPSSGAPPPSKKKKKK;
(SEQ ID NO: 116)
HGEGTFTSDLSKQMEEEAVRLFIEWLKQGGPSKEIISKKKKKK;
(SEQ ID NO: 117)
HGEGTFTSDLSKQLEEEAVRLFIEWLKNGGPSSGAPPSKKKKKK;
and
(SEQ ID NO: 118)
HGEGTFTSDLSKQMEEEAVRLFIEWLKNGGPSSGAPPSKKKKKK.

154. The method according to claim 140, wherein the ABD is selected from the group consisting of: SEQ ID NOs: 301-463, 500-502, and 862-950.

155. The method according to claim 140, wherein the engineered polypeptide is selected from the group consisting of:
(SEQ ID NO: 727)
HGEGTFTSDLSKQLEEEAVRLFIEWLKQGGPSKEIISTGGGGSASGSLAE
AKEAANAELDSYGVSDFYKRLIDKAKTVEGVEALKDAILAALP;
(SEQ ID NO: 728)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAALP;
(SEQ ID NO: 729)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAAL;
(SEQ ID NO: 730)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAALP;
(SEQ ID NO: 731)
HGEGTFTSDLSKQMEEEAVRLFIEWLKNGGPSSGAPPPSGGSLAEAKEAA
NAELDSYGVSDFYKRLIDKAKTVEGVEALKDAILAALP;
(SEQ ID NO: 732)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAALP;
(SEQ ID NO: 733)
HGEGTFTSDLSKQLEEEAVRLFIEWLKNGGPSSGAPPPSGGSLAEAKEAA
NAELDSYGVSDFYKRLIDKAKTVEGVEALKDAILAALP;
(SEQ ID NO: 734)
HGEGTFTSDLSKQLEEEAVRLFIEWLKQGGPSKEIISTGGGGSASGSLAE
AKEAANAELDSYGVSDFYKRLIDKAKTVEGVEALKDAILAAL;
(SEQ ID NO: 735)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAAL;
(SEQ ID NO: 736)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
(SEQ ID NO: 737)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAAL;
(SEQ ID NO: 738)
HGEGTFTSDLSKQMEEEAVRLFIEWLKNGGPSSGAPPPSGGSLAEAKEAA
NAELDSYGVSDFYKRLIDKAKTVEGVEALKDAILAAL;
(SEQ ID NO: 739)
HGEGTFTSDLSKQLEEEAVRLFIEWLKNGGPSSGAPPPSGGSLAEAKEAA
NAELDSYGVSDFYKRLIDKAKTVEGVEALKDAILAAL;
(SEQ ID NO: 740)
HGEGTFTSDLSKQLEEEAVRLFIEWLKQGGPSKEIISTGGGGSASGSLAE
AKEAANAELDSYGVSDFYKRLIDKAKTVEGVEALKDAILAA;
(SEQ ID NO: 741)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAA;
(SEQ ID NO: 742)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAA;
(SEQ ID NO: 743)
HGEGTFTSDLSKQMEEEAVRLFIEWLKNGGPSSGAPPPSGGSLAEAKEAA
NAELDSYGVSDFYKRLIDKAKTVEGVEALKDAILAA;
(SEQ ID NO: 744)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
and
(SEQ ID NO: 745)
HGEGTFTSDLSKQLEEEAVRLFIEWLKNGGPSSGAPPPSGGSLAEAKEAA
NAELDSYGVSDFYKRLIDKAKTVEGVEALKDAILAA.

156. The pharmaceutical composition of claim 117, wherein the engineered polypeptide further comprises a first linker (L1) covalently linking said HD1 and said ABD.

157. The pharmaceutical composition of claim 117, wherein the HD1 is exendin-4(1-28) (SEQ ID NO: 980), exendin-4(1-29) (SEQ ID NO: 981), exendin-4(1-30) (SEQ ID NO: 680), exendin-4(1-31) (SEQ ID NO: 982) or exendin-4(1-32) (SEQ ID NO: 983).

158. The pharmaceutical composition of claim 117, wherein the HD1 is selected from the group of sequences consisting of:
(SEQ ID NO: 2)
HGEGTFTSDLSKQMEEEAVRLFIEWLKNGGPSSGAPPPS;
(SEQ ID NO: 3)
HGEGTFTSDLSKQLEEEAVRLFIEWLKNGGPSSGAPPPS;
(SEQ ID NO: 4)
HGEGTFTSDLSKQLEEEAVRLFIEWLKQGGPSKEIIS;
(SEQ ID NO: 111)
HGEGTFTSDLSKQMEEEAVRLFIEWLKQGGPSKEIIS;
(SEQ ID NO: 112)
HGEGTFTSDLSKQLEEEAVRLFIEWLKNGGPSSGAPPS;
(SEQ ID NO: 113)
HGEGTFTSDLSKQMEEEAVRLFIEWLKNGGPSSGAPPS;
(SEQ ID NO: 114)
HGEGTFTSDLSKQLEEEAVRLFIEWLKQGGPSKEIISKKKKKK;
(SEQ ID NO: 115)
HGEGTFTSDLSKQMEEEAVRLFIEWLKNGGPSSGAPPPSKKKKKK;
(SEQ ID NO: 116)
HGEGTFTSDLSKQMEEEAVRLFIEWLKQGGPSKEIISKKKKKK;
(SEQ ID NO: 117)
HGEGTFTSDLSKQLEEEAVRLFIEWLKNGGPSSGAPPSKKKKKK;
and
(SEQ ID NO: 118)
HGEGTFTSDLSKQMEEEAVRLFIEWLKNGGPSSGAPPSKKKKKK.

159. The pharmaceutical composition of claim 117, wherein the ABD is selected from the group consisting of: SEQ ID NOs: 301-463, 500-502, and 862-950.

160. The pharmaceutical composition of claim 117, wherein the engineered polypeptide is selected from the group consisting of:
(SEQ ID NO: 727)
HGEGTFTSDLSKQLEEEAVRLFIEWLKQGGPSKEIISTGGGGSASGSLAE
AKEAANAELDSYGVSDFYKRLIDKAKTVEGVEALKDAILAALP;
(SEQ ID NO: 728)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAALP;
(SEQ ID NO: 729)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAAL;
(SEQ ID NO: 730)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAALP;
(SEQ ID NO: 731)
HGEGTFTSDLSKQMEEEAVRLFIEWLKNGGPSSGAPPPSGGSLAEAKEAA
NAELDSYGVSDFYKRLIDKAKTVEGVEALKDAILAALP;
(SEQ ID NO: 732)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAALP;
(SEQ ID NO: 733)
HGEGTFTSDLSKQLEEEAVRLFIEWLKNGGPSSGAPPPSGGSLAEAKEAA
NAELDSYGVSDFYKRLIDKAKTVEGVEALKDAILAALP;
(SEQ ID NO: 734)
HGEGTFTSDLSKQLEEEAVRLFIEWLKQGGPSKEIISTGGGGSASGSLAE
AKEAANAELDSYGVSDFYKRLIDKAKTVEGVEALKDAILAAL;
(SEQ ID NO: 735)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAAL;
(SEQ ID NO: 736)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
(SEQ ID NO: 737)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAAL;
(SEQ ID NO: 738)
HGEGTFTSDLSKQMEEEAVRLFIEWLKNGGPSSGAPPPSGGSLAEAKEAA
NAELDSYGVSDFYKRLIDKAKTVEGVEALKDAILAAL;
(SEQ ID NO: 739)
HGEGTFTSDLSKQLEEEAVRLFIEWLKNGGPSSGAPPPSGGSLAEAKEAA
NAELDSYGVSDFYKRLIDKAKTVEGVEALKDAILAAL;
(SEQ ID NO: 740)
HGEGTFTSDLSKQLEEEAVRLFIEWLKQGGPSKEIISTGGGGSASGSLAE
AKEAANAELDSYGVSDFYKRLIDKAKTVEGVEALKDAILAA;
(SEQ ID NO: 741)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAA;
(SEQ ID NO: 742)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAA;
(SEQ ID NO: 743)
HGEGTFTSDLSKQMEEEAVRLFIEWLKNGGPSSGAPPPSGGSLAEAKEAA
NAELDSYGVSDFYKRLIDKAKTVEGVEALKDAILAA;
(SEQ ID NO: 744)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
and
(SEQ ID NO: 745)
HGEGTFTSDLSKQLEEEAVRLFIEWLKNGGPSSGAPPPSGGSLAEAKEAA
XAEAKEAANAELFYKRLIDKAKTVEGVEALKDAILAA.


SEQUENCE LISTING (988 sequences)

SEQ ID NO: 1 - protein, 39 aa, Heloderma horridum
  May be C-term NH2
HSDGTFTSDLSKQMEEEAVRLFIEWLKNGGPSSGAPPPS

SEQ ID NO: 2 - protein, 39 aa, Heloderma suspectum
  May be C-term NH2
HGEGTFTSDLSKQMEEEAVRLFIEWLKNGGPSSGAPPPS

SEQ ID NO: 3 - protein, 39 aa, Heloderma suspectum
  May be C-term NH2
HGEGTFTSDLSKQLEEEAVRLFIEWLKNGGPSSGAPPPS

SEQ ID NO: 4 - protein, 37 aa, Artificial Sequence
  Description of Artificial Sequence: Synthetic polypeptide
HGEGTFTSDLSKQLEEEAVRLFIEWLKQGGPSKEIIS

SEQ ID NO: 5 - protein, 31 aa, Unknown
  Description of Unknown: Glucagon-like peptide
HAEGTFTSDVSSYLEGQAAKEFIAWLVKGRG

SEQ ID NO: 6 - protein, 37 aa, Rattus sp.
KCNTATCATQRLANFLVRSSNNLGPVLPPTNVGSNTY

SEQ ID NO: 7 - protein, 37 aa, Homo sapiens [sequence omitted]

SEQ ID NO: 8 - protein, 37 aa, Homo sapiens
KCNTATCATQRLANFLVHSSNNFGPILPPTNVGSNTY

SEQ ID NO: 9 - protein, 32 aa, Homo sapiens
CGNLSTCMLGTYTQDFNKFHTFPQTAIGVGAP

SEQ ID NO: 10 - protein, 32 aa, Oncorhynchus sp.
CSNLSTCVLGKLSQELHKLQTYPRTNTGSGTP

SEQ ID NO: 11 - protein, 32 aa, Oncorhynchus sp.
KCNTATCVLGRLSQELHRLQTYPRTNTGSNTY

SEQ ID NO: 12 - protein, 28 aa, Artificial Sequence
  Description of Artificial Sequence: Synthetic peptide
HGEGTFTSDLSKQMEEEAVRLFIEWLKN

SEQ ID NO: 13-48 - skipped (36 entries)

SEQ ID NO: 49 - protein, 6 aa, Artificial Sequence
  Description of Artificial Sequence: Synthetic 6xHis tag
HHHHHH

SEQ ID NO: 50-110 - skipped (61 entries)

SEQ ID NO: 111 - protein, 37 aa, Artificial Sequence
  Description of Artificial Sequence: Synthetic polypeptide
HGEGTFTSDLSKQMEEEAVRLFIEWLKQGGPSKEIIS

SEQ ID NO: 112 - protein, 38 aa, Artificial Sequence
  Description of Artificial Sequence: Synthetic polypeptide
HGEGTFTSDLSKQLEEEAVRLFIEWLKNGGPSSGAPPS

SEQ ID NO: 113 - protein, 38 aa, Artificial Sequence
  Description of Artificial Sequence: Synthetic polypeptide
HGEGTFTSDLSKQMEEEAVRLFIEWLKNGGPSSGAPPS

SEQ ID NO: 114 - protein, 43 aa, Artificial Sequence
  Description of Artificial Sequence: Synthetic polypeptide
HGEGTFTSDLSKQLEEEAVRLFIEWLKQGGPSKEIISKKKKKK

SEQ ID NO: 115 - protein, 45 aa, Artificial Sequence
  Description of Artificial Sequence: Synthetic polypeptide
HGEGTFTSDLSKQMEEEAVRLFIEWLKNGGPSSGAPPPSKKKKKK

SEQ ID NO: 116 - protein, 43 aa, Artificial Sequence
  Description of Artificial Sequence: Synthetic polypeptide
HGEGTFTSDLSKQMEEEAVRLFIEWLKQGGPSKEIISKKKKKK

SEQ ID NO: 117 - protein, 44 aa, Artificial Sequence
  Description of Artificial Sequence: Synthetic polypeptide
HGEGTFTSDLSKQLEEEAVRLFIEWLKNGGPSSGAPPSKKKKKK

SEQ ID NO: 118 - protein, 44 aa, Artificial Sequence
  Description of Artificial Sequence: Synthetic polypeptide
HGEGTFTSDLSKQMEEEAVRLFIEWLKNGGPSSGAPPSKKKKKK

SEQ ID NO: 119-124 - skipped (6 entries)

SEQ ID NO: 125 - protein, 4 aa, Artificial Sequence
  Description of Artificial Sequence: Synthetic peptide
GGGG

SEQ ID NO: 126 - protein, 5 aa, Artificial Sequence
  Description of Artificial Sequence: Synthetic peptide
GGGGG

SEQ ID NO: 127 - protein, 7 aa, Artificial Sequence
  Description of Artificial Sequence: Synthetic peptide
  misc_feature (1)..(6): This region may encompass 1 to 6 residues
GGGGGGE

SEQ ID NO: 128 - protein, 7 aa, Artificial Sequence
  Description of Artificial Sequence: Synthetic peptide
  misc_feature (1)..(6): This region may encompass 1 to 6 residues
GGGGGGK

SEQ ID NO: 129 - protein, 7 aa, Artificial Sequence
  Description of Artificial Sequence: Synthetic peptide
  misc_feature (1)..(6): This region may encompass 1 to 6 residues
GGGGGGD

SEQ ID NO: 130 - protein, 7 aa, Artificial Sequence
  Description of Artificial Sequence: Synthetic peptide
  misc_feature (1)..(6): This region may encompass 1 to 6 residues
GGGGGGR

SEQ ID NO: 131 - protein, 8 aa, Artificial Sequence
  Description of Artificial Sequence: Synthetic peptide
GGGKGGGG

SEQ ID NO: 132 - protein, 8 aa, Artificial Sequence
  Description of Artificial Sequence: Synthetic peptide
GGGNGSGG

SEQ ID NO: 133 - protein, 8 aa, Artificial Sequence
  Description of Artificial Sequence: Synthetic peptide
GGGCGGGG

SEQ ID NO: 134 - protein, 5 aa, Artificial Sequence
  Description of Artificial Sequence: Synthetic peptide
GPNGG

SEQ ID NO: 135 - protein, 12 aa, Artificial Sequence
  Description of Artificial Sequence: Synthetic peptide
  misc_feature (1)..(12): This sequence may encompass 1 to 6 “Gly Ser” repeats
GSGSGSGSGSGS

SEQ ID NO: 136 - protein, 18 aa, Artificial Sequence
  Description of Artificial Sequence: Synthetic peptide
  misc_feature (1)..(18): This sequence may encompass 1 to 6 “Gly Gly Ser” repeats
GGSGGSGGSGGSGGSGGS

SEQ ID NO: 137 - protein, 24 aa, Artificial Sequence
  Description of Artificial Sequence: Synthetic peptide
  misc_feature (1)..(24): This sequence may encompass 1 to 6 “Gly Gly Gly Ser” repeats
GGGSGGGSGGGSGGGSGGGSGGGS

SEQ ID NO: 138 - protein, 30 aa, Artificial Sequence
  Description of Artificial Sequence: Synthetic polypeptide
  misc_feature (1)..(30): This sequence may encompass 1 to 6 “Gly Gly Gly Gly Ser” repeats
GGGGSGGGGSGGGGSGGGGSGGGGSGGGGS

SEQ ID NO: 139 - protein, 20 aa, Artificial Sequence
  Description of Artificial Sequence: Synthetic peptide
  misc_feature (1)..(20): This sequence may encompass 1 to 10 “Gly Glu” repeats
GEGEGEGEGEGEGEGEGEGE

SEQ ID NO: 140 - protein, 30 aa, Artificial Sequence
  Description of Artificial Sequence: Synthetic polypeptide
  misc_feature (1)..(30): This sequence may encompass 1 to 10 “Gly Gly Glu” repeats
GGEGGEGGEGGEGGEGGEGGEGGEGGEGGE

SEQ ID NO: 141 - protein, 40 aa, Artificial Sequence
  Description of Artificial Sequence: Synthetic polypeptide
  misc_feature (1)..(40): This sequence may encompass 1 to 10 “Gly Gly Gly Glu” repeats
GGGEGGGEGGGEGGGEGGGEGGGEGGGEGGGEGGGEGGGE

SEQ ID NO: 142 - protein, 50 aa, Artificial Sequence
  Description of Artificial Sequence: Synthetic polypeptide
  misc_feature (1)..(50): This sequence may encompass 1 to 10 “Gly Gly Gly Gly Glu” repeats
GGGGEGGGGEGGGGEGGGGEGGGGEGGGGEGGGGEGGGGEGGGGEGGGGE

SEQ ID NO: 143 - protein, 20 aa, Artificial Sequence
  Description of Artificial Sequence: Synthetic peptide
  misc_feature (1)..(20): This sequence may encompass 1 to 10 “Gly Asp” repeats
GDGDGDGDGDGDGDGDGDGD

SEQ ID NO: 144 - protein, 30 aa, Artificial Sequence
  Description of Artificial Sequence: Synthetic polypeptide
  misc_feature (1)..(30): This sequence may encompass 1 to 10 “Gly Gly Asp” repeats
GGDGGDGGDGGDGGDGGDGGDGGDGGDGGD

SEQ ID NO: 145 - protein, 40 aa, Artificial Sequence
  Description of Artificial Sequence: Synthetic polypeptide
  misc_feature (1)..(40): This sequence may encompass 1 to 10 “Gly Gly Gly Asp” repeats
GGGDGGGDGGGDGGGDGGGDGGGDGGGDGGGDGGGDGGGD

SEQ ID NO: 146 - protein, 50 aa, Artificial Sequence
  Description of Artificial Sequence: Synthetic polypeptide
  misc_feature (1)..(50): This sequence may encompass 1 to 10 “Gly Gly Gly Gly Asp” repeats
GGGGDGGGGDGGGGDGGGGDGGGGDGGGGDGGGGDGGGGDGGGGDGGGGD

SEQ ID NO: 147 - protein, 20 aa, Artificial Sequence
  Description of Artificial Sequence: Synthetic peptide
  misc_feature (1)..(20): This sequence may encompass 1 to 10 “Gly Lys” repeats
GKGKGKGKGKGKGKGKGKGK

SEQ ID NO: 148 - protein, 30 aa, Artificial Sequence
  Description of Artificial Sequence: Synthetic polypeptide
  misc_feature (1)..(30): This sequence may encompass 1 to 10 “Gly Gly Lys” repeats
GGKGGKGGKGGKGGKGGKGGKGGKGGKGGK

SEQ ID NO: 149 - protein, 40 aa, Artificial Sequence
  Description of Artificial Sequence: Synthetic polypeptide
  misc_feature (1)..(40): This sequence may encompass 1 to 10 “Gly Gly Gly Lys” repeats
GGGKGGGKGGGKGGGKGGGKGGGKGGGKGGGKGGGKGGGK

SEQ ID NO: 150 - protein, 50 aa, Artificial Sequence
  Description of Artificial Sequence: Synthetic polypeptide
  misc_feature (1)..(50): This sequence may encompass 1 to 10 “Gly Gly Gly Gly Lys” repeats
GGGGKGGGGKGGGGKGGGGKGGGGKGGGGKGGGGKGGGGKGGGGKGGGGK

SEQ ID NO: 151 - protein, 20 aa, Artificial Sequence
  Description of Artificial Sequence: Synthetic peptide
  misc_feature (1)..(20): This sequence may encompass 1 to 10 “Gly Arg” repeats
GRGRGRGRGRGRGRGRGRGR

SEQ ID NO: 152 - protein, 30 aa, Artificial Sequence
  Description of Artificial Sequence: Synthetic polypeptide
  misc_feature (1)..(30): This sequence may encompass 1 to 10 “Gly Gly Arg” repeats
GGRGGRGGRGGRGGRGGRGGRGGRGGRGGR

SEQ ID NO: 153 - protein, 40 aa, Artificial Sequence
  Description of Artificial Sequence: Synthetic polypeptide
  misc_feature (1)..(40): This sequence may encompass 1 to 10 “Gly Gly Gly Arg” repeats
GGGRGGGRGGGRGGGRGGGRGGGRGGGRGGGRGGGRGGGR

SEQ ID NO: 154 - protein, 50 aa, Artificial Sequence
  Description of Artificial Sequence: Synthetic polypeptide
  misc_feature (1)..(50): This sequence may encompass 1 to 10 “Gly Gly Gly Gly Arg” repeats
GGGGRGGGGRGGGGRGGGGRGGGGRGGGGRGGGGRGGGGRGGGGRGGGGR

SEQ ID NO: 155 - protein, 50 aa, Artificial Sequence
  Description of Artificial Sequence: Synthetic polypeptide
  misc_feature (1)..(50): This sequence may encompass 1 to 10 “Glu Ala Ala Ala Lys” repeats
EAAAKEAAAKEAAAKEAAAKEAAAKEAAAKEAAAKEAAAKEAAAKEAAAK

SEQ ID NO: 156-158 - skipped (3 entries)

SEQ ID NO: 159 - protein, 47 aa, Artificial Sequence
  Description of Artificial Sequence: Synthetic polypeptide
GLASAKEAANAELDCYGVSDFYKRLIDKAKTVEGVEALKDAILAALP

SEQ ID NO: 160 - protein, 47 aa, Artificial Sequence
  Description of Artificial Sequence: Synthetic polypeptide
  C-term NH2
ALASAKEAANAELDCYGVSDFYKRLIDKAKTVEGVEALKDAILAALP

SEQ ID NO: 161-191 - skipped (31 entries)

SEQ ID NO: 192 - protein, 4 aa, Artificial Sequence
  Description of Artificial Sequence: Synthetic peptide
GGGS

SEQ ID NO: 193 - protein, 7 aa, Artificial Sequence
  Description of Artificial Sequence: Synthetic peptide
TGGGGAS

SEQ ID NO: 194 - protein, 8 aa, Artificial Sequence
  Description of Artificial Sequence: Synthetic peptide
TGGGGGAS

SEQ ID NO: 195 - protein, 8 aa, Artificial Sequence
  Description of Artificial Sequence: Synthetic peptide
TGGGGSAS

SEQ ID NO: 196-299 - skipped (104 entries)

SEQ ID NO: 300 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIXKAKTVEGVEALKXXILXXLP

SEQ ID NO: 301 - protein, 46 aa, Artificial Sequence
  Description of Artificial Sequence: Synthetic polypeptide
LASAKEAANAELDAYGVSDFYKRLIDKAKTVEGVEALKDAILAALP

SEQ ID NO: 302 - protein, 46 aa, Artificial Sequence
  Description of Artificial Sequence: Synthetic polypeptide
LASAKEAANSELDAYGVSDFYKRLIDKAKTVEGVEALKDAILAALP

SEQ ID NO: 303 - protein, 46 aa, Artificial Sequence
  Description of Artificial Sequence: Synthetic polypeptide
LASAKESANSELDAYGVSDFYKRLIDKAKTVEGVEALKDAILAALP

SEQ ID NO: 304 - protein, 46 aa, Artificial Sequence
  Description of Artificial Sequence: Synthetic polypeptide
LASAKESANAELDAYGVSDFYKRLIDKAKTVEGVEALKDAILAALP

SEQ ID NO: 305 - protein, 46 aa, Artificial Sequence
  Description of Artificial Sequence: Synthetic polypeptide
LASAKSAANAELDAYGVSDFYKRLIDKAKTVEGVEALKDAILAALP

SEQ ID NO: 306 - protein, 46 aa, Artificial Sequence
  Description of Artificial Sequence: Synthetic polypeptide
LASAKSAANSELDAYGVSDFYKRLIDKAKTVEGVEALKDAILAALP

SEQ ID NO: 307 - protein, 46 aa, Artificial Sequence
  Description of Artificial Sequence: Synthetic polypeptide
LASAKEAANAELDSYGVSDFYKRLIDKAKTVEGVEALKDAILAALP

SEQ ID NO: 308 - protein, 46 aa, Artificial Sequence
  Description of Artificial Sequence: Synthetic polypeptide
LASAKEAANSELDSYGVSDFYKRLIDKAKTVEGVEALKDAILAALP

SEQ ID NO: 309 - protein, 46 aa, Artificial Sequence
  Description of Artificial Sequence: Synthetic polypeptide
LASAKESANSELDSYGVSDFYKRLIDKAKTVEGVEALKDAILAALP

SEQ ID NO: 310 - protein, 46 aa, Artificial Sequence
  Description of Artificial Sequence: Synthetic polypeptide
LASAKESANAELDSYGVSDFYKRLIDKAKTVEGVEALKDAILAALP

SEQ ID NO: 311 - protein, 46 aa, Artificial Sequence
  Description of Artificial Sequence: Synthetic polypeptide
LASAKSAANAELDSYGVSDFYKRLIDKAKTVEGVEALKDAILAALP

SEQ ID NO: 312 - protein, 46 aa, Artificial Sequence
  Description of Artificial Sequence: Synthetic polypeptide
LASAKSAANSELDSYGVSDFYKRLIDKAKTVEGVEALKDAILAALP

SEQ ID NO: 313 - protein, 46 aa, Artificial Sequence
  Description of Artificial Sequence: Synthetic polypeptide
LAEAKEAANAELDSYGVSDFYKRLIDKAKTVEGVEALKDAILAALP

SEQ ID NO: 314 - protein, 46 aa, Artificial Sequence
  Description of Artificial Sequence: Synthetic polypeptide
LAEAKEAANSELDSYGVSDFYKRLIDKAKTVEGVEALKDAILAALP

SEQ ID NO: 315 - protein, 46 aa, Artificial Sequence
  Description of Artificial Sequence: Synthetic polypeptide
LAEAKESANSELDSYGVSDFYKRLIDKAKTVEGVEALKDAILAALP

SEQ ID NO: 316 - protein, 46 aa, Artificial Sequence
  Description of Artificial Sequence: Synthetic polypeptide
LAEAKESANAELDSYGVSDFYKRLIDKAKTVEGVEALKDAILAALP

SEQ ID NO: 317 - protein, 46 aa, Artificial Sequence
  Description of Artificial Sequence: Synthetic polypeptide
LAEAKSAANAELDSYGVSDFYKRLIDKAKTVEGVEALKDAILAALP

SEQ ID NO: 318 - protein, 46 aa, Artificial Sequence
  Description of Artificial Sequence: Synthetic polypeptide
LAEAKSAANSELDSYGVSDFYKRLIDKAKTVEGVEALKDAILAALP

SEQ ID NO: 319 - protein, 46 aa, Artificial Sequence
  Description of Artificial Sequence: Synthetic polypeptide
LAEAKEAANAELDAYGVSDFYKRLIDKAKTVEGVEALKDAILAALP

SEQ ID NO: 320 - protein, 46 aa, Artificial Sequence
  Description of Artificial Sequence: Synthetic polypeptide
LAEAKEAANSELDAYGVSDFYKRLIDKAKTVEGVEALKDAILAALP

SEQ ID NO: 321 - protein, 46 aa, Artificial Sequence
  Description of Artificial Sequence: Synthetic polypeptide
LAEAKESANSELDAYGVSDFYKRLIDKAKTVEGVEALKDAILAALP

SEQ ID NO: 322 - protein, 46 aa, Artificial Sequence
  Description of Artificial Sequence: Synthetic polypeptide
LAEAKESANAELDAYGVSDFYKRLIDKAKTVEGVEALKDAILAALP

SEQ ID NO: 323 - protein, 46 aa, Artificial Sequence
  Description of Artificial Sequence: Synthetic polypeptide
LAEAKSAANAELDAYGVSDFYKRLIDKAKTVEGVEALKDAILAALP

SEQ ID NO: 324 - protein, 46 aa, Artificial Sequence
  Description of Artificial Sequence: Synthetic polypeptide
LAEAKSAANSELDAYGVSDFYKRLIDKAKTVEGVEALKDAILAALP

SEQ ID NO: 325 - protein, 46 aa, Artificial Sequence
  Description of Artificial Sequence: Synthetic polypeptide
LAQAKEAANAELDAYGVSDFYKRLIDKAKTVEGVEALKDAILAALP

SEQ ID NO: 326 - protein, 46 aa, Artificial Sequence
  Description of Artificial Sequence: Synthetic polypeptide
LAQAKEAANSELDAYGVSDFYKRLIDKAKTVEGVEALKDAILAALP

SEQ ID NO: 327 - protein, 46 aa, Artificial Sequence
  Description of Artificial Sequence: Synthetic polypeptide
LAQAKESANSELDAYGVSDFYKRLIDKAKTVEGVEALKDAILAALP

SEQ ID NO: 328 - protein, 46 aa, Artificial Sequence
  Description of Artificial Sequence: Synthetic polypeptide
LAQAKESANAELDAYGVSDFYKRLIDKAKTVEGVEALKDAILAALP

SEQ ID NO: 329 - protein, 46 aa, Artificial Sequence
  Description of Artificial Sequence: Synthetic polypeptide
LAQAKSAANAELDAYGVSDFYKRLIDKAKTVEGVEALKDAILAALP

SEQ ID NO: 330 - protein, 46 aa, Artificial Sequence
  Description of Artificial Sequence: Synthetic polypeptide
LAQAKSAANSELDAYGVSDFYKRLIDKAKTVEGVEALKDAILAALP

SEQ ID NO: 331 - protein, 46 aa, Artificial Sequence
  Description of Artificial Sequence: Synthetic polypeptide
LAQAKEAANAELDSYGVSDFYKRLIDKAKTVEGVEALKDAILAALP

SEQ ID NO: 332 - protein, 46 aa, Artificial Sequence
  Description of Artificial Sequence: Synthetic polypeptide
LAQAKEAANSELDSYGVSDFYKRLIDKAKTVEGVEALKDAILAALP

SEQ ID NO: 333 - protein, 46 aa, Artificial Sequence
  Description of Artificial Sequence: Synthetic polypeptide
LAQAKESANSELDSYGVSDFYKRLIDKAKTVEGVEALKDAILAALP

SEQ ID NO: 334 - protein, 46 aa, Artificial Sequence
  Description of Artificial Sequence: Synthetic polypeptide
LAQAKESANAELDSYGVSDFYKRLIDKAKTVEGVEALKDAILAALP

SEQ ID NO: 335 - protein, 46 aa, Artificial Sequence
  Description of Artificial Sequence: Synthetic polypeptide
LAQAKSAANAELDSYGVSDFYKRLIDKAKTVEGVEALKDAILAALP

SEQ ID NO: 336 - protein, 46 aa, Artificial Sequence
  Description of Artificial Sequence: Synthetic polypeptide
LAQAKSAANSELDSYGVSDFYKRLIDKAKTVEGVEALKDAILAALP

SEQ ID NO: 337 - protein, 46 aa, Artificial Sequence
  Description of Artificial Sequence: Synthetic polypeptide
LASAKEAANAELDCYGVSDFYKRLIDKAKTVEGVEALKDAILAALP

SEQ ID NO: 338 - protein, 46 aa, Artificial Sequence
  Description of Artificial Sequence: Synthetic polypeptide
LASAKEAANSELDCYGVSDFYKRLIDKAKTVEGVEALKDAILAALP

SEQ ID NO: 339 - protein, 46 aa, Artificial Sequence
  Description of Artificial Sequence: Synthetic polypeptide
LASAKESANSELDCYGVSDFYKRLIDKAKTVEGVEALKDAILAALP

SEQ ID NO: 340 - protein, 46 aa, Artificial Sequence
  Description of Artificial Sequence: Synthetic polypeptide
LASAKESANAELDCYGVSDFYKRLIDKAKTVEGVEALKDAILAALP

SEQ ID NO: 341 - protein, 46 aa, Artificial Sequence
  Description of Artificial Sequence: Synthetic polypeptide
LASAKSAANAELDCYGVSDFYKRLIDKAKTVEGVEALKDAILAALP

SEQ ID NO: 342 - protein, 46 aa, Artificial Sequence
  Description of Artificial Sequence: Synthetic polypeptide
LASAKSAANSELDCYGVSDFYKRLIDKAKTVEGVEALKDAILAALP

SEQ ID NO: 343 - protein, 46 aa, Artificial Sequence
  Description of Artificial Sequence: Synthetic polypeptide
LASAKEAANAELDKYGVSDFYKRLIDKAKTVEGVEALKDAILAALP

SEQ ID NO: 344 - protein, 46 aa, Artificial Sequence
  Description of Artificial Sequence: Synthetic polypeptide
LASAKEAANSELDKYGVSDFYKRLIDKAKTVEGVEALKDAILAALP

SEQ ID NO: 345 - protein, 46 aa, Artificial Sequence
  Description of Artificial Sequence: Synthetic polypeptide
LASAKESANSELDKYGVSDFYKRLIDKAKTVEGVEALKDAILAALP

SEQ ID NO: 346 - protein, 46 aa, Artificial Sequence
  Description of Artificial Sequence: Synthetic polypeptide
LASAKESANAELDKYGVSDFYKRLIDKAKTVEGVEALKDAILAALP

SEQ ID NO: 347 - protein, 46 aa, Artificial Sequence
  Description of Artificial Sequence: Synthetic polypeptide
LASAKSAANAELDKYGVSDFYKRLIDKAKTVEGVEALKDAILAALP

SEQ ID NO: 348 - protein, 46 aa, Artificial Sequence
  Description of Artificial Sequence: Synthetic polypeptide
LASAKSAANSELDKYGVSDFYKRLIDKAKTVEGVEALKDAILAALP

SEQ ID NO: 349 - protein, 46 aa, Artificial Sequence
  Description of Artificial Sequence: Synthetic polypeptide
LAEAKEAANAELDCYGVSDFYKRLIDKAKTVEGVEALKDAILAALP

SEQ ID NO: 350 - protein, 46 aa, Artificial Sequence
  Description of Artificial Sequence: Synthetic polypeptide
LAEAKEAANSELDCYGVSDFYKRLIDKAKTVEGVEALKDAILAALP

SEQ ID NO: 351 - protein, 46 aa, Artificial Sequence
  Description of Artificial Sequence: Synthetic polypeptide
LAEAKESANSELDCYGVSDFYKRLIDKAKTVEGVEALKDAILAALP

SEQ ID NO: 352 - protein, 46 aa, Artificial Sequence
  Description of Artificial Sequence: Synthetic polypeptide
LAEAKESANAELDCYGVSDFYKRLIDKAKTVEGVEALKDAILAALP

SEQ ID NO: 353 - protein, 46 aa, Artificial Sequence
  Description of Artificial Sequence: Synthetic polypeptide
LAEAKSAANAELDCYGVSDFYKRLIDKAKTVEGVEALKDAILAALP

SEQ ID NO: 354 - protein, 46 aa, Artificial Sequence
  Description of Artificial Sequence: Synthetic polypeptide
LAEAKSAANSELDCYGVSDFYKRLIDKAKTVEGVEALKDAILAALP

SEQ ID NO: 355 - protein, 46 aa, Artificial Sequence
  Description of Artificial Sequence: Synthetic polypeptide
LAEAKEAANAELDKYGVSDFYKRLIDKAKTVEGVEALKDAILAALP

SEQ ID NO: 356 - protein, 46 aa, Artificial Sequence
  Description of Artificial Sequence: Synthetic polypeptide
LAEAKEAANSELDKYGVSDFYKRLIDKAKTVEGVEALKDAILAALP

SEQ ID NO: 357 - protein, 46 aa, Artificial Sequence
  Description of Artificial Sequence: Synthetic polypeptide
LAEAKESANSELDKYGVSDFYKRLIDKAKTVEGVEALKDAILAALP

SEQ ID NO: 358 - protein, 46 aa, Artificial Sequence
  Description of Artificial Sequence: Synthetic polypeptide
LAEAKESANAELDKYGVSDFYKRLIDKAKTVEGVEALKDAILAALP

SEQ ID NO: 359 - protein, 46 aa, Artificial Sequence
  Description of Artificial Sequence: Synthetic polypeptide
LAEAKSAANAELDKYGVSDFYKRLIDKAKTVEGVEALKDAILAALP

SEQ ID NO: 360 - protein, 46 aa, Artificial Sequence
  Description of Artificial Sequence: Synthetic polypeptide
LAEAKSAANSELDKYGVSDFYKRLIDKAKTVEGVEALKDAILAALP

SEQ ID NO: 361 - protein, 46 aa, Artificial Sequence
  Description of Artificial Sequence: Synthetic polypeptide
LAQAKEAANAELDCYGVSDFYKRLIDKAKTVEGVEALKDAILAALP

SEQ ID NO: 362 - protein, 46 aa, Artificial Sequence
  Description of Artificial Sequence: Synthetic polypeptide
LAQAKEAANSELDCYGVSDFYKRLIDKAKTVEGVEALKDAILAALP

SEQ ID NO: 363 - protein, 46 aa, Artificial Sequence
  Description of Artificial Sequence: Synthetic polypeptide
LAQAKESANSELDCYGVSDFYKRLIDKAKTVEGVEALKDAILAALP

SEQ ID NO: 364 - protein, 46 aa, Artificial Sequence
  Description of Artificial Sequence: Synthetic polypeptide
LAQAKESANAELDCYGVSDFYKRLIDKAKTVEGVEALKDAILAALP

SEQ ID NO: 365 - protein, 46 aa, Artificial Sequence
  Description of Artificial Sequence: Synthetic polypeptide
LAQAKSAANAELDCYGVSDFYKRLIDKAKTVEGVEALKDAILAALP

SEQ ID NO: 366 - protein, 46 aa, Artificial Sequence
  Description of Artificial Sequence: Synthetic polypeptide
LAQAKSAANSELDCYGVSDFYKRLIDKAKTVEGVEALKDAILAALP

SEQ ID NO: 367 - protein, 46 aa, Artificial Sequence
  Description of Artificial Sequence: Synthetic polypeptide
LAQAKEAANAELDKYGVSDFYKRLIDKAKTVEGVEALKDAILAALP

SEQ ID NO: 368 - protein, 46 aa, Artificial Sequence
  Description of Artificial Sequence: Synthetic polypeptide
LAQAKEAANSELDKYGVSDFYKRLIDKAKTVEGVEALKDAILAALP

SEQ ID NO: 369 - protein, 46 aa, Artificial Sequence
  Description of Artificial Sequence: Synthetic polypeptide
LAQAKESANSELDKYGVSDFYKRLIDKAKTVEGVEALKDAILAALP

SEQ ID NO: 370 - protein, 46 aa, Artificial Sequence
  Description of Artificial Sequence: Synthetic polypeptide
LAQAKESANAELDKYGVSDFYKRLIDKAKTVEGVEALKDAILAALP

SEQ ID NO: 371 - protein, 46 aa, Artificial Sequence
  Description of Artificial Sequence: Synthetic polypeptide
LAQAKSAANAELDKYGVSDFYKRLIDKAKTVEGVEALKDAILAALP

SEQ ID NO: 372 - protein, 46 aa, Artificial Sequence
  Description of Artificial Sequence: Synthetic polypeptide
LAQAKSAANSELDKYGVSDFYKRLIDKAKTVEGVEALKDAILAALP

SEQ ID NO: 373 - protein, 46 aa, Artificial Sequence
  Description of Artificial Sequence: Synthetic polypeptide
LACAKEAANAELDCYGVSDFYKRLIDKAKTVEGVEALKDAILAALP

SEQ ID NO: 374 - protein, 46 aa, Artificial Sequence
  Description of Artificial Sequence: Synthetic polypeptide
LACAKEAANSELDCYGVSDFYKRLIDKAKTVEGVEALKDAILAALP

SEQ ID NO: 375 - protein, 46 aa, Artificial Sequence
  Description of Artificial Sequence: Synthetic polypeptide
LACAKESANSELDCYGVSDFYKRLIDKAKTVEGVEALKDAILAALP

SEQ ID NO: 376 - protein, 46 aa, Artificial Sequence
  Description of Artificial Sequence: Synthetic polypeptide
LACAKESANAELDCYGVSDFYKRLIDKAKTVEGVEALKDAILAALP

SEQ ID NO: 377 - protein, 46 aa, Artificial Sequence
  Description of Artificial Sequence: Synthetic polypeptide
LACAKSAANAELDCYGVSDFYKRLIDKAKTVEGVEALKDAILAALP

SEQ ID NO: 378 - protein, 46 aa, Artificial Sequence
  Description of Artificial Sequence: Synthetic polypeptide
LACAKSAANSELDCYGVSDFYKRLIDKAKTVEGVEALKDAILAALP

SEQ ID NO: 379 - protein, 46 aa, Artificial Sequence
  Description of Artificial Sequence: Synthetic polypeptide
LACAKEAANAELDKYGVSDFYKRLIDKAKTVEGVEALKDAILAALP

SEQ ID NO: 380 - protein, 46 aa, Artificial Sequence
  Description of Artificial Sequence: Synthetic polypeptide
LACAKEAANSELDKYGVSDFYKRLIDKAKTVEGVEALKDAILAALP

SEQ ID NO: 381 - protein, 46 aa, Artificial Sequence
  Description of Artificial Sequence: Synthetic polypeptide
LACAKESANSELDKYGVSDFYKRLIDKAKTVEGVEALKDAILAALP

SEQ ID NO: 382 - protein, 46 aa, Artificial Sequence
  Description of Artificial Sequence: Synthetic polypeptide
LACAKESANAELDKYGVSDFYKRLIDKAKTVEGVEALKDAILAALP

SEQ ID NO: 383 - protein, 46 aa, Artificial Sequence
  Description of Artificial Sequence: Synthetic polypeptide
LACAKSAANAELDKYGVSDFYKRLIDKAKTVEGVEALKDAILAALP

SEQ ID NO: 384 - protein, 46 aa, Artificial Sequence
  Description of Artificial Sequence: Synthetic polypeptide
LACAKSAANSELDKYGVSDFYKRLIDKAKTVEGVEALKDAILAALP

SEQ ID NO: 385 - protein, 46 aa, Artificial Sequence
  Description of Artificial Sequence: Synthetic polypeptide
LACAKEAANAELDSYGVSDFYKRLIDKAKTVEGVEALKDAILAALP

SEQ ID NO: 386 - protein, 46 aa, Artificial Sequence
  Description of Artificial Sequence: Synthetic polypeptide
LACAKEAANSELDSYGVSDFYKRLIDKAKTVEGVEALKDAILAALP

SEQ ID NO: 387 - protein, 46 aa, Artificial Sequence
  Description of Artificial Sequence: Synthetic polypeptide
LACAKESANSELDSYGVSDFYKRLIDKAKTVEGVEALKDAILAALP

SEQ ID NO: 388 - protein, 46 aa, Artificial Sequence
  Description of Artificial Sequence: Synthetic polypeptide
LACAKESANAELDSYGVSDFYKRLIDKAKTVEGVEALKDAILAALP

SEQ ID NO: 389 - protein, 46 aa, Artificial Sequence
  Description of Artificial Sequence: Synthetic polypeptide
LACAKSAANAELDSYGVSDFYKRLIDKAKTVEGVEALKDAILAALP

SEQ ID NO: 390 - protein, 46 aa, Artificial Sequence
  Description of Artificial Sequence: Synthetic polypeptide
LACAKSAANSELDSYGVSDFYKRLIDKAKTVEGVEALKDAILAALP

SEQ ID NO: 391 - protein, 46 aa, Artificial Sequence
  Description of Artificial Sequence: Synthetic polypeptide
LACAKEAANAELDAYGVSDFYKRLIDKAKTVEGVEALKDAILAALP

SEQ ID NO: 392 - protein, 46 aa, Artificial Sequence
  Description of Artificial Sequence: Synthetic polypeptide
LACAKEAANSELDAYGVSDFYKRLIDKAKTVEGVEALKDAILAALP

SEQ ID NO: 393 - protein, 46 aa, Artificial Sequence
  Description of Artificial Sequence: Synthetic polypeptide
LACAKESANSELDAYGVSDFYKRLIDKAKTVEGVEALKDAILAALP

SEQ ID NO: 394 - protein, 46 aa, Artificial Sequence
  Description of Artificial Sequence: Synthetic polypeptide
LACAKESANAELDAYGVSDFYKRLIDKAKTVEGVEALKDAILAALP

SEQ ID NO: 395 - protein, 46 aa, Artificial Sequence
  Description of Artificial Sequence: Synthetic polypeptide
LACAKSAANAELDAYGVSDFYKRLIDKAKTVEGVEALKDAILAALP

SEQ ID NO: 396 - protein, 46 aa, Artificial Sequence
  Description of Artificial Sequence: Synthetic polypeptide
LACAKSAANSELDAYGVSDFYKRLIDKAKTVEGVEALKDAILAALP

SEQ ID NO: 397 - protein, 46 aa, Artificial Sequence
  Description of Artificial Sequence: Synthetic polypeptide
LAQAKCAANAELDAYGVSDFYKRLIDKAKTVEGVEALKDAILAALP

SEQ ID NO: 398 - protein, 46 aa, Artificial Sequence
  Description of Artificial Sequence: Synthetic polypeptide
LAQAKCAANSELDAYGVSDFYKRLIDKAKTVEGVEALKDAILAALP

SEQ ID NO: 399 - protein, 46 aa, Artificial Sequence
  Description of Artificial Sequence: Synthetic polypeptide
LAQAKCSANSELDAYGVSDFYKRLIDKAKTVEGVEALKDAILAALP

SEQ ID NO: 400 - protein, 46 aa, Artificial Sequence
  Description of Artificial Sequence: Synthetic polypeptide
LAQAKCSANAELDAYGVSDFYKRLIDKAKTVEGVEALKDAILAALP

SEQ ID NO: 401 - protein, 46 aa, Artificial Sequence
  Description of Artificial Sequence: Synthetic polypeptide
LAQAKCAANAELDCYGVSDFYKRLIDKAKTVEGVEALKDAILAALP

SEQ ID NO: 402 - protein, 46 aa, Artificial Sequence
  Description of Artificial Sequence: Synthetic polypeptide
LAQAKCAANSELDCYGVSDFYKRLIDKAKTVEGVEALKDAILAALP

SEQ ID NO: 403 - protein, 46 aa, Artificial Sequence
  Description of Artificial Sequence: Synthetic polypeptide
LAQAKCAANAELDSYGVSDFYKRLIDKAKTVEGVEALKDAILAALP

SEQ ID NO: 404 - protein, 46 aa, Artificial Sequence
  Description of Artificial Sequence: Synthetic polypeptide
LAQAKCAANSELDSYGVSDFYKRLIDKAKTVEGVEALKDAILAALP

SEQ ID NO: 405 - protein, 46 aa, Artificial Sequence
  Description of Artificial Sequence: Synthetic polypeptide
LAQAKCSANSELDSYGVSDFYKRLIDKAKTVEGVEALKDAILAALP

SEQ ID NO: 406 - protein, 46 aa, Artificial Sequence
  Description of Artificial Sequence: Synthetic polypeptide
LAQAKCSANAELDSYGVSDFYKRLIDKAKTVEGVEALKDAILAALP

SEQ ID NO: 407 - protein, 46 aa, Artificial Sequence
  Description of Artificial Sequence: Synthetic polypeptide
LAQAKCAANAELDKYGVSDFYKRLIDKAKTVEGVEALKDAILAALP

SEQ ID NO: 408 - protein, 46 aa, Artificial Sequence
  Description of Artificial Sequence: Synthetic polypeptide
LAQAKCAANSELDKYGVSDFYKRLIDKAKTVEGVEALKDAILAALP

SEQ ID NO: 409 - protein, 46 aa, Artificial Sequence
  Description of Artificial Sequence: Synthetic polypeptide
LASAKCAANAELDAYGVSDFYKRLIDKAKTVEGVEALKDAILAALP

SEQ ID NO: 410 - protein, 46 aa, Artificial Sequence
  Description of Artificial Sequence: Synthetic polypeptide
LASAKCAANSELDAYGVSDFYKRLIDKAKTVEGVEALKDAILAALP

SEQ ID NO: 411 - protein, 46 aa, Artificial Sequence
  Description of Artificial Sequence: Synthetic polypeptide
LASAKCSANSELDAYGVSDFYKRLIDKAKTVEGVEALKDAILAALP

SEQ ID NO: 412 - protein, 46 aa, Artificial Sequence
  Description of Artificial Sequence: Synthetic polypeptide
LASAKCSANAELDAYGVSDFYKRLIDKAKTVEGVEALKDAILAALP

SEQ ID NO: 413 - protein, 46 aa, Artificial Sequence
  Description of Artificial Sequence: Synthetic polypeptide
LASAKCAANAELDCYGVSDFYKRLIDKAKTVEGVEALKDAILAALP

SEQ ID NO: 414 - protein, 46 aa, Artificial Sequence
  Description of Artificial Sequence: Synthetic polypeptide
LASAKCAANSELDCYGVSDFYKRLIDKAKTVEGVEALKDAILAALP

SEQ ID NO: 415 - protein, 46 aa, Artificial Sequence
  Description of Artificial Sequence: Synthetic polypeptide
LASAKCAANAELDSYGVSDFYKRLIDKAKTVEGVEALKDAILAALP

SEQ ID NO: 416 - protein, 46 aa, Artificial Sequence
  Description of Artificial Sequence: Synthetic polypeptide
LASAKCAANSELDSYGVSDFYKRLIDKAKTVEGVEALKDAILAALP

SEQ ID NO: 417 - protein, 46 aa, Artificial Sequence
  Description of Artificial Sequence: Synthetic polypeptide
LASAKCSANSELDSYGVSDFYKRLIDKAKTVEGVEALKDAILAALP

SEQ ID NO: 418 - protein, 46 aa, Artificial Sequence
  Description of Artificial Sequence: Synthetic polypeptide
LASAKCSANAELDSYGVSDFYKRLIDKAKTVEGVEALKDAILAALP

SEQ ID NO: 419 - protein, 46 aa, Artificial Sequence
  Description of Artificial Sequence: Synthetic polypeptide
LASAKCAANAELDKYGVSDFYKRLIDKAKTVEGVEALKDAILAALP

SEQ ID NO: 420 - protein, 46 aa, Artificial Sequence
  Description of Artificial Sequence: Synthetic polypeptide
LASAKCAANSELDKYGVSDFYKRLIDKAKTVEGVEALKDAILAALP

SEQ ID NO: 421 - protein, 46 aa, Artificial Sequence
  Description of Artificial Sequence: Synthetic polypeptide
LAEAKCAANAELDAYGVSDFYKRLIDKAKTVEGVEALKDAILAALP

SEQ ID NO: 422 - protein, 46 aa, Artificial Sequence
  Description of Artificial Sequence: Synthetic polypeptide
LAEAKCAANSELDAYGVSDFYKRLIDKAKTVEGVEALKDAILAALP

SEQ ID NO: 423 - protein, 46 aa, Artificial Sequence
  Description of Artificial Sequence: Synthetic polypeptide
LAEAKCSANSELDAYGVSDFYKRLIDKAKTVEGVEALKDAILAALP

SEQ ID NO: 424 - protein, 46 aa, Artificial Sequence
  Description of Artificial Sequence: Synthetic polypeptide
LAEAKCSANAELDAYGVSDFYKRLIDKAKTVEGVEALKDAILAALP

SEQ ID NO: 425 - protein, 46 aa, Artificial Sequence
  Description of Artificial Sequence: Synthetic polypeptide
LAEAKCAANAELDCYGVSDFYKRLIDKAKTVEGVEALKDAILAALP

SEQ ID NO: 426 - protein, 46 aa, Artificial Sequence
  Description of Artificial Sequence: Synthetic polypeptide
LAEAKCAANSELDCYGVSDFYKRLIDKAKTVEGVEALKDAILAALP

SEQ ID NO: 427 - protein, 46 aa, Artificial Sequence
  Description of Artificial Sequence: Synthetic polypeptide
LAEAKCAANAELDSYGVSDFYKRLIDKAKTVEGVEALKDAILAALP

SEQ ID NO: 428 - protein, 46 aa, Artificial Sequence
  Description of Artificial Sequence: Synthetic polypeptide
LAEAKCAANSELDSYGVSDFYKRLIDKAKTVEGVEALKDAILAALP

SEQ ID NO: 429 - protein, 46 aa, Artificial Sequence
  Description of Artificial Sequence: Synthetic polypeptide
LAEAKCSANSELDSYGVSDFYKRLIDKAKTVEGVEALKDAILAALP

SEQ ID NO: 430 - protein, 46 aa, Artificial Sequence
  Description of Artificial Sequence: Synthetic polypeptide
LAEAKCSANAELDSYGVSDFYKRLIDKAKTVEGVEALKDAILAALP

SEQ ID NO: 431 - protein, 46 aa, Artificial Sequence
  Description of Artificial Sequence: Synthetic polypeptide
LAEAKCAANAELDKYGVSDFYKRLIDKAKTVEGVEALKDAILAALP

SEQ ID NO: 432 - protein, 46 aa, Artificial Sequence
  Description of Artificial Sequence: Synthetic polypeptide
LAEAKCAANSELDKYGVSDFYKRLIDKAKTVEGVEALKDAILAALP

SEQ ID NO: 433 - protein, 46 aa, Artificial Sequence
  Description of Artificial Sequence: Synthetic polypeptide
LACAKCAANAELDAYGVSDFYKRLIDKAKTVEGVEALKDAILAALP

SEQ ID NO: 434 - protein, 46 aa, Artificial Sequence
  Description of Artificial Sequence: Synthetic polypeptide
LACAKCAANSELDAYGVSDFYKRLIDKAKTVEGVEALKDAILAALP

SEQ ID NO: 435 - protein, 46 aa, Artificial Sequence
  Description of Artificial Sequence: Synthetic polypeptide
LACAKCSANSELDAYGVSDFYKRLIDKAKTVEGVEALKDAILAALP

SEQ ID NO: 436 - protein, 46 aa, Artificial Sequence
  Description of Artificial Sequence: Synthetic polypeptide
LACAKCSANAELDAYGVSDFYKRLIDKAKTVEGVEALKDAILAALP

SEQ ID NO: 437 - protein, 46 aa, Artificial Sequence
  Description of Artificial Sequence: Synthetic polypeptide
LACAKCAANAELDCYGVSDFYKRLIDKAKTVEGVEALKDAILAALP

SEQ ID NO: 438 - protein, 46 aa, Artificial Sequence
  Description of Artificial Sequence: Synthetic polypeptide
LACAKCAANSELDCYGVSDFYKRLIDKAKTVEGVEALKDAILAALP

SEQ ID NO: 439 - protein, 46 aa, Artificial Sequence
  Description of Artificial Sequence: Synthetic polypeptide
LACAKCAANAELDSYGVSDFYKRLIDKAKTVEGVEALKDAILAALP

SEQ ID NO: 440 - protein, 46 aa, Artificial Sequence
  Description of Artificial Sequence: Synthetic polypeptide
LACAKCAANSELDSYGVSDFYKRLIDKAKTVEGVEALKDAILAALP

SEQ ID NO: 441 - protein, 46 aa, Artificial Sequence
  Description of Artificial Sequence: Synthetic polypeptide
LACAKCSANSELDSYGVSDFYKRLIDKAKTVEGVEALKDAILAALP

SEQ ID NO: 442 - protein, 46 aa, Artificial Sequence
  Description of Artificial Sequence: Synthetic polypeptide
LACAKCSANAELDSYGVSDFYKRLIDKAKTVEGVEALKDAILAALP

SEQ ID NO: 443 - protein, 46 aa, Artificial Sequence
  Description of Artificial Sequence: Synthetic polypeptide
LACAKCAANAELDKYGVSDFYKRLIDKAKTVEGVEALKDAILAALP

SEQ ID NO: 444 - protein, 46 aa, Artificial Sequence
  Description of Artificial Sequence: Synthetic polypeptide
LACAKCAANSELDKYGVSDFYKRLIDKAKTVEGVEALKDAILAALP

SEQ ID NO: 445 - protein, 49 aa, Artificial Sequence
  Description of Artificial Sequence: Synthetic polypeptide
GSLASAKEAANAELDSYGVSDFYKRLIDKAKTVEGVEALKDAILAALPG

SEQ ID NO: 446 - protein, 49 aa, Artificial Sequence
  Description of Artificial Sequence: Synthetic polypeptide
GSLAEAKEAANAELDSYGVSDFYKRLIDKAKTVEGVEALKDAILAALPG

SEQ ID NO: 447 - protein, 49 aa, Artificial Sequence
  Description of Artificial Sequence: Synthetic polypeptide
GSLASAKEAANAELDCYGVSDFYKRLIDKAKTVEGVEALKDAILAALPG

SEQ ID NO: 448 - protein, 49 aa, Artificial Sequence
  Description of Artificial Sequence: Synthetic polypeptide
GSLAEAKEAANAELDCYGVSDFYKRLIDKAKTVEGVEALKDAILAALPG

SEQ ID NO: 449 - protein, 50 aa, Artificial Sequence
  Description of Artificial Sequence: Synthetic polypeptide
GSLASAKEAANAELDSYGVSDFYKRLIDKAKTVEGVEALKDAILAALPCG

SEQ ID NO: 450 - protein, 50 aa, Artificial Sequence
  Description of Artificial Sequence: Synthetic polypeptide
GSLAEAKEAANAELDSYGVSDFYKRLIDKAKTVEGVEALKDAILAALPCG

SEQ ID NO: 451 - protein, 50 aa, Artificial Sequence
  Description of Artificial Sequence: Synthetic polypeptide
GCSLASAKEAANAELDKYGVSDFYKRLIDKAKTVEGVEALKDAILAALPG

SEQ ID NO: 452 - protein, 50 aa, Artificial Sequence
  Description of Artificial Sequence: Synthetic polypeptide
GCSLAEAKEAANAELDKYGVSDFYKRLIDKAKTVEGVEALKDAILAALPG

SEQ ID NO: 453 - protein, 47 aa, Artificial Sequence
  Description of Artificial Sequence: Synthetic polypeptide
GLAEAKVLANRELDKYGVSDYYKNLINNAKTVEGVKALIDEILAALP

SEQ ID NO: 454 - protein, 49 aa, Artificial Sequence
  Description of Artificial Sequence: Synthetic polypeptide
GSSLAEAKVLANRELDKYGVSDFYKRLINKAKTVEGVEALKLHILAALP

SEQ ID NO: 455 - protein, 49 aa, Artificial Sequence
  Description of Artificial Sequence: Synthetic polypeptide
GSSLASAKEAANAELDAYGVSDFYKRLIDKAKTVEGVEALKDAILAALP

SEQ ID NO: 456 - protein, 49 aa, Artificial Sequence
  Description of Artificial Sequence: Synthetic polypeptide
GSSLASAKEAANAELDSYGVSDFYKRLIDKAKTVEGVEALKDAILAALP

SEQ ID NO: 457 - protein, 47 aa, Artificial Sequence
  Description of Artificial Sequence: Synthetic polypeptide
GLASAKEAANAELDSYGVSDFYKRLIDKAKTVEGVEALKDAILAALP

SEQ ID NO: 458 - protein, 47 aa, Artificial Sequence
  Description of Artificial Sequence: Synthetic polypeptide
GLAEAKEAANAELDSYGVSDFYKRLIDKAKTVEGVEALKDAILAALP

SEQ ID NO: 459 - protein, 47 aa, Artificial Sequence; identical to SEQ ID NO: 159
  Description of Artificial Sequence: Synthetic polypeptide

SEQ ID NO: 460 - protein, 47 aa, Artificial Sequence; identical to SEQ ID NO: 160
  Description of Artificial Sequence: Synthetic polypeptide

SEQ ID NO: 461 - protein, 49 aa, Artificial Sequence
  Description of Artificial Sequence: Synthetic polypeptide
GSSLASAKEAANAELDKYGVSDFYKRLIDKAKTVEGVEALKDAILAALP

SEQ ID NO: 462 - protein, 48 aa, Artificial Sequence
  Description of Artificial Sequence: Synthetic polypeptide
GSLASAKEAANAELDSYGVSDFYKRLIDKAKTVEGVEALKDAILAALP

SEQ ID NO: 463 - protein, 48 aa, Artificial Sequence
  Description of Artificial Sequence: Synthetic polypeptide
GSLAEAKEAANAELDSYGVSDFYKRLIDKAKTVEGVEALKDAILAALP

SEQ ID NO: 464-499 - skipped (36 entries)

SEQ ID NO: 500 - protein, 47 aa, Artificial Sequence
  Description of Artificial Sequence: Synthetic polypeptide
SLAEAKEAANAELDSYGVSDFYKRLIDKAKTVEGVEALKDAILAALP

SEQ ID NO: 501 - protein, 47 aa, Artificial Sequence
  Description of Artificial Sequence: Synthetic polypeptide
LAEAKEAANAELDCYGVSDFYKRLIDKAKTVEGVEALKDAILAALPG

SEQ ID NO: 502 - protein, 48 aa, Artificial Sequence
  Description of Artificial Sequence: Synthetic polypeptide
SLAEAKEAANAELDCYGVSDFYKRLIDKAKTVEGVEALKDAILAALPG

SEQ ID NO: 503-599 - skipped (97 entries)

SEQ ID NO: 600 - protein, 27 aa, Artificial Sequence
  Description of Artificial Sequence: Synthetic peptide
MGSSHHHHHHLQSSGVDLGTENLYFQG

SEQ ID NO: 601-677 - skipped (77 entries)

SEQ ID NO: 678 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (45)..(46): May or may not be present
LAXAKXXANXELDXYGVSDFYKRLIDKAKTVEGVEALKDAILAALP

SEQ ID NO: 679 - protein, 46 aa, Artificial Sequence
  Description of Artificial Sequence: Synthetic polypeptide
  MOD_RES (6)..(6): Val or Glu
  MOD_RES (7)..(7): Leu, Glu or Asp
  MOD_RES (9)..(9): Asn, Leu or Ile
  MOD_RES (10)..(10): Arg or Lys
  MOD_RES (13)..(13): Asp or Lys
  MOD_RES (20)..(20): Tyr or Phe
  MOD_RES (23)..(23): Asn, Arg or Ser
  MOD_RES (24)..(24): Val, Ile, Leu, Met, Phe or Tyr
  MOD_RES (26)..(26): Asn, Ser, Glu or Asp
  MOD_RES (27)..(27): Arg, Lys or Asn
  MOD_RES (29)..(29): Lys or Arg
  MOD_RES (35)..(35): Asp, Asn, Gln, Glu, His, Ser, Arg or Lys
  MOD_RES (38)..(38): Lys, Ile or Thr
  MOD_RES (39)..(39): Asn, Ser, Thr, Gly, His, Leu or Asp
  MOD_RES (40)..(40): His, Glu or Asp
LAEAKXXAXXELXKYGVSDXYKXXIXXAXTVEGVXALXXXILAALP

SEQ ID NO: 680 - protein, 30 aa, Artificial Sequence
  Description of Artificial Sequence: Synthetic polypeptide
HGEGTFTSDLSKQMEEEAVRLFIEWLKNGG

SEQ ID NO: 681 - protein, 4 aa, Artificial Sequence
  Description of Artificial Sequence: Synthetic peptide
  MOD_RES (3)..(3): May or may not be present
  C-term OH or NH2
GGPS

SEQ ID NO: 682 - protein, 5 aa, Artificial Sequence
  Description of Artificial Sequence: Synthetic peptide
  MOD_RES (3)..(3): May or may not be present
  C-term OH or NH2
GGPSS

SEQ ID NO: 683 - protein, 6 aa, Artificial Sequence
  Description of Artificial Sequence: Synthetic peptide
  MOD_RES (3)..(3): May or may not be present
  C-term OH or NH2
GGPSSG

SEQ ID NO: 684 - protein, 7 aa, Artificial Sequence
  Description of Artificial Sequence: Synthetic peptide
  MOD_RES (3)..(3): May or may not be present
  C-term OH or NH2
GGPSSGA

SEQ ID NO: 685 - protein, 8 aa, Artificial Sequence
  Description of Artificial Sequence: Synthetic peptide
  MOD_RES (3)..(3): May or may not be present
  MOD_RES (8)..(8): May or may not be present
  C-term OH or NH2
GGPSSGAP

SEQ ID NO: 686 - protein, 9 aa, Artificial Sequence
  Description of Artificial Sequence: Synthetic peptide
  MOD_RES (3)..(3): May or may not be present
  MOD_RES (8)..(9): May or may not be present
  C-term OH or NH2
GGPSSGAPP

SEQ ID NO: 687 - protein, 10 aa, Artificial Sequence
  Description of Artificial Sequence: Synthetic peptide
  MOD_RES (3)..(3): May or may not be present
  MOD_RES (8)..(10): May or may not be present
  C-term OH or NH2
GGPSSGAPPP

SEQ ID NO: 688 - protein, 50 aa, Artificial Sequence
  Description of Artificial Sequence: Synthetic polypeptide
  N-term H
KKKKKKHGEGTFTSDLSKQMEEEAVRLFIEWLKNGGPSSGAPPSKKKKKK

SEQ ID NO: 689 - protein, 38 aa, Artificial Sequence
  Description of Artificial Sequence: Synthetic polypeptide
  N-term H
  C-term NH2
HGEGTFTSDLSKQMEEEAVRLFIEWLWLKNGGPSSGAS

SEQ ID NO: 690 - protein, 42 aa, Artificial Sequence
  Description of Artificial Sequence: Synthetic polypeptide
  N-term H
KKKKKKHGEGTFTSDLSKQMEEEAVRLFIEWLKNGGPSSGAS

SEQ ID NO: 691 - protein, 42 aa, Artificial Sequence
  Description of Artificial Sequence: Synthetic polypeptide
  N-term H
NEEEEEHGEGTFTSDLSKQMEEEAVRLFIEWLKNGGPSSGAS

SEQ ID NO: 692 - protein, 42 aa, Artificial Sequence
  Description of Artificial Sequence: Synthetic polypeptide
HGEGTFTSDLSKQMEEEAVRLFIEWLKNGGPSSGASKKKKKK

SEQ ID NO: 693 - protein, 48 aa, Artificial Sequence
  Description of Artificial Sequence: Synthetic polypeptide
  N-term H
KKKKKKHGEGTFTSDLSKQMEEEAVRLFIEWLKNGGPSSGASKKKKKK

SEQ ID NO: 694 - protein, 48 aa, Artificial Sequence
  Description of Artificial Sequence: Synthetic polypeptide
NEEEEEHGEGTFTSDLSKQMEEEAVRLFIEWLKNGGPSSGASKKKKKK

SEQ ID NO: 695 - protein, 6 aa, Artificial Sequence
  Description of Artificial Sequence: Synthetic peptide
KKKKKK

SEQ ID NO: 696 - protein, 49 aa, Artificial Sequence
  Description of Artificial Sequence: Synthetic polypeptide
SGSLAEAKEAANAELDSYGVSDFYKRLIDKAKTVEGVEALKDAILAALP

SEQ ID NO: 697 - protein, 48 aa, Artificial Sequence
  Description of Artificial Sequence: Synthetic polypeptide
SGSLAEAKEAANAELDSYGVSDFYKRLIDKAKTVEGVEALKDAILAAL

SEQ ID NO: 698 - protein, 48 aa, Artificial Sequence
  Description of Artificial Sequence: Synthetic polypeptide
SGSLAEAKEAANAELDSYGVSDFYKRLIDKAKTVEGVEALKDAILAAP

SEQ ID NO: 699 - protein, 47 aa, Artificial Sequence
  Description of Artificial Sequence: Synthetic polypeptide
SGSLAEAKEAANAELDSYGVSDFYKRLIDKAKTVEGVEALKDAILAA

SEQ ID NO: 700 - protein, 47 aa, Artificial Sequence
  Description of Artificial Sequence: Synthetic polypeptide
GSLAEAKEAANAELDSYGVSDFYKRLIDKAKTVEGVEALKDAILAAL

SEQ ID NO: 701 - protein, 47 aa, Artificial Sequence
  Description of Artificial Sequence: Synthetic polypeptide
GSLAEAKEAANAELDCYGVSDFYKRLIDKAKTVEGVEALKDAILAAL

SEQ ID NO: 702 - protein, 46 aa, Artificial Sequence
  Description of Artificial Sequence: Synthetic polypeptide
SLAEAKEAANAELDSYGVSDFYKRLIDKAKTVEGVEALKDAILAAL

SEQ ID NO: 703 - protein, 46 aa, Artificial Sequence
  Description of Artificial Sequence: Synthetic polypeptide
SLAEAKEAANAELDCYGVSDFYKRLIDKAKTVEGVEALKDAILAAL

SEQ ID NO: 704 - protein, 45 aa, Artificial Sequence
  Description of Artificial Sequence: Synthetic polypeptide
LAEAKEAANAELDSYGVSDFYKRLIDKAKTVEGVEALKDAILAAL

SEQ ID NO: 705 - protein, 45 aa, Artificial Sequence
  Description of Artificial Sequence: Synthetic polypeptide
LAEAKEAANAELDCYGVSDFYKRLIDKAKTVEGVEALKDAILAAL

SEQ ID NO: 706 - protein, 47 aa, Artificial Sequence
  Description of Artificial Sequence: Synthetic polypeptide
GSSLASAKEAANAELDAYGVSDFYKRLIDKAKTVEGVEALKDAILAA

SEQ ID NO: 707 - protein, 47 aa, Artificial Sequence
  Description of Artificial Sequence: Synthetic polypeptide
GSSLASAKEAANAELDSYGVSDFYKRLIDKAKTVEGVEALKDAILAA

SEQ ID NO: 708 - protein, 45 aa, Artificial Sequence
  Description of Artificial Sequence: Synthetic polypeptide
GLASAKEAANAELDSYGVSDFYKRLIDKAKTVEGVEALKDAILAA

SEQ ID NO: 709 - protein, 45 aa, Artificial Sequence
  Description of Artificial Sequence: Synthetic polypeptide
GLAEAKEAANAELDSYGVSDFYKRLIDKAKTVEGVEALKDAILAA

SEQ ID NO: 710 - protein, 45 aa, Artificial Sequence
  Description of Artificial Sequence: Synthetic polypeptide
GLASAKEAANAELDCYGVSDFYKRLIDKAKTVEGVEALKDAILAA

SEQ ID NO: 711 - protein, 45 aa, Artificial Sequence
  Description of Artificial Sequence: Synthetic polypeptide
ALASAKEAANAELDCYGVSDFYKRLIDKAKTVEGVEALKDAILAA

SEQ ID NO: 712 - protein, 47 aa, Artificial Sequence
  Description of Artificial Sequence: Synthetic polypeptide
GSSLASAKEAANAELDKYGVSDFYKRLIDKAKTVEGVEALKDAILAA

SEQ ID NO: 713 - protein, 46 aa, Artificial Sequence
  Description of Artificial Sequence: Synthetic polypeptide
GSLASAKEAANAELDSYGVSDFYKRLIDKAKTVEGVEALKDAILAA

SEQ ID NO: 714 - protein, 46 aa, Artificial Sequence
  Description of Artificial Sequence: Synthetic polypeptide
GSLAEAKEAANAELDSYGVSDFYKRLIDKAKTVEGVEALKDAILAA

SEQ ID NO: 715 - protein, 46 aa, Artificial Sequence
  Description of Artificial Sequence: Synthetic polypeptide
GSLAEAKEAANAELDCYGVSDFYKRLIDKAKTVEGVEALKDAILAA

SEQ ID NO: 716 - protein, 44 aa, Artificial Sequence
  Description of Artificial Sequence: Synthetic polypeptide
LAEAKEAANAELDSYGVSDFYKRLIDKAKTVEGVEALKDAILAA

SEQ ID NO: 717 - protein, 45 aa, Artificial Sequence
  Description of Artificial Sequence: Synthetic polypeptide
SLAEAKEAANAELDSYGVSDFYKRLIDKAKTVEGVEALKDAILAA

SEQ ID NO: 718 - protein, 44 aa, Artificial Sequence
  Description of Artificial Sequence: Synthetic polypeptide
LAEAKEAANAELDCYGVSDFYKRLIDKAKTVEGVEALKDAILAA

SEQ ID NO: 719 - protein, 45 aa, Artificial Sequence
  Description of Artificial Sequence: Synthetic polypeptide
SLAEAKEAANAELDCYGVSDFYKRLIDKAKTVEGVEALKDAILAA

SEQ ID NO: 720 - protein, 49 aa, Artificial Sequence
  Description of Artificial Sequence: Synthetic polypeptide
AGSLAEAKEAANAELDSYGVSDFYKRLIDKAKTVEGVEALKDAILAALP

SEQ ID NO: 721 - protein, 15 aa, Artificial Sequence
  Description of Artificial Sequence: Synthetic peptide
GGGGSGGGGSGGGGS

SEQ ID NO: 722 - protein, 18 aa, Artificial Sequence
  Description of Artificial Sequence: Synthetic peptide
GGEGGEGGEGGEGGEGGE

SEQ ID NO: 723 - protein, 18 aa, Artificial Sequence
  Description of Artificial Sequence: Synthetic peptide
GGKGGKGGKGGKGGKGGK

SEQ ID NO: 724 - protein, 15 aa, Artificial Sequence
  Description of Artificial Sequence: Synthetic peptide
EAAAKEAAAKEAAAK

SEQ ID NO: 725 - protein, 20 aa, Artificial Sequence
  Description of Artificial Sequence: Synthetic peptide
EAAAKEAAAKEAAAKEAAAK

SEQ ID NO: 726 - protein, 25 aa, Artificial Sequence
  Description of Artificial Sequence: Synthetic peptide
EAAAKEAAAKEAAAKEAAAKEAAAK

SEQ ID NO: 727 - protein, 93 aa, Artificial Sequence
  Description of Artificial Sequence: Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 728 - protein, 95 aa, Artificial Sequence
  Description of Artificial Sequence: Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAALP

SEQ ID NO: 729 - protein, 94 aa, Artificial Sequence
  Description of Artificial Sequence: Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAAL

SEQ ID NO: 730 - protein, 95 aa, Artificial Sequence
  Description of Artificial Sequence: Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 731 - protein, 88 aa, Artificial Sequence
  Description of Artificial Sequence: Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAALP

SEQ ID NO: 732 - protein, 95 aa, Artificial Sequence
  Description of Artificial Sequence: Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 733 - protein, 88 aa, Artificial Sequence
  Description of Artificial Sequence: Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAALP

SEQ ID NO: 734 - protein, 92 aa, Artificial Sequence
  Description of Artificial Sequence: Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAAL

SEQ ID NO: 735 - protein, 94 aa, Artificial Sequence
  Description of Artificial Sequence: Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAAL

SEQ ID NO: 736 - protein, 93 aa, Artificial Sequence
  Description of Artificial Sequence: Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAA

SEQ ID NO: 737 - protein, 94 aa, Artificial Sequence
  Description of Artificial Sequence: Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAAL

SEQ ID NO: 738 - protein, 87 aa, Artificial Sequence
  Description of Artificial Sequence: Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAAL

SEQ ID NO: 739 - protein, 87 aa, Artificial Sequence
  Description of Artificial Sequence: Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAAL

SEQ ID NO: 740 - protein, 91 aa, Artificial Sequence
  Description of Artificial Sequence: Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAA

SEQ ID NO: 741 - protein, 93 aa, Artificial Sequence
  Description of Artificial Sequence: Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAA

SEQ ID NO: 742 - protein, 93 aa, Artificial Sequence
  Description of Artificial Sequence: Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAA

SEQ ID NO: 743 - protein, 86 aa, Artificial Sequence
  Description of Artificial Sequence: Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAA

SEQ ID NO: 744 - protein, 93 aa, Artificial Sequence; identical to SEQ ID NO: 736
  Description of Artificial Sequence: Synthetic polypeptide

SEQ ID NO: 745 - protein, 86 aa, Artificial Sequence
  Description of Artificial Sequence: Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAA

SEQ ID NO: 746 - protein, 92 aa, Artificial Sequence
  Description of Artificial Sequence: Synthetic polypeptide
HGEGTFTSDLSKQLEEEAVRLFIEWLKQGGPSKEIISTGGGGSASSLAEAKEAANAELDSYGVSDFYKRLIDKAKTVEGVEALKDAILAALP

SEQ ID NO: 747 - protein, 91 aa, Artificial Sequence
  Description of Artificial Sequence: Synthetic polypeptide
HGEGTFTSDLSKQLEEEAVRLFIEWLKQGGPSKEIISTGGGGSASSLAEAKEAANAELDSYGVSDFYKRLIDKAKTVEGVEALKDAILAAL

SEQ ID NO: 748 - protein, 91 aa, Artificial Sequence
  Description of Artificial Sequence: Synthetic polypeptide
HGEGTFTSDLSKQLEEEAVRLFIEWLKQGGPSKEIISTGGGGSASLAEAKEAANAELDSYGVSDFYKRLIDKAKTVEGVEALKDAILAALP

SEQ ID NO: 749 - protein, 90 aa, Artificial Sequence
  Description of Artificial Sequence: Synthetic polypeptide
HGEGTFTSDLSKQLEEEAVRLFIEWLKQGGPSKEIISTGGGGSASLAEAKEAANAELDSYGVSDFYKRLIDKAKTVEGVEALKDAILAAL

SEQ ID NO: 750 - protein, 88 aa, Artificial Sequence
  Description of Artificial Sequence: Synthetic polypeptide
HGEGTFTSDLSKQLEEEAVRLFIEWLKQGGPSKEIISGGGGSLAEAKEAANAELDSYGVSDFYKRLIDKAKTVEGVEALKDAILAALP

SEQ ID NO: 751 - protein, 87 aa, Artificial Sequence
  Description of Artificial Sequence: Synthetic polypeptide
HGEGTFTSDLSKQLEEEAVRLFIEWLKQGGPSKEIISGGGGSLAEAKEAANAELDSYGVSDFYKRLIDKAKTVEGVEALKDAILAAL

SEQ ID NO: 752 - protein, 87 aa, Artificial Sequence
  Description of Artificial Sequence: Synthetic polypeptide
HGEGTFTSDLSKQLEEEAVRLFIEWLKQGGPSKEIISGGGSLAEAKEAANAELDSYGVSDFYKRLIDKAKTVEGVEALKDAILAALP

SEQ ID NO: 753 - protein, 86 aa, Artificial Sequence
  Description of Artificial Sequence: Synthetic polypeptide
HGEGTFTSDLSKQLEEEAVRLFIEWLKQGGPSKEIISGGGSLAEAKEAANAELDSYGVSDFYKRLIDKAKTVEGVEALKDAILAAL

SEQ ID NO: 754 - protein, 86 aa, Artificial Sequence
  Description of Artificial Sequence: Synthetic polypeptide
HGEGTFTSDLSKQLEEEAVRLFIEWLKQGGPSKEIISGGGLAEAKEAANAELDSYGVSDFYKRLIDKAKTVEGVEALKDAILAALP

SEQ ID NO: 755 - protein, 85 aa, Artificial Sequence
  Description of Artificial Sequence: Synthetic polypeptide
HGEGTFTSDLSKQLEEEAVRLFIEWLKQGGPSKEIISGGGLAEAKEAANAELDSYGVSDFYKRLIDKAKTVEGVEALKDAILAAL

SEQ ID NO: 756 - protein, 86 aa, Artificial Sequence
  Description of Artificial Sequence: Synthetic polypeptide
HGEGTFTSDLSKQLEEEAVRLFIEWLKQGGPSKEIISGGSLAEAKEAANAELDSYGVSDFYKRLIDKAKTVEGVEALKDAILAALP

SEQ ID NO: 757 - protein, 85 aa, Artificial Sequence
  Description of Artificial Sequence: Synthetic polypeptide
HGEGTFTSDLSKQLEEEAVRLFIEWLKQGGPSKEIISGGSLAEAKEAANAELDSYGVSDFYKRLIDKAKTVEGVEALKDAILAAL

SEQ ID NO: 758 - protein, 94 aa, Artificial Sequence
  Description of Artificial Sequence: Synthetic polypeptide
HGEGTFTSDLSKQLEEEAVRLFIEWLKNGGPSSGAPPKSTGGGGSASSLAEAKEAANAELDSYGVSDFYKRLIDKAKTVEGVEALKDAILAALP

SEQ ID NO: 759 - protein, 93 aa, Artificial Sequence
  Description of Artificial Sequence: Synthetic polypeptide
HGEGTFTSDLSKQLEEEAVRLFIEWLKNGGPSSGAPPKSTGGGGSASSLAEAKEAANAELDSYGVSDFYKRLIDKAKTVEGVEALKDAILAAL

SEQ ID NO: 760 - protein, 93 aa, Artificial Sequence
  Description of Artificial Sequence: Synthetic polypeptide
HGEGTFTSDLSKQLEEEAVRLFIEWLKNGGPSSGAPPKSTGGGGSASLAEAKEAANAELDSYGVSDFYKRLIDKAKTVEGVEALKDAILAALP

SEQ ID NO: 761 - protein, 92 aa, Artificial Sequence
  Description of Artificial Sequence: Synthetic polypeptide
HGEGTFTSDLSKQLEEEAVRLFIEWLKNGGPSSGAPPKSTGGGGSASLAEAKEAANAELDSYGVSDFYKRLIDKAKTVEGVEALKDAILAAL

SEQ ID NO: 762 - protein, 90 aa, Artificial Sequence
  Description of Artificial Sequence: Synthetic polypeptide
HGEGTFTSDLSKQLEEEAVRLFIEWLKNGGPSSGAPPKSGGGGSLAEAKEAANAELDSYGVSDFYKRLIDKAKTVEGVEALKDAILAALP

SEQ ID NO: 763 - protein, 89 aa, Artificial Sequence
  Description of Artificial Sequence: Synthetic polypeptide
HGEGTFTSDLSKQLEEEAVRLFIEWLKNGGPSSGAPPKSGGGGSLAEAKEAANAELDSYGVSDFYKRLIDKAKTVEGVEALKDAILAAL

SEQ ID NO: 764 - protein, 89 aa, Artificial Sequence
  Description of Artificial Sequence: Synthetic polypeptide
HGEGTFTSDLSKQLEEEAVRLFIEWLKNGGPSSGAPPKSGGGSLAEAKEAANAELDSYGVSDFYKRLIDKAKTVEGVEALKDAILAALP

SEQ ID NO: 765 - protein, 88 aa, Artificial Sequence
  Description of Artificial Sequence: Synthetic polypeptide
HGEGTFTSDLSKQLEEEAVRLFIEWLKNGGPSSGAPPKSGGGSLAEAKEAANAELDSYGVSDFYKRLIDKAKTVEGVEALKDAILAAL

SEQ ID NO: 766 - protein, 88 aa, Artificial Sequence
  Description of Artificial Sequence: Synthetic polypeptide
HGEGTFTSDLSKQLEEEAVRLFIEWLKNGGPSSGAPPKSGGGLAEAKEAANAELDSYGVSDFYKRLIDKAKTVEGVEALKDAILAALP

SEQ ID NO: 767 - protein, 87 aa, Artificial Sequence
  Description of Artificial Sequence: Synthetic polypeptide
HGEGTFTSDLSKQLEEEAVRLFIEWLKNGGPSSGAPPKSGGGLAEAKEAANAELDSYGVSDFYKRLIDKAKTVEGVEALKDAILAAL

SEQ ID NO: 768 - protein, 88 aa, Artificial Sequence
  Description of Artificial Sequence: Synthetic polypeptide
HGEGTFTSDLSKQLEEEAVRLFIEWLKNGGPSSGAPPKSGGSLAEAKEAANAELDSYGVSDFYKRLIDKAKTVEGVEALKDAILAALP

SEQ ID NO: 769 - protein, 87 aa, Artificial Sequence
  Description of Artificial Sequence: Synthetic polypeptide
HGEGTFTSDLSKQLEEEAVRLFIEWLKNGGPSSGAPPKSGGSLAEAKEAANAELDSYGVSDFYKRLIDKAKTVEGVEALKDAILAAL

SEQ ID NO: 770 - protein, 87 aa, Artificial Sequence
  Description of Artificial Sequence: Synthetic polypeptide
HGEGTFTSDLSKQLEEEAVRLFIEWLKNGGPSSGAPPKSGSLAEAKEAANAELDSYGVSDFYKRLIDKAKTVEGVEALKDAILAALP

SEQ ID NO: 771 - protein, 86 aa, Artificial Sequence
  Description of Artificial Sequence: Synthetic polypeptide
HGEGTFTSDLSKQLEEEAVRLFIEWLKNGGPSSGAPPKSGSLAEAKEAANAELDSYGVSDFYKRLIDKAKTVEGVEALKDAILAAL

SEQ ID NO: 772 - protein, 86 aa, Artificial Sequence
  Description of Artificial Sequence: Synthetic polypeptide
HGEGTFTSDLSKQLEEEAVRLFIEWLKNGGPSSGAPPKSGLAEAKEAANAELDSYGVSDFYKRLIDKAKTVEGVEALKDAILAALP

SEQ ID NO: 773 - protein, 85 aa, Artificial Sequence
  Description of Artificial Sequence: Synthetic polypeptide
HGEGTFTSDLSKQLEEEAVRLFIEWLKNGGPSSGAPPKSGLAEAKEAANAELDSYGVSDFYKRLIDKAKTVEGVEALKDAILAAL

SEQ ID NO: 774 - protein, 95 aa, Artificial Sequence
  Description of Artificial Sequence: Synthetic polypeptide
HGEGTFTSDLSKQMEEEAVRLFIEWLKNGGPSSGAPPKSTGGGGSASGSLAEAKEAANAELDSYGVSDFYKRLIDKAKTVEGVEALKDAILAALP

SEQ ID NO: 775 - protein, 94 aa, Artificial Sequence
  Description of Artificial Sequence: Synthetic polypeptide
HGEGTFTSDLSKQMEEEAVRLFIEWLKNGGPSSGAPPKSTGGGGSASGSLAEAKEAANAELDSYGVSDFYKRLIDKAKTVEGVEALKDAILAAL

SEQ ID NO: 776 - protein, 94 aa, Artificial Sequence
  Description of Artificial Sequence: Synthetic polypeptide
HGEGTFTSDLSKQMEEEAVRLFIEWLKNGGPSSGAPPKSTGGGGSASSLAEAKEAANAELDSYGVSDFYKRLIDKAKTVEGVEALKDAILAALP

SEQ ID NO: 777 - protein, 93 aa, Artificial Sequence
  Description of Artificial Sequence: Synthetic polypeptide
HGEGTFTSDLSKQMEEEAVRLFIEWLKNGGPSSGAPPKSTGGGGSASSLAEAKEAANAELDSYGVSDFYKRLIDKAKTVEGVEALKDAILAAL

SEQ ID NO: 778 - protein, 93 aa, Artificial Sequence
  Description of Artificial Sequence: Synthetic polypeptide
HGEGTFTSDLSKQMEEEAVRLFIEWLKNGGPSSGAPPKSTGGGGSASLAEAKEAANAELDSYGVSDFYKRLIDKAKTVEGVEALKDAILAALP

SEQ ID NO: 779 - protein, 92 aa, Artificial Sequence
  Description of Artificial Sequence: Synthetic polypeptide
HGEGTFTSDLSKQMEEEAVRLFIEWLKNGGPSSGAPPKSTGGGGSASLAEAKEAANAELDSYGVSDFYKRLIDKAKTVEGVEALKDAILAAL

SEQ ID NO: 780 - protein, 90 aa, Artificial Sequence
  Description of Artificial Sequence: Synthetic polypeptide
HGEGTFTSDLSKQMEEEAVRLFIEWLKNGGPSSGAPPKSGGGGSLAEAKEAANAELDSYGVSDFYKRLIDKAKTVEGVEALKDAILAALP

SEQ ID NO: 781 - protein, 89 aa, Artificial Sequence
  Description of Artificial Sequence: Synthetic polypeptide
HGEGTFTSDLSKQMEEEAVRLFIEWLKNGGPSSGAPPKSGGGGSLAEAKEAANAELDSYGVSDFYKRLIDKAKTVEGVEALKDAILAAL

SEQ ID NO: 782 - protein, 89 aa, Artificial Sequence
  Description of Artificial Sequence: Synthetic polypeptide
HGEGTFTSDLSKQMEEEAVRLFIEWLKNGGPSSGAPPKSGGGSLAEAKEAANAELDSYGVSDFYKRLIDKAKTVEGVEALKDAILAALP

SEQ ID NO: 783 - protein, 88 aa, Artificial Sequence
  Description of Artificial Sequence: Synthetic polypeptide
HGEGTFTSDLSKQMEEEAVRLFIEWLKNGGPSSGAPPKSGGGSLAEAKEAANAELDSYGVSDFYKRLIDKAKTVEGVEALKDAILAAL

SEQ ID NO: 784 - protein, 88 aa, Artificial Sequence
  Description of Artificial Sequence: Synthetic polypeptide
HGEGTFTSDLSKQMEEEAVRLFIEWLKNGGPSSGAPPKSGGGLAEAKEAANAELDSYGVSDFYKRLIDKAKTVEGVEALKDAILAALP

SEQ ID NO: 785 - protein, 87 aa, Artificial Sequence
  Description of Artificial Sequence: Synthetic polypeptide
HGEGTFTSDLSKQMEEEAVRLFIEWLKNGGPSSGAPPKSGGGLAEAKEAANAELDSYGVSDFYKRLIDKAKTVEGVEALKDAILAAL

SEQ ID NO: 786 - protein, 88 aa, Artificial Sequence
  Description of Artificial Sequence: Synthetic polypeptide
HGEGTFTSDLSKQMEEEAVRLFIEWLKNGGPSSGAPPKSGGSLAEAKEAANAELDSYGVSDFYKRLIDKAKTVEGVEALKDAILAALP

SEQ ID NO: 787 - protein, 87 aa, Artificial Sequence
  Description of Artificial Sequence: Synthetic polypeptide
HGEGTFTSDLSKQMEEEAVRLFIEWLKNGGPSSGAPPKSGGSLAEAKEAANAELDSYGVSDFYKRLIDKAKTVEGVEALKDAILAAL

SEQ ID NO: 788 - protein, 87 aa, Artificial Sequence
  Description of Artificial Sequence: Synthetic polypeptide
HGEGTFTSDLSKQMEEEAVRLFIEWLKNGGPSSGAPPKSGSLAEAKEAANAELDSYGVSDFYKRLIDKAKTVEGVEALKDAILAALP

SEQ ID NO: 789 - protein, 86 aa, Artificial Sequence
  Description of Artificial Sequence: Synthetic polypeptide
HGEGTFTSDLSKQMEEEAVRLFIEWLKNGGPSSGAPPKSGSLAEAKEAANAELDSYGVSDFYKRLIDKAKTVEGVEALKDAILAAL

SEQ ID NO: 790 - protein, 86 aa, Artificial Sequence
  Description of Artificial Sequence: Synthetic polypeptide
HGEGTFTSDLSKQMEEEAVRLFIEWLKNGGPSSGAPPKSGLAEAKEAANAELDSYGVSDFYKRLIDKAKTVEGVEALKDAILAALP

SEQ ID NO: 791 - protein, 85 aa, Artificial Sequence
  Description of Artificial Sequence: Synthetic polypeptide
HGEGTFTSDLSKQMEEEAVRLFIEWLKNGGPSSGAPPKSGLAEAKEAANAELDSYGVSDFYKRLIDKAKTVEGVEALKDAILAAL

SEQ ID NO: 792 - protein, 94 aa, Artificial Sequence
  Description of Artificial Sequence: Synthetic polypeptide
HGEGTFTSDLSKQLEEEAVRLFIEWLKNGGPSSGAPPPSTGGGGSASSLAEAKEAANAELDSYGVSDFYKRLIDKAKTVEGVEALKDAILAALP

SEQ ID NO: 793 - protein, 93 aa, Artificial Sequence
  Description of Artificial Sequence: Synthetic polypeptide
HGEGTFTSDLSKQLEEEAVRLFIEWLKNGGPSSGAPPPSTGGGGSASSLAEAKEAANAELDSYGVSDFYKRLIDKAKTVEGVEALKDAILAAL

SEQ ID NO: 794 - protein, 93 aa, Artificial Sequence
  Description of Artificial Sequence: Synthetic polypeptide
HGEGTFTSDLSKQLEEEAVRLFIEWLKNGGPSSGAPPPSTGGGGSASLAEAKEAANAELDSYGVSDFYKRLIDKAKTVEGVEALKDAILAALP

SEQ ID NO: 795 - protein, 92 aa, Artificial Sequence
  Description of Artificial Sequence: Synthetic polypeptide
HGEGTFTSDLSKQLEEEAVRLFIEWLKNGGPSSGAPPPSTGGGGSASLAEAKEAANAELDSYGVSDFYKRLIDKAKTVEGVEALKDAILAAL

SEQ ID NO: 796 - protein, 90 aa, Artificial Sequence
  Description of Artificial Sequence: Synthetic polypeptide
HGEGTFTSDLSKQLEEEAVRLFIEWLKNGGPSSGAPPPSGGGGSLAEAKEAANAELDSYGVSDFYKRLIDKAKTVEGVEALKDAILAALP

SEQ ID NO: 797 - protein, 89 aa, Artificial Sequence
  Description of Artificial Sequence: Synthetic polypeptide
HGEGTFTSDLSKQLEEEAVRLFIEWLKNGGPSSGAPPPSGGGGSLAEAKEAANAELDSYGVSDFYKRLIDKAKTVEGVEALKDAILAAL

SEQ ID NO: 798 - protein, 89 aa, Artificial Sequence
  Description of Artificial Sequence: Synthetic polypeptide
HGEGTFTSDLSKQLEEEAVRLFIEWLKNGGPSSGAPPPSGGGSLAEAKEAANAELDSYGVSDFYKRLIDKAKTVEGVEALKDAILAALP

SEQ ID NO: 799 - protein, 88 aa, Artificial Sequence
  Description of Artificial Sequence: Synthetic polypeptide
HGEGTFTSDLSKQLEEEAVRLFIEWLKNGGPSSGAPPPSGGGSLAEAKEAANAELDSYGVSDFYKRLIDKAKTVEGVEALKDAILAAL

SEQ ID NO: 800 - protein, 88 aa, Artificial Sequence
  Description of Artificial Sequence: Synthetic polypeptide
HGEGTFTSDLSKQLEEEAVRLFIEWLKNGGPSSGAPPPSGGGLAEAKEAANAELDSYGVSDFYKRLIDKAKTVEGVEALKDAILAALP

SEQ ID NO: 801 - protein, 87 aa, Artificial Sequence
  Description of Artificial Sequence: Synthetic polypeptide
HGEGTFTSDLSKQLEEEAVRLFIEWLKNGGPSSGAPPPSGGGLAEAKEAANAELDSYGVSDFYKRLIDKAKTVEGVEALKDAILAAL

SEQ ID NO: 802 - protein, 87 aa, Artificial Sequence
  Description of Artificial Sequence: Synthetic polypeptide
HGEGTFTSDLSKQLEEEAVRLFIEWLKNGGPSSGAPPPSGSLAEAKEAANAELDSYGVSDFYKRLIDKAKTVEGVEALKDAILAALP

SEQ ID NO: 803 - protein, 86 aa, Artificial Sequence
  Description of Artificial Sequence: Synthetic polypeptide
HGEGTFTSDLSKQLEEEAVRLFIEWLKNGGPSSGAPPPSGSLAEAKEAANAELDSYGVSDFYKRLIDKAKTVEGVEALKDAILAAL

SEQ ID NO: 804 - protein, 86 aa, Artificial Sequence
  Description of Artificial Sequence: Synthetic polypeptide
HGEGTFTSDLSKQLEEEAVRLFIEWLKNGGPSSGAPPPSGLAEAKEAANAELDSYGVSDFYKRLIDKAKTVEGVEALKDAILAALP

SEQ ID NO: 805 - protein, 85 aa, Artificial Sequence
  Description of Artificial Sequence: Synthetic polypeptide
HGEGTFTSDLSKQLEEEAVRLFIEWLKNGGPSSGAPPPSGLAEAKEAANAELDSYGVSDFYKRLIDKAKTVEGVEALKDAILAAL

SEQ ID NO: 806 - protein, 94 aa, Artificial Sequence
  Description of Artificial Sequence: Synthetic polypeptide
HGEGTFTSDLSKQMEEEAVRLFIEWLKNGGPSSGAPPPSTGGGGSASSLAEAKEAANAELDSYGVSDFYKRLIDKAKTVEGVEALKDAILAALP

SEQ ID NO: 807 - protein, 93 aa, Artificial Sequence
  Description of Artificial Sequence: Synthetic polypeptide
HGEGTFTSDLSKQMEEEAVRLFIEWLKNGGPSSGAPPPSTGGGGSASSLAEAKEAANAELDSYGVSDFYKRLIDKAKTVEGVEALKDAILAAL

SEQ ID NO: 808 - protein, 93 aa, Artificial Sequence
  Description of Artificial Sequence: Synthetic polypeptide
HGEGTFTSDLSKQMEEEAVRLFIEWLKNGGPSSGAPPPSTGGGGSASLAEAKEAANAELDSYGVSDFYKRLIDKAKTVEGVEALKDAILAALP

SEQ ID NO: 809 - protein, 92 aa, Artificial Sequence
  Description of Artificial Sequence: Synthetic polypeptide
HGEGTFTSDLSKQMEEEAVRLFIEWLKNGGPSSGAPPPSTGGGGSASLAEAKEAANAELDSYGVSDFYKRLIDKAKTVEGVEALKDAILAAL

SEQ ID NO: 810 - protein, 90 aa, Artificial Sequence
  Description of Artificial Sequence: Synthetic polypeptide
HGEGTFTSDLSKQMEEEAVRLFIEWLKNGGPSSGAPPPSGGGGSLAEAKEAANAELDSYGVSDFYKRLIDKAKTVEGVEALKDAILAALP

SEQ ID NO: 811 - protein, 89 aa, Artificial Sequence
  Description of Artificial Sequence: Synthetic polypeptide
HGEGTFTSDLSKQMEEEAVRLFIEWLKNGGPSSGAPPPSGGGGSLAEAKEAANAELDSYGVSDFYKRLIDKAKTVEGVEALKDAILAAL

SEQ ID NO: 812 - protein, 89 aa, Artificial Sequence
  Description of Artificial Sequence: Synthetic polypeptide
HGEGTFTSDLSKQMEEEAVRLFIEWLKNGGPSSGAPPPSGGGSLAEAKEAANAELDSYGVSDFYKRLIDKAKTVEGVEALKDAILAALP

SEQ ID NO: 813 - protein, 88 aa, Artificial Sequence
  Description of Artificial Sequence: Synthetic polypeptide
HGEGTFTSDLSKQMEEEAVRLFIEWLKNGGPSSGAPPPSGGGSLAEAKEAANAELDSYGVSDFYKRLIDKAKTVEGVEALKDAILAAL

SEQ ID NO: 814 - protein, 88 aa, Artificial Sequence
  Description of Artificial Sequence: Synthetic polypeptide
HGEGTFTSDLSKQMEEEAVRLFIEWLKNGGPSSGAPPPSGGGLAEAKEAANAELDSYGVSDFYKRLIDKAKTVEGVEALKDAILAALP

SEQ ID NO: 815 - protein, 87 aa, Artificial Sequence
  Description of Artificial Sequence: Synthetic polypeptide
HGEGTFTSDLSKQMEEEAVRLFIEWLKNGGPSSGAPPPSGGGLAEAKEAANAELDSYGVSDFYKRLIDKAKTVEGVEALKDAILAAL

SEQ ID NO: 816 - protein, 87 aa, Artificial Sequence
  Description of Artificial Sequence: Synthetic polypeptide
HGEGTFTSDLSKQMEEEAVRLFIEWLKNGGPSSGAPPPSGSLAEAKEAANAELDSYGVSDFYKRLIDKAKTVEGVEALKDAILAALP

SEQ ID NO: 817 - protein, 86 aa, Artificial Sequence
  Description of Artificial Sequence: Synthetic polypeptide
HGEGTFTSDLSKQMEEEAVRLFIEWLKNGGPSSGAPPPSGSLAEAKEAANAELDSYGVSDFYKRLIDKAKTVEGVEALKDAILAAL

SEQ ID NO: 818 - protein, 86 aa, Artificial Sequence
  Description of Artificial Sequence: Synthetic polypeptide
HGEGTFTSDLSKQMEEEAVRLFIEWLKNGGPSSGAPPPSGLAEAKEAANAELDSYGVSDFYKRLIDKAKTVEGVEALKDAILAALP

SEQ ID NO: 819 - protein, 85 aa, Artificial Sequence
  Description of Artificial Sequence: Synthetic polypeptide
HGEGTFTSDLSKQMEEEAVRLFIEWLKNGGPSSGAPPPSGLAEAKEAANAELDSYGVSDFYKRLIDKAKTVEGVEALKDAILAAL

SEQ ID NO: 820 - protein, 100 aa, Artificial Sequence
  Description of Artificial Sequence: Synthetic polypeptide
HGEGTFTSDLSKQLEEEAVRLFIEWLKNGGPSSGAPPSKKKKKKTGGGGSASGSLAEAKEAANAELDSYGVSDFYKRLIDKAKTVEGVEALKDAILAALP

SEQ ID NO: 821 - protein, 99 aa, Artificial Sequence
  Description of Artificial Sequence: Synthetic polypeptide
HGEGTFTSDLSKQLEEEAVRLFIEWLKNGGPSSGAPPSKKKKKKTGGGGSASGSLAEAKEAANAELDSYGVSDFYKRLIDKAKTVEGVEALKDAILAAL

SEQ ID NO: 822 - protein, 99 aa, Artificial Sequence
  Description of Artificial Sequence: Synthetic polypeptide
HGEGTFTSDLSKQLEEEAVRLFIEWLKNGGPSSGAPPSKKKKKKTGGGGSASSLAEAKEAANAELDSYGVSDFYKRLIDKAKTVEGVEALKDAILAALP

SEQ ID NO: 823 - protein, 98 aa, Artificial Sequence
  Description of Artificial Sequence: Synthetic polypeptide
HGEGTFTSDLSKQLEEEAVRLFIEWLKNGGPSSGAPPSKKKKKKTGGGGSASSLAEAKEAANAELDSYGVSDFYKRLIDKAKTVEGVEALKDAILAAL

SEQ ID NO: 824 - protein, 98 aa, Artificial Sequence
  Description of Artificial Sequence: Synthetic polypeptide
HGEGTFTSDLSKQLEEEAVRLFIEWLKNGGPSSGAPPSKKKKKKTGGGGSASLAEAKEAANAELDSYGVSDFYKRLIDKAKTVEGVEALKDAILAALP

SEQ ID NO: 825 - protein, 97 aa, Artificial Sequence
  Description of Artificial Sequence: Synthetic polypeptide
HGEGTFTSDLSKQLEEEAVRLFIEWLKNGGPSSGAPPSKKKKKKTGGGGSASLAEAKEAANAELDSYGVSDFYKRLIDKAKTVEGVEALKDAILAAL

SEQ ID NO: 826 - protein, 95 aa, Artificial Sequence
  Description of Artificial Sequence: Synthetic polypeptide
HGEGTFTSDLSKQLEEEAVRLFIEWLKNGGPSSGAPPSKKKKKKGGGGSLAEAKEAANAELDSYGVSDFYKRLIDKAKTVEGVEALKDAILAALP

SEQ ID NO: 827 - protein, 94 aa, Artificial Sequence
  Description of Artificial Sequence: Synthetic polypeptide
HGEGTFTSDLSKQLEEEAVRLFIEWLKNGGPSSGAPPSKKKKKKGGGGSLAEAKEAANAELDSYGVSDFYKRLIDKAKTVEGVEALKDAILAAL

SEQ ID NO: 828 - protein, 94 aa, Artificial Sequence
  Description of Artificial Sequence: Synthetic polypeptide
HGEGTFTSDLSKQLEEEAVRLFIEWLKNGGPSSGAPPSKKKKKKGGGSLAEAKEAANAELDSYGVSDFYKRLIDKAKTVEGVEALKDAILAALP

SEQ ID NO: 829 - protein, 93 aa, Artificial Sequence
  Description of Artificial Sequence: Synthetic polypeptide
HGEGTFTSDLSKQLEEEAVRLFIEWLKNGGPSSGAPPSKKKKKKGGGSLAEAKEAANAELDSYGVSDFYKRLIDKAKTVEGVEALKDAILAAL

SEQ ID NO: 830 - protein, 93 aa, Artificial Sequence
  Description of Artificial Sequence: Synthetic polypeptide
HGEGTFTSDLSKQLEEEAVRLFIEWLKNGGPSSGAPPSKKKKKKGGGLAEAKEAANAELDSYGVSDFYKRLIDKAKTVEGVEALKDAILAALP

SEQ ID NO: 831 - protein, 92 aa, Artificial Sequence
  Description of Artificial Sequence: Synthetic polypeptide
HGEGTFTSDLSKQLEEEAVRLFIEWLKNGGPSSGAPPSKKKKKKGGGLAEAKEAANAELDSYGVSDFYKRLIDKAKTVEGVEALKDAILAAL

SEQ ID NO: 832 - protein, 93 aa, Artificial Sequence
  Description of Artificial Sequence: Synthetic polypeptide
HGEGTFTSDLSKQLEEEAVRLFIEWLKNGGPSSGAPPSKKKKKKGGSLAEAKEAANAELDSYGVSDFYKRLIDKAKTVEGVEALKDAILAALP

SEQ ID NO: 833 - protein, 92 aa, Artificial Sequence
  Description of Artificial Sequence: Synthetic polypeptide
HGEGTFTSDLSKQLEEEAVRLFIEWLKNGGPSSGAPPSKKKKKKGGSLAEAKEAANAELDSYGVSDFYKRLIDKAKTVEGVEALKDAILAAL

SEQ ID NO: 834 - protein, 92 aa, Artificial Sequence
  Description of Artificial Sequence: Synthetic polypeptide
HGEGTFTSDLSKQLEEEAVRLFIEWLKNGGPSSGAPPSKKKKKKGSLAEAKEAANAELDSYGVSDFYKRLIDKAKTVEGVEALKDAILAALP

SEQ ID NO: 835 - protein, 91 aa, Artificial Sequence
  Description of Artificial Sequence: Synthetic polypeptide
HGEGTFTSDLSKQLEEEAVRLFIEWLKNGGPSSGAPPSKKKKKKGSLAEAKEAANAELDSYGVSDFYKRLIDKAKTVEGVEALKDAILAAL

SEQ ID NO: 836 - protein, 91 aa, Artificial Sequence
  Description of Artificial Sequence: Synthetic polypeptide
HGEGTFTSDLSKQLEEEAVRLFIEWLKNGGPSSGAPPSKKKKKKGLAEAKEAANAELDSYGVSDFYKRLIDKAKTVEGVEALKDAILAALP

SEQ ID NO: 837 - protein, 90 aa, Artificial Sequence
  Description of Artificial Sequence: Synthetic polypeptide
HGEGTFTSDLSKQLEEEAVRLFIEWLKNGGPSSGAPPSKKKKKKGLAEAKEAANAELDSYGVSDFYKRLIDKAKTVEGVEALKDAILAAL

SEQ ID NO: 838 - protein, 113 aa, Artificial Sequence
  Description of Artificial Sequence: Synthetic polypeptide
MAHHHHHHVGTGSNENLYFQHGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 839 - protein, 252 aa, Artificial Sequence
  Description of Artificial Sequence: Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 840 - protein, 115 aa, Artificial Sequence
  Description of Artificial Sequence: Synthetic polypeptide
MAHHHHHHVGTGSNENLYFQHGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 841 - protein, 254 aa, Artificial Sequence
  Description of Artificial Sequence: Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 842 - protein, 115 aa, Artificial Sequence
  Description of Artificial Sequence: Synthetic polypeptide
MAHHHHHHVGTGSNENLYFQHGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 843 - protein, 254 aa, Artificial Sequence
  Description of Artificial Sequence: Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 844 - protein, 4 aa, Artificial Sequence
  Description of Artificial Sequence: Synthetic peptide
MGSS

SEQ ID NO: 845 - protein, 4 aa, Artificial Sequence
  Description of Artificial Sequence: Synthetic peptide
GSSL

SEQ ID NO: 846 - protein, 111 aa, Artificial Sequence
  Description of Artificial Sequence: Synthetic polypeptide
MAHHHHHHVGTGSNENLYFQHGEGTFTSDLSKQLEEEAVRLFIEWLKQGGPSKEIISTGGGGSASLAEAKVLANRELDKYGVSDFYKRLINKAKTVEGVEALKLHILAALP

SEQ ID NO: 847 - protein, 84 aa, Artificial Sequence
  Description of Artificial Sequence: Synthetic polypeptide
HGEGTFTSDLSKQLEEEAVRLFIEWLKNTGGGGSASGSLAEAKEAANAELDSYGVSDFYKRLIDKAKTVEGVEALKDAILAALP

SEQ ID NO: 848 - protein, 6 aa, Artificial Sequence
  Description of Artificial Sequence: Synthetic peptide
TGGGGS

SEQ ID NO: 849 - protein, 10 aa, Artificial Sequence
  Description of Artificial Sequence: Synthetic peptide
TGGGGSGGGS

SEQ ID NO: 850 - protein, 14 aa, Artificial Sequence
  Description of Artificial Sequence: Synthetic peptide
TGGGGSGGGSGGGS

SEQ ID NO: 851 - protein, 18 aa, Artificial Sequence
  Description of Artificial Sequence: Synthetic peptide
TGGGGSGGGSGGGSGGGS

SEQ ID NO: 852 - protein, 22 aa, Artificial Sequence
  Description of Artificial Sequence: Synthetic peptide
TGGGGSGGGSGGGSGGGSGGGS

SEQ ID NO: 853 - protein, 6 aa, Artificial Sequence
  Description of Artificial Sequence: Synthetic peptide
GGGSAS

SEQ ID NO: 854 - protein, 10 aa, Artificial Sequence
  Description of Artificial Sequence: Synthetic peptide
GGGSGGGSAS

SEQ ID NO: 855 - protein, 14 aa, Artificial Sequence
  Description of Artificial Sequence: Synthetic peptide
GGGSGGGSGGGSAS

SEQ ID NO: 856 - protein, 18 aa, Artificial Sequence
  Description of Artificial Sequence: Synthetic peptide
GGGSGGGSGGGSGGGSAS

SEQ ID NO: 857 - protein, 22 aa, Artificial Sequence
  Description of Artificial Sequence: Synthetic peptide
GGGSGGGSGGGSGGGSGGGSAS

SEQ ID NO: 858 - protein, 12 aa, Artificial Sequence
  Description of Artificial Sequence: Synthetic peptide
TGGGGSGGGSAS

SEQ ID NO: 859 - protein, 16 aa, Artificial Sequence
  Description of Artificial Sequence: Synthetic peptide
TGGGGSGGGSGGGSAS

SEQ ID NO: 860 - protein, 20 aa, Artificial Sequence
  Description of Artificial Sequence: Synthetic peptide
TGGGGSGGGSGGGSGGGSAS

SEQ ID NO: 861 - protein, 24 aa, Artificial Sequence
  Description of Artificial Sequence: Synthetic peptide
TGGGGSGGGSGGGSGGGSGGGSAS

SEQ ID NO: 862 - protein, 48 aa, Artificial Sequence
  Description of Artificial Sequence: Synthetic polypeptide
GSLAQAKEAANAELDSYGVSDFYKRLIDKAKTVEGVEALKDAILAALP

SEQ ID NO: 863 - protein, 48 aa, Artificial Sequence
  Description of Artificial Sequence: Synthetic polypeptide
GSLAEAKEAANRELDSYGVSDFYKRLIDKAKTVEGVEALKDAILAALP

SEQ ID NO: 864 - protein, 48 aa, Artificial Sequence
  Description of Artificial Sequence: Synthetic polypeptide
GSLAQAKEAANRELDSYGVSDFYKRLIDKAKTVEGVEALKDAILAALP

SEQ ID NO: 865 - protein, 48 aa, Artificial Sequence
  Description of Artificial Sequence: Synthetic polypeptide
GSLAEAKEAANRELDAYGVSDFYKRLIDKAKTVEGVEALKDAILAALP

SEQ ID NO: 866 - protein, 48 aa, Artificial Sequence
  Description of Artificial Sequence: Synthetic polypeptide
GSLAEAKVLANRELDKYGVSDFYKRLIDKAKTVEGVEALKDAILAALP

SEQ ID NO: 867 - protein, 47 aa, Artificial Sequence
  Description of Artificial Sequence: Synthetic polypeptide
SLAQAKEAANAELDSYGVSDFYKRLIDKAKTVEGVEALKDAILAALP

SEQ ID NO: 868 - protein, 47 aa, Artificial Sequence
  Description of Artificial Sequence: Synthetic polypeptide
SLAEAKEAANRELDSYGVSDFYKRLIDKAKTVEGVEALKDAILAALP

SEQ ID NO: 869 - protein, 47 aa, Artificial Sequence
  Description of Artificial Sequence: Synthetic polypeptide
SLAQAKEAANRELDSYGVSDFYKRLIDKAKTVEGVEALKDAILAALP

SEQ ID NO: 870 - protein, 47 aa, Artificial Sequence
  Description of Artificial Sequence: Synthetic polypeptide
SLAEAKEAANRELDAYGVSDFYKRLIDKAKTVEGVEALKDAILAALP

SEQ ID NO: 871 - protein, 47 aa, Artificial Sequence
  Description of Artificial Sequence: Synthetic polypeptide
SLAEAKVLANRELDKYGVSDFYKRLIDKAKTVEGVEALKDAILAALP

SEQ ID NO: 872 - protein, 46 aa, Artificial Sequence
  Description of Artificial Sequence: Synthetic polypeptide
LAEAKEAANRELDSYGVSDFYKRLIDKAKTVEGVEALKDAILAALP

SEQ ID NO: 873 - protein, 46 aa, Artificial Sequence
  Description of Artificial Sequence: Synthetic polypeptide
LAQAKEAANRELDSYGVSDFYKRLIDKAKTVEGVEALKDAILAALP

SEQ ID NO: 874 - protein, 46 aa, Artificial Sequence
  Description of Artificial Sequence: Synthetic polypeptide
LAEAKEAANRELDAYGVSDFYKRLIDKAKTVEGVEALKDAILAALP

SEQ ID NO: 875 - protein, 46 aa, Artificial Sequence
  Description of Artificial Sequence: Synthetic polypeptide
LAEAKVLANRELDKYGVSDFYKRLIDKAKTVEGVEALKDAILAALP

SEQ ID NO: 876 - protein, 48 aa, Artificial Sequence
  Description of Artificial Sequence: Synthetic polypeptide
GSLAQAKEAANAELDSYGVSDFYKRLIEKAKTVEGVEALKDAILAALP

SEQ ID NO: 877 - protein, 48 aa, Artificial Sequence
  Description of Artificial Sequence: Synthetic polypeptide
GSLAEAKEAANRELDSYGVSDFYKRLIEKAKTVEGVEALKDAILAALP

SEQ ID NO: 878 - protein, 48 aa, Artificial Sequence
  Description of Artificial Sequence: Synthetic polypeptide
GSLAQAKEAANRELDSYGVSDFYKRLIEKAKTVEGVEALKDAILAALP

SEQ ID NO: 879 - protein, 48 aa, Artificial Sequence
  Description of Artificial Sequence: Synthetic polypeptide
GSLAEAKEAANRELDAYGVSDFYKRLIEKAKTVEGVEALKDAILAALP

SEQ ID NO: 880 - protein, 48 aa, Artificial Sequence
  Description of Artificial Sequence: Synthetic polypeptide
GSLAEAKVLANRELDKYGVSDFYKRLIEKAKTVEGVEALKDAILAALP

SEQ ID NO: 881 - protein, 47 aa, Artificial Sequence
  Description of Artificial Sequence: Synthetic polypeptide
SLAQAKEAANAELDSYGVSDFYKRLIEKAKTVEGVEALKDAILAALP

SEQ ID NO: 882 - protein, 47 aa, Artificial Sequence
  Description of Artificial Sequence: Synthetic polypeptide
SLAEAKEAANRELDSYGVSDFYKRLIEKAKTVEGVEALKDAILAALP

SEQ ID NO: 883 - protein, 47 aa, Artificial Sequence
  Description of Artificial Sequence: Synthetic polypeptide
SLAQAKEAANRELDSYGVSDFYKRLIEKAKTVEGVEALKDAILAALP

SEQ ID NO: 884 - protein, 47 aa, Artificial Sequence
  Description of Artificial Sequence: Synthetic polypeptide
SLAEAKEAANRELDAYGVSDFYKRLIEKAKTVEGVEALKDAILAALP

SEQ ID NO: 885 - protein, 47 aa, Artificial Sequence
  Description of Artificial Sequence: Synthetic polypeptide
SLAEAKVLANRELDKYGVSDFYKRLIEKAKTVEGVEALKDAILAALP

SEQ ID NO: 886 - protein, 46 aa, Artificial Sequence
  Description of Artificial Sequence: Synthetic polypeptide
LAQAKEAANAELDSYGVSDFYKRLIEKAKTVEGVEALKDAILAALP

SEQ ID NO: 887 - protein, 46 aa, Artificial Sequence
  Description of Artificial Sequence: Synthetic polypeptide
LAEAKEAANRELDSYGVSDFYKRLIEKAKTVEGVEALKDAILAALP

SEQ ID NO: 888 - protein, 46 aa, Artificial Sequence
  Description of Artificial Sequence: Synthetic polypeptide
LAQAKEAANRELDSYGVSDFYKRLIEKAKTVEGVEALKDAILAALP

SEQ ID NO: 889 - protein, 46 aa, Artificial Sequence
  Description of Artificial Sequence: Synthetic polypeptide
LAEAKEAANRELDAYGVSDFYKRLIEKAKTVEGVEALKDAILAALP

SEQ ID NO: 890 - protein, 46 aa, Artificial Sequence
  Description of Artificial Sequence: Synthetic polypeptide
LAEAKVLANRELDKYGVSDFYKRLIEKAKTVEGVEALKDAILAALP

SEQ ID NO: 891 - protein, 48 aa, Artificial Sequence
  Description of Artificial Sequence: Synthetic polypeptide
GSLAQAKEAANAELDSYGVSDFYKRLIEKAKTVEGVEALKEAILAALP

SEQ ID NO: 892 - protein, 48 aa, Artificial Sequence
  Description of Artificial Sequence: Synthetic polypeptide
GSLAEAKEAANRELDSYGVSDFYKRLIEKAKTVEGVEALKEAILAALP

SEQ ID NO: 893 - protein, 48 aa, Artificial Sequence
  Description of Artificial Sequence: Synthetic polypeptide
GSLAQAKEAANRELDSYGVSDFYKRLIEKAKTVEGVEALKEAILAALP

SEQ ID NO: 894 - protein, 48 aa, Artificial Sequence
  Description of Artificial Sequence: Synthetic polypeptide
GSLAEAKEAANRELDAYGVSDFYKRLIEKAKTVEGVEALKEAILAALP

SEQ ID NO: 895 - protein, 48 aa, Artificial Sequence
  Description of Artificial Sequence: Synthetic polypeptide
GSLAEAKVLANRELDKYGVSDFYKRLIEKAKTVEGVEALKEAILAALP

SEQ ID NO: 896 - protein, 47 aa, Artificial Sequence
  Description of Artificial Sequence: Synthetic polypeptide
SLAQAKEAANAELDSYGVSDFYKRLIEKAKTVEGVEALKEAILAALP

SEQ ID NO: 897 - protein, 47 aa, Artificial Sequence
  Description of Artificial Sequence: Synthetic polypeptide
SLAEAKEAANRELDSYGVSDFYKRLIEKAKTVEGVEALKEAILAALP

SEQ ID NO: 898 - protein, 47 aa, Artificial Sequence
  Description of Artificial Sequence: Synthetic polypeptide
SLAQAKEAANRELDSYGVSDFYKRLIEKAKTVEGVEALKEAILAALP

SEQ ID NO: 899 - protein, 47 aa, Artificial Sequence
  Description of Artificial Sequence: Synthetic polypeptide
SLAEAKEAANRELDAYGVSDFYKRLIEKAKTVEGVEALKEAILAALP

SEQ ID NO: 900 - protein, 47 aa, Artificial Sequence
  Description of Artificial Sequence: Synthetic polypeptide
SLAEAKVLANRELDKYGVSDFYKRLIEKAKTVEGVEALKEAILAALP

SEQ ID NO: 901 - protein, 46 aa, Artificial Sequence
  Description of Artificial Sequence: Synthetic polypeptide
LAQAKEAANAELDSYGVSDFYKRLIEKAKTVEGVEALKEAILAALP

SEQ ID NO: 902 - protein, 46 aa, Artificial Sequence
  Description of Artificial Sequence: Synthetic polypeptide
LAEAKEAANRELDSYGVSDFYKRLIEKAKTVEGVEALKEAILAALP

SEQ ID NO: 903 - protein, 46 aa, Artificial Sequence
  Description of Artificial Sequence: Synthetic polypeptide
LAQAKEAANRELDSYGVSDFYKRLIEKAKTVEGVEALKEAILAALP

SEQ ID NO: 904 - protein, 46 aa, Artificial Sequence
  Description of Artificial Sequence: Synthetic polypeptide
LAEAKEAANRELDAYGVSDFYKRLIEKAKTVEGVEALKEAILAALP

SEQ ID NO: 905 - protein, 46 aa, Artificial Sequence
  Description of Artificial Sequence: Synthetic polypeptide
LAEAKVLANRELDKYGVSDFYKRLIEKAKTVEGVEALKEAILAALP

SEQ ID NO: 906 - protein, 48 aa, Artificial Sequence
  Description of Artificial Sequence: Synthetic polypeptide
GSLAQAKEAANAELDSYGVSDFYKRLIDKAKTVEGVEALKDAILASLP

SEQ ID NO: 907 - protein, 48 aa, Artificial Sequence
  Description of Artificial Sequence: Synthetic polypeptide
GSLAEAKEAANRELDSYGVSDFYKRLIDKAKTVEGVEALKDAILASLP

SEQ ID NO: 908 - protein, 48 aa, Artificial Sequence
  Description of Artificial Sequence: Synthetic polypeptide
GSLAQAKEAANRELDSYGVSDFYKRLIDKAKTVEGVEALKDAILASLP

SEQ ID NO: 909 - protein, 48 aa, Artificial Sequence
  Description of Artificial Sequence: Synthetic polypeptide
GSLAEAKEAANRELDAYGVSDFYKRLIDKAKTVEGVEALKDAILASLP

SEQ ID NO: 910 - protein, 48 aa, Artificial Sequence
  Description of Artificial Sequence: Synthetic polypeptide
GSLAEAKVLANRELDKYGVSDFYKRLIDKAKTVEGVEALKDAILASLP

SEQ ID NO: 911 - protein, 47 aa, Artificial Sequence
  Description of Artificial Sequence: Synthetic polypeptide
SLAQAKEAANAELDSYGVSDFYKRLIDKAKTVEGVEALKDAILASLP

SEQ ID NO: 912 - protein, 47 aa, Artificial Sequence
  Description of Artificial Sequence: Synthetic polypeptide
SLAEAKEAANRELDSYGVSDFYKRLIDKAKTVEGVEALKDAILASLP

SEQ ID NO: 913 - protein, 47 aa, Artificial Sequence
  Description of Artificial Sequence: Synthetic polypeptide
SLAQAKEAANRELDSYGVSDFYKRLIDKAKTVEGVEALKDAILASLP

SEQ ID NO: 914 - protein, 47 aa, Artificial Sequence
  Description of Artificial Sequence: Synthetic polypeptide
SLAEAKEAANRELDAYGVSDFYKRLIDKAKTVEGVEALKDAILASLP

SEQ ID NO: 915 - protein, 47 aa, Artificial Sequence
  Description of Artificial Sequence: Synthetic polypeptide
SLAEAKVLANRELDKYGVSDFYKRLIDKAKTVEGVEALKDAILASLP

SEQ ID NO: 916 - protein, 46 aa, Artificial Sequence
  Description of Artificial Sequence: Synthetic polypeptide
LAQAKEAANAELDSYGVSDFYKRLIDKAKTVEGVEALKDAILASLP

SEQ ID NO: 917 - protein, 46 aa, Artificial Sequence
  Description of Artificial Sequence: Synthetic polypeptide
LAEAKEAANRELDSYGVSDFYKRLIDKAKTVEGVEALKDAILASLP

SEQ ID NO: 918 - protein, 46 aa, Artificial Sequence
  Description of Artificial Sequence: Synthetic polypeptide
LAQAKEAANRELDSYGVSDFYKRLIDKAKTVEGVEALKDAILASLP

SEQ ID NO: 919 - protein, 46 aa, Artificial Sequence
  Description of Artificial Sequence: Synthetic polypeptide
LAEAKEAANRELDAYGVSDFYKRLIDKAKTVEGVEALKDAILASLP

SEQ ID NO: 920 - protein, 46 aa, Artificial Sequence
  Description of Artificial Sequence: Synthetic polypeptide
LAEAKVLANRELDKYGVSDFYKRLIDKAKTVEGVEALKDAILASLP

SEQ ID NO: 921 - protein, 48 aa, Artificial Sequence
  Description of Artificial Sequence: Synthetic polypeptide
GSLAQAKEAANAELDSYGVSDFYKRLIDKAKTVEGVEALKDAILAELP

SEQ ID NO: 922 - protein, 48 aa, Artificial Sequence
  Description of Artificial Sequence: Synthetic polypeptide
GSLAEAKEAANRELDSYGVSDFYKRLIDKAKTVEGVEALKDAILAELP

SEQ ID NO: 923 - protein, 48 aa, Artificial Sequence
  Description of Artificial Sequence: Synthetic polypeptide
GSLAQAKEAANRELDSYGVSDFYKRLIDKAKTVEGVEALKDAILAELP

SEQ ID NO: 924 - protein, 48 aa, Artificial Sequence
  Description of Artificial Sequence: Synthetic polypeptide
GSLAEAKEAANRELDAYGVSDFYKRLIDKAKTVEGVEALKDAILAELP

SEQ ID NO: 925 - protein, 48 aa, Artificial Sequence
  Description of Artificial Sequence: Synthetic polypeptide
GSLAEAKVLANRELDKYGVSDFYKRLIDKAKTVEGVEALKDAILAELP

SEQ ID NO: 926 - protein, 47 aa, Artificial Sequence
  Description of Artificial Sequence: Synthetic polypeptide
SLAQAKEAANAELDSYGVSDFYKRLIDKAKTVEGVEALKDAILAELP

SEQ ID NO: 927 - protein, 47 aa, Artificial Sequence
  Description of Artificial Sequence: Synthetic polypeptide
SLAEAKEAANRELDSYGVSDFYKRLIDKAKTVEGVEALKDAILAELP

SEQ ID NO: 928 - protein, 47 aa, Artificial Sequence
  Description of Artificial Sequence: Synthetic polypeptide
SLAQAKEAANRELDSYGVSDFYKRLIDKAKTVEGVEALKDAILAELP

SEQ ID NO: 929 - protein, 47 aa, Artificial Sequence
  Description of Artificial Sequence: Synthetic polypeptide
SLAEAKEAANRELDAYGVSDFYKRLIDKAKTVEGVEALKDAILAELP

SEQ ID NO: 930 - protein, 47 aa, Artificial Sequence
  Description of Artificial Sequence: Synthetic polypeptide
SLAEAKVLANRELDKYGVSDFYKRLIDKAKTVEGVEALKDAILAELP

SEQ ID NO: 931 - protein, 46 aa, Artificial Sequence
  Description of Artificial Sequence: Synthetic polypeptide
LAQAKEAANAELDSYGVSDFYKRLIDKAKTVEGVEALKDAILAELP

SEQ ID NO: 932 - protein, 46 aa, Artificial Sequence
  Description of Artificial Sequence: Synthetic polypeptide
LAEAKEAANRELDSYGVSDFYKRLIDKAKTVEGVEALKDAILAELP

SEQ ID NO: 933 - protein, 46 aa, Artificial Sequence
  Description of Artificial Sequence: Synthetic polypeptide
LAQAKEAANRELDSYGVSDFYKRLIDKAKTVEGVEALKDAILAELP

SEQ ID NO: 934 - protein, 46 aa, Artificial Sequence
  Description of Artificial Sequence: Synthetic polypeptide
LAEAKEAANRELDAYGVSDFYKRLIDKAKTVEGVEALKDAILAELP

SEQ ID NO: 935 - protein, 46 aa, Artificial Sequence
  Description of Artificial Sequence: Synthetic polypeptide
LAEAKVLANRELDKYGVSDFYKRLIDKAKTVEGVEALKDAILAELP

SEQ ID NO: 936 - protein, 48 aa, Artificial Sequence
  Description of Artificial Sequence: Synthetic polypeptide
GSLAQAKEAANAELDSYGVSDFYKRLIDKAKTVEGVEALKDAILKALP

SEQ ID NO: 937 - protein, 48 aa, Artificial Sequence
  Description of Artificial Sequence: Synthetic polypeptide
GSLAEAKEAANRELDSYGVSDFYKRLIDKAKTVEGVEALKDAILKALP

SEQ ID NO: 938 - protein, 48 aa, Artificial Sequence
  Description of Artificial Sequence: Synthetic polypeptide
GSLAQAKEAANRELDSYGVSDFYKRLIDKAKTVEGVEALKDAILKALP

SEQ ID NO: 939 - protein, 48 aa, Artificial Sequence
  Description of Artificial Sequence: Synthetic polypeptide
GSLAEAKEAANRELDAYGVSDFYKRLIDKAKTVEGVEALKDAILKALP

SEQ ID NO: 940 - protein, 48 aa, Artificial Sequence
  Description of Artificial Sequence: Synthetic polypeptide
GSLAEAKVLANRELDKYGVSDFYKRLIDKAKTVEGVEALKDAILKALP

SEQ ID NO: 941 - protein, 47 aa, Artificial Sequence
  Description of Artificial Sequence: Synthetic polypeptide
SLAQAKEAANAELDSYGVSDFYKRLIDKAKTVEGVEALKDAILKALP

SEQ ID NO: 942 - protein, 47 aa, Artificial Sequence
  Description of Artificial Sequence: Synthetic polypeptide
SLAEAKEAANRELDSYGVSDFYKRLIDKAKTVEGVEALKDAILKALP

SEQ ID NO: 943 - protein, 47 aa, Artificial Sequence
  Description of Artificial Sequence: Synthetic polypeptide
SLAQAKEAANRELDSYGVSDFYKRLIDKAKTVEGVEALKDAILKALP

SEQ ID NO: 944 - protein, 47 aa, Artificial Sequence
  Description of Artificial Sequence: Synthetic polypeptide
SLAEAKEAANRELDAYGVSDFYKRLIDKAKTVEGVEALKDAILKALP

SEQ ID NO: 945 - protein, 47 aa, Artificial Sequence
  Description of Artificial Sequence: Synthetic polypeptide
SLAEAKVLANRELDKYGVSDFYKRLIDKAKTVEGVEALKDAILKALP

SEQ ID NO: 946 - protein, 46 aa, Artificial Sequence
  Description of Artificial Sequence: Synthetic polypeptide
LAQAKEAANAELDSYGVSDFYKRLIDKAKTVEGVEALKDAILKALP

SEQ ID NO: 947 - protein, 46 aa, Artificial Sequence
  Description of Artificial Sequence: Synthetic polypeptide
LAEAKEAANRELDSYGVSDFYKRLIDKAKTVEGVEALKDAILKALP

SEQ ID NO: 948 - protein, 46 aa, Artificial Sequence
  Description of Artificial Sequence: Synthetic polypeptide
LAQAKEAANRELDSYGVSDFYKRLIDKAKTVEGVEALKDAILKALP

SEQ ID NO: 949 - protein, 46 aa, Artificial Sequence
  Description of Artificial Sequence: Synthetic polypeptide
LAEAKEAANRELDAYGVSDFYKRLIDKAKTVEGVEALKDAILKALP

SEQ ID NO: 950 - protein, 46 aa, Artificial Sequence
  Description of Artificial Sequence: Synthetic polypeptide
LAEAKVLANRELDKYGVSDFYKRLIDKAKTVEGVEALKDAILKALP

SEQ ID NO: 951 - protein, 46 aa, Artificial Sequence
  Description of Artificial Sequence: Synthetic polypeptide
  MOD_RES (3)..(3): Glu, Ser, Gln or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKSXANXELDXYGVSDFYKRLIXKAKTVEGVEALKXXILXXLP

SEQ ID NO: 952 - protein, 46 aa, Artificial Sequence
  Description of Artificial Sequence: Synthetic polypeptide
  MOD_RES (3)..(3): Glu, Ser, Gln or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKEXANXELDXYGVSDFYKRLIXKAKTVEGVEALKXXILXXLP

SEQ ID NO: 953 - protein, 46 aa, Artificial Sequence
  Description of Artificial Sequence: Synthetic polypeptide
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LASAKXXANXELDXYGVSDFYKRLIXKAKTVEGVEALKXXILXXLP

SEQ ID NO: 954 - protein, 46 aa, Artificial Sequence
  Description of Artificial Sequence: Synthetic polypeptide
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAEAKXXANXELDXYGVSDFYKRLIXKAKTVEGVEALKXXILXXLP

SEQ ID NO: 955 - protein, 46 aa, Artificial Sequence
  Description of Artificial Sequence: Synthetic polypeptide
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAQAKXXANXELDXYGVSDFYKRLIXKAKTVEGVEALKXXILXXLP

SEQ ID NO: 956 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXAANXELDXYGVSDFYKRLIXKAKTVEGVEALKXXILXXLP

SEQ ID NO: 957 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXSANXELDXYGVSDFYKRLIXKAKTVEGVEALKXXILXXLP

SEQ ID NO: 958 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANAELDXYGVSDFYKRLIXKAKTVEGVEALKXXILXXLP

SEQ ID NO: 959 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANSELDXYGVSDFYKRLIXKAKTVEGVEALKXXILXXLP

SEQ ID NO: 960 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANRELDXYGVSDFYKRLIXKAKTVEGVEALKXXILXXLP

SEQ ID NO: 961 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDSYGVSDFYKRLIXKAKTVEGVEALKXXILXXLP

SEQ ID NO: 962 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDCYGVSDFYKRLIXKAKTVEGVEALKXXILXXLP

SEQ ID NO: 963 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDKYGVSDFYKRLIXKAKTVEGVEALKXXILXXLP

SEQ ID NO: 964 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDAYGVSDFYKRLIXKAKTVEGVEALKXXILXXLP

SEQ ID NO: 965 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIDKAKTVEGVEALKXXILXXLP

SEQ ID NO: 966 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIEKAKTVEGVEALKXXILXXLP

SEQ ID NO: 967 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIXKAKTVEGVEALKDXILXXLP

SEQ ID NO: 968 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIXKAKTVEGVEALKEXILXXLP

SEQ ID NO: 969 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIXKAKTVEGVEALKXAILXXLP

SEQ ID NO: 970 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (43)..(43): Ala or Lys
  MOD_RES (44)..(44): Ala, Ser or Glu
  MOD_RES (45)..(46): May or may not be present
LAXAKXXANXELDXYGVSDFYKRLIXKAKTVEGVEALKXEILXXLP

SEQ ID NO: 971 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (44)..(44): Ala, Ser or Glu
  MOD_RES (45)..(46): May or may not be present
LAXAKXXANXELDXYGVSDFYKRLIXKAKTVEGVEALKXXILAXLP

SEQ ID NO: 972 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (44)..(44): Ala, Ser or Glu
  MOD_RES (45)..(46): May or may not be present
LAXAKXXANXELDXYGVSDFYKRLIXKAKTVEGVEALKXXILKXLP

SEQ ID NO: 973 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (45)..(46): May or may not be present
LAXAKXXANXELDXYGVSDFYKRLIXKAKTVEGVEALKXXILXALP

SEQ ID NO: 974 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (45)..(46): May or may not be present
LAXAKXXANXELDXYGVSDFYKRLIXKAKTVEGVEALKXXILXSLP

SEQ ID NO: 975 - protein, 46 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (45)..(46): May or may not be present
LAXAKXXANXELDXYGVSDFYKRLIXKAKTVEGVEALKXXILXELP

SEQ ID NO: 976 - protein, 46 aa, Artificial Sequence; identical to SEQ ID NO: 300
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (46)..(46): May or may not be present

SEQ ID NO: 977 - protein, 45 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(45): May or may not be present
LAXAKXXANXELDXYGVSDFYKRLIXKAKTVEGVEALKXXILXXP

SEQ ID NO: 978 - protein, 46 aa, Artificial Sequence; identical to SEQ ID NO: 300
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(45): May or may not be present

SEQ ID NO: 979 - protein, 45 aa, Artificial Sequence
  Description of Artificial Sequence: Synthetic polypeptide
  MOD_RES (3)..(3): Glu, Ser, Gln or Cys
  MOD_RES (6)..(6): Glu, Ser or Cys
  MOD_RES (7)..(7): Ala or Ser
  MOD_RES (10)..(10): Ala, Ser or Arg
  MOD_RES (14)..(14): Ala, Ser, Cys or Lys
  MOD_RES (26)..(26): Asp or Glu
  MOD_RES (39)..(39): Asp or Glu
  MOD_RES (40)..(40): Ala or Glu
  MOD_RES (43)..(43): Ala or Lys
  MOD_RES (44)..(44): Ala, Ser or Glu
  MOD_RES (45)..(45): May or may not be present
LAXAKXXANXELDXYGVSDFYKRLIXKAKTVEGVEALKXXILXXL

SEQ ID NO: 980 - protein, 28 aa, Heloderma suspectum
HGEGTFTSDLSKQMEEEAVRLFIEWLKN

SEQ ID NO: 981 - protein, 29 aa, Heloderma suspectum
HGEGTFTSDLSKQMEEEAVRLFIEWLKNG

SEQ ID NO: 982 - protein, 31 aa, Heloderma suspectum
HGEGTFTSDLSKQMEEEAVRLFIEWLKNGGP

SEQ ID NO: 983 - protein, 32 aa, Heloderma suspectum
HGEGTFTSDLSKQMEEEAVRLFIEWLKNGGPS

SEQ ID NO: 984 - protein, 39 aa, Artificial Sequence; identical to SEQ ID NO: 2
  Description of Artificial Sequence: Synthetic polypeptide
  MOD_RES (1)..(1): This position may be 4-imidazoacetyl, des-amino-histidyl, imidazopropionyl, beta-hydroxy-imidazopropionyl, N-dimethyl- histidyl, or beta-carboxy-imidazopropionyl

SEQ ID NO: 985 - protein, 85 aa, Artificial Sequence
  Description of Artificial Sequence: Synthetic polypeptide
HGEGTFTSDLSKQLEEEAVRLFIEWLKQGGPSKEIISGSLAEAKEAANAELDSYGVSDFYKRLIDKAKTVEGVEALKDAILAALP

SEQ ID NO: 986 - protein, 84 aa, Artificial Sequence
  Description of Artificial Sequence: Synthetic polypeptide
HGEGTFTSDLSKQLEEEAVRLFIEWLKQGGPSKEIISGSLAEAKEAANAELDSYGVSDFYKRLIDKAKTVEGVEALKDAILAAL

SEQ ID NO: 987 - protein, 84 aa, Artificial Sequence
  Description of Artificial Sequence: Synthetic polypeptide
HGEGTFTSDLSKQLEEEAVRLFIEWLKQGGPSKEIISGLAEAKEAANAELDSYGVSDFYKRLIDKAKTVEGVEALKDAILAALP

SEQ ID NO: 988 - protein, 83 aa, Artificial Sequence
  Description of Artificial Sequence: Synthetic polypeptide
HGEGTFTSDLSKQLEEEAVRLFIEWLKQGGPSKEIISGLAEAKEAANAELDSYGVSDFYKRLIDKAKTVEGVEALKDAILAAL